(19) United States
(12) Patent Application Publication
(10) Pub. No.: US 2026/0015669 A1
(43) Pub. Date: Jan. 15, 2026

(54) COMPOSITIONS AND METHODS FOR DETECTING AND TREATING TUMORS AND/OR CANCERS ASSOCIATED WITH BRAF AND/OR MAP2K1 VARIANTS

(71) Applicant: North Carolina State University, Raleigh, NC (US)

(72) Inventors: Matthew Breen, Cary, NC (US); Rachael Thomas, Holly Springs, NC (US)

(73) Assignee: North Carolina State University, Raleigh, NC (US)

(21) Appl. No.: 18/865,324

(22) PCT Filed: May 10, 2023
(86) PCT No.: PCT/US2023/066842

§ 371 Date: Nov. 12, 2024

Related U.S. Application Data
(60) Provisional application No. 63/340,326, filed on May 10, 2022.

(51) Int. Cl. C12Q 1/6886; A61K 31/437; A61K 31/44; A61K 31/506; A61K 31/517; A61K 31/519

(52) U.S. Cl. CPC C12Q 1/6886; A61K 31/437; A61K 31/44; A61K 31/506; A61K 31/517; A61K 31/519; C12Q 2600/156

(57) ABSTRACT

Provided are methods for detecting urogenital malignancies in dogs. In some embodiments, the methods include identifying a deletion or single nucleotide substitution within a BRAF gene and/or within a MAP2K1 gene present in or isolated from a biological sample from a. dog, wherein the presence of the deletion or single nucleotide substitution within the BRAF gene and/or within the MAP2K1 gene detects a urogenital malignancy, optionally transitional cell carcinoma/urothelial carcinoma, in the dog. In some embodiments, the deletion is within exon 12 of BRAF gene, optionally within the amino acid sequence KMLNVTAPTPQQL (SEQ ID NO: 3), and/or in within exon 2 or 3 of a M.AP2K1 gene, optionally within the amino acid sequence FLTQKQKVGE (SEQ ID NO: 4).

### CROSS REFERENCE TO RELATED APPLICATIONS

The presently disclosed subject matter claims priority to and the benefit of U.S. Provisional Patent Application Ser. No. 63/340,326, filed May 10, 2022, the disclosure of which is herein incorporated by reference in its entirety.

### REFERENCE TO SEQUENCE LISTING XML

The Sequence Listing XML associated with the instant disclosure has been electronically submitted to the United States Patent and Trademark Office via the Patent Center as a 220,129 byte UTF-8-encoded XML file created on May 10, 2023 and entitled “297_355_PCT.xml”. The Sequence Listing submitted via Patent Center is hereby incorporated by reference in its entirety.

### TECHNICAL FIELD

The presently disclosed subject matter pertains to methods for detecting mutations associated with tumors and/or cancers in dogs and treating the same on the basis of the particular mutations detected. Thus, in some embodiments the presently disclosed subject matter pertains to methods for detecting BRAF and/or MAP2K1 mutations associated with urogenital malignancies in dogs and in some embodiments differentially treating the dogs based on their genotypes with respect to BRAF and/or MAP2K1.

### BACKGROUND

Urothelial carcinoma (UC, also referred to as transitional cell carcinoma or TCC) is the most common canine urogenital cancer, with more than 60,000 dogs developing the disease each year in the United States (Knapp et al., 2014; Knapp et al., 2019). The incidence of the disease is markedly elevated in several popular breeds, most notably the Scottish Terrier (20-fold increased risk for invasive UC compared to mixed breed dogs) and also beagles, Shetland sheepdogs and West Highland white terriers (3 to 6-fold increased risk; Knapp et al., 2014; Knapp et al., 2019). Tumors typically are accompanied by non-specific clinical signs (including pollakiuria, hematuria, stranguria) that are shared with more common and/or readily treatable conditions such as urinary tract infection, bladder stones, benign polyps, and cystitis. As a consequence, canine UC is frequently diagnosed at an advanced stage. Ultimately the patient may lose the ability to urinate due to bladder outlet obstruction caused by the enlarging mass, requiring urgent decompression. At the time of diagnosis, the vast majority of tumors have already invaded the detrusor muscle, and median survival is typically less than 12 months with existing treatment options (reviewed in Knapp et al., 2014; Knapp et al., 2019).

Conventional cytological methods for diagnosis are often inconclusive due to limited sample availability, the variable appearance of normal epithelial cells, and the presence of neutrophilic infiltration in response to secondary bacterial infection. Ultrasonography has limited sensitivity for detecting small masses and urethral lesions, and for distinction between UC and benign lesions (Heng et al., 2022). Histopathologic evaluation of tissue biopsies remains the gold standard, but is invasive, technically challenging, and expensive to perform. To address these limitations, we developed a urine-based method for detecting canine UC that uses droplet digital PCR (ddPCR) analysis for identification of exfoliated tumor cells bearing a specific activating mutation in exon 15 of the BRAF gene (Mochizuki et al., 2015). This mutation results in a valine-to-glutamic acid substitution at codon 595 (V595E) within the activating domain of the BRAF serine/threonine protein kinase. This leads to constitutive activation of the RAS/RAF/MAPK pathway and concomitant upregulation of critical cellular processes including cell growth, survival, and proliferation.

Through analysis of hundreds of specimens we have shown that our BRAF V595E ddPCR assay detects canine UC with 85% sensitivity and >99% specificity, and it has gained wide acceptance as a robust and reliable tool in both clinical and research settings (Wiley et al., 2019; Butty et al., 2021; Clerc-Renaud et al., 2021; Guillen et al., 2021; Rossman et al., 2021). The assay is capable of detecting a single BRAF V595E mutation among 4,000 wild-type alleles (a fractional abundance of 0.025%), which largely overcomes the diluting effect of contaminating inflammatory cells. This offers a powerful means for timely detection of UC, and also identifies tumors that may be responsive to BRAF inhibitor therapy.

Given that ˜85% of canine UC cases harbor the BRAF V595E mutation (Decker et al., 2015; Mochizuki et al., 2015), published datasets are skewed heavily toward cases bearing this variant; consequently the biological and clinical significance of its absence in the remaining 15% of cases has not yet been established. This is in part due to the typically delayed diagnosis of UC in dogs, which confounds the ability to determine the precise order and relative clinical significance of individual molecular events. If they represent early-stage tumors in which the BRAF V595E mutation has not yet emerged, then their study has tremendous potential to yield methods for early detection and to identify somatic alterations that drive tumor progression. Conversely, if tumors without this mutation represent a distinct clinical subtype, their study may reveal other therapeutic targets and indicate a need for molecular subclassification for determining optimal treatment strategies.

Canine and human UC share many clinicopathologic parallels and there is evidence also for shared genomic alterations that are suggestive of conserved pathogenic mechanisms (Shapiro et al., 2015b; Ramsey et al., 2017; Dhawan et al., 2018; Parker et al., 2020). The canine BRAF V595E variant is orthologous to the oncogenic BRAF V600E mutation that is highly recurrent in several human cancers, particularly malignant melanoma (˜50% of cases) and also thyroid and colorectal carcinoma. Interestingly, while one in three human UC cases bear somatic alterations that activate the RTK/RAS/RAF pathway, only 3-4% exhibit variants within the BRAF gene, and only ˜1% harbor the V600E mutation (Forbes et al., 2008; Cancer Genome Atlas Research, 2014; Forbes et al., 2016; AACR Project GENIE Consortium, 2017).

Furthermore, over 75% of canine UC cases show evidence of muscular invasion at the time of diagnosis, compared to only 20-30% of human cases (Knapp et al., 2014; Knowles & Hurst, 2015). As a consequence, cross-species studies have largely involved comparison of canine invasive UC with more superficial human UC cases. One study of a very high-risk human UC cohort with a 2-year metastasis rate of 55% identified BRAF mutations in as many as 25% of cases (Longo et al., 2016). The marked enrichment of mutated BRAF in these human cases provides an opportunity to examine whether canine UC cases bearing BRAF mutation more closely recapitulate the clinicopathologic and molecular characteristics of advanced/metastatic human tumors, and in turn, whether canine tumors with wild-type BRAF resemble lower-grade, localized disease in people. If so, then this would allow the more prevalent category of disease in each species to serve as a model for the rarer category in the other species, in a reciprocal manner.

To address this question it is necessary to generate a comprehensive bank of genomic sequence data for the small minority of canine UC cases without BRAF V595E mutation. In an earlier study we showed that regional copy number gains of dog chromosomes 13 and 36 were present in 97% and 84% of canine UC cases, respectively, and losses on chromosome 19 were evident in 77% of cases (Shapiro et al., 2015b). Over 75% of cases exhibited all three of these DNA copy number aberrations (CNAs) and >93% showed two or more. None were evident in non-neoplastic controls, including cases of urinary tract infection/cystitis and benign bladder polyps (Mochizuki et al., 2016). We developed a ddPCR assay for detection of these three CNAs and established that if two or more were detected in exfoliated cells recovered from canine urine specimens, the sensitivity and specificity to indicate the presence of a UC was >99% (Mochizuki et al., 2015). In this report we leverage the power of these molecular tools, in combination with whole exome sequencing (WES) analysis, to investigate the frequency and distribution of somatic alterations in specimens with and without the BRAF V595E mutation.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for detecting urogenital malignancies in dogs. In some embodiments, the methods comprise, consist essentially of, or consist of identifying a deletion or nucleotide substitution within exon 12 of BRAF gene and/or in exon 2 or 3 of a MAP2K1 gene present in or isolated from a biological sample from the dog, wherein the presence of the deletion or nucleotide substitution within exon 12 of the BRAF gene and/or within exon 2 or 3 of the MAP2K1 gene detects the urogenital malignancy in the dog. In some embodiments, the urogenital malignancy is transitional cell carcinoma/urothelial carcinoma (TCC/UC). In some embodiments, the deletion within exon 12 of the BRAF gene results in a deletion of one or more of the amino acids present within the amino acid sequence KMLNVTAPTPQQL (SEQ ID NO: 3) of a BRAF polypeptide encoded by the BRAF gene. In some embodiments, the deletion within exon 12 of the BRAF gene results in a deletion of one or more amino acids selected from the group consisting of NVTAP (SEQ ID NO: 9), LNVT (SEQ ID NO: 10), LNVTAP (SEQ ID NO: 11), NVTAPT (SEQ ID NO: 12), and TAPT (SEQ ID NO: 13), optionally wherein the deletion is accompanied by an insertion of one or more, optionally one, amino acid. In some embodiments, the deletion and accompanying insertion is selected from the group consisting of a deletion of LNVT (SEQ ID NO: 10) and an insertion of an F, a deletion of LNVTAP (SEQ ID NO: 11) and insertion of an F, and a deletion of NVTAPT (SEQ ID NO: 12) and an insertion of a K. In some embodiments, the deletion within exon 2 of the MAP2K1 gene results in a deletion of one or more of the amino acids present within the amino acid sequence FLTQKQKVGE (SEQ ID NO: 4) of a MAP2K1 polypeptide encoded by the MAP2K1 gene. In some embodiments, the deletion within exon 2 of the MAP2K1 gene results in a deletion of the amino acids FLTQKQ (SEQ ID NO: 14), optionally wherein the deletion is accompanied by an insertion of one or more, optionally one, amino acid. In some embodiments, the insertion is an insertion of an L In some embodiments, the nucleotide substitution results in exon 2 of MAP2K1 results in a Q56P substitution, a K57E substitution, a K57N substitution, or any combination thereof. In some embodiments, the deletion within exon 3 of the MAP2K1 gene results in a deletion of P105 and/or A106 of a MAP2K1 polypeptide encoded by the MAP2K1 gene. In some embodiments, the deletion within exon 3 of the MAP2K1 gene results in a deletion of P105 and A106 of a MAP2K1 polypeptide encoded by the MAP2K1 gene.

The presently disclosed subject matter also relates in some embodiments to methods for differentially treating dogs with urogenital malignancies. In some embodiments, the methods comprise, consist essentially of, or consist of (a) identifying whether the dog has a V595E mutation in a BRAF gene or a deletion within exon 12 of the BRAF gene; and (b1) if the dog has a V595E mutation in a BRAF gene, administering a first-generation BRAF inhibitor, optionally vemurafenib and/or dabrafenib; or (b2) if the dog has a deletion within exon 12 of the BRAF gene, a nucleotide substation within exon 12 of the BRAF gene, or any combination thereof, administering an MEK inhibitor, optionally trametinib, optionally in combination with a broad-acting BRAF inhibitor, optionally sorafenib and/or AZ628. In some embodiments, the identifying comprises assaying nucleic acids present in or isolated from a biological sample isolated from the dog.

In some embodiments of the presently disclosed methods, the identifying step employs a method selected from the group consisting of ddPCR, Sanger sequencing, next generation sequencing, capillary electrophoresis, or any combination thereof. In some embodiments, the biological sample is a urine sample, cells isolated from the urinary tract of the dog, a biopsy specimen, or a combination thereof.

In some embodiments, the presently disclosed methods further comprise administering one or more additional therapies to the dog, optionally wherein the one or more additional therapies are selected from the group consisting of surgery, radiation therapy, and an additional chemotherapy.

The presently disclosed subject matter also relates in some embodiments to methods for simultaneously identifying the presence or absence of a BRAF V595E mutation in a canine or a BRAF V600E mutation in a human and one or more of a deletion within exon 12 of the BRAF gene, a deletion or single nucleotide substitution in exon 2 of a MAP2K1 gene, and a deletion in exon 3 of a MAP2K1 gene present in or isolated from a biological sample from a canine or a human, In some embodiments, the methods comprise, consist essentially of, or consist of (a) obtaining a biological sample from the canine or the human, wherein biological sample from the canine or the human comprises DNA sequences that comprise the BRAF V595E or BRAF V600E coding sequence, and one or more of exon 12 of the BRAF gene, exon 2 of the MAP2K1 gene, and exon 3 of the MAP2K1 gene; (b) amplifying a region of each of the DNA sequences that comprise the BRAF V595E or BRAF V600E coding sequence, and one or more of exon 12 of the BRAF gene, exon 2 of the MAP2K1 gene, and exon 3 of the MAP2K1 gene from the genomic DNA with a plurality of primers, wherein the plurality of primers comprises (i) a first set of primers that together flank a first region of interest that comprise the BRAF V595E or BRAF V600E coding sequence, wherein the first set of primers comprises at least two primers that bind to the same strand of the genomic DNA, differ in sequence with respect to at least one nucleotide and are designed to detect the presence of a nucleotide substitution present in the first region of interest and a third primer that binds to the opposite strand of the genomic DNA to identify the presence or absence of the BRAF V595E or BRAF V600E mutation; and (ii) one or more additional sets of primers that flank additional regions of interest in exon 12 of the BRAF gene, exon 2 of the MAP2K1 gene, exon 3 of the MAP2K1 gene, or any combination thereof, wherein the one or more additional sets of primers are designed to amplify regions of interest in exon 12 of the BRAF gene, exon 2 of the MAP2K1 gene, exon 3 of the MAP2K1 gene, or any combination thereof, and further wherein the first set of primers and each of the one or more additional sets of primers are designed to amplify fragments that can be detectably distinguished from each other; and (c) detecting a difference in the amplified fragments, whereby the presence or absence of a BRAF V595E or BRAF V600E mutation and one or more of a deletion within exon 12 of the BRAF gene, a deletion or single nucleotide substitution in exon 2 of a MAP2K1 gene, and a deletion in exon 3 of a MAP2K1 gene present in or isolated from a biological sample from the canine or the human is identified. In some embodiments, the least two primers that bind to the same strand of the genomic DNA to amplify the subsequence of the BRAF gene that includes the V595E/V600E mutation are allele-specific primers and are also of different sizes such that the amplification product that results from a BRAF V595/V600 allele and the amplification product that results from a BRAF E595/E600 allele are of different sizes. In some embodiments, the amplification products that result from amplification of exon 12 of the BRAF gene, exon 2 of the MAP2K1 gene, and exon 3 of the MAP2K1 gene are also of different sizes from each other and also of the amplification products that result from a BRAF V595/V600 allele and from a BRAF E595/E600 allele.

In some embodiments, the presently disclosed methods further comprising separating the amplification products that correspond to the BRAF V595E/V600E alleles, exon 12 of the BRAF gene, exon 2 of the MAP2K1 gene, and exon 3 of the MAP2K1 gene simultaneously in a single capillary electrophoresis such that the single capillary electrophoresis permits detection of the presence or absence of each of a BRAF V595/V600 allele, a BRAF E595/E600 allele, a wild type exon 12 of the BRAF gene, any indel within exon 12 of the BRAF gene, a wild type exon 2 of the MAP2K1 gene, any indel or single nucleotide substitution within exon 2 of the MAP2K1 gene, a wild type exon 3 of the MAP2K1 gene, and any indel within exon 3 of the MAP2K1 gene due to each of the BRAF V595/V600 allele, the BRAF E595/E600 allele, the wild type exon 12 of the BRAF gene, an indel within exon 12 of the BRAF gene, the wild type exon 2 of the MAP2K1 gene, an indel or single nucleotide substitution within exon 2 of the MAP2K1 gene, the wild type exon 3 of the MAP2K1 gene, and an indel within exon 3 of the MAP2K1 gene generating amplification products of that all differ from each other.

In some embodiments of the presently disclosed methods, the deletion within exon 12 of the BRAF gene results in a deletion of one or more of the amino acids present within the amino acid sequence KMLNVTAPTPQQL (SEQ ID NO: 3) of a BRAF polypeptide encoded by the BRAF gene. In some embodiments, the deletion within exon 12 of the BRAF gene results in a deletion of one or more amino acids selected from the group consisting of NVTAP (SEQ ID NO: 9), LNVT (SEQ ID NO: 10), LNVTAP (SEQ ID NO: 11), NVTAPT (SEQ ID NO: 12), and TAPT (SEQ ID NO: 13), optionally wherein the deletion is accompanied by an insertion of one or more, optionally one, amino acid. In some embodiments, the deletion and accompanying insertion is selected from the group consisting of a deletion of LNVT (SEQ ID NO: 10) and an insert of an F, a deletion of LNVTAP (SEQ ID NO: 11) and insertion of an F, and a deletion of NVTAPT (SEQ ID NO: 12) and an insertion of a K. In some embodiments, the deletion within exon 2 of the MAP2K1 gene results in a deletion of one or more of the amino acids present within the amino acid sequence FLTQKQKVGE (SEQ ID NO: 4) of a MAP2K1 polypeptide encoded by the MAP2K1 gene. In some embodiments, the deletion within exon 2 of the MAP2K1 gene results in a deletion of the amino acids FLTQKQ (SEQ ID NO: 14), optionally wherein the deletion is accompanied by an insertion of one or more, optionally one, amino acid. In some embodiments, the insertion is an insertion of an L. In some embodiments, the nucleotide substitution results in exon 2 of MAP2K1 results in a Q56P substitution, a K57E substitution, a K57N substitution, or any combination thereof. In some embodiments, the deletion within exon 3 of the MAP2K1 gene results in a deletion of P105 and/or A106 of a MAP2K1 polypeptide encoded by the MAP2K1 gene. In some embodiments, the deletion within exon 3 of the MAP2K1 gene results in a deletion of P105 and A106 of a MAP2K1 polypeptide encoded by the MAP2K1 gene.

Thus, it is an object of the presently disclosed subject matter to provide compositions and methods for detecting mutations associated with urogenital malignancies in dogs and/or for treating the same on the basis of the particular mutations detected.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1: Overview of recurrently mutated genes within the study cohort. This oncoplot summarizes whole exome sequencing data for 26 genes that were mutated in two or more samples from the panel of eight POS^{V595E} and 28 UD^{V595E} cases. The nature of the alteration in each sample is shown by differential shading. The horizontal plot at the top shows the number and nature of variants of these genes identified in each individual sample.

FIG. 2: Comparison of BRAF V595E variant frequency obtained using different techniques. The X axis indicates the fractional abundance of the mutant allele in nine specimens as determined by ddPCR analysis, and the Y axis shows the variant allele frequency of the mutation in the same samples as determined by whole exome sequencing analysis. The dashed line indicates the line of best fit, demonstrating the strong correlation in the data obtained by both methods (R² value=0.979) across a wide range of values. The sample denoted as control has a 4.3% fractional abundance of the BRAF V595E variant as determined by ddPCR, and was included in whole exome analysis solely for comparison of mutant allele frequencies generated by both methods at the low end of the range of values.

FIGS. 3A-3C: Summary of BRAF exon 12 and MAP2K1 exon 2 deletions identified in UD^{V595E} specimens. (FIG. 3A) Partial alignment of the deletion hotspot in canine BRAF exon 12 with its human ortholog shows complete conservation of amino acid sequence and only a single nucleotide difference. Horizontal arrows indicate the deleted regions identified within seven of the UD^{V595E} specimens, spanning either nine or 15 nucleotides (2/7 and 5/7 specimens, respectively). Deletions resulting in loss of entire codons are shown with dotted lines, and disruptive deletions with solid lines. Each region is annotated to indicate the amino acid sequence change resulting from the deletion (e.g., ΔNVTAP). The variant allele frequency is shown beside the left arrowhead for each case. Codon numbering in the dog BRAF gene is assigned relative to Ensembl Transcript ENSCAFT00000006306 (SEQ ID NO: 5), to maintain consistency with previous studies describing the V595E variant. (FIG. 3B) The canine and human deletion hotspots in MAP2K1 exon 2 also show complete conservation of amino acid sequence, with three nucleotide differences. Five samples showed deletions spanning 15 nucleotides. A sixth sample (UD-102) showed a single base change (A>G) resulting in a K57E alteration. Codon numbering in the dog MAP2K1 gene is assigned relative to the amino acid sequence encoded by Ensembl Transcript (SEQ ID NO: 51). (FIG. 3C) The canine and human nucleotide sequences in MAP2K exon 2 showing three (3) single base changes resulting in the Q56P mutation (A>C at nucleotide position 30,720,187), the K57E mutation (A>G at nucleotide position 30,720,189), and the K57N mutation (A>G at nucleotide position 30,720,191) of chromosome 30. Codon numbering in the dog MAP2K1 gene is assigned relative to the amino acid sequence encoded by Ensembl Transcript (SEQ ID NO: 51).

FIGS. 4A and 4B: Detection of DNA sequence variants within the BRAF and MAP2K1 genes using capillary electrophoresis. (FIG. 4A) Fluorescent peaks represent amplicons generated for each of the four genomic targets in a normal (non-neoplastic) control sample. Numbers above each peak indicate the size of the amplicon in basepairs, determined by reference to the DNA size ladder shown at the bottom. In the normal control sample, only one peak is evident for each of the four targets, consistent with a normal wild-type sequence. (FIG. 4B) Corresponding results obtained from tumor samples. The upper three plots show a second peak, indicating the presence of a smaller amplicon resulting from a deletion event. In the fourth plot, the second peak indicates the presence of a mutant BRAF V595E allele. Note that each of the four plots on the right side of the figure is derived from a different tumor specimen, since studies to date have shown these sequence alterations to be mutually exclusive events. BPS=base pair size RFU=relative florescence units

FIG. 5: Potential opportunities for using molecular subclassification as a mechanism for treatment stratification. This simplified oncoplot shows the distribution of BRAF and MAP2K1 alterations within the sample cohort, shown in context with the site of action of RAF and MEK in the MAPK pathway. The four categories of variants are annotated to show potential therapeutic strategies, based on extrapolation of data from human studies.

FIGS. 6A and 6B: Summary of variants identified in each sample. (FIG. 6A) The total number of variants identified in each sample is shown as a stacked column plot, with each column subdivided to indicate the number of variants of each category. (FIG. 6B) The same information is shown as a 100% stacked column plot, with each column subdivided to show the percentage contribution of variants of each category as a proportion of all variants identified.

FIGS. 7A-7E; Statistical comparison of the number of mutations identified in POS^{V595E} vs UD^{V595E} samples. (FIG. 7A) Comparison of the total number of mutations identified in POS^{V595E} vs UD^{V595E} samples. The solid horizontal line shows the mean number of mutations identified across all 36 samples combined, and dotted lines indicate the mean value within each sample group. Quantile box plots summarize the variation in the number of mutations identified within each of the two sample groups. The chart is annotated to show the p-values obtained for the comparison of the mean number of mutations identified in each sample group (two-sample t test), and for the variance in the number of mutations identified in each sample group (two-sided F test). (FIGS. 7B-7E) show similar charts for comparisons of the number of mutations of each category (missense variants, FIG. 7B; in-frame insertions/deletions, FIG. 7C; frameshift variants, FIG. 7D; and stop gained/lost variants, FIG. 7E) observed in each sample group.

FIG. 8: Validation of in-frame deletions in BRAF exon 12. Aligned Sanger sequencing traces for the seven UD^{V595E} samples support the short deletions identified in BRAF exon 12 using WES analysis. Each sample was sequenced from both directions flanking the aberrant interval, which is delineated by a box. The resulting amino acid changes are shown to the right. A trace from a non-neoplastic control sample is shown at the top of the alignment for comparison.

FIG. 9: Assessment of capillary electrophoresis assay performance for specimens with varying fractional abundance of the BRAF V595E mutation. ddPCR was used to determine the fractional abundance of the BRAF V595E mutation in 34 histologically-validated formalin-fixed canine UC biopsies. Fractional abundance ranged from 55.7% to 0.2%. Evaluation of the same DNA specimens using the presently disclosed capillary electrophoresis (CE) assay provided positive detection of the BRAF V595E mutation in 31/34 cases (91%). The three specimens in which V595E was not evident were those with the smallest proportion of mutant alleles as determined by ddPCR (3.5%, 2.8%, and 0.2%). As such, the limit of detection of the CE assay for detection of BRAF V595E lies between a fractional abundance of 7.9% and 3.5%.

FIGS. 10A-10C: Detection of Q56P and K57E in UC Samples and one wild-type control. (FIG. 10A) Screening of Q56P in Samples 1-3 and a wild type control. (FIG. 10B) Screening of K57E in Samples 1-3 and a wild type control. These analyses identified Q56P in Sample 1 (WES VAF=57%, ddPCR FA=58%) and K57E in Sample 2 (WES VAF=36%, ddPCR FA=38%). FIG. 10C provides independent data obtained by triplexing the assays employed in FIGS. 10A and 10B into one reaction showing how these variants can be detected accurately within the single triplex assay. The atypical ddPCR profile for Sample 3 was investigated using Sanger sequencing analysis, which revealed a K57N variant that was not detected using the reagents employed for screening for K57E.

### BRIEF DESCRIPTION OF THE TABLES

Table 1: Signalment data and ddPCR assay data for the sample cohort. The sample ID prefix UD denotes specimens in which the BRAF V595E variant was undetected, and the prefix POS denotes samples that tested positive for this variant by ddPCR analysis. Where detected, the fractional abundance of the variant is shown as a percentage. The detection threshold represents the lowest fractional abundance of the variant that was capable of being detected by the assay for each individual sample (Mochizuki et al., 2015). This value provides confidence that UD^{V395E} specimens are classified correctly down to a level of no greater than a single mutant allele among 1000 total alleles.

Table 2: Primer sequences and predicted amplicon sizes. BRAF and MAP2K1 target regions are listed with the corresponding primer sequences and exemplary fluorophore tags. The underlined sequence in SEQ ID NO: 21 represents the 17 nucleotides that allow size discrimination between the BRAF V595 wild-type and mutant alleles. The lower case nucleotide represents the additional sequence mismatch introduced into the forward primer for the mutant allele to prevent spurious binding with the wild-type allele. The last two columns indicate the expected amplicon sizes obtained from wild-type alleles, and the observed amplicon sizes from the study cohort.

Table 3: Primer sequences for use in analysis of MAP2K1 target regions, particularly with respect to mutations at amino acids 56 and 57.

### BRIEF DESCRIPTION OF THE SEQUENCE LISTING

SEQ ID NO: 1 is an exemplary nucleotide sequence that corresponds to codons 478-490 of canine BRAF polypeptide.

SEQ ID NO: 2 is an exemplary nucleotide sequence that corresponds to codons 483-495 of a human BRAF polypeptide.

SEQ ID NO: 3 is the 13 amino acid subsequence of the canine and human BRAF polypeptides encoded by SEQ ID NOs: 1 and 2.

SEQ ID NO: 4 is a subsequence of a canine MAP2K1 amino acid sequence. It corresponds to amino acids 53-62 of Accession No. NP_001041559.2 of the GENBANK® biosequence database (Canis lupus familiaris dual specificity mitogen-activated protein kinase kinase 1). This sequence is encoded by nucleotides 212-241 of Accession No. NM_001048094.2 of the GENBANK® biosequence database.

SEQ ID NO: 5 is the amino acid sequence encoded by Ensembl Transcript ENSCAFT00000006306. It corresponds to amino acids 43-803 of Accession No. XP_038308743.1 of the GENBANK® biosequence database.

SEQ ID NO: 6 is the amino acid sequence of a canine MAP2K1 polypeptide as set forth in Accession No. NP_001041559.2 of the GENBANK® biosequence database.

SEQ ID NO: 7 is an exemplary nucleotide sequence of the canine BRAF genetic locus.

SEQ ID NO: 8 is an exemplary nucleotide sequence of the canine MAP2K1 genetic locus.

SEQ ID NOs: 9-13 are the amino acids sequences encoded by nucleotide deletions within exon 12 of the canine BRAF gene.

SEQ ID NO: 14 is the amino acid sequence encoded by a nucleotide deletion within exon 2 of the canine MAP2K1 gene.

SEQ ID NOs: 15 and 16 are the nucleotide sequences of exemplary oligonucleotide primers that can be employed together in the compositions and methods of the presently disclosed subject matter to amplify exon 12 of the BRAF gene. Chromosomal positions and wild type and mutant amplicon sizes are as set forth in Table 2.

SEQ ID NOs: 17 and 18 are the nucleotide sequences of exemplary oligonucleotide primers that can be employed together in the compositions and methods of the presently disclosed subject matter to amplify exon 2 of the MAP2K1 gene. Chromosomal positions and wild type and mutant amplicon sizes are as set forth in Table 2.

SEQ ID NOs: 19 and 20 are the nucleotide sequences of exemplary oligonucleotide primers that can be employed together in the compositions and methods of the presently disclosed subject matter to amplify exon 3 of the MAP2K1 gene. Chromosomal positions and wild type and mutant amplicon sizes are as set forth in Table 2.

SEQ ID NOs: 21 and 22 are the nucleotide sequences of exemplary oligonucleotide primers that can be employed together in the compositions and methods of the presently disclosed subject matter to amplify the wild type V595 allele of the BRAF gene. Chromosomal positions and wild type and mutant amplicon sizes are as set forth in Table 2.

SEQ ID NOs: 23 and 24 are the nucleotide sequences of exemplary oligonucleotide primers that can be employed together in the compositions and methods of the presently disclosed subject matter to amplify the mutant V595 allele of the BRAF gene. Chromosomal positions and wild type and mutant amplicon sizes are as set forth in Table 2.

SEQ ID Nos: 25-29 are the nucleotide sequences of exemplary oligonucleotide primers that can be employed together in the compositions and methods of the presently disclosed subject matter to amplify the subsequences of the MAP2K1 gene. SEQ ID Nos: 28 and 29 are specific for the Q56P and K27E variants, respectively.

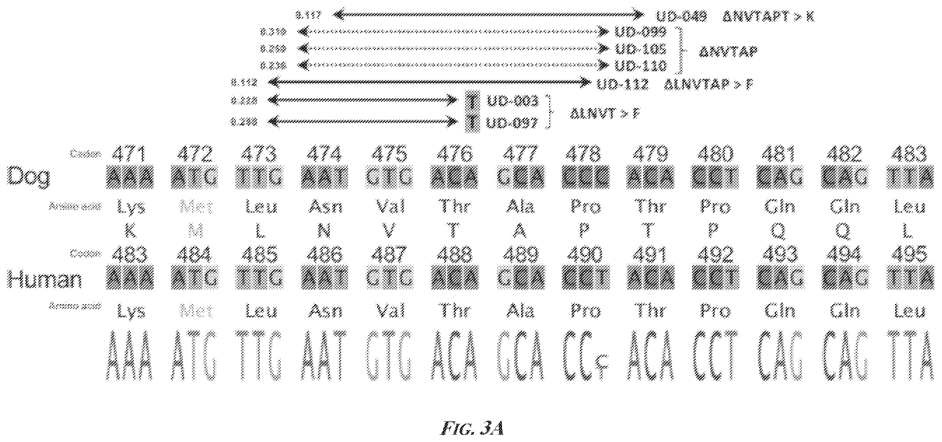

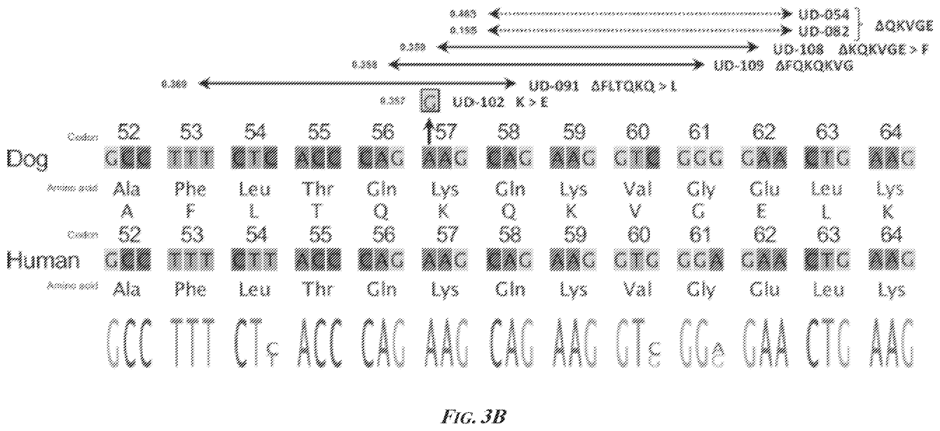

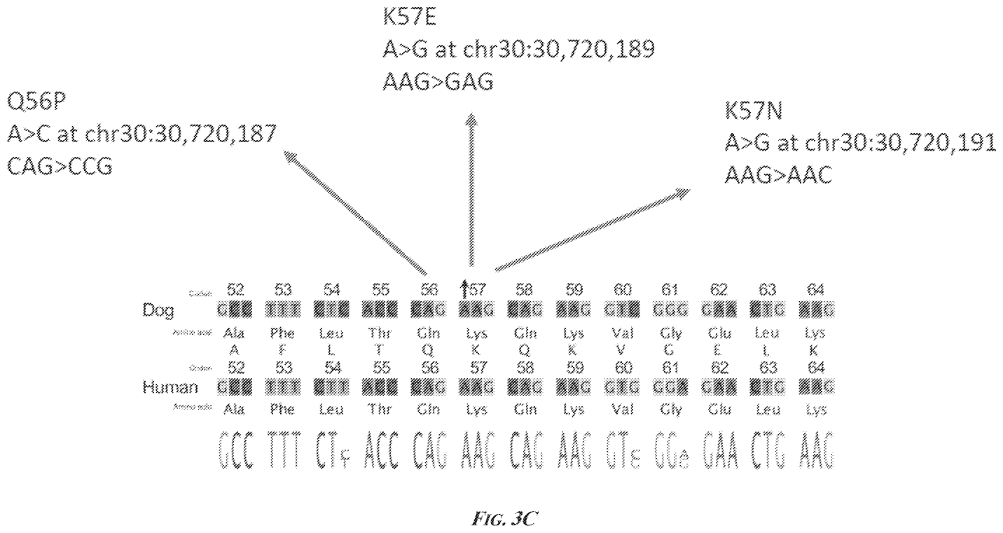

SEQ ID NO: 30 is the nucleotide sequence of a subsequence of the canine BRAF exon 12 sequence depicted in FIG. 3A.

SEQ ID NO: 31 is the amino acid sequence encoded by SEQ ID NOs: 30 and 32 as shown in FIG. 3A.

SEQ ID NO: 32 is the nucleotide sequence of a subsequence of the human BRAF exon 12 sequence depicted in FIG. 3A.

SEQ ID NO: 33 is the nucleotide sequence of a subsequence of the canine MAP2K1 exon 2 sequence depicted in FIGS. 3B and 3C.

SEQ ID NO: 34 is the amino acid sequence encoded by SEQ ID NOs: 33 and 35 as shown in FIGS. 3B and 3C.

SEQ ID NO: 35 is the nucleotide sequence of a subsequence of the human MAP2K1 exon 2 sequence depicted in FIGS. 3B and 3C.

SEQ ID NOs: 36-50 are nucleotide sequences depicted in FIG. 8. Particularly, SEQ ID NOs: 36-50 correspond to the sequences shown for Control_F and Control_R, UD_003_F, UD_003_R, UD_097_F, UD_097_R, US_112_F, US_112_R, US_099_F, US_099_R, US_105_F, US_105_R, US_110_F, US_110_R, US_049_F, and US_049_R, respectively.

SEQ ID NO: 51 is the amino acid sequence encoded by Ensembl Transcript ENSCAFT00000043934.

### DETAILED DESCRIPTION

### I. General Considerations

The mitogen-activated protein kinase (MAPK) signal transduction pathway is dysregulated in many human cancers. Binding of an extracellular ligand to its receptor tyrosine kinase (for example, the growth factor EGF and its receptor EGFR) induces a cascade of phosphorylation events that results in the initiation of proteins that regulate a variety of cellular processes including cell growth, survival, and proliferation.

Somatic mutations of these genes can therefore induce inappropriate constitutive activation of the MAPK pathway, with concomitant upregulation of these critical cellular processes. These mutations are highly recurrent in a wide variety of human cancers including thyroid cancers, malignant melanomas, and colorectal cancers, and represent key therapeutic targets. Over 95% of somatic alterations impacting BRAF, a serine/threonine protein kinase, are the result of a single nucleotide change, a T>A base alteration in exon 15 of the BRAF gene (Tate et al., 2019). This mutation causes substitution of the normal valine residue at codon 600 for glutamic acid (denoted as V600E). This region encodes the activation region of the protein, which normally is held in an inactive confirmation by a glycine-rich loop encoded by exon 11. Mutations in either of these regions can therefore convert the protein into its active conformation.

We, and others, have shown the canine equivalent of the human BRAF V600E mutation to occur in approximately 85% of dog transitional cell carcinomas (TCC) of the urinary bladder (also referred to as urothelial carcinomas; UC; see e.g., Decker et al., 2015; Mochizuki et al., 2015a). This T>A transversion at nucleotide 8,296,284 on dog chromosome 16 (cfa16: 8,296,284, within the canFam3.1 dog genome reference assembly) results in substitution of valine for glutamic acid, and is termed BRAF V595E. We subsequently developed a non-invasive DNA-based assay that can detect the dog BRAF V595E variant in urine specimens with forensic-level sensitivity (Mochizuki et al., 2015b). Specimens in which the mutation is detected are referred to as BRAF-POS, and the assay we developed is now used widely in a clinical setting for dogs presenting with clinical signs suggestive of the presence of a tumor (hematuria, stranguria, pollakiuria etc.). As these signs typically are non-specific and often shared with other more benign conditions, such as urinary tract infections or polyps, detection of the BRAF V595 mutation in canine urine specimens therefore provides compelling evidence supporting the presence of a TCC/UC. Moreover, in human medicine, the BRAF V600E mutation represents a powerful therapeutic target, and ongoing studies are investigating whether the same applies to canine V595E (Rossman et al., 2021). It is not yet clear whether canine TCC/UC cases without a BRAF V595E mutation represent a distinct molecular subtype with contrasting clinical behavior and outcome.

We have shown also that canine TCC/UC recurrently presents with highly characteristic DNA copy number variations (CNVs) involving chromosomes 13, 19 and 36: regional gains of chromosomes 13 and 36 were present in 97% and 84% of cases, respectively, and losses on chromosome 19 were evident in 77% of cases. Statistical evaluation indicated that if two or more of these three aberrations are detected in exfoliated cells recovered from urine specimens, the sensitivity and specificity to indicate the presence of TCC/UC was >99% (Mochizuki et al., 2015). This finding was filed as an invention disclosure to ORC in 2013 (ID14-087) and has since been the subject of issued patents in the U.S. (U.S. Pat. No. 10,450,612), Japan (JP 6492100), and Europe (EP 3 068 906, validated in the United Kingdom, Germany, and France), and a patent pending in Australia (AU 2014348428), each of which is incorporated herein by reference in its entirety.

We developed a second assay designed to detect TCC/UC cases bearing genomic signatures of copy number imbalance based on the three characteristic CNVs described above (referred to as CNV-POS cases; Mochizuki et al., 2016). This CNV assay uses the same technology we optimized to detect the BRAF V600E mutation and can be utilized in a clinical setting for dogs with signs consistent with the presence of a TCC/UC where no BRAF V595E mutation is detected (i.e. BRAF-undetected, or BRAF-UD). These cases are therefore referred to as BRAF-UD/CNV-POS. To investigate the potential for distinct molecular subtypes with contrasting clinical behavior and outcome, we initiated a study focusing on BRAF-UD/CNV-POS cases to compare their genome-wide profiles of somatic variation with tumors bearing BRAF V595E, and to identify alternative therapeutic targets.

Summarily, molecular profiling studies have shown that 85% of canine urothelial carcinomas (UC) harbor an activating BRAF V595E mutation, which is orthologous to the V600E variant found in several human cancer subtypes. In dogs, this mutation provides both a powerful diagnostic marker and a potential therapeutic target; however, due to their relative infrequency, the remaining 15% of cases remain understudied at the molecular level. We performed whole exome sequencing analysis of 28 canine urine sediments exhibiting the characteristic DNA copy number signatures of canine UC, in which the BRAF V595E mutation was undetected (UD^{V595E} samples). Among these we identified 13 specimens (46%) harboring short in-frame deletions within either BRAF exon 12 (7/28 cases) or MAP2K1 exons 2 or 3 (6/28 cases). Orthologous variants occur in several human cancer subtypes and confer structural changes to the protein product that are predictive of response to different classes of small molecular MAPK pathway inhibitors. DNA damage response and repair genes, and chromatin modifiers were also recurrently mutated in UD^{V595E} specimens, as were genes that are positive predictors of immunotherapy response in human cancers. The presently disclosed findings suggest that short in-frame deletions within BRAF exon 12 and MAP2K1 exons 2 and 3 in UD^{V595E} cases and also amino acid substitutions (including but not limited to Q56P, K57E, and K57N) are alternative MAPK-pathway activating events that may have significant therapeutic implications for selecting first-line treatment for canine UC. We developed a simple, cost-effective capillary electrophoresis genotyping assay for detection of these deletions in parallel with the BRAF V595E mutation. The identification of these deletion events in dogs now offers a compelling cross-species platform in which to study the relationship between somatic alteration, protein conformation and therapeutic sensitivity.

### II. Definitions

Unless otherwise defined, all technical and scientific terms used herein have the same meaning as commonly understood by one of ordinary skill in the art to which the presently disclosed subject matter pertains. For clarity of the present specification, certain definitions are presented herein below.

Following long-standing patent law convention, the terms “a” and “an” mean “one or more” when used in this application, including in the claims.

The term “amino acid” is used interchangeably with “amino acid residue”, and may refer to a free amino acid and to an amino acid residue of a peptide. It will be apparent from the context in which the term is used whether it refers to a free amino acid or a residue of a peptide. Amino acids can be classified into seven groups on the basis of the side chain: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

As used herein, amino acids are represented by the full name thereof, by the three letter code corresponding thereto, or by the one-letter code corresponding thereto, as indicated below:

Table of Amino Acids and Functionally Equivalent Codons

| Amino | 3-Letter | 1-Letter | |
|---|---|---|---|
| Acid | Code | Code | Codons |
| Alanine | Ala | A | GCA; GCC; GCG; GCU |
| Cysteine | Cys | C | UGC; UGU |
| Aspartic Acid | Asp | D | GAC; GAU |
| Glutamic acid | Glu | E | GAA; GAG |
| Phenylalanine | Phe | F | UUC; UUU |
| Glycine | Gly | G | GGA; GGC; GGG; GGU |
| Histidine | His | H | CAC; CAU |
| Isoleucine | Ile | I | AUA; AUC; AUU |
| Lysine | Lys | K | AAA; AAG |
| Leucine | Leu | L | UUA; UUG; CUA; CUC; |
| | | | CUG; CUU |
| Methionine | Met | M | AUG |
| Asparagine | Asn | N | AAC; AAU |
| Proline | Pro | P | CCA; CCC; CCG; CCU |
| Glutamine | Gln | Q | CAA; CAG |
| Arginine | Arg | R | AGA; AGG; CGA; CGC; |
| | | | CGG; CGU |
| Serine | Ser | S | ACG; AGU; UCA; UCC; |
| | | | UCG; UCU |
| Threonine | Thr | T | ACA; ACC; ACG; ACU |
| Valine | Val | V | GUA; GUC; GUG; GUU |
| Tryptophan | Trp | W | UGG |
| Tyrosine | Tyr | Y | UAC; UAU |

As used herein, the terms “associated with” and “operatively linked” refer to two nucleic acid sequences that are related physically or functionally. For example, a promoter or regulatory DNA sequence is said to be “associated with” a DNA sequence that encodes an RNA or a polypeptide if the two sequences are operatively linked or situated such that the regulator DNA sequence can affect the expression level of the coding or structural DNA sequence.

As used herein, the term “chimera” refers to a nucleic acid or polypeptide that encodes or comprises domains or other features that are derived from different nucleic acids or polypeptides or are in a position relative to each other that is not naturally occurring.

As used herein, the term “chimeric construct” refers to a recombinant nucleic acid molecule in which a promoter or regulatory nucleic acid sequence is operatively linked to, or associated with, a nucleic acid sequence that codes for an mRNA or which is expressed as a polypeptide, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

As used herein, the term “co-factor” refers to a natural reactant, such as an organic molecule or a metal ion, required in an enzyme-catalyzed reaction. A co-factor can be, for example, NAD (P), riboflavin (including FAD and FMN), folate, molybdopterin, thiamin, biotin, lipoic acid, pantothenic acid and coenzyme A, S-adenosylmethionine, pyridoxal phosphate, ubiquinone, and menaquinone. In some embodiments, a co-factor can be regenerated and reused.

As used herein, the terms “coding sequence” and “open reading frame” (ORF) are used interchangeably and refer to a nucleic acid sequence that is transcribed into RNA such as mRNA, rRNA, tRNA, snRNA, sense RNA, or antisense RNA. In some embodiments, the RNA is then translated in vivo or in vitro to produce a polypeptide.

As used herein, the term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction. Unless specifically indicated to the contrary, the term “complementary” as used herein refers to 100% complementarity throughout the length of at least one of the two antiparallel nucleotide sequences.

As used herein, the terms “domain” and “feature”, when used in reference to a polypeptide or amino acid sequence, refers to a subsequence of an amino acid sequence that has a particular biological function. Domains and features that have a particular biological function include, but are not limited to, ligand binding, nucleic acid binding, catalytic activity, substrate binding, and polypeptide-polypeptide interacting domains. Similarly, when used herein in reference to a nucleic acid sequence, a “domain” or “feature” is that subsequence of the nucleic acid sequence that encodes a domain or feature of a polypeptide. Particularly with reference to a nucleic acid molecule, a “domain” or “feature” is also intended to encompass nucleotide sequences that have a function apart from encoding a domain or feature of a polypeptide. For example, a nucleotide sequence that binds a polypeptide can also be a “domain” (in this case, a protein binding domain).

As used herein, the term “expression cassette” refers to a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA in the sense or antisense direction.

The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression. Typically, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant, the promoter can also be specific to a particular cell type, tissue, organ, or stage of development.

As used herein, the term “fragment” refers to a sequence that comprises a subset of another sequence. When used in the context of a nucleic acid or amino acid sequence, the terms “fragment” and “subsequence” are used interchangeably. A fragment of a nucleic acid sequence can be any number of nucleotides that is less than that found in another nucleic acid sequence, and thus includes, but is not limited to, the sequences of an exon or intron, a promoter, an enhancer, an origin of replication, a 5′ or 3′ untranslated region, a coding region, and/or a polypeptide binding domain. It is understood that a fragment or subsequence can also comprise less than the entirety of a nucleic acid sequence, for example, a portion of an exon or intron, promoter, enhancer, etc. Similarly, a fragment or subsequence of an amino acid sequence can be any number of residues that is less than that found in a naturally occurring polypeptide, and thus includes, but is not limited to, domains, features, repeats, etc. Also similarly, it is understood that a fragment or subsequence of an amino acid sequence need not comprise the entirety of the amino acid sequence of the domain, feature, repeat, etc. A fragment can also be a “functional fragment”, in which the fragment retains a specific biological function of the nucleic acid sequence or amino acid sequence of interest. For example, a functional fragment of a transcription factor can include, but is not limited to, a DNA binding domain, a transactivating domain, or both. Similarly, a functional fragment of a receptor tyrosine kinase includes, but is not limited to a ligand binding domain, a kinase domain, an ATP binding domain, and combinations thereof.

As used herein, the term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The terms “heterologous”, “recombinant”, and “exogenous”, when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or other recombinant techniques. The terms also include non-naturally occurring multiple copies of a naturally occurring DNA sequence. Thus, the terms refer to a DNA segment that is foreign to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

An “endogenous” or “native” nucleic acid (or amino acid) sequence is a nucleic acid (or amino acid) sequence naturally associated with a host cell into which it is introduced. In this context, the terms “heterologous” and “endogenous” are antonymous.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. The phrase “bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

As used herein, the term “inhibitor” refers to a chemical substance that inactivates or decreases the biological activity of a polypeptide such as a biosynthetic and catalytic activity, receptor, signal transduction polypeptide, structural gene product, or transport polypeptide. The term “herbicide” (or “herbicidal compound”) is used herein to define an inhibitor applied to a plant at any stage of development, whereby the herbicide inhibits the growth of the plant or kills the plant.

As used herein, the term “isolated”, when used in the context of an isolated nucleic acid or an isolated polypeptide, is a nucleic acid or polypeptide that, by the hand of man, exists apart from its native environment and is therefore not a product of nature. An nucleic acid molecule or polypeptide can exist in a purified form or can exist in a non-native environment such as, for example, in a transgenic host cell.

As used herein, the term “mature polypeptide” refers to a polypeptide from which the transit peptide, signal peptide, and/or propeptide portions have been removed.

As used herein, the term “minimal promoter” refers to the smallest piece of a promoter, such as a TATA element, that can support any transcription. A minimal promoter typically has greatly reduced promoter activity in the absence of upstream or downstream activation. In the presence of a suitable transcription factor, a minimal promoter can function to permit transcription.

As used herein, the term “native” refers to a gene that is naturally present in the genome of an untransformed plant cell. Similarly, when used in the context of a polypeptide, a “native polypeptide” is a polypeptide that is encoded by a native gene of an untransformed plant cell's genome. Thus, the terms “native” and “endogenous” are synonymous.

As used herein, the term “naturally occurring” refers to an object that is found in nature as distinct from being artificially produced or manipulated by man. For example, a polypeptide or nucleotide sequence that is present in an organism (including a virus) in its natural state, which has not been intentionally modified or isolated by man in the laboratory, is naturally occurring. As such, a polypeptide or nucleotide sequence is considered “non-naturally occurring” if it is encoded by or present within a recombinant molecule, even if the amino acid or nucleic acid sequence is identical to an amino acid or nucleic acid sequence found in nature.

As used herein, the term “nucleic acid” refers to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues (Batzer et al., 1991; Ohtsuka et al., 1985; Rossolini et al., 1994). The terms “nucleic acid” or “nucleic acid sequence” can also be used interchangeably with gene, cDNA, and mRNA encoded by a gene.

As used herein, the phrase “percent identical”, in the context of two nucleic acid or polypeptide sequences, refers to two or more sequences or subsequences that have in some embodiments 60%, in some embodiments 70%, in some embodiments 80%, in some embodiments 90%, in some embodiments 95%, and in some embodiments at least 99% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981, by the homology alignment algorithm disclosed in Needleman & Wunsch, 1970, by the search for similarity method disclosed in Pearson & Lipman, 1988, by computerized implementations of these algorithms (GAP, BESTFIT, FASTA, and TFASTA in the GCG® WISCONSIN PACKAGE®, available from Accelrys, Inc., San Diego, California, United States of America), or by visual inspection. See generally, Altschul et al., 1990; Ausubel et al., 2002 and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

The term “substantially identical”, in the context of two nucleotide or amino acid sequences, refers to two or more sequences or subsequences that have in some embodiments at least about 60% nucleotide or amino acid identity, in some embodiments at least about 65% nucleotide or amino acid identity, in some embodiments at least about 70% nucleotide or amino acid identity, in some embodiments at least about 75% nucleotide or amino acid identity, in some embodiments at least about 80% nucleotide or amino acid identity, in some embodiments at least about 85% nucleotide or amino acid identity, in some embodiments at least about 90% nucleotide or amino acid identity, in some embodiments at least about 91% nucleotide or amino acid identity, in some embodiments at least about 92% nucleotide or amino acid identity, in some embodiments at least about 93% nucleotide or amino acid identity, in some embodiments at least about 94% nucleotide or amino acid identity, in some embodiments at least about 95% nucleotide or amino acid identity, in some embodiments at least about 96% nucleotide or amino acid identity, in some embodiments at least about 97% nucleotide or amino acid identity, in some embodiments at least about 98% nucleotide or amino acid identity, in some embodiments at least about 99% nucleotide or amino acid identity, and in some embodiments at least about 100% nucleotide or amino acid identity, when compared and aligned for maximum correspondence, as measured using one of the above-referenced sequence comparison algorithms or by visual inspection. In one example, the substantial identity exists in nucleotide or amino acid sequences of at least 50 residues, in another example in nucleotide or amino acid sequence of at least about 100 residues, in another example in nucleotide or amino acid sequences of at least about 150 residues, and in yet another example in nucleotide or amino acid sequences comprising complete coding sequences or complete amino acid sequences.

In some embodiments, two nucleic acid or amino acid sequences that are substantially identical also have the same function. In these embodiments, the phrase “the same function” applies to two or more nucleic acid molecules or polypeptides that perform the same biochemical role in either different cell types in the same plant, similar cell types in the same plant, similar cell types in different plants, or even different cell types in different plants. Exemplary functions include, but are not limited to kinase activity, phosphatase activity, nucleic acid binding activity, heat shock activity, and any other enzymatic activity. Two nucleic acids or polypeptides are also deemed to have “the same function” if they participate in the same step of a biochemical pathway, bind to the same, or similar substrates, or produce the same or similar products as a result of their biochemical activities. Exemplary non-limited pathways in which the nucleic acids and polypeptides of the presently disclosed subject matter can function include carbon metabolism, photosynthesis, signal transduction, cell growth, reproduction, disease processes, gene regulation, and resistance to geminivirus-associated disease and its consequences.

In one aspect, polymorphic sequences can be substantially identical sequences. The term “polymorphic” refers to the two or more genetically determined alternative sequences or alleles in a population. An allelic difference can be as small as one base pair. Nonetheless, one of ordinary skill in the art would recognize that the polymorphic sequences correspond to the same gene.

Another indication that two nucleotide sequences are substantially identical is that the two molecules specifically or substantially hybridize to each other under conditions of medium or high stringency. In the context of nucleic acid hybridization, two nucleic acid sequences being compared can be designated a “probe sequence” and a “target sequence”. A “probe sequence” is a reference nucleic acid molecule, and a “target sequence” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in some embodiments at least an about 14 to 40 nucleotide sequence of a nucleic acid molecule of the presently disclosed subject matter. In one example, probes comprise 14 to 20 nucleotides, or even longer where desired, such as 30, 40, 50, 60, 100, 200, 300, or 500 nucleotides or up to the full length (for example, the full complement). Such fragments can be readily prepared by, for example, directly synthesizing the fragment by chemical synthesis, by application of nucleic acid amplification technology, or by introducing selected sequences into recombinant vectors for recombinant production.

The phrase “hybridizing substantially to” refers to complementary hybridization between a probe nucleic acid molecule and a target nucleic acid molecule and embraces minor mismatches (for example, polymorphisms) that can be accommodated by reducing the stringency of the hybridization and/or wash media to achieve the desired hybridization.

“Stringent hybridization conditions” and “stringent hybridization wash conditions” in the context of nucleic acid hybridization experiments such as Southern (Southern et al., 1991) and Northern blot analyses, are both sequence- and environment-dependent. Longer sequences hybridize specifically at higher temperatures. An extensive guide to the hybridization of nucleic acids is found in Tijssen, 1993. Generally, high stringency hybridization and wash conditions are selected to be about 5° C. lower than the thermal melting point (Tm) for the specific sequence at a defined ionic strength and pH. Typically, under “highly stringent conditions” a probe will hybridize specifically to its target subsequence, but to no other sequences. Similarly, medium stringency hybridization and wash conditions are selected to be more than about 5° C. lower than the Tm for the specific sequence at a defined ionic strength and pH. Exemplary medium stringency conditions include hybridizations and washes as for high stringency conditions, except that the temperatures for the hybridization and washes are in some embodiments 8° C., in some embodiments 10° C., in some embodiments 12° C., and in some embodiments 15° C. lower than the Tm for the specific sequence at a defined ionic strength and pH.

The Tm is the temperature (under defined ionic strength and pH) at which 50% of the target sequence hybridizes to a perfectly matched probe. Very stringent conditions are selected to be equal to the Tm for a particular probe. An example of highly stringent hybridization conditions for Southern or Northern Blot analysis of complementary nucleic acids having more than about 100 complementary residues is overnight hybridization in 6×SSC (or 6×SSPE)/0.5% SDS at 65° C., or the same solution including 50% formamide at 42° C. An example of highly stringent wash conditions is 15 minutes in 0.1× standard saline citrate (SSC), 0.1% (w/v) SDS at 65° C. Another example of highly stringent wash conditions is 15 minutes in 0.2×SSC buffer at 65° C. (see Sambrook & Russell, 2001 for a description of SSC buffer and other stringency conditions). Often, a high stringency wash is preceded by a lower stringency wash to remove background probe signal. An example of medium stringency wash conditions for a duplex of more than about 100 nucleotides is 15 minutes in 1×SSC at 55° C. Another example of medium stringency wash conditions for a duplex of more than about 100 nucleotides is 15 minutes in 1×SSC at 50° C. Another example of medium stringency wash conditions for a duplex of more than about 100 nucleotides is 15 minutes in 1×SSC at 45° C. An example of medium stringency wash conditions for a duplex of more than about 100 nucleotides is 15 minutes in 1×SSC at 40° C. Another example of medium stringency wash for a duplex of more than about 100 nucleotides is 15 minutes in 4-6×SSC at 40° C. Another example of medium stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na+ ion, typically about 0.01 to 1M Na+ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm ethylene diamine tetraacetic acid (EDTA), 1% BSA at 50° C. followed by washing in 2×SSC, 0.1% SDS at 50° C.; in another example, a probe and target sequence hybridize in 7% SDS, 0.5 M NaPO₄, 1 mm EDTA, 1% BSA at 50° C. followed by washing in 1×SSC, 0.1% SDS at 50° C.; in another example, a probe and target sequence hybridize in 7% SDS, 0.5 M NaPO₄, 1 mm EDTA, 1% BSA at 50° C. followed by washing in 0.5×SSC, 0.1% SDS at 50° C.; in another example, a probe and target sequence hybridize in 7% SDS, 0.5 M NaPO₄, 1 mm EDTA, 1% BSA at 50° C. followed by washing in 0.1×SSC, 0.1% SDS at 50° C.; in yet another example, a probe and target sequence hybridize in 7% SDS, 0.5 M NaPO₄, 1 mm EDTA, 1% BSA at 50° C. followed by washing in 0.1×SSC, 0.1% SDS at 65° C. In some embodiments, hybridization conditions comprise hybridization in a roller tube for at least 12 hours at 42° C. In each of the above conditions, the sodium phosphate hybridization buffer can be replaced by a hybridization buffer comprising 6×SSC (or 6×SSPE), 5×Denhardt's reagent, 0.5% SDS, and 100 g/ml carrier DNA, including 0-50% formamide, with hybridization and wash temperatures chosen based upon the desired stringency. Other hybridization and wash conditions are known to those of skill in the art (see also Sambrook & Russell, 2001; Ausubel et al., 2002; and Ausubel et al., 2003, each of which is incorporated herein in its entirety). As is known in the art, the addition of formamide in the hybridization solution reduces the Tm by about 0.4° C. Thus, high stringency conditions include the use of any of the above solutions and 0% formamide at 65° C., or any of the above solutions plus 50% formamide at 42° C.

As used herein, the terms “purified” and “isolated”, when applied to a nucleic acid or polypeptide, denotes that the nucleic acid or polypeptide is essentially free of other cellular components with which it is associated in the natural state. It can be in a homogeneous state although it can be in either a dry or aqueous solution. Purity and homogeneity are typically determined using analytical chemistry techniques such as polyacrylamide gel electrophoresis or high performance liquid chromatography. A polypeptide that is the predominant species present in a preparation is substantially purified. The term “purified” denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 99% pure.

As used herein, the term “significant increase” refers to an increase in activity (for example, enzymatic activity) that is larger than the margin of error inherent in the measurement technique, in some embodiments an increase by about 2 fold or greater over a baseline activity (for example, the activity of the wild type enzyme in the presence of the inhibitor), in some embodiments an increase by about 5 fold or greater, and in some embodiments an increase by about 10 fold or greater.

As used herein, the terms “significantly less” and “significantly reduced” refer to a result (for example, an amount of a product of an enzymatic reaction) that is reduced by more than the margin of error inherent in the measurement technique, in some embodiments a decrease by about 2 fold or greater with respect to a baseline activity (for example, the activity of the wild type enzyme in the absence of the inhibitor), in some embodiments, a decrease by about 5 fold or greater, and in some embodiments a decrease by about 10 fold or greater.

As used herein, the terms “specific binding” and “immunological cross-reactivity” refer to an indicator that two molecules are substantially identical. An indication that two nucleic acid sequences or polypeptides are substantially identical is that the polypeptide encoded by the first nucleic acid is immunologically cross reactive with, or specifically binds to, the polypeptide encoded by the second nucleic acid. Thus, a polypeptide is typically substantially identical to a second polypeptide, for example, where the two polypeptides differ only by conservative substitutions.

The phrase “specifically (or selectively) binds to an antibody”, or “specifically (or selectively) immunoreactive with”, when referring to a polypeptide or peptide, refers to a binding reaction which is determinative of the presence of the polypeptide in the presence of a heterogeneous population of polypeptides and other biologics. Thus, under designated immunoassay conditions, the specified antibodies bind to a particular polypeptide and do not bind in a significant amount to other polypeptides present in the sample. Specific binding to an antibody under such conditions can require an antibody that is selected for its specificity for a particular polypeptide. For example, antibodies raised to the polypeptide with the amino acid sequence encoded by any of the nucleic acid sequences of the presently disclosed subject matter can be selected to obtain antibodies specifically immunoreactive with that polypeptide and not with other polypeptides except for polymorphic variants. A variety of immunoassay formats can be used to select antibodies specifically immunoreactive with a particular polypeptide. For example, solid phase ELISA immunoassays, Western blots, or immunohistochemistry are routinely used to select monoclonal antibodies specifically immunoreactive with a polypeptide. See Harlow & Lane, 1988, for a description of immunoassay formats and conditions that can be used to determine specific immunoreactivity. Typically, a specific or selective reaction will be at least twice background signal or noise and more typically more than 10 to 100 times background.

As used herein, the term “subsequence” refers to a sequence of nucleic acids or amino acids that comprises a part of a longer sequence of nucleic acids or amino acids (e.g., polypeptide), respectively.

As used herein, the term “suitable growth conditions” refers to growth conditions that are suitable for a certain desired outcome, for example, the production of a recombinant polypeptide or the expression of a nucleic acid molecule.

As used herein, the term “transformation” refers to a process for introducing heterologous DNA into a plant cell, plant tissue, or plant. Transformed plant cells, plant tissue, or plants are understood to encompass not only the end product of a transformation process, but also transgenic progeny thereof.

As used herein, the terms “transformed”, “transgenic”, and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

As used herein, the term “viability” refers to a fitness parameter of a plant. Plants are assayed for their homozygous performance of plant development, indicating which polypeptides are essential for plant growth.

### III. Methods for Detecting and Treating Urogenital Malignancies

The identification of various mutations in the canine genome that are associated with urogenital malignancies allows for the detection and/or treatment of the urogenital malignancies based on the particular mutation or mutations that are present in a given subject. Thus, the presently disclosed subject matter relates in some embodiments to methods for detecting urogenital malignancies, and relates in some embodiments to methods for treating urogenital malignancies. In some embodiments, the presently disclosed subject matter relates to differentially diagnosing and treating urogenital malignancies in dogs based on the particular genotypes of the dogs.

### III.A. Methods for Detecting Urogenital Malignancies

In some embodiments, the presently disclosed subject matter relates to methods for detecting urogenital malignancies in dogs. In some embodiments, the urogenital malignancy is transitional cell carcinoma/urothelial carcinoma (TCC/UC).

In some embodiments, the methods comprise, consist essentially of, or consist of identifying a deletion and/or a nucleotide substitution (in some embodiments, a single nucleotide substitution) within exon 12 of BRAF gene and/or in exon 2 and/or exon 3 of a MAP2K1 gene present in or isolated from a biological sample from the dog, wherein the presence of the deletion and/or the nucleotide substitution within exon 12 of the BRAF gene and/or within exon 2 and/or 3 of the MAP2K1 gene detects the urogenital malignancy in the dog. In some embodiments, the urogenital malignancy is transitional cell carcinoma/urothelial carcinoma (TCC/UC).

As used herein, the term “BRAF” refers to the B-Raf proto-oncogene, serine/threonine kinase gene and its products, including nucleic acid products (e.g., RNAs such as but not limited to messenger RNAs (mRNAs)) and amino acid products (e.g., peptides and polypeptides). The canine BRAF genetic locus/gene is located on chromosome 16, from nucleotides 8,222,909-8,318,179 of the CanFam3.1 (GCF_000002285.3) assembly (Accession No. NC_006598.3 of the GENBANK® biosequence database). This GENBANK® Accession No. has been updated to the current assembly known as Dog10K_Boxer_Tasha (GCF_000002285.5; Accession No. NC_006598.4), and the canine BRAF genetic locus/gene is located on chromosome 16 from nucleotides 8,989,501-9,160,850 of this later assembly. Both of these GENBANK® Accession Nos. are incorporated by reference in their entireties, including all annotations included therewith.

With reference to the CanFam3.1 (GCF_000002285.3) assembly, several mRNA gene products are disclosed including the following:

Exemplary Canine BRAF Gene Products

| | | Amino |
|---|---|---|
| | Nucleotide | Acid |
| transcript | Accession | Accession |
| variant | No. | No. |
| X1 | XM_005629550.3 | XP_005629607.1 |
| X2 | XM_005629551.3 | XP_005629608.1 |
| X3 | XM_022403735.1 | XP_022259443.1 |
| X4 | XM_022403736.1 | XP_022259444.1 |

In some embodiments, BRAF mutations that are associated with urogenital malignancy including but not limited to transitional cell carcinoma/urothelial carcinoma (TCC/UC) are present within exon 12 of the BRAF gene. In some embodiments, the deletion is of one or more of the amino acids present within the amino acid sequence KMLNVTAPTPQQL (SEQ ID NO: 3) of a BRAF polypeptide encoded by the BRAF gene. More particularly, in some embodiments the deletion within exon 12 of the BRAF gene product results in a deletion of one or more amino acids selected from the group consisting of NVTAP (SEQ ID NO: 9), LNVT (SEQ ID NO: 10), LNVTAP (SEQ ID NO: 11), NVTAPT (SEQ ID NO: 12), and TAPT (SEQ ID NO: 13).

In some embodiments the deletion is accompanied by an insertion of one or more, optionally one, amino acid. Exemplary amino acid deletions/insertions in exon 12 of the canine BRAF gene include a deletion of the tetrapeptide LNVT (SEQ ID NO: 10) and an insert of an F, a deletion of the hexapeptide LNVTAP (SEQ ID NO: 11) and insertion of an F, and a deletion of hexapeptide NVTAPT (SEQ ID NO: 12) and an insertion of a K.

As used herein, the term “MAP2K1” refers to the mitogen-activated protein kinase kinase 1 gene and its products, including nucleic acid products (e.g., RNAs such as but not limited to messenger RNAs (mRNAs)) and amino acid products (e.g., peptides and polypeptides). The canine MAP2K1 genetic locus/gene is located on chromosome 30, from nucleotides 30,683,192-30,760,479 of the CanFam3.1 (GCF_000002285.3) assembly (Accession No. NC_006598.3 of the GENBANK® biosequence database. This GENBANK® Accession No. has been updated to the current assembly known as Dog10K_Boxer_Tasha (GCF_000002285.5; Accession No. NC_006598.4), and the canine MAP2K1 genetic locus/gene is located on chromosome 30 from nucleotides 30,613,296-30,690,593 of this later assembly. Both of these GENBANK® Accession Nos. are incorporated by reference in their entireties, including all annotations included therewith.

With reference to the CanFam3.1 (GCF_000002285.3) assembly, an exemplary MAP2K1 mRNA gene product is disclosed as Accession No. NM 001048094.2 of the GENBANK® biosequence database, which encodes a polypeptide that is disclosed as Accession No. NP_001041559.2 of the GENBANK® biosequence database.

In some embodiments, MAP2K1 mutations that are associated with urogenital malignancy including but not limited to transitional cell carcinoma/urothelial carcinoma (TCC/UC) are present within exon 2 and/or exon 3 of the MAP2K1 gene. In some embodiments, the deletion is of one or more of the amino acids present within the amino acid sequence FLTQKQKVGE (SEQ ID NO: 4) of a MAP2K1 polypeptide encoded by exon 2 of the MAP2K1 gene. By way of example and not limited, in some embodiments the deletion within exon 2 of the MAP2K1 gene product results in a deletion of the amino acids FLTQKQ (SEQ ID NO: 14). In some embodiments, the deletion is accompanied by an insertion of one or more, optionally one, amino acid. In some embodiments, the insertion is an insertion of an L. In some embodiments, the mutation within exon 2 of MAP2K1 results in a Q56P substitution. In some embodiments, the mutation within exon 2 of MAP2K1 results in a K57E substitution. In some embodiments, the mutation within exon 2 of MAP2K1 results in a K57N substitution. Alternatively or in addition, in some embodiments the deletion is within exon 3 of the MAP2K1 gene. In some embodiments, the deletion is of P105 and/or A106 of the MAP2K1 polypeptide encoded by the MAP2K1 gene.

In some embodiments, a mutation in a dog is detected by assaying nucleic acids present in or isolated from a biological sample isolated from the dog. Any technique that can be employed for determining a genotype for a dog can be employed within the methods of the presently disclosed subject matter, including but not limited to droplet digital PCR (ddPCR), next generation sequencing, or a combination thereof.

The detection methods of the presently disclosed subject matter can be applied to any biological sample isolated from the subject that would be expected to contain nucleic acids, particularly that contain cells that comprise genomic DNA. In some embodiments, the biological sample is a urine sample, cells isolated from the urinary tract of the dog, a biopsy specimen, or a combination thereof. Methods for isolating biological samples and nucleic acids present therein are known.

### III.B. Methods for Differentially Treating Urogenital Malignancies in Dogs and Humans

In addition to the deletion and/or nucleotide substitution mutations in BRAF and/or MAP2K1 disclosed herein, other BRAF mutations that are associated with urogenital malignancies are also known to occur in dogs and suspected of occurring in humans. Particularly, a mutation that gives rise to a substitution of a valine (V) with a glutamic acid (E) at amino acid 595 of the canine BRAF gene (referred to herein as “V595E”) is known. A corresponding mutation in humans has also been described and is referred to as V600E (i.e., human amino acid 600 is equivalent to canine amino acid 595). The V595E mutation in canines (V600E in humans) is referred to as a class I mutation. Class II mutations occur mainly in the activation segment at codons other than V595/600, or in the P-loop region of the protein. These also function in a RAS-independent manner, but as activated dimers. Tumors with Class II mutations typically have limited sensitivity to vemurafenib but are more commonly sensitive to inhibitors of the MEK protein, such as trametinib, potentially in combination with broad-acting BRAF inhibitors such as sorafenib. Class III mutations typically have impaired kinase activity and are RAS dependent, and increase signaling through the MAPK pathway due to enhanced binding of RAS and subsequent activation of CRAF (another member of the RAF family of serine/threonine receptor kinases). These often are also sensitive to MEK inhibitors and broad-acting BRAF inhibitors, but may also be treated with other receptor tyrosine kinase inhibitors. Class II and III mutations may also be sensitive to inhibitors that specifically target dimerized RAF (Chen et al., 2016). The relative proportion of class I, II and III BRAF mutations shows tremendous variation between different human cancer subtypes, and research is ongoing to clarify the clinically predictive significance of these differences (Chakraborty et al., 2016; Chen et al., 2016; Foster et al., 2016; Dankner et al., 2018).

### III.C. Combination Therapies

Given the different responses and sensitivities to therapeutically active molecules of Class I vs. Class II/III mutations, the ability to differentiate between dogs/humans who carry Class I or Class II/III mutations also provides an approach to differentially treating dogs/humans who carry various mutations. As such, in some embodiments the presently disclosed subject matter relates to methods for differentially treating dogs/humans with urogenital malignancies based on the genotypes of the dogs/humans with respect to the BRAF gene. By way of example and not limitation, in some embodiments the methods comprise, consist essentially of, or consist or identifying whether the dog/human has a V595E/V600E mutation in a BRAF gene and/or a deletion within exon 12 of the BRAF gene; and then based on the presence or absence of the BRAF V595E mutation and/or a deletion within exon 12 of the BRAF, providing a differential treatment to the dog/human. In some embodiments, if the dog/human has a V595E/V600E mutation in a BRAF gene, administering a first-generation BRAF inhibitor, optionally vemurafenib and/or dabrafenib. Alternatively or in addition, in some embodiments if the dog/human has a deletion within exon 12 of the BRAF gene, administering an MEK inhibitor, optionally trametinib, optionally in combination with a broad-acting BRAF inhibitor, optionally sorafenib and/or AZ628.

In addition to the combination treatments described herein above that can be administered to dogs/humans that have both Class I and Class II/III mutations, additional combination therapies can be administered that are not based strictly on the BRAF and/or MAP2K1 genotypes of the subjects. Thus, in some embodiments the methods of the presently disclosed subject matter further comprise, consist essentially of, or consist of administering one or more additional therapies to the dog, optionally wherein the one or more additional therapies are selected from the group consisting of surgery, radiation therapy, and an additional chemotherapy.

### IV. Methods for Simultaneously Identifying Substitution Mutations and Indels in Mammalian BRAF and MAP2K1 Genes

In some embodiments, the presently disclosed subject matter relates to methods for simultaneously identifying substitution mutations and indels in mammalian BRAF and MAP2K1 genes. In some embodiments, the presently disclosed methods rely on PCR amplification of subsequences of mammalian BRAF and MAP2K1 genes followed by simultaneous analysis of the resulting amplification products, which in some embodiments can be accomplished with capillary electrophoresis (CE). In some embodiments, oligonucleotide primers that are employed for identifying substitution mutations and indels in mammalian BRAF and MAP2K1 genes are selected from the group consisting of SEQ ID NOs: 15 and 16 (BRAF exon 12), SEQ ID NOs: 17 and 18 (MAP2K1 exon 2), SEQ ID NOs: 19 and 20 (MAP2K1 exon 3), SEQ ID NOs: 21 and 22 (wild type BRAF V595), SEQ ID NOs: 23 and 24 (mutant BRAF V595), and SEQ ID NOs: 25-29 (MAP2K1 exon 2), particularly SEQ ID NO: 28 to identify the Q56P variant and SEQ ID NO: 29 to identify the K57E variant.

Accordingly, in some embodiments the presently disclosed subject matter relates to methods for simultaneously identifying the presence or absence of a BRAF V595E mutation in a canine or a BRAF V600E mutation in a human and one or more of a deletion within exon 12 of the BRAF gene, a deletion and/or a nucleotide substitution (in some embodiments, a single nucleotide substitution) in exon 2 of a MAP2K1 gene, and a deletion in exon 3 of a MAP2K1 gene present in or isolated from a biological sample from a canine or a human, the methods comprising, consisting essentially of, or consisting of obtaining a biological sample from the canine or the human, wherein biological sample from the canine or the human comprises DNA sequences that comprise the BRAF V595E or BRAF V600E coding sequence, and one or more of exon 12 of the BRAF gene, exon 2 of the MAP2K1 gene, and exon 3 of the MAP2K1 gene; amplifying a region of each of the DNA sequences that comprise the BRAF V595E or BRAF V600E coding sequence, and one or more of exon 12 of the BRAF gene, exon 2 of the MAP2K1 gene, and exon 3 of the MAP2K1 gene from the genomic DNA with a plurality of primers, wherein the plurality of primers comprises a first set of primers that together flank a first region of interest that comprise the BRAF V595E or BRAF V600E coding sequence, wherein the first set of primers comprises at least two primers that bind to the same strand of the genomic DNA, differ in sequence with respect to at least one nucleotide and are designed to detect the presence of a nucleotide substitution present in the first region of interest and a third primer that binds to the opposite strand of the genomic DNA to identify the presence or absence of the BRAF V595E or BRAF V600E mutation; and one or more additional sets of primers that flank additional regions of interest in exon 12 of the BRAF gene, exon 2 of the MAP2K1 gene, exon 3 of the MAP2K1 gene, or any combination thereof, wherein the one or more additional sets of primers are designed to amplify regions of interest in exon 12 of the BRAF gene, exon 2 of the MAP2K1 gene, exon 3 of the MAP2K1 gene, or any combination thereof, and further wherein the first set of primers and each of the one or more additional sets of primers are designed to amplify fragments that can be detectably distinguished from each other; and detecting a difference in the amplified fragments, whereby the presence or absence of a BRAF V595E or BRAF V600E mutation and one or more of a deletion within exon 12 of the BRAF gene, a deletion and/or a nucleotide substitution (including but not limited to a single nucleotide substitution) in exon 2 of a MAP2K1 gene, and a deletion in exon 3 of a MAP2K1 gene present in or isolated from a biological sample from the canine or the human is identified.

The presently disclosed methods are based in some embodiments on designing sets of amplification primers that provide amplification products that differ in size such that particular amplification product sizes can be distinguished when separated, for example, by capillary electrophoresis (CE), wherein each individual amplification product is indicative of the presence of a particular allele. Thus, in some embodiments the least two primers that bind to the same strand of the genomic DNA to amplify the subsequence of the BRAF gene that includes the V595E/V600E mutation are allele-specific primers and are also of different sizes such that the amplification product that results from a BRAF V595/V600 allele and the amplification product that results from a BRAF E595/E600 allele are of different sizes. Allele-specific primers are understood to be primers that are designed to bind to and thus provide amplification of one particular allele of a given genetic locus. The BRAF V595E/V600E mutation provides just such an example, wherein a first allele-specific primer can be designed that amplifies only the BRAF V595/V600 allele and a second allele-specific primer can be designed that amplifies only the BRAF E595/E600 allele. Other examples include primers that can be employed for detecting the presence of a MAP2K1 Q56 allele, a MAP2K1 P56 allele, a MAP2K1 K57 allele, a MAP2K1 E57 allele, and/or a MAP2K1 N57 allele. Typical allele-specific primers employ one or more allele-specific nucleotides, which in some embodiments are primers that employ one or more nucleotides at or near their 3′ ends that will only hybridize to and thus amplify one allele or another. If an amplification product is generated with a given allele-specific primer, that means that that allele is present in the nucleic acid sample that was amplified. When there are two (or more) different alleles being assayed, however, the design of the allele-specific primers generally results in the sizes of the amplification products from the various alleles being identical or nearly so since the “forward” primers typically differ only with respect to certain allele-specific nucleotides at or near their 3′ ends. As a result, allele-specific PCR typically cannot be multiplexed because the amplification products cannot be distinguished on the basis of size.

This shortcoming is addressed by certain embodiments of the presently disclosed subject matter, in which the lengths of the forward primers for the various alleles are themselves of different sizes, resulting in amplification products that are themselves of different sizes. Provided that the size differences are sufficient, multiplex allele-specific PCR analysis is thus possible.

Therefore, in some embodiments of the presently disclosed methods, the amplification product that results from a BRAF V595/V600 allele and the amplification product that results from a BRAF E595/E600 allele are of different sizes because the BRAF V595/V600 allele forward primer is of a different size than the BRAF E595/E600 allele forward primer.

In addition to determining the presence or absence of BRAF V595/V600 alleles and BRAF E595/E600 alleles, in some embodiments indels and/or nucleotide substitutions (in some embodiments, single nucleotide substitutions) involving exon 12 of the BRAF gene, exon 2 of the MAP2K1 gene, and/or exon 3 of the MAP2K1 gene are also assayed along with the BRAF V595/V600 alleles and BRAF E595/E600 alleles. In order to assay all of these various combinations of alleles, the primers that are designed for assaying exon 12 of the BRAF gene, exon 2 of the MAP2K1 gene, and/or exon 3 of the MAP2K1 gene are also designed to generate amplification products of different sizes from each other and also of the amplification products that result from a BRAF V595/V600 allele and from a BRAF E595/E600 allele. Thus, with proper primer set designs, all of the various alleles that result from both substitution mutations and indels of BRAF and MAP2K1 can be identified using a single separation medium, including but not limited to CE. As such, in some embodiments the presently disclosed methods further comprise, consist essentially of, or consist of separating the amplification products that correspond to the BRAF V595E/V600E alleles, exon 12 of the BRAF gene, exon 2 of the MAP2K1 gene, and exon 3 of the MAP2K1 gene simultaneously in a single capillary electrophoresis such that the single capillary electrophoresis permits detection of the presence or absence of each of a BRAF V595/V600 allele, a BRAF E595/E600 allele, a wild type exon 12 of the BRAF gene, any indel within exon 12 of the BRAF gene, a wild type exon 2 of the MAP2K1 gene, an indel and/or nucleotide substitution within exon 2 of the MAP2K1 gene, a wild type exon 3 of the MAP2K1 gene, and an indel in exon 3 of the MAP2K1 gene due to each of the BRAF V595/V600 allele, the BRAF E595/B600 allele, the wild type exon 12 of the BRAF gene, an indel in exon 12 of the BRAF gene, the wild type exon 2 of the MAP2K1 gene, an indel and/or nucleotide substitution in exon 2 of the MAP2K1 gene, the wild type exon 3 of the MAP2K1 gene, and an indel in exon 3 of the MAP2K1 gene generating amplification products that all differ from each other in some detectable manner such as, but not limited to, by size separation and/or by inclusion of a detectable moiety such as a fluorophore.

Various indels, and/or nucleotide substitutions (such as but not limited to single nucleotide substitutions), in some embodiments deletions, in some embodiments in frame deletions in exon 12 of BRAF can be identified using the presently disclosed methods. In some embodiments, the deletion within exon 12 of the BRAF gene results in a deletion of one or more of the amino acids present within the amino acid sequence KMLNVTAPTPQQL (SEQ ID NO: 3) of a BRAF polypeptide encoded by the BRAF gene. In some embodiments, the deletion within exon 12 of the BRAF gene results in a deletion of one or more amino acids selected from the group consisting of NVTAP (SEQ ID NO: 9), LNVT (SEQ ID NO: 10), LNVTAP (SEQ ID NO: 11), NVTAPT (SEQ ID NO: 12), and TAPT (SEQ ID NO: 13), optionally wherein the deletion is accompanied by an insertion of one or more, optionally one, amino acid. In some embodiments, the deletion and accompanying insertion is selected from the group consisting of a deletion of LNVT (SEQ ID NO: 10) and an insert of an F, a deletion of LNVTAP (SEQ ID NO: 11) and insertion of an F, and a deletion of NVTAPT (SEQ ID NO: 12) and an insertion of a K.

Similarly, various indels and/or nucleotide substitutions (such as but not limited to single nucleotide substitutions), in some embodiments deletions in exon 2 of the MAP2K1 gene can be identified using the presently disclosed methods. In some embodiments, a deletion of one or more of the amino acids present within the amino acid sequence FLTQKQKVGE (SEQ ID NO: 4) of a MAP2K1 polypeptide encoded by the MAP2K1 gene. In some embodiments, the deletion within exon 2 of the MAP2K1 gene results in a deletion of the amino acids FLTQKQ (SEQ ID NO: 14), optionally wherein the deletion is accompanied by an insertion of one or more, optionally one, amino acid. In some embodiments, the insertion is an insertion of an L. In some embodiments, the mutation within exon 2 of MAP2K1 results in a Q56P substitution. In some embodiments, the mutation within exon 2 of MAP2K1 results in a K57E substitution. In some embodiments, the mutation within exon 2 of MAP2K1 results in a K57N substitution.

Also similarly, various indels, in some embodiments deletions in exon 3 of the MAP2K1 gene, can be identified using the presently disclosed methods. In some embodiments, the deletion within exon 3 of the MAP2K1 gene results in a deletion of P105 and/or A106 of a MAP2K1 polypeptide encoded by the MAP2K1 gene. In some embodiments, the deletion within exon 3 of the MAP2K1 gene results in a deletion of P105 and A106 of a MAP2K1 polypeptide encoded by the MAP2K1 gene.

Summarily, in some embodiments the presently disclosed subject matter compositions and methods can be employed to simultaneously determine what alleles a given subject might have with respect to a BRAF V595E or BRAF V600E mutation, indels involving exon 12 of the BRAF gene, indels and/or nucleotide substitutions involving exon 2 of the MAP2K1 gene, and/or indels involving exon 3 of the MAP2K1 gene, or any combination thereof, wherein PCR primers can be designed such that amplification products derived from these loci can be distinguished from each other all together in one run (such as but not limited to a CE run). Given the speedy and relatively inexpensive nature of PCR and CE separation, the compositions and methods of the presently disclosed subject matter provide a great advantage and cost savings for identifying what alleles a given subject might have with respect to the BRAF and MAP2K1 loci, which can also provide a basis for making informed treatment decisions in the case of urogenital malignancies in mammals.

### EXAMPLES

The following Examples have been included to illustrate modes of the presently disclosed subject matter. Certain aspects of the following Examples are described in terms of techniques and procedures found or contemplated by the present co-inventors to work well in the practice of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods for the Examples

Standard recombinant DNA and molecular cloning techniques used here are well known in the art and are disclosed in Silhavy et al., 1984; Reiter et al., 1992; Schultz et al., 1998; Sambrook & Russell, 2001; Ausubel et al., 2002; and Ausubel et al., 2003.

Sample preparation and ddPCR analysis of clinical specimens. Free-catch urine specimens were obtained from dogs exhibiting non-specific clinical signs including hematuria, stranguria, and pollakiuria. Owners of each dog provided consent for samples from their dog to be included in research studies, and associated clinical records were reviewed by a board-certified small animal veterinary internist. Exfoliated cells were pelleted by centrifugation, and rinsed with sterile PBS, and processed for DNA extraction using a MAXWELL™ RSC Instrument and Cell DNA Purification Kit (Promega, Madison WI). DNA samples were screened using ddPCR analysis for the presence of the dog BRAF V595E mutation, a T>A substitution at nucleotide 8,296,284 on dog chromosome 16, denoted as cfa16: 8,296,284, using the criteria described in Mochizuki et al., 2015.

Briefly, specimens in which BRAF V595E was detected (hereafter referred to as ‘POS^{V595E}’) were analyzed to determine the percentage fractional abundance of the variant (calculated as [A/(A+B)] where A=number of copies/μl of the mutant allele and B=number of copies/μl of the wild-type allele). Specimens that exhibited fewer than five single-occupancy BRAF V595E mutant-positive droplets and a minimum of 5000 BRAF wild-type droplets were classified as ‘undetected’ (hereafter referred to as ‘UD^{V595E}’). A detection threshold was determined for each sample (calculated as [5/(A+B]), providing a measure of the lowest BRAF V595E fractional abundance that the assay is capable of detecting for that individual sample. All specimens were then screened with the canine UC CNA ddPCR assay as described previously (Mochizuki et al., 2016). Briefly, this assay calculates the copy number of discrete regions of cfa13 and cfa36 relative to a region of cfa19. Specimens with a relative DNA copy number ratio >1.2 for both the cfa13/cfa19 signature and the cfa36/cfa19 signature were classified as CNA-positive (‘POS^{CNA}’).

Whole exome sequencing analysis. Two categories of POS^{CNA} samples were selected for further characterization using WES analysis. Twenty-eight UD^{V595E} specimens were selected in which the BRAF V595E mutant allele was undetected with a detection threshold <0.1%. These were assigned the prefix ‘UD-’. Eight POS^{V595E} specimens were selected that showed a high fractional abundance of the BRAF V595E mutation (>40%). These were assigned the prefix ‘POS-’. For each specimen, ˜25 ng of DNA were sheared to yield a mean fragment size of ˜300 bp using a Covaris S220 Ultrasonicator (Covaris, Woburn MA). Libraries were prepared using a KAPA Biosystems HyperPrep Kit (Roche Nimblegen, Pleasanton CA) incorporating a unique dual-indexed barcode adaptor for each specimen. Due to the limited quantity of starting DNA available, size-selection was omitted. Solution-based target enrichment was performed independently (without pooling) for each sample using the Roche Nimblegen SeqCap EZ HyperCap workflow v.2.3 with Custom Developer Probes encompassing 52.9 Mb of dog exomic sequence (canine Exome-1.0 design, (Broeckx et al., 2014). Library quality, fragment size range and yield were assessed before and after target enrichment using a 2200 TapeStation (Agilent Technologies, Santa Clara, California, United States of America) and a Nanodrop One spectrophotometer (Thermo Scientific, Waltham, Massachusetts, United States of America), following the manufacturer's recommendations. Blood-derived DNA samples from 12 non-neoplastic controls were processed in parallel using the same workflow. Also included was a specimen with a low fractional abundance of the BRAF V595E mutation (4.3%), to act solely as an internal control for comparison of ddPCR and WES data for this variant. This control sample was denoted ‘low-FA’.

Exome-enriched libraries were pooled at equimolar concentrations, loaded onto a NovaSeq 6000 S4 flow-cell (Illumina, San Diego, California, United States of America) and sequenced with 150 bp paired-end reads (NC State University Genomic Sciences Laboratory, Raleigh, North Carolina, United States of America). Fastq files were processed with a custom pipeline incorporating optimized tools from the Sentieon Genomics suite v.202010.02 (Sentieon Inc., San Jose, California, United States of America) based on GATK best practices (Van der Auwera et al., 2013). Briefly, raw reads were aligned to the canFam3.1 dog reference sequence assembly (Lindblad-Toh et al., 2005) using Sentieon bwa-mem, and duplicate reads were marked using Dedup and Locus Collector. Base quality score recalibration was performed using the dog dbSNP v151 database. Indel realignment was performed in parallel with variant calling using the haplotype-based Sentieon TNscope caller with default parameters (Freed et al., 2018). This follows the general principles of the GATK Haplotype Caller and Mutect2 with enhanced sensitivity for detection of low-penetrance somatic variation (Freed et al., 2018; Pei et al., 2021). TNscope analysis referenced a pool of all variants identified in the 12 non-neoplastic specimens, which were processed using the same library preparation workflow and sequenced on the same Illumina flowcell.

Downstream variant filtering was performing using VarSeq v.2.2.3 (Golden Helix, Boseman MT). Briefly, variants that passed Sentieon TNscope's variant calling filters were imported and annotated for gene content (Ensembl Genes release 100; Hoeppner et al., 2014), sequence ontology (Eilbeck et al., 2005; Hunt et al., 2018) and overlap with previously identified canine germline and somatic variants (dbSNP build 151; Sherry et al., 2001; Plassais et al., 2019). The Cancer Gene Census database Sondka et al., 2018) was used to annotate the dog orthologs of genes within which mutations are causally implicated in human cancers. Variants that were evident in >5% of samples from the dataset generated by (Plassais et al., 2019) were excluded from further analysis. Intergenic and intronic variants, and short tandem repeats were also excluded. Variant sites were required to show a read depth of 30 or greater, with a minimum of five reads for the alternate allele. Variants that met any of the following criteria were also excluded from further analysis, following GATK Best Practices recommendations (Van der Auwera et al., 2013; for single nucleotide variants (SNVs): QD<2, QUAL<30, SOR>3, FS>60, MQRankSum<−12.5, ReadPosRankSum<−8 and for indels: QD<2, QUAL<30, FS>200, ReadPosRankSum<−20). Statistical analyses were performed using JMP Pro v15.2.0 (SAS Institute Inc., Cary NC). After testing for normality, two-sample t-tests were performed to determine whether there was a significant difference in the mean number of mutations identified in POS^{V595E} and UD^{V595E} cases (p<0.05). The mean number of each category of mutations was compared between sample groups in the same manner. A two-sided F-test was used to determine whether the variance of these parameters within the two sample groups was significantly different (p<0.05). Selected variants were validated by conventional Sanger sequencing analysis of samples from the WES cohort.

Validation and distribution assessment of selected variants. Four target regions were evaluated using capillary electrophoresis (CE) to detect sequence alterations based on differential amplicon size. Three of these regions harbored short deletion events in a subset of samples and are described in the Results section. PCR primer pairs were designed to flank each of these regions to permit discrimination between wild-type and mutant alleles based on differential amplicon size. Primers were designed such that there would be no overlap in the size of the resulting amplicons for the four target regions, when amplified from either tumor or normal (wild type) DNA. The fourth target evaluated was the site of the BRAF V595E mutation. One primer was designed to the normal dog genome assembly reference sequence, with the 3′ end matching the wild-type T nucleotide. An additional 17 nucleotides of non-canine sequence were added to the 5′ end of this wild-type primer, to result in generation of a larger amplicon from the wild-type allele compared to the mutant allele. A second primer was designed with the 3′ end matching the A nucleotide of the V595E mutation, and with the fourth nucleotide from the 3′ end altered from the wild-type C into a G. This introduced a second site of mismatch with the wild-type allele, ensuring amplification only from the mutant allele. Both allele-specific BRAF V595 primers were tagged with the same 5′ fluorophore. A third, common primer was designed to the opposite DNA strand. This strategy was used to enable distinction between the wild-type allele and the mutant allele using a combination of allele-specific PCR and differential amplicon size, while using only a single fluorophore.

The forward primer for each of the four target regions was modified with a different 5′ fluorophore (6-FAM™, VIC®, NED™ or PET®), and was purified by high-performance liquid chromatography (Thermo Fisher Scientific, Waltham MA). Target regions were amplified by conventional end-point PCR on non-neoplastic controls and test samples from the WES cohort, to verify the ability to detect known alterations. Each target region was amplified in independent reactions for each sample using Promega GOTAQ® Colorless Master Mix (final concentration 1×), 0.4 μM final concentration of each primer and ˜5 ng template DNA, in a total volume of 10 μl. Thermal cycling was performed using a C1000 Touch system (Bio-Rad, Hercules CA) using the following conditions: initial denaturation at 95° C. for 2 minutes, followed by 30 cycles of 94° C. for 30 seconds, 60° C. for 30 seconds, 72° C. for 30 seconds, and a final extension at 72° C. for 30 minutes. Amplicons were pooled for each sample, and the resulting pool was diluted with an equal volume of ultrapure water. Next, 1 μl of the diluted pool was combined with 10 μl HIDI™ formamide and 0.5 μl of GENESCAN™ 600 LIZ™ Size Standard v.2 (Thermo Fisher Scientific), denatured and loaded onto an Applied Biosystems 3730 xl Genetic Analyzer. OSIRIS v2.16 software (available from the website of the National Library of Medicine, National Center for Biotechnology Information (NCBI)) was used to verify the expected size of the amplicon for each target region, by reference to the DNA size standard. See also Goor et al., 2011; Goor et al., 2020.

### Example 1

### Sample Population

Urine-derived DNA samples from 36 dogs were analyzed using WES analysis, and signalment data are provided in Table 1. Across all cases, there was a bias toward female dogs (28 female, seven male, and one of unknown sex); however the proportion of dogs of each sex within sample groups (UD^{V595E} vs POS^{V595E}) was not significantly different (p=1.0, two-tailed Fisher's exact test). A total of 18 different breeds were represented among the 26 dogs that were reported as purebred by the owner. Only three breeds were represented by more than a single case (beagle, Boston terrier and Labrador retriever). The mean age of dogs in each sample group was not significantly different (10.6 years for UD^{V595E} cases and 11.6 years for POS^{V595E} cases, two-sample t test, p=0.20).

### Example 2

### Overview of Sequencing Metrics and Variant Frequency

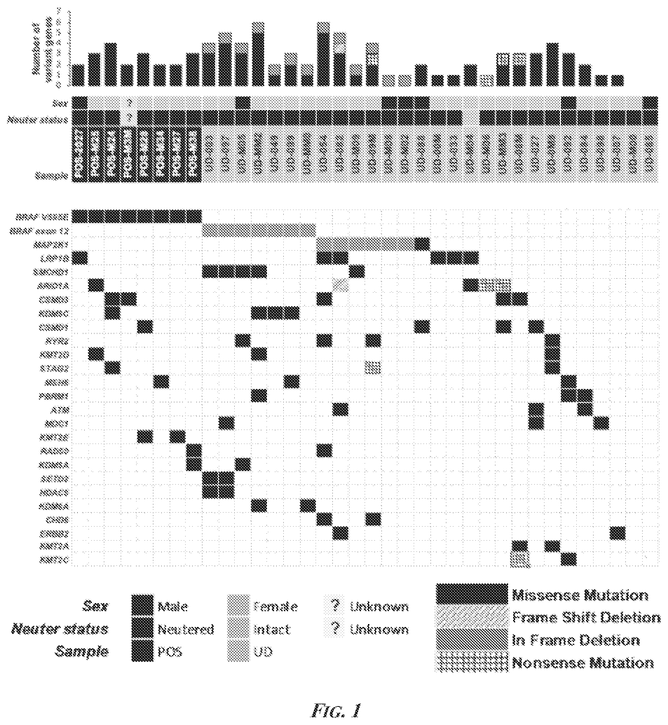

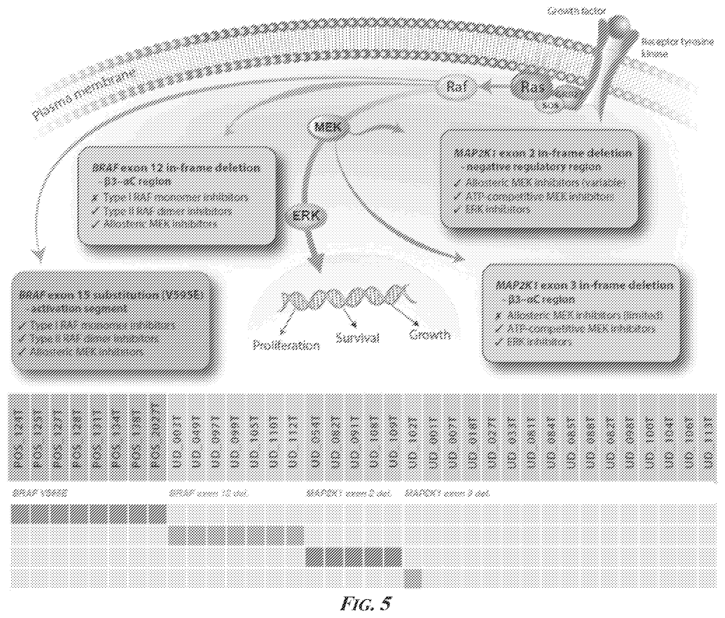

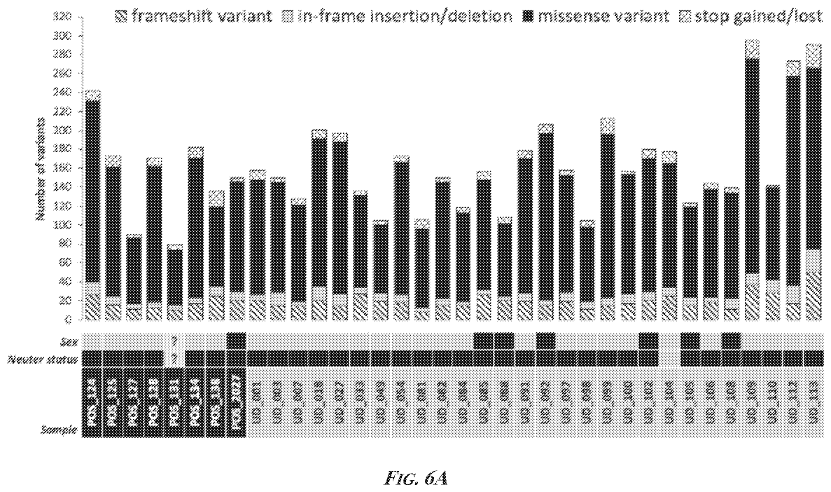

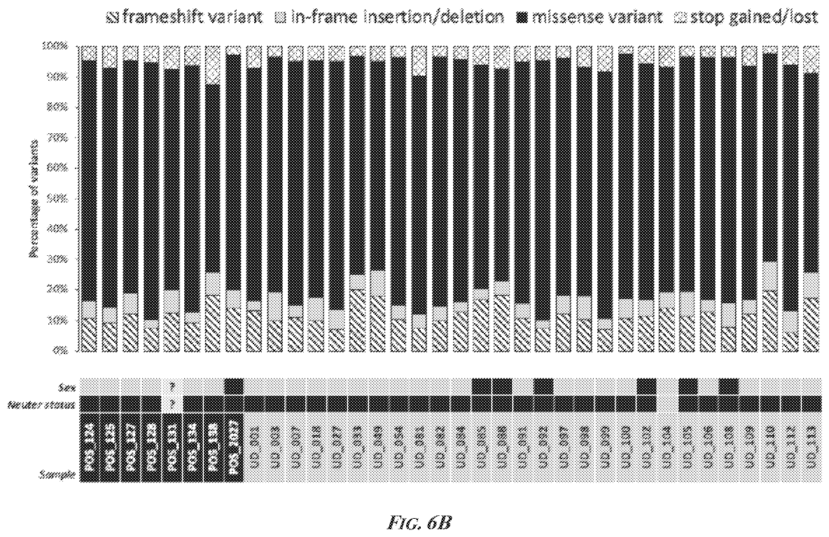

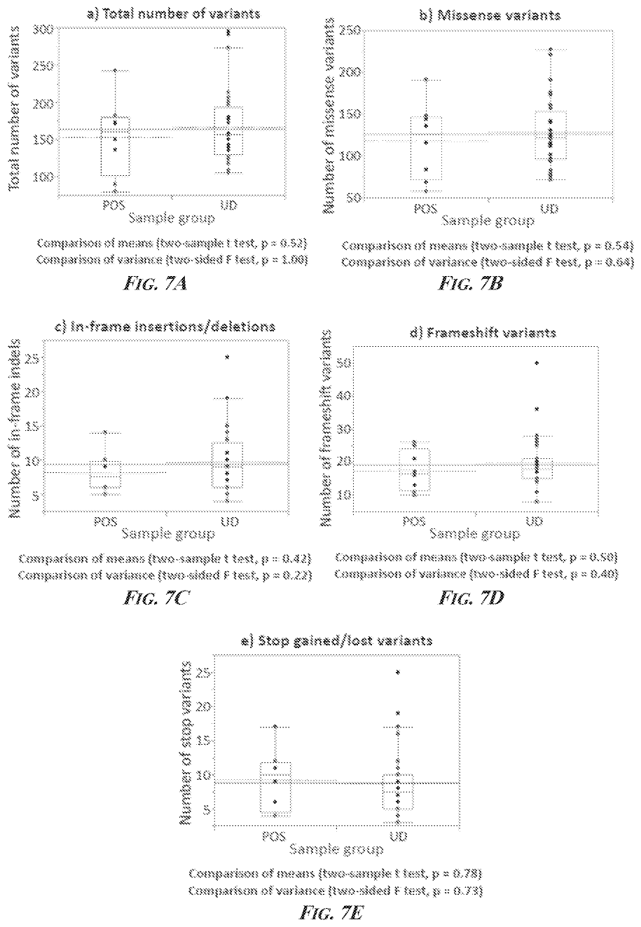

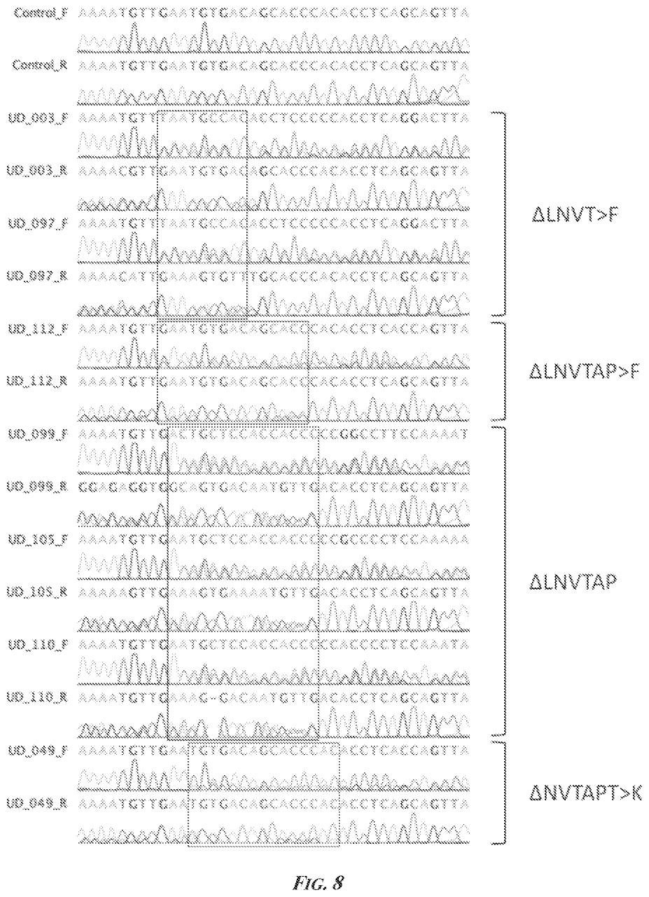

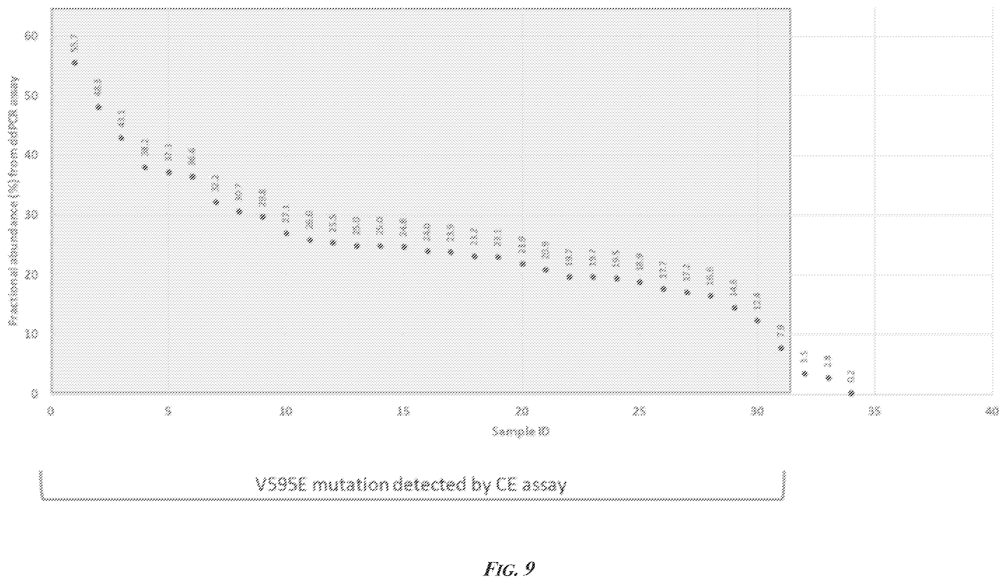

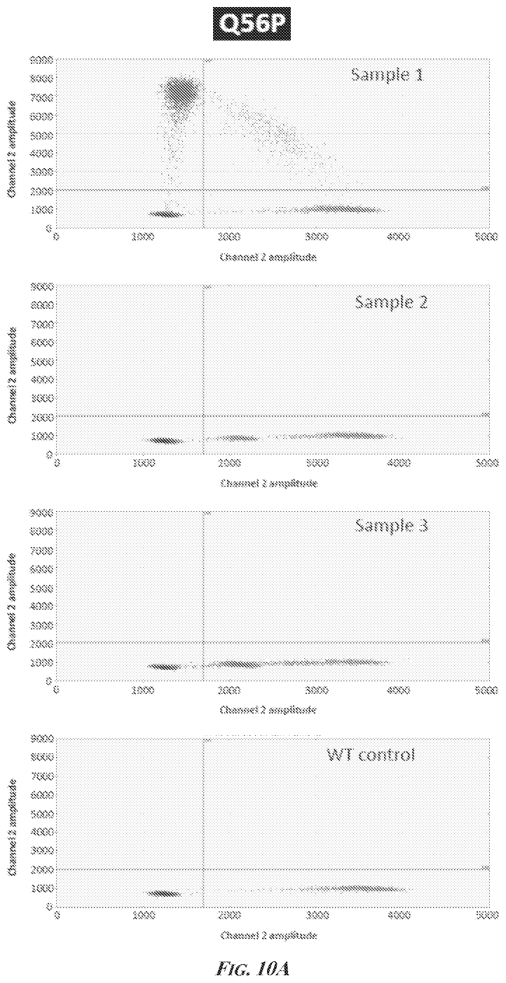

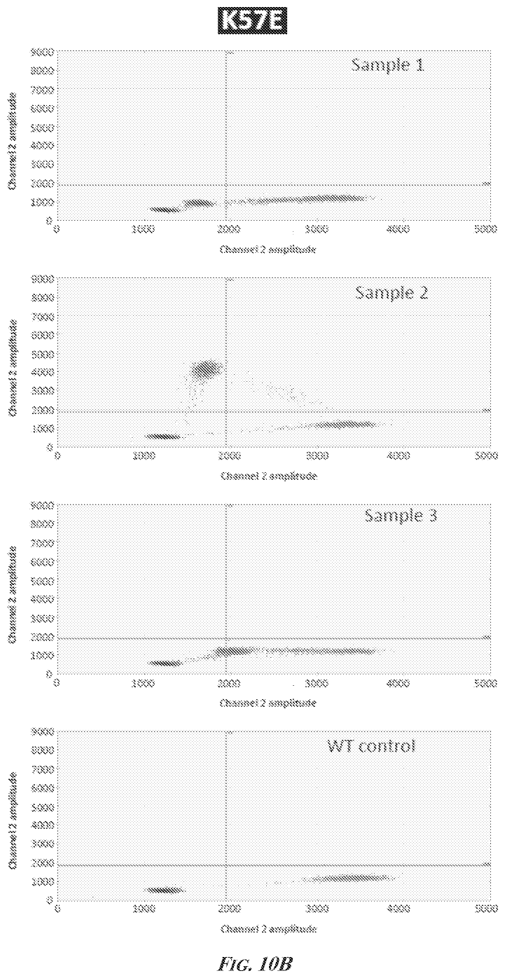

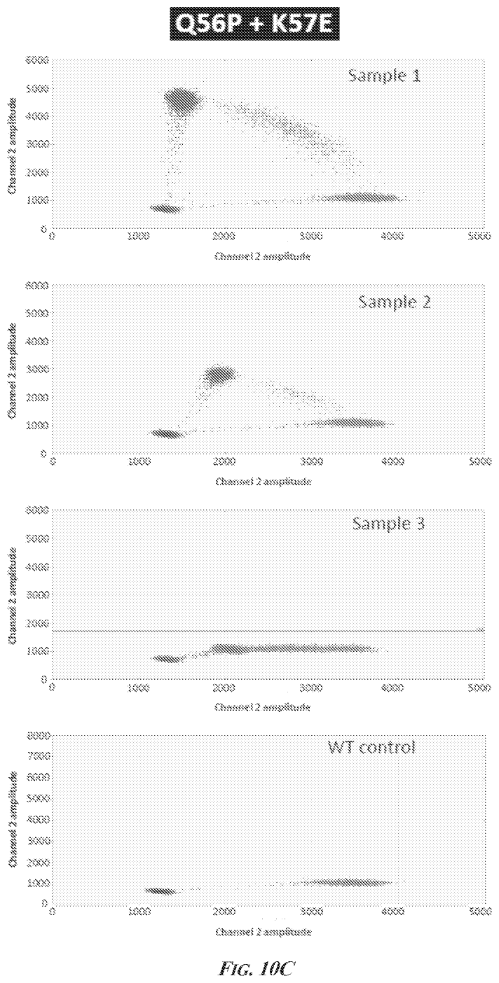

On average 144 million read pairs were generated for each library with more than 90% of bases scored at Q30 or above. All samples had at least 100× coverage at >66% of bases across all intervals targeted by the 52.9 Mb whole exome capture bait panel (range 66.5-92.4%, median 87.9%). The mean coverage across all samples (urine-derived DNA samples and non-neoplastic controls) was 299× (range 157-433×, median 297×). After filtering, the number of non-synonymous variants identified ranged from 80-295 per sample (mean=164, median 157), equivalent to a mean tumor mutation burden of 3.1 mutations/Mb. Within the eight POS^{V595E} samples the range was 80-242 variants per sample (mean=153, median 161) compared to 109-295 in the 28 UD^{V595E} samples (mean=167, median 156). These parameters were examined for normality by visual inspection to determine severity of skew and presence of outliers, and using the Shapiro-Wilk test we found no evidence of non-normality (p=0.2298). There was no significant difference in the mean number of mutations detected in POS^{V595E} and UD^{V595E} cases (two-sample t test, p=0.52), and there was no significant difference in the variability of the number of mutations observed between POS^{V595E} and UD^{V595E} cases (two-sided F test, p=1.00). Similar comparisons for each category of mutation (frameshift, in-frame insertion/deletion, missense and gain/loss of stop) identified no significant differences in the mean or variability between POS^{V595E} and UD^{V595E} cases. FIG. 1 provides a summary of non-synonymous mutations of key genes identified in two or more specimens within the sample cohort, which are described below. Additional details on variant frequency and distribution are provided in FIGS. 6 and 7.

### Example 3

Detection of Somatic Mutations within Exon 15 of the Dog BRAF Gene

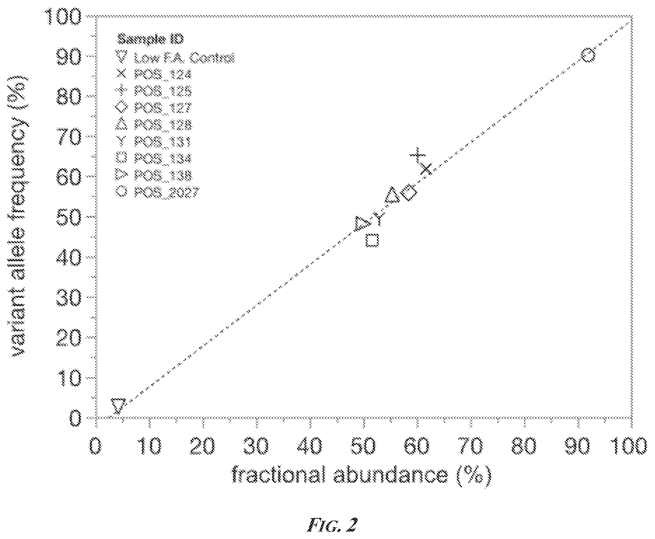

Mean sequence depth across all samples along the length of BRAF exon 15 ranged from 143× to 666× (median 373×). The V595E variant was identified in each of the eight POS^{V595E} samples, with variant allele frequency (VAF) ranging from 44.0% to 90.1%. The VAF for the low-FA control sample was 2.7% (total read depth 409×), compared to 4.3% from ddPCR analysis. Data obtained for this variant from ddPCR and WES analysis showed a strong correlation in all eight POS^{V595E} samples and the low-FA control (R²=0.979, Table 1, FIG. 2). There was no evidence of this variant in WES data from the remaining test samples and non-neoplastic controls, consistent with ddPCR analysis, and no other BRAF exon 15 variants were detected in any samples.

### Example 4

### Identification of BRAF Sequence Alterations Outside Exon 15

Evaluation of the entire BRAF gene revealed short deletions in exon 12 in 7/28 (25%) UD^{V595E} cases within the interval cfa16: 8,276,702-719 (FIGS. 3 and 8). Each was an in-frame deletion of either nine or 15 nucleotides (2/7 and 5/7 cases, respectively). Three samples (UD-099, UD-105 and UD-110) shared the same deletion of 15 nucleotides (cfa16: 8,276,703-717), resulting in elimination of codons 481-485 (relative to Ensembl Transcript ENSCAFT00000006306; SEQ ID NO: 5). This is hereafter denoted as ΔNVTAP based on the single letter codes for the deleted amino acid sequence. The remaining four deletions were disruptive. Of these, two samples (UD-003 and UD-097) shared the same deletion of nine nucleotides (codons 480-483, cfa16: 8,276,702-711) followed by an A>T substitution, leading to replacement of four amino acids by a single phenylalanine residue (denoted as ΔLNVT>F). The two remaining deletion events were each found in a single sample and spanned 15 nucleotides. These resulted in alteration of codons 480-485 in UD-112 (ΔLNVTAP>F), and codons 474-479 in UD-049 (ΔNVTAPT>K), within the intervals cfa16:8,276,702-716 and cfa16: 8,276,705-719, respectively. No deletions were identified within this region in any of the remaining UD^{V595E} samples, nor in any POS^{V595} cases or control samples. No other variants were evident elsewhere within the BRAF gene in any UC case or control specimen.

### Example 5

Sequence Evaluation of Upstream Genes from the RAS/RAF/MAPK Pathway

Other members of the RAF family were assessed for evidence of somatic alteration. A single non-synonymous SNV was located in ARAF, a missense C>T substitution within the kinase domain in exon 16, resulting in P528S (POS-124). No putative somatic variants were detected in RAF1/CRAF or in members of the RAS gene family (HRAS, KRAS, NRAS). Growth factor receptors and their ligands were then examined. The EGF gene was disrupted in a single sample, a premature stop codon in exon 14 (W704*, POS-128). Sample POS-124 showed two missense substitutions within exon 28 of the associated receptor gene EGFR (H1069Y and P1088A). Two missense variants were identified in the receptor tyrosine kinase gene ERBB2, one each in samples UD-007 (exon 2, L37P) and UD-082 (exon 7, G292R). Single missense mutations were identified in each of FGFR1 (UD-081), FGF5 (UD-104) and FGF6 (UD-088). No variants were identified in genes encoding PDGF/PDGFR or VEGF/VEGFR.

### Example 6

### Assessment of Downstream MAPK Pathway Genes

Analysis of downstream MAPK pathway members identified six samples exhibiting short deletion events in MAP2K1 (which encodes the MEK1 protein kinase). These were restricted to UD^{V595E} cases that showed no deletions in BRAF exon 12 (6/28, 21%). Of these, five were 15 bp deletions in MAP2K1 exon 2 within the interval cfa30: 30,720,179-206. In sample UD-091 this comprised a disruptive in-frame deletion of codons F53 to Q58 (relative to Ensembl Transcript ENSCAFT00000043934; SEQ ID NO: 51) and formation of a new leucine residue (cfa30: 30,720,179-193, ΔFLTQKQ>L). The remaining four variants detected were non-disruptive in-frame deletions. Sample UD-109 showed deletion of cfa30: 30,720,188-202, resulting in loss of codons Q56 to G61 (ΔQKQKVG). Two further samples (UD-054 and UD-082) showed the same deletion of cfa30: 30,720,192-206, resulting in loss of codons Q58 to E62 (ΔQKVGE), and the fifth (UD-108) showed deletion of cfa30: 30,720,190-204, resulting in loss of codons K57 to E62 (ΔKQKVGE). Also within this interval was a single missense substitution resulting in K57B in sample UD-088. The sixth MAP2K1 deletion event occurred in exon 3, within which sample UD-102 exhibited a 6 bp deletion resulting in loss of codons P105 and A106 (APA, cfa30: 30,721,656-661). No other alterations were identified in MAP2K1, and none were evident in MAP2K2 (MEK2) or the downstream pathway members MAPK1 (ERK) or MAPK3 (ERK1). RAS can also regulate critical cellular processes via the P13K/AKT/mTOR signaling cascade and so key members of this pathway were examined. A single instance of TP53 mutation was identified, within exon 7 (UD-091) resulting in C230S. No variants were evident in the key cancer genes PTEN, MTOR, AKT1, TSC1/2 or MDM2, or in genes encoding components of PI3K.

### Example 7

### Mutations of Epigenetic Modifiers, DNA Repair Genes and Chromatin Organizers

Epigenetic modifiers showed evidence of recurrent alteration. Among histone-modifying genes, missense mutations were identified in the lysine demethylase genes KDM1A (POS-128, three variants), KDM5A (POS-138, UD-105), KDM5C (POS-124, UD-049, UD-099 and UD-112) and KDM64 (UD-110 and UD-112). Missense mutations were also identified in the lysine methyltransferase genes KMT24 (UD-018) and KMT2C (UD-092), while sample UD-081 showed a missense mutation in the former and a premature stop in the latter. KMT2D showed missense mutations in UD-018 (two variants), UD-112 and POS-125, and KMT2E was mutated in POS-127 and POS-128. Two samples (UD-003, UD-097) shared the same missense mutation in SETD2 (also known as KMT3A), resulting in S509C, and EZH2 (also known as KMT6A) was mutated in UD-091. Missense mutations also occurred in the histone acetylation genes EP300 (UD-113), HDAC5 (UD-003 and UD-097, which shared the same alteration), HDAC7 (UD-100) and HDAC9 (UD-018), and in the histone acetyltransferase gene KAT6B (UD-018). Key DNA methylation genes, including DNMT3A and TET2, showed no evidence of mutation.

Among DNA repair genes three showed alterations in more than a single sample. The mismatch repair gene MSH6 showed missense mutations in three samples, within exon 4 (UD-092, two variants, and UD-099) and exon 5 (POS-134). The double-strand break repair gene RAD50 was mutated in both UD-054 and POS-138. Also of note, missense mutations of the ATM serine/threonine kinase gene, an initiator of DNA damage response, occurred in three cases (UD-027, UD-082 and UD-084). MDC1 (Mediator of DNA Damage Checkpoint 1) also showed missense mutations in three UD^{V595E} samples (UD-027, UD-097 and UD-098).

There was also recurrent disruption of chromatin remodeling genes. Loss of function alterations of ARID1A were identified in three samples, comprising nonsense mutations in exon 1 (UD-106) and exon 4 (UD-113), and a frameshift deletion in UD-082 (exon 20). Missense mutations of ARID1A were detected in a further two samples, within exon 3 (POS-125) and exon 12 (UD-104). A nonsense mutation was identified in SMARCC1 (UD-113), while three samples had missense mutations in SWI/SNF complex gene PBRM1 (UD-084, UD-092 and UD-112). Members of the Chromodomain Helicase DNA Binding Protein family were recurrently altered, including CHD4 (UD-092), CHD5 (POS-125), CHD6 (UD-091 with two mutations, and UD-054) and CHD7 (UD-084). Four UD^{V595E} samples (UD-003, UD-097, UD-105 and UD-109) shared the same mismatch alteration in exon 2 of SMCHD1 (Structural Maintenance Of Chromosomes Flexible Hinge Domain Containing 1) resulting in N94T, and a fourth was identified in UD-112 (exon 7).

### Example 8

### Additional Genes Exhibiting Recurrent Alterations

Aside from members of the Ras/Raf/MAPK pathway, LRP1B (LDL Receptor Related Protein 1B) was the most frequently disrupted gene identified in the present study. Six missense mutations were identified among five samples within this gene, with two variants in POS-2027 and one each in UD-001, UD-033, UD-054, UD-082 and UD-104. CSMD3 (CUB And Sushi Multiple Domains 3) was also among the most frequently mutated genes within the present study, with missense mutations evident in five samples (POS-124, POS-131, UD-054, UD-081 and UD-113 (two variants). The related gene CSMD1 showed missense mutations in four additional samples (POS-128, UD-027, UD-088 and UD-113). Four UD^{V595E} samples (UD-018, UD-054, UD-091 and UD-105 with two variants) exhibited missense mutations within the RYR2 gene (Ryanodine Receptor 2) gene. STAG2 (Stromal Antigen 2) was altered in three samples (missense mutations in UD-018 and POS-124, and a gain of stop in UD-091). Notably, among these four recurrently mutated genes, no samples shared the same variant, nor was the same codon altered in multiple samples.

### Example 9

Development of Combinatorial Assays for Detecting in-Frame Deletions in BRAF and MAP2K1

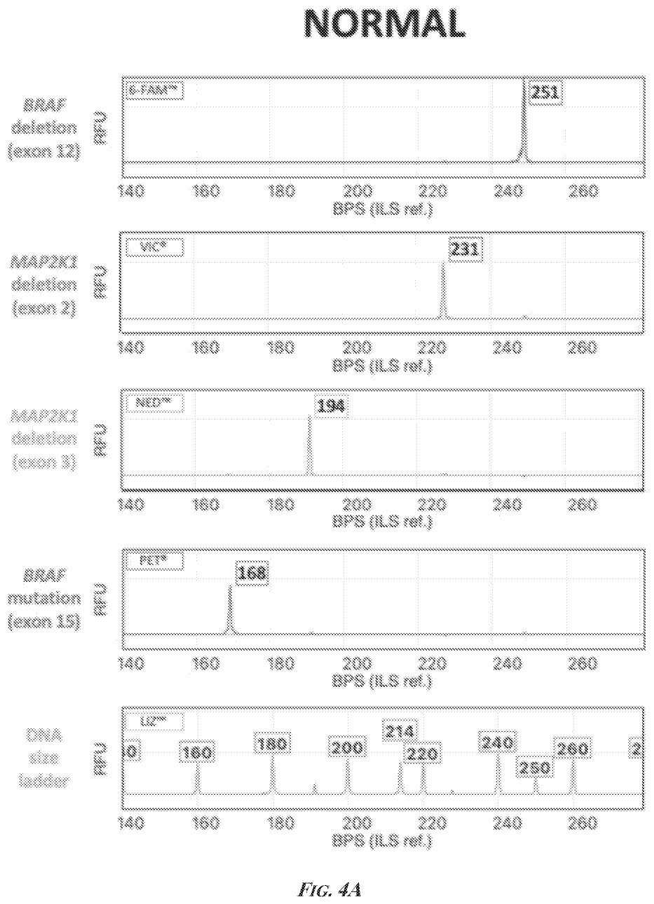

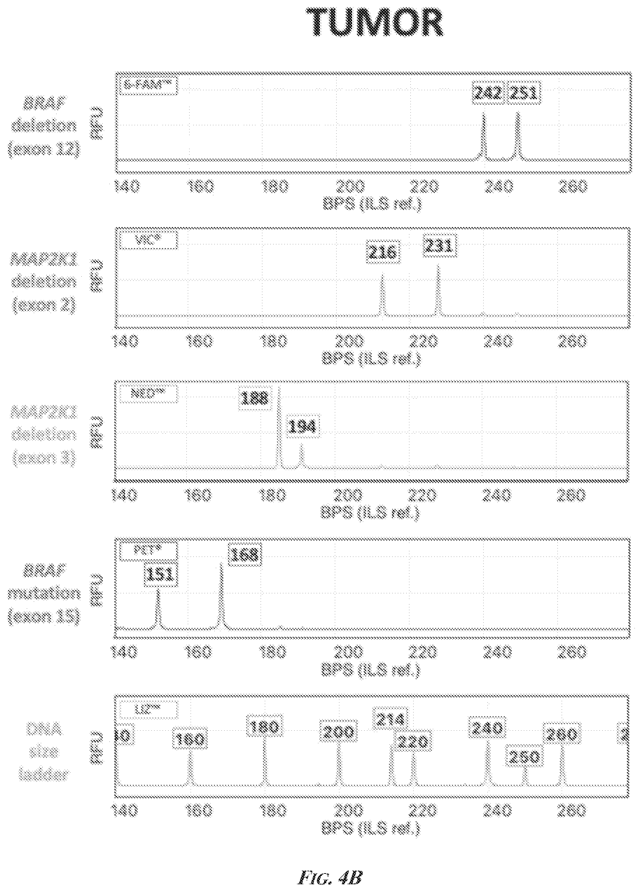

Regions of BRAF exon 12 and MAP2K1 exons 2 and 3 were investigated using novel CE assays to verify the presence of the deletions identified in sequenced samples, and to determine their frequency in additional specimens. A fourth assay was developed for distinction between wild-type and mutant alleles at the BRAF V595E locus. FIG. 4a shows an example of the resulting data obtained using DNA isolated from a normal (non-neoplastic) specimen. A single peak of fluorescent signal was present for each amplified product, the size of which was consistent with wild-type sequence. FIG. 4b shows data from specimens that showed sequence alterations within these intervals in WES analysis. CE assay results were consistent with the presence of the BRAF V595E mutation in POS-138, a 9 bp deletion within BRAF exon 12 in UD-097, a 15 bp deletion within MAP2K1 exon 2 in UD-109 and a 6 bp deletion within MAP2K1 exon 3 in UD-102. The remaining 10 samples that showed BRAF exon 12 or MAP2K1 exon 2/3 deletions in WES analysis were analyzed in the same manner, and the resulting data from both approaches were fully concordant.

### Discussion of the Examples

Previous studies have identified an activating BRAF V595E mutation in 85% of canine UC cases, highlighting the MAPK pathway as a compelling target for inhibitor therapy; however the remaining 15% of cases remain largely uncharacterized at the molecular level. In normal cells, the Ras/Raf/MAPK pathway is initiated by an extracellular stimulus, such as the binding of a growth factor to its receptor tyrosine kinase. This leads to activation of RAS, resulting in phosphorylation and dimerization of RAF. Activated RAF then induces activation of MEK1/2, which in turn stimulates ERK1/2 to regulate a wide variety of cellular processes. In human cancers the orthologous BRAF V600E mutation confers an effective target for small molecule inhibitor therapies. The mutation causes the BRAF protein to mimic the conformational changes that normally occur during dimerization, allowing it to act as a monomer without prior need for RAS activation. This typically results in a ˜500-fold increase in kinase activity compared to wild-type BRAF (reviewed in Dankner et al., 2018). V600E can be targeted by BRAF monomer inhibitors such as vemurafenib and dabrafenib that act on the so-called ‘αC-out/DFG-in’ structural conformation of the active protein kinase. Ongoing studies are investigating whether vemurafenib induces a similar therapeutic effect in canine UC cases bearing the BRAF V595E variant (Rossman et al., 2021). The remarkably high incidence of BRAF V595E mutation in canine UC has resulted in a biased focus on genomic profiling of tumors bearing this variant. This study aimed to identify recurrent variants among cases without the BRAF V595E variant that have potential to extend existing diagnostic strategies and/or to provide alternative candidates for additional trials of targeted therapies.

Comparison of methods for detection of BRAF V595E mutations. ddPCR and WES data showed complete correlation in terms of BRAF V595E variant status, with each sample classified as either positive or undetected by both methods. Furthermore, quantitative assessment of the VAF for this site in WES data showed strong correlation with the FA determined by ddPCR analysis across a wide range of values, supporting the validity of integrating results from both methods for this variant. No alternative mutations of codon V595 were identified that might result in a similar phenotype but fail to be detected using the ddPCR assay. Furthermore, since no BRAF exon 15 variants other than V595E were identified in any samples there is no evidence that absence of a positive ddPCR result in UD^{V595E} cases reflects failure to amplify the mutant allele due to sequence mismatches with either primers or probe.

BRAF is one of three evolutionarily conserved serine/threonine kinases that link RAS to MEK. The remaining two paralogs, ARAF and RAF1 (also known as CRAF) are less potent activators of MEK, and are rarely mutated in human cancers (Nolan et al., 2021). Furthermore, while BRAF mutations are reported in only 1.4% of human urinary tract cancers, the V600E variant constitutes 95.9% of amino acid substitutions found within this gene across all human cancers (Tate et al., 2019). The absence of BRAF point mutations other than V595E in this study, and the lack of evidence for recurrent alterations in ARAF and RAF1, is therefore globally consistent with observations in human cancers. We therefore investigated other MAPK pathway-related genes for mutations that might also confer susceptibility to inhibitor therapies.

Evaluation of upstream genes within the Ras/Raf/MAPK pathway. RAS acts as the intermediary between the extracellular stimulus and the RAF kinase, and is encoded by three isoforms (HRAS, KRAS and NRAS). These are among the most frequently mutated genes across all human cancers (Tate et al., 2019). In a previous study, Sanger sequencing analysis of 29 POS^{V595E} and 10 UD^{V595E} canine UC samples failed to identify any mutations of these genes (Mochizuki & Breen, 2017). While the use of conventional Sanger sequencing analysis would have limited the ability to detect low frequency variants in that study, the absence of HRAS, KRAS or NRAS mutations in the present WES study supports these earlier findings.

Human invasive UC often shows aberrations of receptor tyrosine kinases and ligands that act as the initial stimulus for MAPK pathway activity, offering several potential therapeutic targets. Activating alterations involving the ErbB receptor tyrosine kinase family are recurrent, primarily as a result of somatic mutation of ERBB2 and ERBB3 (˜10% of cases) and also increased copy number of ERBB2 and EGFR (Cancer Genome Atlas Research, 2014, Tate et al., 2019). Mutations of EGF and EGFR were each found in only a single sample from our cohort and did not coincide with sites that are altered frequently in human cancers (Tate et al., 2019), and no variants were identified in ErbB family members. Prior studies have, however, reported frequent upregulation of EGFR and ERBB2 in canine UC (Dhawan et al., 2015; Hanazono et al., 2015; Maeda et al., 2018; Millanta et al., 2018; Cronise et al., 2019; Parker et al., 2020). Immunohistochemistry studies have detected overexpression of the Her2 protein produced by ERBB2 in ˜60% of cases (Millanta et al., 2018; Tsuboi et al., 2019). More recently, increased DNA copy number of ERBB2 has been reported in 35% of dog UC cases (Sakai et al., 2020), which suggests that upregulation may be driven by aberrant gene dosage rather than somatic mutation. FGFR3 is also among the most frequently mutated genes in human UC, with somatic variants identified in ˜26% of all cases (Tate et al., 2019). Notably, several studies have shown a significantly lower incidence of FGFR3 mutation in human high-grade invasive UD compared to low-grade cases (8-12% and 79-83% of cases, respectively) (Cancer Genome Atlas Research, 2014; Hurst et al., 2017; Pietzak et al., 2017). The absence of FGFR3 mutation in the present study concurs with the fact that the vast majority of canine UC cases are invasive at the time of diagnosis.

Evidence for disruption of related pathways. Altered receptor tyrosine kinases such as EGFR and ERBB2 can also stimulate oncogenic activation of the PI3K/AKT/mTOR pathway, which interplays with the Ras/Raf/MAPK cascade in regulation of critical cellular processes. Disruption of the RTK/Ras/PI3K pathway occurs in ˜72% of human high grade invasive UC (Cancer Genome Atlas Research, 2014); however in many cases this is due to aberrant PI3K (Cancer Genome Atlas Research, 2014; Tate et al., 2019; Liow & Tran, 2020). PIK3CA is the most commonly mutated of the PI3K subunit genes across all human cancers, and while infrequent in UC (5-10% of all UC cases and 20% of high-grade invasive cases), mutations of PIK3CA are associated with a poor prognosis (Cancer Genome Atlas Research, 2014; Tate et al., 2019; Liow & Tran, 2020). The vast majority of PIK3CA somatic alterations occur within two hotspots located in exon 9 (codons E542K and E545H) and one in exon 20 (H1047R/L). These regions are highly conserved between the human and dog genomes, and are often mutated in canine hemangiosarcomas and mammary carcinomas (Megquier et al., 2019; Kim, 2020); however PIK3CA was not mutated in our dog cohort, nor were genes encoding other components of PI3K.

Interestingly, the pattern of TP53 alteration in human UC shows the inverse of FGFR3, with TP53 mutations reported in ˜50% of high-grade invasive tumors compared to 15% of low-grade non-invasive tumors (Sjodahl et al., 2011; Cancer Genome Atlas Research, 2014; Hurst et al., 2017; Pietzak et al., 2017). Overall, it is estimated that the p53/cell cycle pathway is inactivated in approximately 90% of human UC, primarily through mutation of TP53 and/or RB1 (Cancer Genome Atlas Research, 2014; Robertson et al., 2018). In our study, no instances of RB1 mutation were identified, and only a single TP53 mutation was evident. This is consistent with a recent study of 11 canine UC cases in which no TP53 mutations were identified using either WES or RNAseq analysis (Cronise et al., 2021).

Further comparisons with prior studies of canine and human UC. Despite the use of independent sample cohorts and different analytical strategies, our study showed several parallels with prior reports of the genomics of canine UC. As with our specific comparison of ddPCR and WES analysis for detecting the BRAF V595E variant, this replication provides confidence for the integration of data from different sources. This is of particular importance where sample resources are restricted due to the relative infrequency of the diagnosis, and where the frequency of any given variant within the population is low. Several earlier studies of canine UC have reported a low incidence of recurrently mutated genes other than BRAF. In one recent study of 11 dog UC samples, mutations of protein-coding regions were identified in 32 cancer-related genes; however only five of these (BRAF, LRP1B, CUL3, RNF213 and MSH2) genes were mutated in more than a single sample, and only two were mutated in more than two samples (BRAF 4/11 samples, LRP1B 3/11 samples; Cronise et al., 2021). Similarly, prior dog UC reports noted absence of variants in genes that are frequently altered in human UC, including CDKN2A, FGFR3, HRAS, MDM2, PIK3CA and TP53 (Cronise et al., 2021), which was consistent with our findings in the present study.

Conversely, our detection of variants within genes that were recurrently mutated in prior dog UC studies further elevates their candidacy for involvement in disease pathogenesis, particularly where there is similar evidence from human tumors. Among these is CSMD3 (CUB and Sushi multiple domains 3), which encodes a protein whose function is not yet understood. CSMD3 was mutated in five samples from the present study (14% of cases, comprising 2/8 POS^{V595E} and 3/28 UD^{V595E} samples), and was also highlighted as a recurrently mutated gene in bladder cancers of both dogs and people (Ramsey et al., 2017). While rarely altered in human cancers in general (<0.5% of all cases), the incidence of CSMD3 disruption in UC (˜14% of cases) is among the highest across cancer subtypes, along with ovarian, breast, gastric and colon carcinomas (AACR Project GENIE Consortium, 2017; Tate et al., 2019). A recent study in human ovarian cancers showed a significant correlation between CSMD3 mutation, elevated tumor mutation burden and shorter overall survival. Similar observations have been reported for CSMD1 in human gastric cancer (Wang et al., 2021), which was also mutated in multiple samples in the present study (11% of cases, 1/8 POS^{V595E} and 3/28 UD^{V595E} samples). The relatively high incidence of CSMD3 mutation in both human and canine UC, and the correlation with outcome in other human cancers, renders this gene a worthy candidate for an association with UC pathogenesis.

Four UD^{V595E} samples (14%) exhibited missense mutations within the RYR2 gene, a calcium channel component. Ramsey identified RYR2 mutations via RNAseq analysis of seven canine UC cases, including one that was orthologous to a human UC driver mutation candidate (Ramsey et al., 2017). Recurrent mutations of RYR2 have been reported in several human carcinomas, including squamous cell carcinoma of the oral cavity (Patel et al., 2021) and lung (Xie et al., 2021), esophageal adenocarcinoma (Liu et al., 2021) and breast cancer (Cimas et al., 2020). Collectively, these studies present evidence that RYR2 mutation predicts a positive response to immunotherapy in multiple human cancers. Canine invasive UC has been identified as a pertinent model for driving the development and evaluation of novel immunotherapeutic agents, based on its cellular and genomic composition (Knapp et al., 2019). The RYR2 gene should therefore be evaluated for potential predictive value in this context.

Sporadic missense mutations of LRP1B have been reported previously in canine bladder cancers (Ramsey et al., 2017; Cronise et al., 2021). In the present study LRP1B mutations occurred in six samples (17% of the cohort), of which five were UD^{V595E} (18%). LRP1B, a putative tumor suppressor gene, encodes a member of the low density lipoprotein (LDL) receptor family, and is among the most frequently altered genes in human cancers, by a variety of both genetic and epigenetic mechanisms (Principe et al., 2021). Somatic mutations of LRP1B are estimated to occur in 12% of all human cancer cases, and in more than 20% of cases of certain tumor types, including bladder cancers (Brown et al., 2021). Furthermore, deletion of the region encoding LRP1B has been defined as a hallmark of high grade human UC, with allelic loss of this site in 49% of grade 3 tumors versus 8% of grade 1 tumors (Langbein et al., 2002). Similarly, LRP1B lies in a region of highly recurrent deletion in canine UC (Shapiro, 2015 #1). Of particular note, LRP1B mutation has been shown to be a positive predictor of response to immune checkpoint inhibitor therapy in multiple human cancer subtypes (Brown, 2021 #115]. Both LRP1B and RYR2 are therefore logical candidates for consideration as positive predictors of immunotherapy response in canine trials of this treatment modality.

Three DNA damage response and repair genes were mutated in more than two samples, MSH6, MDC1 and ATM. MDC1 is involved in several processes relating to DNA damage, including checkpoint-mediated cell cycle arrest in response to double-stranded DNA breaks, and activation of ATM Serine/Threonine Kinase. Genomic alterations of MDC1 in human cancers have been shown to increase sensitivity to DNA damaging chemotherapeutic reagents including doxorubicin and cisplatin (Ruff et al., 2020). Similarly, ATM acts as a sensor of DNA damage and cell cycle checkpoint via regulation of genes including TP53 and BRCA1. The AIM gene is altered in ˜6% of all human cancers and in ˜11% of human urothelial carcinomas (AACR Project GENIE Consortium, 2017), and showed missense mutations in three samples in the present study, all of which were UD^{V595E} specimens. Recent studies in human UC patients (Yin et al., 2018; Yi et al., 2020) reported that the presence of ATM mutations confers a significantly greater benefit from treatment with immune checkpoint inhibitors, and elevates sensitivity to a total of 29 drug therapies, including the first-line treatment cisplatin. The presence of recurrent mutations of these DNA repair genes in canine UC may therefore open up the possibly for PARP inhibitor therapy in a proportion of cases, as has been suggested for human UC (Bronimann et al., 2020).

Several studies note a high prevalence of mutations of chromatin modifiers in human UC, including histone demethylases and methyltransferases (Cancer Genome Atlas Research, 2014; Robertson et al., 2018; Tate et al., 2019; Liow & Tran, 2020). Similarly, the present study highlights several epigenetic factors as targets of recurrent mutation in canine UC, particularly histone demethylase and methyltransferase genes and the chromatin remodeling gene ARID1A. Mutations in KDM6A and ARID1A have been reported as the most frequently altered chromatin modifying genes in human UC regardless of tumor stage or grade, suggesting that they are early events (Pietzak et al., 2017). We identified STAG2 alterations in three samples. STAG2 is another chromatin regulator that is frequently altered in many human cancers, including UC, in which it acts as a tumor suppressor (Richart et al., 2021). One of the primary functions of STAG2 is in regulating the cohesion and segregation of sister chromatids, and it has been suggested that disruption of this gene may be associated with the high incidence of aneuploidy in human UC. We are not aware of prior reports of STAG2 alteration in canine UC, but the functional involvement of this gene is plausible given that these tumors show a remarkably high incidence of chromosome imbalance (Shapiro et al., 2015a; Shapiro et al., 2015b; Mochizuki et al., 2016).

SMCHD1 was among the most frequently mutated genes identified in the study. Missense variants were found in 18% of UD^{V595E} samples, but were absent from the eight POS^{V595E} samples. SMCHD1 plays a role in epigenetic silencing via regulation of chromatin architecture, and in DNA repair in response to double-strand breaks. Mutations of SMCHD1 are infrequent in human cancers; however a recent report defined a model in which somatic alterations of three genes, including SMCHD1, are predictive of overall survival in human bladder cancer (Ning et al., 2020). Interestingly, while human SMCHD1 shows no evidence of mutational hotspots (Tate et al., 2019), four canine UC samples shared the same variant, at the site orthologous to residue N86 in the human gene. This therefore constitutes the second most frequently mutated site in the present study, after BRAF V595E.

Although the infrequency of recurrent mutations of the same gene precludes statistical combinatorial analysis, FIG. 1 highlights several patterns that warrant further analysis in larger cohorts. At the level of growth factors and ligands that activate the MAPK pathway, mutations of FGFR/R and ERBB2 genes were found only in UD^{V595E} samples (5/28, 18%); while EGF/EGFR mutations were identified only in POS^{V595E} samples (2/8, 25%). Mutations of the DNA repair genes ATM and MDC1 occurred only in UD^{V595E} samples (six mutations among 5/28 samples, 18%). Among the chromatin remodeling genes ARID1A, SMARCC1, PBRM1, SMCHD1 and CHD4/5/6/7 were 19 instances of mutation, of which 17 were found in UD^{V595E} samples. Furthermore, five samples (four of which were UD^{V595E}) had mutations in two chromatin remodeling genes. These early findings suggest that UD^{V595E} samples may be enriched in mutations involving DNA repair genes and chromatin-remodeling genes. Of particular note, each of the five samples with SMCHD1 mutations also showed either BRAF or MAP2K1 deletions. The coincident nature of these events indicates the potential to define additional molecular subtypes within the UD^{V595E} cohort that are based on combinatorial assessment of mutational signatures in multiple genes.

BRAF and MAPK2K1 deletions are recurrent in specimens without BRAF V595E mutations. The most critical finding from the present study is the detection of mutually exclusive short in-frame deletions in the BRAF and MAP2K1 genes in 13/28 (46%) UD^{V595E} samples. Seven samples showed either 9 bp or 15 bp deletions in BRAF exon 12. This region encodes the β3-αC loop of the kinase domain, which human studies show to be involved in the mechanism that allows BRAF to switch between an active and an inactive state. Deletions that induce shortening of the loop limit the conformational flexibility of the protein, locking it in a RAS-independent, constitutively active form, with kinase activity reaching a peak with the deletion of five amino acids from the β3-αC loop (Foster et al., 2016). These mutations may therefore indicate alternative mechanisms for MAPK pathway activation in canine UC, aside from the BRAF V595E point mutation.

While rare in general, in-frame deletions within BRAF exon 12 have been reported in a small number of human cancer subtypes, the majority of which eliminate the region extending from amino acid residue N486 to P490 (termed ΔNVTAP, FIG. 3). This human deletion variant (hΔNVTAP, assigned COSMIC Genomic Mutation ID COSV56100024) is equivalent to elimination of N481-P485 from dog BRAF (cNVTAP), which was the most common of these deletions identified in the present study. Similarly, the 15 bp deletion identified in UD 112 (cΔLNVTAP>F) is orthologous to the previously described human variant with COSMIC Genomic Mutation ID COSV104608678 (hΔLNVTAP>F). One study (Foster et al., 2016) reported deletions of this nature in ˜1% of pancreatic carcinomas, of which 11 were of the ΔNVTAP type. A recent study identified ΔNVTAP in 20/69 (29%) cases of human adult Langerhans cell histiocytosis (LCH), an inflammatory myeloid neoplasm that is strongly linked to aberrant RAF-MEK-ERK activation (Jouenne et al., 2020). Interestingly, 25/69 (36%) cases from the same human LCH cohort harbored the BRAF V600E mutation, but only a single case presented with both of these variants concurrently. Sporadic examples of BRAF exon 12 in-frame deletions have also been reported in human myeloid neoplasms, lung carcinomas, colon carcinomas and prostatic carcinomas, in which they are mutually exclusive from V600E mutations, and also from mutations of RAS genes (Estep et al., 2007; Chakraborty et al., 2016; Chen et al., 2016; Foster et al., 2016; Abida et al., 2017; Zehir et al., 2017; Wrzeszczynski et al., 2019; Paziewska et al., 2020; Ren et al., 2021).

In human cancers, the ΔNVTAP variant exhibits MAPK pathway signaling activity comparable to that of BRAF V600E (Foster et al., 2016). Functional studies using human cancer cell lines have shown that, in contrast to V600E mutants, ΔNVTAP mutants are resistant to BRAF monomer inhibitors such as vemurafenib and dabrafenib that target the ‘αC-out/DFG-in’ structural conformation of the active protein kinase. Instead, mutants with exon 12 in-frame deletions are susceptible to inhibitors that target the ‘αC-in/DFG-out’ conformation, such as the pan-RAF dimer inhibitors AZ628 and LY3009120, as well as allosteric MEK inhibitors such as cobimetinib and trametinib (Chakraborty et al., 2016; Chen et al., 2016; Foster et al., 2016; Niu et al., 2019). Moreover, introduction of the ΔNVTAP alteration into human cells bearing the BRAF V600E mutation has been shown to confer resistance to vemurafenib in vitro, reinforcing the significance of this deletion for therapeutic response (Foster et al., 2016). Consequently, there is increasing emphasis on classification of BRAF alterations in human tumors into categories that are predictive of response to different therapeutic compounds, based on the specific protein conformations they target (Dankner et al., 2018).

We examined other genes within the MAPK pathway for evidence of similar alterations, and identified two intervals exhibiting short in-frame deletions in the dog MAP2K1 gene. MAP2K1 encodes the MEK1 protein kinase, which is activated by BRAF and which subsequently activates the downstream ERK protein, stimulating cell growth, proliferation, and survival. MEK therefore offers an alternative mechanism for inducing aberrant ERK activation in the absence of mutant BRAF. Four mutational hotspots have been identified in human MEK1. Two of these, spanning exons 2 and 3, harbor short deletions in a small subset of cancers, namely the negative regulatory region known as inhibitory helix A, and the catalytic β3-αC loop region of the kinase domain (Yuan et al., 2018). While generally infrequent, oncogenic deletions within MAP2K1 exons 2 and 3 are enriched in a small subset of human cancers including certain melanocytic lesions and particularly pediatric LCH (˜30% of cases), and are mutually exclusive from the BRAF V600E variant (Brown et al., 2014; Nelson et al., 2015; Chakraborty et al., 2016; Nann et al., 2019; Williams et al., 2020). These two hotspots of MAP2K7 deletion in human cancers are orthologous to the regions of recurrent deletion identified in our canine UC cohort.

Normally, the negative regulatory region interacts with the kinase domain of the MEK1 protein, causing it to become stabilized in an inactive conformation; thus disruption of the negative regulatory region can trigger MEK1 kinase activity. Functional analyses have shown that a variety of point mutations and deletions within this region result in aberrant ERK phosphorylation consistent with constitutive activity. This includes the K57E variant that we identified in a single canine sample, which corresponds to the most frequently altered MAP2K1 codon identified in human cancers, and which has been associated with emergence of resistance to BRAF monomer inhibitors in human cells (Tate et al., 2019). Similarly, the canine ΔFLTQKQ>L and ΔQKQKVG variants identified in the present study are orthologous to human ΔFLTQKQ>L (spanning MEK1 residues F53-Q58) and ΔQKQKVG (Q57-G61), which have been assigned COSMIC Genomic Mutation IDs COSV61072289 and COSV61072263, respectively (Lee et al., 2017). Deletions spanning the negative regulatory region have been associated with the onset of resistance to BRAF monomer inhibitors in human patients, but tumors with these variants are typically responsive to allosteric MEK1 inhibitors and ERK inhibitors (Gao et al., 2018).

The mutational hotspot within human MAP2K1 exon 3 is highly conserved with the β3-αC loop region of BRAF, as well as other protein kinases including EGFR and HER2 (Chen et al., 2016; Foster et al., 2016). Similarly, deletion within these regions cause shortening of the loop to yield an activated conformation, leading to RAF-independent MAPK pathway activation at a level determined both by the size and the site of the deletion (Yuan et al., 2018). Deletions within the MEK1 catalytic kinase domain confer variable response to different MEK inhibitors, and can lead to resistance to allosteric MEK1 inhibitor therapy. Promising results have been observed using ATP-competitive selective MEK inhibitors and ERK inhibitors to target these mutations, as well as those within the negative regulatory region (Gao et al., 2018). Thus, somatic deletions within the β3-αC loop regions of RAF and MEK proteins confer a closely related impact on drug susceptibility in human cancers. In turn, as with BRAF, MAP2K1 mutations are increasingly categorized according to their impact on MEK1 conformation and consequent therapeutic response.

These findings identify potential mechanisms for MAPK pathway activation in canine UC without BRAF V595E mutation that may also have therapeutic implications. The absence of detectable mutations in MAPK pathway members within the remaining cases may be for a variety of reasons. Mutations may exist at a fractional abundance that lies below the limits of detection of WES analysis, or may occur in regions that are not captured effectively by the exome baits used in this study. Pathway disruption may be induced by mechanisms other than somatic gene mutation, such as the altered activity of distant regulatory elements or methylation. Alternatively, these may represent molecularly distinct forms of canine UC arising through dysregulation of alternative pathways.

Aside from BRAF V595E there is relatively limited evidence for recurrent alterations shared between published studies of canine UC. It is likely that a combination of factors explains this limited overlap, including the use of different sample types (fresh vs. fixed, archival tissue vs. urine sediment), experimental strategies (WES vs targeted amplicon sequencing vs RNAseq) and methodologies for variant detection and filtering. Additional shared variants may become evident as studies transition toward the use of new dog genome assemblies, which are derived from different individuals and which provide more comprehensive sequence annotation of non-protein-coding regions. This study adds the additional factor of an intentional skew toward specimens without the BRAF V595E mutation. The use of urine-derived DNA samples for WES analysis allowed us avoid formalin-induced sequence artefacts that can be misclassified as somatic mutations. Specimens derived from free-catch urine do not, however, allow determination of the primary site of origin of the tumor, and so it is possible that our study cohort includes lesions from different sites within the urogenital tract. Given the propensity for late-stage diagnosis with local invasion and distant metastasis, however, this confounding factor extends also to histopathologically-validated biopsies, particularly those retrieved at necropsy. In an earlier ddPCR study of 60 abnormal biopsies from the canine urogenital tract with a diagnosis other than UC, eight (13%) showed a relative copy number increase of cfa13 and only a single sample (2%) showed gain of cfa36 (Wise, 2020). None of the 60 samples showed gain of both chromosomes, providing additional support for the presence of a urothelial carcinoma in free-catch urine samples that test positive for both CNV signatures.

The method and criteria by which cases have been classified as lacking the BRAF V595E mutation varies between studies, and have included conventional Sanger sequencing analysis, detection of restriction fragment length polymorphisms, targeted amplicon resequencing and WES analysis (Decker et al., 2015). Sanger sequencing analysis is generally considered to have a sensitivity limit of ˜15%, while next-generation sequencing methods (where sensitivity is heavily dictated by the read-depth and filtering criteria applied) frequently sets a threshold of 5% VAF. Our study benefits from classification by ddPCR analysis using a methodology we have shown to detect mutations reliably down to a fractional abundance of 0.025%. Since the confidence with which absence of a variant can be reported is heavily dependent on the nature and analysis parameters of the detection method used, we elect to use the term ‘undetected’ instead of ‘wild-type’.

Integration of data from prior studies is confounded by inconsistencies in the definition of tumor types as ‘bladder cancer’, ‘transitional cell carcinoma’ and ‘urothelial carcinoma’, with these terms sometimes used interchangeably in the same report. Furthermore, canine bladder cancers have a propensity for invading into adjacent tissues such as the ureter, the prostate gland and the prostatic urethra. Coupled with the fact that these lesions are typically encountered at a late stage, it is challenging to identify the true primary site of origin of the malignancy within the urinary tract, even with full necropsy evaluation. In males, this is complicated further by the potential for a prostatic origin, and indeed it has been estimated that 30% of invasive UC cases in male dogs have prostatic involvement (Knapp et al., 2014; Knapp et al., 2019). To date there are few genomic studies focusing specifically on canine prostate cancers; however, work is underway to catalog their molecular profiles and to determine whether they exhibit molecular signatures distinct from those of bladder tumors (Wiley et al., manuscript in preparation).

### CONCLUSIONS

The investigations disclosed herein suggested that the ˜15% of canine UC cases without BRAF V595E mutation do not harbor a variant of comparable prevalence elsewhere within this gene, nor in the coding region of any other gene within the exome captured. To date little is known of the clinical, anatomical, histologic and prognostic significance of canine UC in which the BRAF V595E variant is undetected. It remains to be determined whether the absence of this mutation in a minority of cases indicates earlier-stage disease that will eventually develop the variant, or whether it constitutes one or more distinct molecular subtypes with somatic alterations in other genes activating alternative pathways. Armed with additional markers for subclassification of canine UC it is now possible to correlate discrete molecular signatures with clinical and histologic features that impact tumor behavior and therapeutic response. This also provides a mechanism to track the accumulation of key somatic alterations from tumor initiation through to progression, to map out the relative order and timeline during which these events emerge. We noted that the breeds represented in the cohort of UD^{V595E} cases showed only modest overlap with those considered to be at high risk of UC (9/28 dogs, comprising seven beagles, one Scottish terrier and one beagle mix). The availability of new markers for UD^{V595E} cases will allow us to investigate, in a larger sample size, whether UD^{V595E} UC and POS^{V595E} UC represent distinct molecular subtypes with differential breed predisposition.

The presently disclosed findings raise the possibility that short in-frame deletions within BRAF exon 12 and MAP2K1 exons 2 and 3, and single nucleotide substitutions in MAP2K1 exon 2, are MAPK-pathway activating events that may have significant therapeutic implications for canine UC. To this end, the combinatorial CE assay described herein provides a rapid, cost-effective and non-invasive screening strategy with potential as a companion diagnostic for veterinary medicine. When considered in context with clinical signs, this may also have utility as a means to monitor dogs during treatment for emergence of these alterations, signaling loss of chemotherapeutic sensitivity and prompting the need to pursue alternative treatments. Logistical and financial challenges may preclude the routine use of alternative MAPK pathway inhibitor therapies in a veterinary setting; however, the identification of canine BRAF and MAP2K1 in-frame deletions may expedite the association of mutational status with drug response, given the relative infrequency of these variants in human tumors. The reported prevalence of BRAF and MAP2K1 in-frame deletions in human cancers is likely to be an underestimate since sequencing technologies and analytic tools are designed primarily for identification of point mutations (Chen et al., 2016). In-frame deletions comprise only 4.3% of all MAP2K1 mutations reported across all human cancers, and only 0.2% of all BRAF mutations (Tate et al., 2019); consequently, they remain relatively understudied. The conservation of BRAF and MAP2K1 deletions in canine UC and human LCH and pancreatic carcinomas suggests there may be synergistic benefit from parallel functional studies of these diverse cancer types, as a means to better understand the broader relationship between somatic alteration, protein conformation and therapeutic sensitivity.

### REFERENCES

All references cited in the instant disclosure, including but not limited to all patents, patent applications and publications thereof, scientific journal articles, and database entries (e.g., GENBANK® biosequence database entries, including all sequences disclosed therein and all annotations thereof), are incorporated herein by reference in their entireties to the extent that they supplement, explain, provide a background for, or teach methodology, techniques, and/or compositions employed herein.
AACR Project GENIE Consortium (2017). AACR Project GENIE: Powering Precision Medicine through an International Consortium. Cancer Discov, 7(8), 818-831. doi:10.1158/2159-8290.CD-17-0151.
Abida et al. (2017). Prospective Genomic Profiling of Prostate Cancer Across Disease States Reveals Germline and Somatic Alterations That May Affect Clinical Decision Making. JCO Precis Oncol, 2017. doi:10.1200/PO.17.00029.
Altschul et al. (1990) Basic local alignment search tool. J Mol Biol 215: 403-410.
Ausubel et al. (2002) Short Protocols in Molecular Biology, Fifth ed. Wiley, New York, New York, United States of America.
Ausubel et al. (2003) Current Protocols in Molecular Biology, John Wylie & Sons, Inc., New York, New York, United States of America.
Batzer et al. (1991) Enhanced evolutionary PCR using oligonucleotides with inosine at the 3′-terminus. Nuc Acids Res 19:5081.
Broeckx et al. (2014). Development and performance of a targeted whole exome sequencing enrichment kit for the dog (Canis familiaris Build 3.1). Sci Rep, 4, 5597. doi:10.1038/srep05597
Bronimann et al. (2020). Poly(ADP-ribose) polymerase inhibitors in prostate and urothelial cancer. Curr Opin Urol, 30(4), 519-526. doi:10.1097/MOU.0000000000000776.
Brown et al. (2014). High prevalence of somatic MAP2K1 mutations in BRAF V600E-negative Langerhans cell histiocytosis. Blood, 124(10), 1655-1658. doi:10.1182/blood-2014-05-577361.
Brown et al. (2021). LRP1B mutations are associated with favorable outcomes to immune checkpoint inhibitors across multiple cancer types. J Immunother Cancer, 9(3). doi:10.1136/jitc-2020-001792.
Butty et al. (2021). Presumptive malignant transformation of chronic polypoid cystitis into an apical transitional cell carcinoma without BRAF mutation in a young female dog. J Vet Intern Med, 35(3), 1551-1557. doi:10.1111/jvim.16107.
Cancer Genome Atlas Research, N. (2014). Comprehensive molecular characterization of urothelial bladder carcinoma. Nature, 507(7492), 315-322. doi:10.1038/nature12965.
Chakraborty et al. (2016). Alternative genetic mechanisms of BRAF activation in Langerhans cell histiocytosis. Blood, 128(21), 2533-2537. doi:10.1182/blood-2016-08-733790.
Chen et al. (2016). Oncogenic BRAF Deletions That Function as Homodimers and Are Sensitive to Inhibition by RAF Dimer Inhibitor LY3009120. Cancer Discov, 6(3), 300-315. doi:10.1158/2159-8290.CD-15-0896.
Cimas et al. (2020). Genomic Mapping Identifies Mutations in RYR2 and AHNAK as Associated with Favorable Outcome in Basal-Like Breast Tumors Expressing PD1/PD-L1. Cancers (Basel), 12(8). doi:10.3390/cancers12082243.
Clerc-Renaud et al. (2021). Treatment of genitourinary carcinoma in dogs using nonsteroidal anti-inflammatory drugs, mitoxantrone, and radiation therapy: A retrospective study. J Vet Intern Med, 35 (2), 1052-1061. doi:10.1111/jvim. 16078.
Cronise et al. (2021). Characterizing the molecular and immune landscape of canine bladder cancer. Vet Comp Oncol. doi:10.1111/vco.12740.
Cronise et al. (2019). Identifying the ErbB/MAPK Signaling Cascade as a Therapeutic Target in Canine Bladder Cancer. Mol Pharmacol, 96(1), 36-46. doi:10.1124/mol.119.115808.
Dankner et al. (2018). Classifying BRAF alterations in cancer: new rational therapeutic strategies for actionable Oncogene, mutations. 37(24), 3183-3199. doi:10.1038/s41388-018-0171-x.
Decker et al. (2015). Homologous Mutation to Human BRAF V600E Is Common in Naturally Occurring Canine Bladder Cancer—Evidence for a Relevant Model System and Urine-Based Diagnostic Test. Mol Cancer Res, 13 (6), 993-1002. doi:10.1158/1541-7786.MCR-14-0689.
Dhawan et al. (2015). Comparative Gene Expression Analyses Identify Luminal and Basal Subtypes of Canine Invasive Urothelial Carcinoma That Mimic Patterns in Human Invasive Bladder Cancer. PLoS One, 10(9), e0136688. doi:10.1371/journal.pone.0136688.
Duval et al. (2014). 13 Whole exome sequence analysis of canine transitional cell carcinoma of the bladder. European Journal of Cancer, 50. doi:10.1016/S0959-8049(14)70139-X.
Eilbeck et al. (2005). The Sequence Ontology: a tool for the unification of genome annotations. Genome Biol, 6(5), R44. doi:10.1186/gb-2005-6-5-44.
Estep et al. (2007). Mutation analysis of BRAF, MEK1 and MEK2 in 15 ovarian cancer cell lines: implications for therapy. PLoS One, 2(12), e1279. doi:10.1371/journal.pone.0001279.
Forbes et al. (2008). The Catalogue of Somatic Mutations in Cancer (COSMIC). Curr Protoc Hum Genet, Chapter 10, Unit 10 11. doi:10.1002/0471142905.hg1011s57.
Forbes et al. (2016). COSMIC: High-Resolution Cancer Genetics Using the Catalogue of Somatic Mutations in Cancer. Curr Protoc Hum Genet, 91, 10 11 11-10 11 37. doi:10.1002/cphg.21.
Foster et al. (2016). Activation Mechanism of Oncogenic Deletion Mutations in BRAF, EGFR, and HER2. Cancer Cell, 29(4), 477-493. doi:10.1016/j.ccell.2016.02.010.
Freed et al. (2018). TNscope: Accurate Detection of Somatic Mutations with Haplotype-based Variant Candidate Detection and Machine Learning Filtering. bioRxiv, 250647. doi:10.1101/250647.
Gao et al. (2018). Allele-Specific Mechanisms of Activation of MEK1 Mutants Determine Their Properties. Cancer Discov, 8(5), 648-661. doi:10.1158/2159-8290.CD-17-1452.
Goor et al. (2011) A Mathematical Approach to the Analysis of Multiplex DNA Profiles. Bull Math Biol 73(8): 1909-1931.
Goor et al. (2021) Novel Method for Accurately Assessing Pull-up Artifacts in STR Analysis. FSI Genetics 51:102410.
Guillen et al. (2021). Molecular pathology in the cancer clinic—where are we now and where are we headed? J Small Anim Pract, 62(7), 507-520. doi:10.1111/jsap.13330.
Hanazono et al. (2015). Epidermal growth factor receptor expression in canine transitional cell carcinoma. J Vet Med Sci, 77(1), 1-6. doi:10.1292/jvms. 14-0032.
Harlow & Lane (1988) Antibodies: A Laboratory Manual, Cold Spring Harbor Laboratory Press, Cold Spring Harbor, New York, United States of America.
Heng et al. (2022) Ultrasonographic detection of apex nodules in the urinary bladder of Scottish Terriers. Vet Radiol Ultrasound 63(2):234-239.
Henikoff & Henikoff (1992) Amino Acid Substitution Matrices from Protein Blocks. Proc Natl Acad Sci USA 89:10915-10919.
Hoeppner et al. (2014). An improved canine genome and a comprehensive catalogue of coding genes and non-coding transcripts. PLoS One, 9(3), e91172. doi:10.1371/journal.pone.0091172.
Hunt et al. (2018). Ensembl variation resources. Database (Oxford), 2018. doi:10.1093/database/bay119.
Hurst et al. (2017). Genomic Subtypes of Non-invasive Bladder Cancer with Distinct Metabolic Profile and Female Gender Bias in KDM6A Mutation Frequency. Cancer Cell, 32(5), 701-715 e707. doi:10.1016/j.ccell.2017.08.005.
Jouenne et al. (2020). Genetic landscape of adult Langerhans cell histiocytosis with lung involvement. Eur Respir J, 55(2). doi:10.1183/13993003.01190-2019.
Karlin & Altschul (1993) Applications and statistics for multiple high-scoring segments in molecular sequences. Proc Natl Acad Sci USA 90(12):5873-5877.
Kim (2020). PIK3CA mutations matter for cancer in dogs. Res Vet Sci, 133, 39-41. doi:10.1016/j.rvsc.2020.09.001
Knapp et al. (2014). Urinary bladder cancer in dogs, a naturally occurring model for cancer biology and drug development. ILAR J, 55(1), 100-118. doi:10.1093/ilar/ilu018.
Knapp et al. (2019). Naturally-Occurring Invasive Urothelial Carcinoma in Dogs, a Unique Model to Drive Advances in Managing Muscle Invasive Bladder Cancer in Humans. Front Oncol, 9, 1493. doi:10.3389/fonc.2019.01493.
Knowles & Hurst (2015). Molecular biology of bladder cancer: new insights into pathogenesis and clinical diversity. Nat Rev Cancer, 15(1), 25-41. doi:10.1038/nrc3817.
Langbein et al. (2002). Alteration of the LRP1B gene region is associated with high grade of urothelial cancer. Lab Invest, 82(5), 639-643. doi:10.1038/labinvest.3780458.
Lee et al. (2017). Real-time genomic profiling of histiocytoses identifies early-kinase domain BRAF alterations while improving treatment outcomes. JCI Insight, 2(3), e89473. doi:10.1172/jci.insight.89473.
Lindblad-Toh et al. (2005). Genome sequence, comparative analysis and haplotype structure of the domestic dog. Nature, 438(7069), 803-819. doi:10.1038/nature04338.
Lindner et al. (2021). Lynch Syndrome: Its Impact on Urothelial Carcinoma. Int J Mol Sci, 22(2). doi:10.3390/ijms22020531.
Liow & Tran (2020). Precision oncology in urothelial cancer. ESMO Open, 5(Suppl 1), e000616. doi:10.1136/esmoopen-2019-000616.
Liu et al. (2021). Association of RYR2 Mutation With Tumor Mutation Burden, Prognosis, and Antitumor Immunity in Patients With Esophageal Adenocarcinoma. Front Genet, 12, 669694. doi:10.3389/fgene.2021.669694.
Longo et al. (2016). Targeted Exome Sequencing of the Cancer Genome in Patients with Very High-risk Bladder Cancer. Eur Urol, 70(5), 714-717. doi:10.1016/j.eururo.2016.07.049.
Maeda et al. (2018). Comprehensive gene expression analysis of canine invasive urothelial bladder carcinoma by RNA-Seq. BMC Cancer, 18(1), 472. doi:10.1186/s12885-018-4409-3.
McGinnis et al. (2018). Frequency of MAP2K1, TP53, and U2AF1 Mutations in BRAF-mutated Langerhans Cell Histiocytosis: Further Characterizing the Genomic Landscape of LCH. Am J Surg Pathol, 42(7), 885-890. doi:10.1097/PAS.0000000000001057.
Megquier et al. (2019). Comparative Genomics Reveals Shared Mutational Landscape in Canine Hemangiosarcoma and Human Angiosarcoma. Mol Cancer Res, 17(12), 2410-2421. doi:10.1158/1541-7786.MCR-19-0221.
Millanta et al. (2018). Overexpression of HER-2 via immunohistochemistry in canine urinary bladder transitional cell carcinoma—A marker of malignancy and possible therapeutic target. Vet Comp Oncol, 16(2), 297-300. doi:10.1111/vco. 12345.
Mizuno et al. (2023) High-Throughput Functional Evaluation of MAP2K1 Variants in Cancer. Mol Cancer Ther 22(2):227-239.
Mochizuki & Breen (2017). Sequence analysis of RAS and RAF mutation hot spots in canine carcinoma. Vet Comp Oncol, 15(4), 1598-1605. doi:10.1111/vco.12275.
Mochizuki et al. (2015). Detection of BRAF Mutation in Urine DNA as a Molecular Diagnostic for Canine Urothelial and Prostatic Carcinoma. PLoS One, 10(12), e0144170. doi:10.1371/journal.pone.0144170.
Mochizuki et al. (2016). Detection of Copy Number Imbalance in Canine Urothelial Carcinoma With Droplet Digital Polymerase Chain Reaction. Vet Pathol, 53(4), 764-772. doi:10.1177/0300985815614975.
Nann et al. (2019) Pediatric Langerhans cell histiocytosis: the impact of mutational profile on clinical progression and late sequelae. Ann Hematol 98(7):1617-1626.
Needleman & Wunsch (1970) A general method applicable to the search for similarities in the amino acid sequence of two proteins. J Mol Biol 48:443-453.
Nelson et al. (2015). MAP2K1 and MAP3K1 mutations in Langerhans cell histiocytosis. Genes Chromosomes Cancer, 54(6), 361-368. doi:10.1002/gcc.22247.
Niu et al. (2019). Resistance mechanism of the oncogenic beta3-alphaC deletion mutation in BRAF kinase to dabrafenib and vemurafenib revealed by molecular dynamics simulations and binding free energy calculations. Chem Biol Drug Des, 93(2), 177-187. doi:10.1111/cbdd. 13399.
Ning et al. (2020). A Three Protein-Coding Gene Prognostic Model Predicts Overall Survival in Bladder Cancer Patients. Biomed Res Int, 2020, 7272960. doi:10.1155/2020/7272960.
Nolan et al. (2021). Hidden Targets in RAF Signalling Pathways to Block Oncogenic RAS Signalling. Genes (Basel), 12(4). doi:10.3390/genes12040553.
Ohtsuka et al. (1985) An alternative approach to deoxyoligonucleotides as hybridization probes by insertion of deoxyinosine at ambiguous codon positions. J Biol Chem 260:2605-2608.
Parker et al. (2020). RNAseq expression patterns of canine invasive urothelial carcinoma reveal two distinct tumor clusters and shared regions of dysregulation with human bladder tumors. BMC Cancer, 20(1), 251. doi:10.1186/s12885-020-06737-0.
Patel et al. (2021). Whole-Exome Sequencing Analysis of Oral Squamous Cell Carcinoma Delineated by Tobacco Usage Habits. Front Oncol, 11, 660696 doi:10.3389/fonc.2021.660696.
Paziewska et al. (2020). Mutational Mosaics of Cell-Free DNA from Pancreatic Cyst Fluids. Dig Dis Sci, 65(8), 2294-2301. doi:10.1007/s10620-019-06043-1.
Pearson & Lipman (1988) Improved tools for biological sequence comparison. Proc Natl Acad Sci USA 85:2444-2448.
Pei et al. (2021). Benchmarking variant callers in next-generation and third-generation sequencing analysis. Brief Bioinform, 22(3). doi:10.1093/bib/bbaa148.
Pietzak et al. (2017). Next-generation Sequencing of Nonmuscle Invasive Bladder Cancer Reveals Potential Biomarkers and Rational Therapeutic Targets. Eur Urol, 72(6), 952-959. doi:10.1016/j.eururo.2017.05.032.
Plassais et al. (2019). Whole genome sequencing of canids reveals genomic regions under selection and variants influencing morphology. Nat Commun, 10(1), 1489. doi:10.1038/s41467-019-09373-w.
Principe et al. (2021). LRP1B: A Giant Lost in Cancer Translation. Pharmaceuticals (Basel), 14(9). doi:10.3390/ph14090836.
Ramsey et al. (2017). Cross-species analysis of the canine and human bladder cancer transcriptome and exome. Genes Chromosomes Cancer, 56(4), 328-343. doi:10.1002/gcc.22441.
Reiter et al. (1992) Genetic Linkage of the Arabidopsis Genome: Methods for Mapping with Recombinant Inbreds and Random Amplified Polymorphic DNAs (RAPDs) in Methods in Arabidopsis Research, World Scientific Press, River Edge, New Jersey, United States of America.
Ren et al. (2021). Activation of the RAS pathway through uncommon BRAF mutations in mucinous pancreatic cysts without KRAS mutation. Mod Pathol, 34(2), 438-444. doi:10.1038/s41379-020-00647-z.
Richart et al. (2021). STAG2 loss-of-function affects short-range genomic contacts and modulates the basal-luminal transcriptional program of bladder cancer cells. Nucleic Acids Res, 49(19), 11005-11021. doi:10.1093/nar/gkab864.
Robertson et al. (2018). Comprehensive Molecular Characterization of Muscle-Invasive Bladder Cancer. Cell, 174(4), 1033. doi:10.1016/j.cell.2018.07.036.
Rossman et al. (2021). Phase I/II trial of vemurafenib in dogs with naturally-occurring, BRAF-mutated urothelial carcinoma. Mol Cancer Ther. doi:10.1158/1535-7163.MCT-20-0893.
Rossolini et al. (1994) Use of deoxyinosine-containing primers vs degenerate primers for polymerase chain reaction based on ambiguous sequence information. Mol Cell Probes 8:91-98
Ruff et al. (2020). Roles for MDC1 in cancer development and treatment. DNA Repair (Amst), 95, 102948. doi:10.1016/j.dnarep.2020.102948.
Sakai et al. (2020). ErbB2 Copy Number Aberration in Canine Urothelial Carcinoma Detected by a Digital Polymerase Chain Reaction Assay. Vet Pathol, 57(1), 56-65. doi:10.1177/0300985819879445.
Sambrook & Russell (2001) Molecular Cloning: A Laboratory Manual, 3rd ed. Cold Spring Harbor Laboratory Press, Cold Spring Harbor, New York, United States of America.
Schultz et al. (1998)—DNA tagging in Arabidopsis thaliana: Cloning by gene disruption, in Plant Molecular Biology Manual, 2^{nd} edition, Gelvin et al. (eds.) Kluwer Academic Publishers, New York, New York, United States of America.
Shapiro et al. (2015a). A cultured approach to canine urothelial carcinoma: molecular characterization of five cell lines. Canine Genet Epidemiol, 2, 15. doi:10.1186/s40575-015-0028-3.
Shapiro et al. (2015b). Canine urothelial carcinoma: genomically aberrant and comparatively relevant. Chromosome Res, 23(2), 311-331. doi:10.1007/s10577-015-9471-y.
Sherry et al. (2001). dbSNP: the NCBI database of genetic variation. Nucleic Acids Res, 29(1), 308-311. doi:10.1093/nar/29.1.308.
Silhavy et al. (1984) Experiments with Gene Fusions, Cold Spring Harbor Laboratory, Cold Spring Harbor, New York, United States of America.
Sjodahl et al. (2011). A systematic study of gene mutations in urothelial carcinoma; inactivating mutations in TSC2 and PIK3R1. PLoS One, 6(4), e18583. doi:10.1371/journal.pone.0018583.
Smith & Waterman (1981) Identification of common molecular subsequences. Adv Appl Math 2:482
Sondka et al. (2018). The COSMIC Cancer Gene Census: describing genetic dysfunction across all human cancers. Nat Rev Cancer, 18(11), 696-705. doi:10.1038/s41568-018-0060-1.
Southern et al. (1991) Identification of an epitope on the P and V proteins of simian virus 5 that distinguishes between two isolates with different biological characteristics. J Gen Virol 72:1551-1557.
Tate et al. (2019). COSMIC: the Catalogue Of Somatic Mutations In Cancer. Nucleic Acids Res, 47(D1), D941-D947. doi:10.1093/nar/gky1015.
Tijssen (1993) Laboratory Techniques in Biochemistry and Molecular Biology-Hybridization with Nucleic Acid Probes. Elsevier, New York, New York, United States of America.
Tsuboi et al. (2019). Assessment of HER2 Expression in Canine Urothelial Carcinoma of the Urinary Bladder. Vet Pathol, 56(3), 369-376. doi:10.1177/0300985818817024.
Van der Auwera et al. (2013). From FastQ data to high confidence variant calls: the Genome Analysis Toolkit best practices pipeline. Curr Protoc Bioinformatics, 43, 11 10 11-33. doi:10.1002/0471250953.bi1110s43.
Wang et al. (2021). Association of CSMD1 with Tumor Mutation Burden and Other Clinical Outcomes in Gastric Cancer. Int J Gen Med, 14, 8293-8299. doi:10.2147/IJGM.S325910.
Wiley et al. (2019). Novel Noninvasive Diagnostics. Vet Clin North Am Small Anim Pract, 49(5), 781-791. doi:10.1016/j.cvsm.2019.05.002.
Williams et al. (2020). Melanoma with in-frame deletion of MAP2K1: a distinct molecular subtype of cutaneous melanoma mutually exclusive from BRAF, NRAS, and NF1 mutations. Mod Pathol, 33(12), 2397-2406. doi:10.1038/s41379-020-0581-5.
Wise (2020) One Health Approach to Environmental Health and Genomics of Sporadic Disease Models for Urogenital Cancers. Doctoral Dissertation, North Carolina State University, Raleigh, North Carolina, United States of America. pp. 213-216.
Wrzeszczynski et al. (2019). Identification of targetable BRAF DeltaN486_P490 variant by whole-genome sequencing leading to dabrafenib-induced remission of a BRAF-mutant pancreatic adenocarcinoma. Cold Spring Harb Mol Case Stud, 5(6). doi:10.1101/mcs.a004424
Xie et al. (2021). Titin Mutation Is Associated With Tumor Mutation Burden and Promotes Antitumor Immunity in Lung Squamous Cell Carcinoma. Front Cell Dev Biol, 9, 761758. doi:10.3389/fcell.2021.761758.
Yaeger & Corcoran (2019) Targeting Alterations in the RAF-MEK Pathway. Cancer Discov 9(3):329-341.
Yi et al. (2020). ATM Mutations Benefit Bladder Cancer Patients Treated With Immune Checkpoint Inhibitors by Acting on the Tumor Immune Microenvironment. Front Genet, 11, 933. doi:10.3389/fgene.2020.00933.
Yin et al. (2018). ATM/RB1 mutations predict shorter overall survival in urothelial cancer. Oncotarget, 9(24), 16891-16898. doi:10.18632/oncotarget.24738.
Yuan et al. (2018). Activating mutations in MEK1 enhance homodimerization and promote tumorigenesis. Sci Signal, 11(554). doi:10.1126/scisignal.aar6795.
Zehir et al. (2017). Mutational landscape of metastatic cancer revealed from prospective clinical sequencing of 10,000 patients. Nat Med, 23(6), 703-713. doi:10.1038/nm.4333.

It will be understood that various details of the presently disclosed subject matter can be changed without departing from the scope of the presently disclosed subject matter. Furthermore, the foregoing description is for the purpose of illustration only, and not for the purpose of limitation.

TABLE 1
Signalment Data and ddPCR Assay Values for the Sample Cohort

| | | | Age at | BRAF V595E | BRAF V595E | | chr36/ | chr13/ |
|---|---|---|---|---|---|---|---|---|
| | | | time of | variant allele | fractional | ddPCR | chr19 | chr19 |
| | | | sample | frequency (%) | abundance | detection | copy | copy |
| Sample | | | submission | from WES | (%) from | threshold | number | number |
| code | Sex | Stated breed | (years) | analysis | ddPCR analysis | (%) | ratio | ratio |
| UD-001 | FS | Border Terrier | 11 | undetected | undetected | 0.04 | 2.95 | 1.64 |
| UD-003 | FS | Beagle | 12 | undetected | undetected | 0.04 | 1.69 | 2.18 |
| UD-007 | FS | American Staffordshire Terrier | 14 | undetected | undetected | 0.05 | 1.83 | 1.84 |
| UD-018 | FS | Labrador mix | 12 | undetected | undetected | 0.04 | 1.80 | 1.80 |
| UD-027 | FS | Rat Terrier | 14 | undetected | undetected | 0.03 | 2.05 | 1.54 |
| UD-033 | FS | Toy Poodle | 12 | undetected | undetected | 0.04 | 1.51 | 2.11 |
| UD-049 | FS | Scottish Terrier | 13 | undetected | undetected | 0.04 | 1.94 | 1.71 |
| UD-054 | FS | Beagle | 8 | undetected | undetected | 0.04 | 1.71 | 1.95 |
| UD-081 | FS | Miniature Dachshund | 9 | undetected | undetected | 0.08 | 2.31 | 1.68 |
| UD-082 | FS | Boston Terrier | 13 | undetected | undetected | 0.04 | 2.26 | 1.71 |
| UD-084 | FS | Mix | 10 | undetected | undetected | 0.05 | 1.70 | 3.10 |
| UD-085 | MN | Labrador Retriever | 13 | undetected | undetected | 0.06 | 2.09 | 1.79 |
| UD-088 | MN | Fox Terrier | 12 | undetected | undetected | 0.08 | 1.96 | 2.12 |
| UD-091 | FS | Great Dane | 8 | undetected | undetected | 0.03 | 3.57 | 3.30 |
| UD-092 | MN | Terrier mix | 11 | undetected | undetected | 0.03 | 3.60 | 2.52 |
| UD-097 | FS | Beagle | 11 | undetected | undetected | 0.04 | 2.36 | 2.91 |
| UD-098 | FS | Boston Terrier | 9 | undetected | undetected | 0.04 | 7.27 | 1.88 |
| UD-099 | FS | Beagle | 8 | undetected | undetected | 0.04 | 3.15 | 1.88 |
| UD-100 | FS | Beagle/Terrier mix | 13 | undetected | undetected | 0.05 | 2.12 | 2.07 |
| UD-102 | MN | German Shepherd mix | 14 | undetected | undetected | 0.05 | 7.00 | 1.90 |
| UD-104 | FI | Bluetick Coonhound | 13 | undetected | undetected | 0.05 | 3.23 | 3.46 |
| UD-105 | MN | Beagle | 13 | undetected | undetected | 0.06 | 2.11 | 2.12 |
| UD-106 | FS | Golden Retriever/Collie mix | 11 | undetected | undetected | 0.06 | 3.74 | 1.60 |
| UD-108 | MN | Bassett Hound | 11 | undetected | undetected | 0.04 | 3.24 | 2.19 |
| UD-109 | FS | Beagle | 12 | undetected | undetected | 0.05 | 2.62 | 2.38 |
| UD-110 | FS | Collie | 12 | undetected | undetected | 0.06 | 3.96 | 3.03 |
| UD-112 | FS | Pomeranian | 14 | undetected | undetected | 0.05 | 5.51 | 1.69 |
| UD-113 | FS | Beagle | 13 | undetected | undetected | 0.06 | 3.58 | 2.45 |
| POS-124 | FS | Coton de Tulear | 13 | 61.7 | 61.7 | 0.05 | 1.73 | 2.29 |
| POS-125 | FS | Labrador mix | 9 | 65.1 | 60.2 | 0.11 | 2.90 | 1.82 |
| POS-127 | FS | Labrador/poodle mix | 8 | 55.8 | 58.5 | 0.02 | 1.66 | 1.82 |
| POS-128 | FS | Miniature Schnauzer | 10 | 55.3 | 55.4 | 0.02 | 2.41 | 1.63 |
| POS 131 | not known | Labrador Retriever | 12 | 49.5 | 53.0 | 0.03 | 2.52 | 1.63 |
| POS-134 | FS | Australian Cattle Dog | 8 | 44.0 | 51.7 | 0.02 | 5.50 | 2.27 |
| POS-138 | FS | Mix | 14 | 48.1 | 50.0 | 0.05 | 8.23 | 1.92 |
| POS-2027 | MN | Yorkshire Terrier/Shih Tzu mix | 11 | 90.1 | 92.0 | 0.05 | 3.06 | 3.72 |

TABLE 2
Exemplary Primer Sequences and Predicted Amplicon Sizes

| | | | | | Wild | Observed |
|---|---|---|---|---|---|---|
| | | | | | Type | Mutant |
| | Fluorophore | | | Amplicon | Amplicon | Amplicon |
| Target | (5′) | Forward Primer (5′-3′) | Reverse Primer (5′-3′) | Location* | Size | Size(s) |
| BRAF | 6FAM | AAATTAATGCAGTTTAGGAAGTTAAGATATCATT | TAAAGCCACCAGCAGCTATCA | chr16: | 251 | 236, 242 |
| exon 12 | | SEQ ID NO: 15 | SEQ ID NO: 16 | 8276587-8276837 | | |
| MAP2K1 | VIC | TGGGACAGGACCAACCT | GAGTGGCCTCACCTTTCTG | chr30: | 231 | 216 |
| exon 2 | | SEQ ID NO: 17 | SEQ ID NO: 18 | 30720093-30720323 | | |
| MAP2K1 | NED | TCTCTTCCTCCCACCTTCTT | GCTGACAGGTTGCCACATA | chr30: | 194 | 188 |
| exon 3 | | SEQ ID NO: 19 | SEQ ID NO: 20 | 30720609-30721802 | | |
| BRAF | PET | CAGGAAACAGCTATGACAATAGGTGATTTTGGTCT | GTCACTCAGTAGCACCTCAGGG | chr16: | 168 | N/A |
| V595 | | AGCCACAGT | SEQ ID NO: 22 | 8296258-8296408 | | |
| (WT) | | SEQ ID NO: 21 | | | | |
| BRAF | PET | AATAGGTGATTTTGGTCTAGCCAgAGA | GTCACTCAGTAGCACCTCAGGG | chr16: | N/A | 151 |
| V595 | | SEQ ID NO: 23 | SEQ ID NO: 24 | 8296258-8296408 | | |
| (mutant) | | | | | | |

*Based on the canFam3 reference genome

TABLE 3
Exemplary Primers for MAP2K1 Analysis

| Reagent | Fluorophore (5′) | Sequence (5′-3′) |
|---|---|---|
| Forward primer | none | GGAGGAGCTGGAGCTTGATG (SEQ ID NO: 25) |
| Reverse primer | none | CACTGATCTTCTCGAAGTCGTCAT (SEQ ID NO: 26) |
| Wild-type control probe | VIC | CCTTTCTCACCCAGAAG (SEQ ID NO: 27) |
| Q56P mutant probe | FAM | TTTCTCACCCCGAAGC (SEQ ID NO: 28) |
| K57E mutant probe | FAM | CCTTTCTCACCCAGGAG (SEQ ID NO: 29) |

1. A method for detecting a urogenital malignancy in a dog, the method comprising identifying a deletion or nucleotide substitution within exon 12 of BRAF gene and/or in exon 2 or 3 of a MAP2K1 gene present in or isolated from a biological sample from the dog, wherein the presence of the deletion or nucleotide substitution within exon 12 of the BRAF gene and/or within exon 2 or 3 of the MAP2K1 gene detects the urogenital malignancy in the dog.

2. The method of claim 1, wherein the urogenital malignancy is transitional cell carcinoma/urothelial carcinoma (TCC/UC).

3. The method of claim 1, wherein the deletion within exon 12 of the BRAF gene results in a deletion of one or more of the amino acids present within the amino acid sequence KMLNVTAPTPQQL (SEQ ID NO: 3) of a BRAF polypeptide encoded by the BRAF gene.

4. The method of claim 3, wherein the deletion within exon 12 of the BRAF gene results in a deletion of one or more amino acids selected from the group consisting of NVTAP (SEQ ID NO: 9), LNVT (SEQ ID NO: 10), LNVTAP (SEQ ID NO: 11), NVTAPT (SEQ ID NO: 12), and TAPT (SEQ ID NO: 13), optionally wherein the deletion is accompanied by an insertion of one or more, optionally one, amino acid.

5. The method of claim 4, wherein the deletion and accompanying insertion is selected from the group consisting of a deletion of LNVT (SEQ ID NO: 10) and an insertion of an F, a deletion of LNVTAP (SEQ ID NO: 11) and insertion of an F, and a deletion of NVTAPT (SEQ ID NO: 12) and an insertion of a K.

6. The method of claim 1, wherein the deletion within exon 2 of the MAP2K1 gene results in a deletion of one or more of the amino acids present within the amino acid sequence FLTQKQKVGE (SEQ ID NO: 4) of a MAP2K1 polypeptide encoded by the MAP2K1 gene.

7. The method of claim 6, wherein the deletion within exon 2 of the MAP2K1 gene results in a deletion of the amino acids FLTQKQ (SEQ ID NO: 14), optionally wherein the deletion is accompanied by an insertion of one or more, optionally one, amino acid.

8. The method of claim 7, wherein the insertion is an insertion of an L

9. The method of claim 1, wherein the nucleotide substitution results in exon 2 of MAP2K1 results in a Q56P substitution, a K57E substitution, a K57N substitution, or any combination thereof.

10. The method of claim 1, wherein the deletion within exon 3 of the MAP2K1 gene results in a deletion of P105 and/or A106 of a MAP2K1 polypeptide encoded by the MAP2K1 gene.

11. The method of claim 10, wherein the deletion within exon 3 of the MAP2K1 gene results in a deletion of P105 and A106 of a MAP2K1 polypeptide encoded by the MAP2K1 gene.

12. A method for differentially treating a dog with a urogenital malignancy, the method comprising:
(a) identifying whether the dog has a V595E mutation in a BRAF gene or a deletion within exon 12 of the BRAF gene; and
(b1) if the dog has a V595E mutation in a BRAF gene, administering a first-generation BRAF inhibitor, optionally vemurafenib and/or dabrafenib; or
(b2) if the dog has a deletion within exon 12 of the BRAF gene, a nucleotide substation within exon 12 of the BRAF gene, or any combination thereof, administering an MEK inhibitor, optionally trametinib, optionally in combination with a broad-acting BRAF inhibitor, optionally sorafenib and/or AZ628.

13. The method of claim 1, wherein the identifying comprises assaying nucleic acids present in or isolated from a biological sample isolated from the dog, wherein the biological sample is a urine sample, cells isolated from the urinary tract of the dog, a biopsy specimen, or a combination thereof.

14. The method of claim 13, wherein the identifying step employs a method selected from the group consisting of ddPCR, Sanger sequencing, next generation sequencing, capillary electrophoresis, or any combination thereof.

15. (canceled)

16. The method of claim 12, further comprising administering one or more additional therapies to the dog, optionally wherein the one or more additional therapies are selected from the group consisting of surgery, radiation therapy, and an additional chemotherapy.

17. A method for simultaneously identifying the presence or absence of a BRAF V595E mutation in a canine or a BRAF V600E mutation in a human and one or more of a deletion within exon 12 of the BRAF gene, a deletion or single nucleotide substitution in exon 2 of a MAP2K1 gene, and a deletion in exon 3 of a MAP2K1 gene present in or isolated from a biological sample from a canine or a human, the method comprising:
(a) obtaining a biological sample from the canine or the human, wherein biological sample from the canine or the human comprises DNA sequences that comprise the BRAF V595E or BRAF V600E coding sequence, and one or more of exon 12 of the BRAF gene, exon 2 of the MAP2K1 gene, and exon 3 of the MAP2K1 gene;
(b) amplifying a region of each of the DNA sequences that comprise the BRAF V595E or BRAF V600E coding sequence, and one or more of exon 12 of the BRAF gene, exon 2 of the MAP2K1 gene, and exon 3 of the MAP2K1 gene from the genomic DNA with a plurality of primers, wherein the plurality of primers comprises:
(i) a first set of primers that together flank a first region of interest that comprise the BRAF V595E or BRAF V600E coding sequence, wherein the first set of primers comprises at least two primers that bind to the same strand of the genomic DNA, differ in sequence with respect to at least one nucleotide and are designed to detect the presence of a nucleotide substitution present in the first region of interest and a third primer that binds to the opposite strand of the genomic DNA to identify the presence or absence of the BRAF V595E or BRAF V600E mutation; and
(ii) one or more additional sets of primers that flank additional regions of interest in exon 12 of the BRAF gene, exon 2 of the MAP2K1 gene, exon 3 of the MAP2K1 gene, or any combination thereof, wherein the one or more additional sets of primers are designed to amplify regions of interest in exon 12 of the BRAF gene, exon 2 of the MAP2K1 gene, exon 3 of the MAP2K1 gene, or any combination thereof, and further wherein the first set of primers and each of the one or more additional sets of primers are designed to amplify fragments that can be detectably distinguished from each other; and
(c) detecting a difference in the amplified fragments,
whereby the presence or absence of a BRAF V595E or BRAF V600E mutation and one or more of a deletion within exon 12 of the BRAF gene, a deletion or single nucleotide substitution in exon 2 of a MAP2K1 gene, and a deletion in exon 3 of a MAP2K1 gene present in or isolated from a biological sample from the canine or the human is identified.

18. The method of claim 17, wherein the least two primers that bind to the same strand of the genomic DNA to amplify the subsequence of the BRAF gene that includes the V595E/V600E mutation are allele-specific primers and are also of different sizes such that the amplification product that results from a BRAF V595/V600 allele and the amplification product that results from a BRAF E595/E600 allele are of different sizes.

19. The method of claim 17, wherein the amplification products that result from amplification of exon 12 of the BRAF gene, exon 2 of the MAP2K1 gene, and exon 3 of the MAP2K1 gene are also of different sizes from each other and also of the amplification products that result from a BRAF V595/V600 allele and from a BRAF E595/E600 allele.

20. The method of claim 18, further comprising separating the amplification products that correspond to the BRAF V595E/V600E alleles, exon 12 of the BRAF gene, exon 2 of the MAP2K1 gene, and exon 3 of the MAP2K1 gene simultaneously in a single capillary electrophoresis such that the single capillary electrophoresis permits detection of the presence or absence of each of a BRAF V595/V600 allele, a BRAF E595/E600 allele, a wild type exon 12 of the BRAF gene, any indel within exon 12 of the BRAF gene, a wild type exon 2 of the MAP2K1 gene, any indel or single nucleotide substitution within exon 2 of the MAP2K1 gene, a wild type exon 3 of the MAP2K1 gene, and any indel within exon 3 of the MAP2K1 gene due to each of the BRAF V595/V600 allele, the BRAF E595/E600 allele, the wild type exon 12 of the BRAF gene, an indel within exon 12 of the BRAF gene, the wild type exon 2 of the MAP2K1 gene, an indel or single nucleotide substitution within exon 2 of the MAP2K1 gene, the wild type exon 3 of the MAP2K1 gene, and an indel within exon 3 of the MAP2K1 gene generating amplification products of that all differ from each other.

21. The method of claim 17, wherein;
(i) the deletion within exon 12 of the BRAF gene results in a deletion of one or more of the amino acids present within the amino acid sequence KMLNVTAPTPQQL (SEQ ID NO: 3) of a BRAF polypeptide encoded by the BRAF gene, optionally wherein the deletion within exon 12 of the BRAF gene results in a deletion of one or more amino acids selected from the group consisting of NVTAP (SEQ ID NO: 9), LNVT (SEQ ID NO: 10), LNVTAP (SEQ ID NO: 11), NVTAPT (SEQ ID NO: 12), and TAPT (SEQ ID NO: 13), optionally wherein the deletion is accompanied by an insertion of one or more amino acids, further optionally wherein the deletion and accompanying insertion is selected from the group consisting of a deletion of LNVT (SEQ ID NO: 10) and an insert of an F, a deletion of LNVTAP (SEQ ID NO: 11) and insertion of an F, and a deletion of NVTAPT (SEQ ID NO: 12) and an insertion of a K; and/or
(ii) the deletion within exon 2 of the MAP2K1 gene results in a deletion of one or more of the amino acids present within the amino acid sequence FLTQKQKVGE (SEQ ID NO: 4) of a MAP2K1 polypeptide encoded by the MAP2K1 gene; and/or
(iii) the deletion within exon 2 of the MAP2K1 gene results in a deletion of the amino acids FLTQKQ (SEQ ID NO: 14), optionally wherein the deletion is accompanied by an insertion of one or more amino acids, optionally wherein the insertion is an insertion of an L; and/or the nucleotide substitution results in exon 2 of MAP2K1 results in a Q56P substitution, a K57E substitution, a K57N substitution, or any combination thereof; and/or
(iv) the deletion within exon 3 of the MAP2K1 gene results in a deletion of P105 and/or A106 of a MAP2K1 polypeptide encoded by the MAP2K1 gene.

22-29. (canceled)


SEQUENCE LISTING (51 sequences)

SEQ ID NO: 1 - DNA, 39 bp, Canis lupus
  source 1..39: mol_type = other DNA; sub_species = familiaris
aaaatgttgaatgtgacagcacccacacctcagcagtta

SEQ ID NO: 2 - DNA, 39 bp, Homo sapiens
  source 1..39: mol_type = other DNA
aaaatgttgaatgtgacagcacctacacctcagcagtta

SEQ ID NO: 3 - protein, 13 aa, Canis lupus
  source 1..13: note = sub_species familiaris
KMLNVTAPTPQQL

SEQ ID NO: 4 - protein, 10 aa, Canis lupus
  source 1..10: note = sub_species familiaris
FLTQKQKVGE

SEQ ID NO: 5 - protein, 761 aa, Canis lupus
  source 1..761: note = sub_species familiaris
VWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDISRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGAPLNHLMRCLRKCQSRTPSPLLHSVPSEIVFDFEPGPVFRGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 6 - protein, 393 aa, Canis lupus
  source 1..393: note = sub_species familiaris
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDVAETPPRPRTPGRPLSSYGMDSRPPMAIFELLDYIVNEPPPKLPSGVFSLEFQDFVNKCLIKNPAERADLKQLMVHAFIKRSDGEEVDFAGWLCSTIGLNQPSTPTHAAGV

SEQ ID NO: 7 - DNA, 95271 bp, Canis lupus
  source 1..95271: mol_type = genomic DNA; sub_species = familiaris
gtgtggaatatcaaacaaatgattaagttgacacaggaacatatagaagccctattggacaagtttggtggggagcataatccaccatcaatatatctggaggtaagcttttgagtatcatatctgttataattttgagaataaaaacatgaatgtatcatgagcatttgtactggtaatattgtttctcttagaggatgagatgagaaaggggaaggtgaattacatttgtctttattatacttcttaattttgtttcacttgttacagtaatatgtggtattttgtaagttggaaaatatccagaataaaggaaaagaggtgtataaacttaaaaatactactctgataaaaaacttattttaaaaagagagagagggaaaacatttagttgtggaggttaggaaactagttaccatattaggatatggggtgaaatttataaatatcttttttccagaacaacccccaccccccagattggtttttgctttcttaataataatttataatacttaaatagctcagggaagtctgggtggctcagtggttgagcatctgcctttggctcagggcagtgatcccagggccctgggattgaatcccccatcgcacctccccccaccccggtggggagcctgcttctccctctgctatgtctctgcctctttctctgtgtctctcatgaataaataaataaaatctttaataaataaatagttcaaagttgcttagctgtactgttttaatagtgcctttaggcttttgtatcaagccttgagcatcattgtaaaatttagagagtagttagtaattgttcttaatactatgacgtagtagttactactggcatcagtttcctcatgtaagtcactcaggttccatgatgaacatgccagtaggaaatctagtagtcgggcttcacagcctatgctgctcaccactgcatatagtgcctgtctctgtaaaagctagcttcagctgtaatcactaatcatacaggaatctagccctaaattacaaatgggcaggttaccatttgcttccaaatgccagttttttaggcatgtattttgatagactcctaaactaaatttctaaattcgtaatctacttgcttttaaagggtacattaaatcttaaaagttaagtttgcatttctaaaataaatttggaagtttgataataaccattaataaactattgtcttcttcatatggcttatcccagagcatactgccagatatcagtactaggactcgaattcatacctctaaagactagttagtgtactttctaatattccttgctgatttacttttatgtctgaggagcgtaagtacaggagtcataaagaggcagagtcctggtgaaaatagtaattctttgttaattagaattagacaacttaaccagctgtttttctgccagccatttctttgtcttgggagattttcctcctgcttagttgtaatacatttacatcagtacctgtgttttgagtatagcaaagattcacttccccctccaaactcactctttcaggctttttactcttgaaattacatttggtgaggacaacatacagcaaaccatacctcatagattatatcatgagcatgtagagccttattctcagtgacaaacactacacaatttctctgtagaaggtgctataatatataagtgaccttggagttacacaggccctttgtttgaatcctagctgtgccttagtttatacagataggttacctaaccttcctgtacctcacctcattggtaaaattggagtactgtcatacatgtagtaatttagggtttacttaagtttataattgcaaaataacatcttcttgacatattgtgctccataattttttgattcattcattcaaatatttattgagtacttgtttatatttatgcttttctaggctttaggaatatagcagaaaaaccactttgtctttaagcaggttgtatttttatcaggggatgacattacaggcaataaacattgaacttacaccatttgatacactatgaagaggagcctgcctggagatgacagtatttttccctggggaaagttgaccagaggaaagacctttgggtactgatacttaaaggcccttcagtgagaaaccaagtctcctcattatccactgaaacccaccattctgctttctttctctaggagtctgattgcgctaggaacctcaaataaatggaattatacaacatttgtccttttgtgacaagcttatttagcataatgtcttcaaggtccatccatgttgtagtataggtcaaaatttccttgctaaataaggctaaataatattgcacattttgttatctgttcatctatcagtggatactagtggttgatcgtttctacctttctgttatcatgattaatgctgatattactccttttgactataaacccagaagtggacttactaaatcatgtggtaattctgtatttcattttttaaacaattgccatactgttttccatatcagcagtcttttacattcctattagcagtacacaggagctccctttactcctcatccctgctgacactttctgtgtgtgtttatataatagctatcctaatgagtgtgaaatggtatttcatggttttgatttgcatttccccatgactaatgttgagcatcttttcatgtgcttattggccattcctattctgtggagaaatgtctaagtcttacccattttttaattgggttgtttgttttgttgagttgtgggagttctttatgtacttcaagtatcagtcctaattagatacatgattacatgatttgcagatatttttttctattctacgggttcccttttcactctgttgattatatcctttgtgcaaaagtttttattttgatgatgtctagcttacttattttttcgtcttgttgcctgtgcttttggtatcatatcccagaaatcattaccagatctagtgtcatgaaactttttttctgttgccctctaagattcataattataattagcccttatattgaggtttctgatccattttgagttaatttctctgtatggtataaggtaaaggttcagcttcattcttttgcatgtatatatccagttttcccagccccatttgctgaaaagaccgtcctttcccattgtatagaaacttttgatatgtgcccttgtcaaaaatcatttgaccatgtatgtgagaatttgtttctgggctcttttcttttccactggtatttatgtccaactttatgccaatttatactatactgatttgattactgtagctttgtaataagttttggaatcaggaagtgtgagacctctaactttgttttacttttttttttttttaaagattttatttattcatgagagacagagagagagagaggcagagacacacaggcagagggagaaccaggctccatgcagggagcccgacacaggactcgattctgggactccaggatcatgccttgggctaaaggcagacgccaaactgctgagccacccagggatcccctttgttttcctttttaggattgttttggctattcagtatcccttgagatttcatattaatttcagggtacatttttctatttctgaaacactttgggattttgatggagattgaattcatagattgctttggatagtatttgacatcttaggagttcttaagtcttaaatataaatggatagctaagggacacctgagtggctccatggttgagcatctgcctttggcacaggtcatggtcctggggtcctgggatagagtcccgcatcacactccctttacagatcctgcttctccctctgcctgtgtctctgcctctctctatgtctctcatgaataagtaaaatcagatagatagatagatagatagatagatagatagatagatagatagataaagaataaccaaatattggaagaaagcttctaataggaatagtaaaaatggccaaaaggaactcagaagcagtgcagagaacagatgaaaacctgaaaatgactataatttacagagataagacagtccatttttgtttcacgactgtacaaaagtcttaatcgagcacaagatagagcttttggtattaaaataattaaaatagtaaaatagtaagtagaaaatgtaaatatatatgtgtatgtttgtctctacatataagtaagtgttgtaagatgaagaaatctctagaaaggcaaataagaacaaagatggaaaatataagaggaagatgagaaaaattagagaataaatctaggagatctataagaattacagctgtttttagctacaaaaaaacaaaataccaaaggagggaaaaaaaaaaaaaaaaaaaaaggacacctgggtggctcagctgttgagcgcctgcctttggcccagggcatcaggacatgatcctagggacccgggattgagtaccgcatcaggctccctgtagggagcctgcttttctctctgcctatgtctctgcttctctctctgtgtctctcatgaataaatgggtaaaatcttaaaaaaaaaaaaaaaaaaaggaaggaaggaaagaaaagaaaagaaaagaaaagaacacctaatcacaagttgcccaaacaagtaggaattttattttcctctcctataacaagaagtatggagtaaattattttaacattggtcctacagctcaaaggtgtcatatgctggggtggagctctgcaatccttttggcttttctgtcatgactgcagcagatctgtatgctttcataggacacctcagagcagcaacccgggatcagagaggctctcttgtgttcggagctctttcaaggaggaagtttttccctccaaatcccctgtaagccttttcctttacatctcatcagctggaatggagtcccatacatccctacaccagtactgtgtacatttcttgaaataagaggctaaaaagtggaagtttttccagtgttcacaatttggcttcagattagtttgagcactggcaagtagaagttatgctagttatttcattccataccattttaacatgagaaatagtgtttcctatatgtttgatgtttccacaggaggataaatcttaacctatcccagcatctcttccttgtaacagaacagagtgaattgagctacatcacgatcagaaatcctcttttctttttctccttgtactaatttaatttaaaaacttttaagtttatttaagcttttaagttgttctagattttctcctaccgacatgaataataattaaaattctgttgattcccttgatgataactatagtgaaatgagctatagtgctctaaagaatttcaaattaaaaaaaaaaagaatttcaaatttgtctttttaatatttggctccctttttaaattatcatgaggaaaagataagtcatttcccagaattttttgtttttctcccaattgttgtctcttcacttatccaacaaattatttgaatcccttctgtttataagatacaatgttagatgctcttctagtagtaaattctttatttggacctgatatatattgaattccagaagtaaagtaatagtgtttttgggagtcaccctgcttatctagtgttacatactgagtcttagcttccacagaatcaatcagtctatctgccttacctacctaccaacctaccagggttccctgcagacagatagatgaaaacagtagtaactcctaacatttatggaattcctgctatataccgggactgttccaagcatatagtagattctttagtctttaacctaccctaagagttacacttcttattttcattttataaagatgaggaaactcaggcgcatagcagttcaataactggctaaggctacacaattagtatatagccaagctctgaatctagacattctgaattcagagtctgctcttaacttctatatcattatgatgctgacagcctttttgatctttaatctgtgtagaatccaggataatgcagagaaattttattaaaatgaaagaatcctagaagcaaggtgcttcctgtggaatgcagagcagttcattaaatagcatttttgggtagtctgcaagatttcattgtatcttgtgagatgaaggttagtaagaataaatttaatgaactaatgataaaatttttttttattttacttctttttcctatttgtttaaagtagatccttagttttcctcccttttttttttttttttccaaatggtggtggcagggccactcattttgggaggggcggctccgtttctgatctttattttttttttatttttatttatttatgatagtcacagagagagagagaagcgcagagacacaggcagagggagaagcaggctccatgcaccgggagcccgatgtgggattcgatcccgggtctccaggatcgcgccctggggcaaaggcaggcgccaaaccgctgcgccacccagggatcccctttcctcccatttttaattgagaaatgcttcacataccataagattcacttttttgaagtataccattcagtggttttatttatttatttattttttaagattttatttattcatgagagatacagagagaaacagacacaggcagagggagaagcaggctctgtgcagcaagcccaatgtgggacttgatccagggaccccgggatcaggccctgagtcaaaggcagacactcaactgctgagccactcaggcatcccagttttttaatatgtgcacagagttgtgcagtcatcaccaattccatcactaattccaaaatattttcttttttaaaaaagatttatttatttattcatgagagacacagagacagagacatagtggaaggagaagcaggctccccgcaggaagcctgatgcaggactcaatcccaggaccccgggatcacaacctgagccaaaggcaaatgctcaaccactgagccacccagttgcccccaaaatatttttattaccccaaaaagagaccctttacccattagcagtcattcctttcagcccctggcaaccactaatctgctttatatttctgtagatttgccttttcggacagttcatgtaatggaatcatacatgatgtggcattttgtgacaggctgttttcatttaacatgttttcaaaattcatccatgttgtagcatgtagcattcattcttttttgttccattgtatgcatatacaaatttcgcttatccatttatcaattgttggatgtttgagttccctcttttttggctatcacaaataaagctattatgctatttattgctctttccttagtaaagtaaaaattggaggatgaaggaagggagccaaagaagcacagaagaggttagagtaattatagtgaaaaatgtaatatcatgatactgaaaaataagtaaaatttttcaggtagtagcgtgaaggactaatttccatgaattgatgatggaattaattggctttgtcttactaatttcttactaacctgaaagaaagaatggagtaattgaatgttaggatttgacaagcaatttggtaaaagaataagaatgagccagagaatcagagatgttattggtggcattgaaatggtgagccacaaagtaggggagttaagacagtgaaggctcagttctaagaaacttgaaaggattaatgttgttcatgcacagattaatgtagtgagatggaaacttaagaagatgtgctcacggggatccctgggtggctcagcagtttggcttctgcctttggcccagagcacggtcctggggtcctgggattgagtcccacatcgggctcccgccatggagcctgcttctccgtctgcctgtgtctctgcctctctctctctgtgtctatcatgaatagataagtaaatcttaaaaaaaaaaaaagaagaagaagatgtgctcagggacaggagaaattccaaattgagatcttctcatcatttggaactcctggatctggaaggtagagtgaaaaataaatactgtaagacataagagatttagagaacttcaaggcaggaatgttaaaatccacattgaatgcagtaaagataatggaaagaaatgagcccagccatgtacagaaggcagacaagtgacttgattgtggctgtgaggatggaggaaggtggaattaagtaaatgctttgtacttggggaaaagaagagctattgataaatgcagtaaaataacttgtaatccacaatggaggaaatagtatattttcaaaaattaacagtatattttcaaaaattcaaattaactaacattttgataactttctgtgaatttgtttacatgtattacttttcctaaggctaactttgtattgtaaatgtactagcctagatgatagtaagatttgatctatgcaggaggtaatttttatcttgtagactgcatgtttatgttctaccaagtgtcagtctttatttttgtagtttgagccccaaacagtgccaggcacgtgtgttttcagtgtatattaatttactgatttttttttgagaacgagagtatgagtgcagggggagaggacaggaagagaggaaatcttaagcaagctccatgctcggcagagccaaaggtagggtttaatctcaagactctgagatcatgacttgaacagaaatcaagagtggatacttaattgactgagccacctgggcatcccttaattgacagatttttaaaggctcaaaaagtggatcatattgaaagagatggccttagatcttggaactgaaagctaaatcaaaatacagcttagaaaattttggattgacttggttgtaatgccctgatgctatgaaatgtttctgggatcaggagggcagctagtaggctataaaggagaggcaattttgatatactttaggctcatatttacctcagttgctcagtttagtattttttctaagatctagcatagcaagtgcagtggtctttattaaaataaaatttctccttcattctcctcaacctttcataattctcccttgcttgtggaatggagttcagacttcatagcatgatatttacaccttctacaagctagtctctcacgtaccttaccagtcttattgtttccttgtattgcgatttttcatttaaatttcacaagcatatattgatcccttgctatgtcccaaatattgaatgggattcaattatattagtctgtcgttttccttctgtgccttgaaatttcctacctctttattttggggcagaattttcctgtacacagtactctcttctcccccacttaactccatctatctaaatgctaacattttaaggccctttccttcctcattgtcaccttgtatcaagaatgttctttgtctgtttctcatgtataacacttaaataaaaatacatctacctgcttctctgcagcccaatagtaagttctgagtgatgggaagtgccattttaaaatctttgattctctcttgtagccttttataaatgtttgtagttattgcatctatgtatggtggtagacagaaatggtgaactggggtgtgggggagagtgattaccactgccttaaagaaccatgagcatatatattccagttcattccatttctttactttcagtatcactctattttcaaaattctttacttctagaataggatatcttatgtggtcatttgacttcgtagctttagtcaaatgaaacttgtagaaaaaaaaactgatgggataatggcccccaaatttcttgactatagggtataaaccactttatcagataaaattttattttctaagcagtatttgataggatcatcagcgatccatagtgaagagaataaagtattttaactaatagctatttcagaaagaagatggagattatacatcttttaagttctattaagattttgttagtaatggtgagatctgacacagtacctgagtcttattttttgacaaatctcttttttaagacctcatctccttttcagcctgtctttctgttgttctttggaggggagaaaggtatgaaactttacatgcctgtaagttttctgaaaaaaataaaattttaatattgtacgtcccttgatgtagtctatagtatttttatcttatgtaagatatgcagtttagaaggtaatttgcaatatgaccagaacattctaaatattgcaattttaaattaacattcatggatattctggacatttatgttttgtcagtttctcagtgcagtgtagtatcttatcacacaagatttatcaattttttaaaaagcttttatttatttattcatgagagacacagagagagagagaggcagagacataggcagagggagaagcaggcttcatgcaggaagcctgatgtggaacttgatcccgggactccaggatcatgccctgagccaaagtcaggcattcaaccactgagctacccaggcatccgaaagatttatcatattaagagtagaagttaatttacaatacttgtagcatttattttgtaatttctaatttaaaaccttgttagctagattataaagagattataatcagacttaaatttcttatttccttttgttactgtcaggaaaaaaatccagatttaacgtaagtctactttattttataggcctatgaagaatacaccagcaaactagatgccctccaacagcgagaacaacagttattggaatccctggggaatggaactgatttttctgtttctagctctgcatcaacggacaccgttacatcttcttcctcttctagcctttcagtgctaccttcatctctttcagtttttcaaaatcccacagatatatcacggagcaatcccaagtcaccacaaaaacctatcgttagagtcttcctgcccaataaacagaggacggtggtgagtcaacttttaatatcatcattgctttttcgttgtgtttttcagaaagagtagttgttgaaactgaaatatgataccactccactctctgtggtcatgtaaatggaagcatccatttcaaccaggagtggaaaagacataattatttggtgccaatgtctgtatttcttaagtggaacaaattacattttgtttagtgtcagatttgacattttaactatcttccaaacttgtacttagactggagtgtggttctagtttagaatcaaaaagaatttcaagatttgagcttgtttgttgtctatcatatgtcaggtaagtttattttttgccctggagaaacccctatttggacttactatcaaatgttggatttttagcgtgtttgattttttttttctctctatatataacttttgttgatcattagtgttaccactgtaacagagtactctgtaagtaacatttaaagcagggaaatctgcacaattttatgggtggtctataagccttgagtgcaacaagtactgtcattggatttaataataatgcacctgtctacatcactactttaaaaaatgtatgtggttatttttgaaattaataaaaagccttttccaaattcaagtgaagtcaaaatagttttaattttttttccctcacacagcattagtagtaatgctggattttcatggaataagtttgacctgtgctgcagtggcctccagcaaggtcagtttgtacctttaaaacaaagggatgtaagtgtcagtgtgtggggatgtaagattaggtcttgagcctgtgtcacaaggtagttggatctcagatcctcacataaagctgagcctttgaagagatttaccctcagaaaaagggtgaactagaagaaaaatctatttactcagcatacataaataacaataaaacttacctgttttcctcagctgtccagataaaggttaaagtttcccttgaattcttcattatagacctgttctcatcatggtgtgggtggtgtccaggatctttcaatccaacaactggtcctggagaggcactaggcaggagcaaatgtaaaacctctttggcctgtctaggtctcgtggaatccacagagataatgcctattgataaactcaaaatcccatgttacaaaataaacaaggaaagagcttatgtgcattagaagacttaagaacaacaagactcctagaaggaaccaaatagaactgctaggtggtcctaaaattttaaactcagtggatgacagtatctataaagtatccttgctaaagatgatttgtgttgttaagcttctgaactttgcattttacaagaaacacgggataaaagaaaaagttaagtaccatgaggaaatggacaaattcagaatttggacattctgtaatacagatggcctgagtcactgtcacagagaacaaaaaacaaattggggattattctacactaaataagactaaagagatgtaaacaaatgcaatatgtgatcctagcttgggactaggatagggatgagctataaaaggcatttttttatgggaaatggcaaatgttgattaactatgttatggaattgataattttcctgaaaatgataacaggaatgtaattatgtagtggaatatctttattcttagaaaatacttttttttttttaatttaggggtaaattgtcatgacacctacaactttttccaccttcttttttgaaataaaacttacacagagtagaatatacaaatctcaaatgtacaacagagtgaccttatacatgtgtaactgccacgcaggtgaagatatagaacatttctagtgcaacttgagaagggttctcccatgccccctccaactcagtaatcacctccaagagtaatcactattcttacttctatctcagtcaatcaatttggaacttcatttcaaatggtaagatagaaaacaaaatgttagagaaaataaatatgaaaaaacatttagaatctaagcagagagaatttggatgttaattatattactccttctaattttctgtgagtgaaattttttacaatagagatggaaaaattcaaacaacttagacgaaaggagaaacagaaggttcaaaggaagcatttgtcactagagaaaaaaactaatgaacataaattgatactgatttgaggaagtagcaaggtaactttaaataaactttcagtatgtcaagtaattacagcatattacagaagaagtatcaaaatgctaagtaaaaaaggatatttttaaaaaactaagtggatgaagtaaaaagtaggttaaaagaattattaaattctagaaagcaagaaaagagactcaaactataaaagagaggataagagatatgaaagatggaatgaacagtttcattatatgcctcagaggagttccagaaagaagagaaataaagtagagcaaaattcaaagaaatgatggctaacatttgtgaaagatgaatctaaagatttgggaaacaaaggccctgaagtgtataaataaatttcagtccataattattagatataacaaaatgaaagaatagttttttcaaaccattgaggaaaaaaaattatctttaaaaaaatcacaattagattatcaaaaatttttcatcatagcagctgtagaagccagaagacaatagagtaatcagaatgctgagaaatgcctgtaatttgcttagctaaaatatgagttgaaggtataatagaggcctttccagatgagaatttacctttgccaggctctgcttgatggacctacccaaaaattactttagaaaaacagttgaatacattaaggagggcacatggtgtggtgagcacagatgttttatgcaactgatgaattattcaaaactacgtcagaaactaatgatgtactctatgttggctaattgaatttaaatataataaataaagttgaatacagagaaaggaatgaaaagtaagaattaatcttagtaacctggaattgggcaatgattttgtaaatatgacaaaagtacaatacataatgaacatattcataaattggatttcattgaaatataaaatttatgcttttttttttaagattttatttatttgagagagaagagagagaacacaagcaggggatggggaatggaagagggagagagagaagcaggctcccctcagagcagggagcccaatgagggccttgatcctggggcccaggatcgtgacctgaaccaaaggcaggcacctaaccaactgagccactcaggcaccccaaaaactatgctttttgaaaagacagtgctaagagtacaaagacaaaccatggactagtctttgcaaaaacacatatgtgattaaggacgtgtatccagagcatacaaagagctcttagaactcaataatgaaaacacaaacacctcaaaaaaatagataaaagttttgaacagatatttcaccaaggaaaatatatagatagtgaataaataaatgagaagatgccagatgtcattagtcactggagaaatgcaaatttaaaggcacaagataacactacacacctacaagaatggctaaaatgaaagagtggcaatgccaactagtggcaaggatgtggaataactggaattttcattcactgctggtatatggtacatatacactttttaaaacaatttgatagttttatttaatacaattccttttaaaatcccaacagtgttttttgcagaaaaggaaaaatctatcctgtaattaatgtggaagcttaaggaatcctgaagaaccaagcaatcttggaaaagagtaaagttggaggtctcacacttcctgatctcaaaacttactacaaacctacagaatggcatagatatacagacataaagatcaatgaaataaagagtccagaaatatattattatgtatatggtcaaaggatatttgacaaaggtatcaaaacccctcagtggggaaaggacagtctcttccacaaatgatgctggcaaaactggatgtgcacatgcaaaacaatgaagttggacccttgccttacaccacatacaaagtcctctcagaatggatcaaagacataaatgtaagagctgaaactataaaacccttagaatgaaacatagaggaaaagcctcacaacgctggatttggcagtagtttcttagctctgacaccaaagcacaggcaacataggtaaaaatagagaagtattttgttatacctcacaaaattagaaacactgaatcaaagaacacaatcatctgagtgaaaagacaatgcagaataaatatttgcaaatgatatatgtgataaagggttaatactcagaatgtatttttgaaaactcctacaactcaataaaaaaagaacaccttaaacaacccaattaagaaaggagaggggaggtgagtagacatttctccaaagagatttacaaatggccaacaaacacatgaaaagatgctcaagagtaatcattaggaatatgcaaatcaaagccacaatgagctaccacctcacacccattagaatggctactatcaaaaagaaaaaaaaaaaaaaaatgaaccagccctggtggctcggtggtttagcgccgccttcagcccagggcaagatcctggagacccaggatcaattcccacgtcaggctccctgcatggagcctgcttctccctctgcctgtgtctttgcctctctctctctctctctctctctctctgtctctgtctgtctctctcatgaataagtaaataaaatattttttaaaaaaagaatgaatagcatgttggcaaggatatggagaaattggaacccttgtgcattgctagtggggatatacacaatggtgtgaccactatagaaaatgtttgaggattcctcaaaaactaagaaaagaattatattatcctgcatttctgcttctggatatatacacaaatgaattgaaagggcttcaaagaggttatttatacacccataattatagcagcattattcacaataaccaaaaatgtgtagtcaacccaagtgtccatcgacagatgcatgaataaacgaaatgtgatatatgcatacagtggaatattattcaaccctcaaaaggaaattctgacacattctacagcattgatgaatctcgagccagtcacaggaagacatactgtattattccacatatatgaggtacctagaatagtcaatttcataaaaacagaaaataggatagtggttataagggggtggagaattgttctttaatggttatagagtttcatttttttcaaaatgaaaactgtcatggagactggttatgaaacagtgtccatgtatttaacatagctaactgtatactttaaagcacttaagatgttaaattttatgttatatgtattttgccacaattaaaaaaaaatagtttggcaattttgtaaaaagttaagcatacacctagtatatgatccagccattcattctacttctagatatttaccaaagagaaagcatatgtccatacaaagatttatacacaattgttcttagttgctttatttgtaataccctcaagctgagtacaagccaaatgttcatcaacagatggttaagcaggctgtggtatatccataaaatggaataccatgtggacaaagagtgcattactggtagacacaacaacatagtcaatctcaaaataatcatgctaagtgaaagtagccagacaaaaatgaaaatgtattttacacctctagttatataaaattctagaacatgcaaactgttctctactgacaggtttttgtttttttaagattgtgtttatctgatcgagcaagtgtaaaagcacggggagcaacagagggagaaggagaagcaggccctctgccaagcagggagcccaaggcagggctccatcccaggatcctgggatcatgacctgagctaaaagtagacacttcactgactgagccactcagacacgcctagtgacagttttagatccgtatttgtgtgggcctggactggagggaggaattacataggagctcaaggaaactttgaggtgtaatagagtacattaattatcttgaatgtggtgataattcattagtatatacataatgtcaaaatgtatcagacaatatacctaaattaatgtagtttattgtagatcagttaaacctcaaatctgtttttttaaaaaaaggaatgaaaagggacatctacctggctttagttggaagaatatgtgactcagggttatgagtttcagccccatgttgggtatagagattacttaaataaaacttaaaaagaataaaaagtaatgacgagttgttagtaatgaaaagaaaattggtagtgttatttatctattacatgaaatatactttaagaagagaaagaagtgttaaggatgagggtcatgctctggcaggagaaaaggattaccttttactttatacttaaacccttcttccacagagctgtttctttgtgatgcttaaaattattgcttagagtgattatatctattttatttagctatttttcatgatggatagttagatatctacaaataattactgggtaaggtatacactcttaatagtgtatgaggcttacttttaccgtgagaccctggccaatacggaatgttaaaatttagaagatattattgatctactaaaatttaaagtatttcaggcccaagatacactcattcatgtatacttttcaatacataagtcatacaacaacaaaaagttaaggaaagtattagactcatccaagtatcccgaaacacaacttgtcctaagcctgtctttttgaattcataatttaaaagaattttttatagattaagacttaactcacgtacagttcactcaaagggtgcactccagtgatttttagtctgttcacagttatacagatgttaccacaatcaatttaagaattttctcaacattccagggcagccccggtggctcagtggtttagcgccgccttcagcccagggcctgatcccagaggcctgggatcaagtcccacgtcaggctccttgcatggagcctgcttctccttctgcctgtgtctctgcctctctctatctctctcacactctctctctctctccccccctccctccctctctctctctccttccctctctctctcatgaataaataaataaaatctttaaaaaaaaaaaaaaaaactttctcagtattccaaaaaagaaaccctatatgcattagtagtcacttaccatttctccccaaccttcccagccctaggcaactacttacttccttttatctctatagatttgcctattcctggacataccatataaatggaatcttggagtgtgtggtcttttgtgactggcttctttgatttagtgagtttccagtgttcttccatgttacagtgtgattcagtccttcatgccttattttatggttgactcatatcctattgtttgtatatactccattgtatttatccatcctgagtgttgtttccactttttggctattgtgaataatgctgctatgagcattccatgtacaagtttttttgtgtggacatatgttttcgttttttattggataaatgtctctagtaatcttttgaggaacagccagaatgtattccaaaacagctgtagcattttactttctcaccagtactgtgtgagggttttagtttttccatctcctcgccagggtatttattgtctttttgattacagccatcctagtgtgtttgaagtggtgtttcattatggctttgatttgcatttccctgataggctgatgatattaagcatcctttcatgtgcttattggctttcatatatctgttttggagaaagaatttccttttatataaatagttttctatggcatgcatagacaaaaataaaataccataggtctgagataagaaaaattataagaactgatgagcactgctctaataatagaaatgggatgtaaatggctaggattatttcttctacagatggcaaaaggtacaagttctaagactatatatatttcagaaggagaaagaatagagattataggaataaaacttaatgtgttttagatccaaaaagaattgtagaagacgaataagcctgagattagaaagtaaaagtaaggtagactaaatgaaagcattataattctaggaatatgatagtagctagcccctagaagtgtttttttatgactctaaaaccagtactaaacaaaaaaccatgcttgaaaattttatatcattttgcaaagcaaggtatttaattgatgactttgagtcaccaatttagcatttatttatttttatttatttattttttttttaccaatttagcatttagaaaagaatgttcataaggttttttaaaacctttttatttagaaaatgtgtttatatagaaaattataatggacagtatatttaaatctgtttgtttagtcacttccaatgaacaaataggaatttttgtcatttttcctttatactacacacatatgcaaatgtggttcccttttttttttttataagattttatttatttattcataagagatcagagagggagagaggcagagacacaggcagaaggagaaacaggctccatgcaaggagcctgacacaggactcgatcccaggtctccaggatcatgccctgagctgaaggtggcgctaaaccgctgagccacccaggctgccccaaatgtggttccctttacccgtcccattactctcctgaaaatctgctgtcccactgtttcccaagaagcagccactctgataaataaaattaattccaaaattttatactttactaaatatatgtatctataagtattttttaaatttatgatagtatgatactatatacaatattcagtaccttgccttttagatttaaaattataatttcaaggtatacgtatgtatatgttgatacatataggtcttattcttcgaatttaactactatggagacttccagcttctgaatataccacagtttatccatttccctatgatggacatgtagattatgtttaattttttttgttattattaaagcctaaaatacgaacatcttatacaggattctttgtatacaagtaacaaggtatttctaagttgtaaaagtggaagtagattcctggggcacgggataggcagatttgattcaaaggttgtaaaagaacaaggaatctggaaaaatatctaaagctactaagttaattttagcatctgtggaatggaagttctgagagcagcttatcagtgactaacatttagtaggtggtctgtaaatattttagaagggatgagtctttgggggggggtgtttaaagattttatttatttcagagaaagagagcaaagcatgagcagggggaggggcaaagggaaaagcagactccccgttgatcagggaaccacgagccgaagctggactcgatcccaggaccctaggataatgacctgggctgaaagcagacactggatgtttcaaaagtattccttcattgtgctgtaattaaagcaggaaatatttgtggaatggaagggataaggagaagctatgttcttgctttagccagtttgggaggaaacatgaagaatacataaaaacactttaagaaaaatgaaaaagaaaaaaacaacagcaaaacttttaactttcttaatagcacaaataagtagttttaatgaaatagattgagtcaagggtcatcactagataggagtaagtggtagtacccccacagaatacccatccctctatttttagcaaaagcagaaacctaagatctacacaagtgaggaaagagagtggggagggtgttaggttctatttctttatagacagaaggaataatagtacccatggcatagtggtttacagtttgtgaaaatgcactcacatcctctcacggtttgtgaagcaaaatactgtagagagcagtattagattatgttgatatttcagttacagtttgatattaataatattatgtggctgtggaacctaggacattcttgaatcctggtaaattctgttagctttcaccaacagtgaaaggacttctttttctgttactagttaagtacctccctgtgcagtactaattaatgaagtaactgattaactttagttttgatctttgatttgatttacttttatagcattttactggtaaaaacttaggatgtttgtaagtcctgtgaccactggttctttgttaaatacaatttgaccaggatgtctgggtggctcagttaagtgtctgactctcgatctcagctcaggtctcgatttcagggtcatgagttcaagccctgagttgggctccacactgggcgtgagacctactttaaaaaatacctatatctatatatctgtctatcttgacctaaagacatactacttaattctaaaattgcttttacattatgaagtttttgtagcgtagtcagcctctttttactttagtaagaccatgttgtaagcagattacatgaagtttttccttcaaaacctaatcattataacagtcctgaaaaaggacttgacatcaggatacattatcataaaatcaggaagccacaaatttagatttatattcaaaacccatacattggcatgtatattttgtgccagtttatgcaaggagatctcagggtatcatgcagtttatttaaagtatatagtgaagactgcattgcaaagaatttaaactaccaaattaatctgctagctcaaataatatgttacaatttgatggccattactttaggaaggtcttttaagttttaagaaaacatttcattatggaaatttttaagtgtacacaaaaggagtagtgtagtgagccatcatgaacccattgataaaagatggtttatattcactttaaaaactaatatgtggggatccctgggtggcgcagtggtttagcacctgcctttggcccagggcgcaatcctggagacccgggatcgaatcccacgtcgggctcccggtgcatggagcctgcttctccctctgcctatgtctctgcctctctctctctctctctctctcactgtgtgcctatcataaattaaataaataaaataaaataaaataaaaactaatatgtgaaagtttagactaatacattttgttctggtccaaattatcctttgcaaaaatattcaggcagttacattgactctagctctttattaagatgggatgcttgttttaaaaattattgtagaaaaaaattatttctcctttgatagcctaatcaaggacatttaattttggaatttgaagaattccatgcataaacctgattcttttgaaagtatcttttcagtcctgtcagcctttaaatatgagacaaatcactcctcccattttcgatcatataattctttcctgtggtaattatataaagaaatcttgtgccctcccatgtttctataatctttctatacatccagtttcctctgaaacttttaggtaactatttttatttgggaggtatatgtgtattttctgggatgttatgggtttttgtttttctttaaagaatttatttattcatgagagacatggagagagagaggcagagacacaggcagagagagaagcaggctccatgtgggagcctgatgtgggactcgatcctggtactcctgtctggatcacgcctgagccaaaggcagacactcaacagctgagccacccaggcgtccctttgcggttgctttttaatgtagctacaagcaaatacaaagatgtattcttatttttccacttctctttgtatacacaagctatcatattatatatatcattctgtattttgtttttaatatttacatagaccctggaaatgctccgttgccatatacagagaaattttaagtattatttttttttacagctatagatttcagtgtatagatgtttatcagaatactatagttataccaaaaagtgtgcttgcctggatcaaaaggaaatttatatatgtaattttgataatagatactttgaaattttgagggatgcctgggtggctctgcagttgagcatctgcctttggctggctcagggcgtagtcctgggatccaggatcaaattccacatcagtctccctgtgggaagcctgcttcttcctctgcctatgtctctgcctctctctgtgtgtgtgtgtgtgtgtgtgtgtgtgtgtgtgtgtgtgtctttctcatgaataaataaatcttaaaaaaaatttttttttgctttataggtcttgcttaattttgcattgctacctgcaatatatgagatacctgtttaccatagcctaatatgatagaacatgttgtcaagcttttgggtttttgccagtctgataattaaacaaaaatattttggcaagtttgagtttactcctagtgatactgaacatcttttcatgtttgaactttcacgtcctttcccatttttctattgggttgttggtctttatctcatatttttagctctttatatattaggaagattacatgcatgttataggagttgcagatattttttatcagtttgcttttgtcttttgactatgctcataagttacaaatatatttttcatagtttgtatatgtctttagactgcttataatttgttttgccatctaaaagatttttagttttttgcagtttcttttatggcagattttgcatcatagttataaaggccttcttaactagaggttataaaggattttgccatgttttcttccagtattttatggtttgtcttttccttcctttttttcaacatttaaattttttacttatttggagttttccctagtctacagtatgaaatacggatgaactttttaactagattggttccagttgtctcagtatggacatctttaccctatttcagatgctgccttaattttattctaatttcctttatgtattagagtctgtttttgtctttttattctgttccattagtctgtcagcctgttgaactggctctatcaattaaggagcaaatagttacaaaaaatcagcctacagtagtgaaatgaaagacttatcactttcccttattgctaagagtggaaaggcatatttttgcctgctgatgaaatagacatatactttggaattaagcgtaattagaagatacatgacaggattgcataagggaaattaccaaaggaactaaggggtagtagatagccatggttcacttgtaaccttatatgccttgttatggtatttgaaatttcccctataagaactggaaaattgtaggaacttgaattttgccccataaattatgaaaaattggagcagactaatgaaatataaaattacattttttttaaagattttatttattcatgagagagagagagaggcagagacataggcagaggaggaagcaggctccatgcagggagcctgatgtgggactcgatcctggaactccaggatcacaccctgggctgaaggcaggcgttaaactgctgagccacccggggatcccctaaaattacattttagaatgaagttctaaatctgaattcagtgggcttctcaacagtctatacatgagcttcaggttctatggacccctgaaattttatgtgtcagagcatctaagaccagaatctcagaagtctttttttaggtgggggaggggcagagggaggagagagaatctttttaaatttttttatttaaatgaaattaacttttttgaaagattttatttattcatgagagacacatgcagagagagaagcagagacacaggcagaagaagaagcaggcttcatgcatagagcctgctgtgggactcaatcctgggacaccgggatcattccctgagccaaaggcagacagacactcaaccgctgagccacccggacgtccatgagcatggaacgtttttctgtttctttgtgcaatcctctatttctaggtgtcctatagttttcagtacaattcatttgcctctttggttaggtttattcccaggtatctaatgggttttggtgcaattgtaaatgggattgattgcttattttctttttttccatctcattgttagtgtatagaaatgcaggtaacttctgggcattggctttatatcctgccactttgctgaattcctgtgcaagttctagcaattttagggtagagtcttttgggttttccacatagagtatcatgtcaggagagacttttaagcaggctccatgcccagtgtggagcccagtgttcagggctcagtcttacgaccttgagatcatgacctgagtcaaaagagtcagaagcttaactgactgagccctcccaggcactccaaattctcagaagtctttgacatacacacacaagttaaaaataccttttttagaaagattcctctggttacatggaatagggtttaaaactaaagatagggttgctgggatccctgggtggcgcagcggtttggcgcctgcctttggcccagggcgcgatcttggagacccgggatcgagtcccacgtcgggctccctgcatggagcctgcttctccctctgcctgtgtctctgcctctctctctctctctctctctctctctctgtgactatcataaataaataaaatctttaaaaaaaataaataaatatagggttgctaatcaagaagtttttgcataaagtatgcaaaatataagtagcagagcctatcaaaatacaaacaacatttttgacagaagtggaacaaataatcctaaaatttgtatgaaaccatggaagatccccaaatagctaaaggaatcttgagaaggaagaacaaagctgaagatatcaaattctagaattcaagatgtactgtaaagctatagtaatcataccatgtatgtagtactggcacaaaaatagacacgtagggcagccccggtggcttagtggattagcaccaccttcagcccagggtgtgatcctggagacccgggattgagtcccacatcggactccctgcatagagcctgcttctccctctgcctgtgtctctgcctctctctctctctctctgtctccatgtgtctcatgaataaataaatgaaatctttaaaaaaaaaatagacatgtagatcaatggaacagaatagagagcctagctataagcccatgtttatatggtaaattaacctatgacaaaggatgcaagcacgaacaatggggaaaagatagtctctttaataaatggtgttgggaaaactgaacagctatatgcaaaacaatgaaactggaccactttcttataccatatacaaaaataaattcaaaatggattaaagacctaagtataatgcctgaaaccataaaactcctagaagaaacatagttttcatatcatcatttcatcaaacatatttgatatctccttagctgatctagttcttcctcctcaagaaaaacaaaagcaaaaattattgggactataccaaaataaaaggcttttacacaacaaaaggaacaaggcaacctactgaagtaagagaagatatttgcaaatgctatgtctgataaggggttaatattcacaatacataaagaacatacagctcaacaccaaaaattccaaataatctaattaaaaattaggcagaggacctgaatagatatttttccaaagtagacctacagatggccaacagacacaggaaaagatgttcaacattgctaatcgttaggaaaatgcaaatcaaaaccacagtgagggttgcctggctggctcagtcagtagagtatgtgacttttttttttttttttaagatttttaaaattcatttattcatgggagacacagagaggcagagacacaggcagagggagaagcaggactccccgtggggagcccagtgcgaggctcggtcccaggaccctgggatcatgacctgagctgaaagtaaatgctcaaccactgagccacccaggtgccctgggtatgcaactcttgatcttgggattgtaacttaaaaataaaacctattaaaaaaaaaaaagaaaaacaatgagataacaccttatgctggtcagaattgctaaaatcaaaagtgtcagcgaggatgtagaatgtagagaaaaaagagttaccatatgctattgatgagactacaaattggcacagtcactggaaaacagtatggaacttcctcagaaaaataaaaatagaaaaacaaacaaaaaacagaatccgacctacagctacagagatcaaactagggatattctattccccagaagggaagagaatagagggataggcaaaatggatgaagggaagtagtagacgtgggcttccagttttggaattaataagtcataaggatgaaaggcaaagggagtgtaatcagtgatattattataatgttatatggtgacagatactagctatacttgtgagcatagagtattgtatagagaagttgtatcatgtggtatacctgaaactgatacgatgtgtgtcaactatattcaaatttaaaaaaatttttaaattgattttgctttaaatgttgacatgctaattcttaatggtgggtatataggaaccggttgtctttttatatgttaaaatatttcataattttaataagcaaaatcaattttgctttaagttggtgaataatagatacttcagtcataattttcctttagtatcaagttagctttacaaatgatgttattatctgacagagtgatcatatgacattttaaaggtattgtaaagtattgaccttttttaaatgttgtggaaaaatatataatttattccctttatctcatattatatatatatatatatatatatatatggatatatatatatatgataccataggtacccgcaagatgtggagttacagtccgggacagtctaaagaaagctctgatgatgagaggtctaatcccagagtgctgtgctgtttacagaattcaggatgggtatggtttgtatgtgatgtgaaattttgtttaaaaaggaaaaaaaaaaccctcagaccttaaactttgggttttctgtagcatctttacccaaacccaaggtattgaaatttaactttagaaaaatatattaagttagcctaaagaaaaccttgtaggccttttgaggagtgaacattaggtattttgtgtgatgatagtgttattctgatgaaatgactatttggagtctgagattcttataatttttgaaccatttaaaatgtggcttatttgaataatggactcttcccccatgcccatatttttaatataaactcatagtttcacaaaaggcttatcaaaattaatattttaagctatttttctttcagaaaagtatctaaaattgtgccaggaccccccaaattttgaatcctgtttttaaaaaaaatatccttttggatattgttgtcccttgagtactctttttttttttttttttttttaatggatagataaatccgtagctgtgattcttttttttttttttttttttggaggggggaccttaggaacaatcaattttctgctgttgtaggtcttttctggagctggcttgaagaaaagagtacatctctttaccctgctgtttgtccaagagtgatacatttatttggggtaaacttaaaaattaatttattgccatttaaatttctaacgatggaagactagggagccaaacctccctcaccattactagcccctcaataaccaattttcatgtcttcagcatgaggtatatgaagatttttaggtgtgataaccaagaaaggctgtgtctacacttttcagagagaagaaaccgattggctgggacactgatatttcctggctcactggagaggaattgcatgtagaagtgttggaaaatgttccgcttaccacacacaactttgtatgtacttttacattttttccaaatgtcaaaaatgtacaagttcttgcacatgaatttttacttagggaatatgaaatatggatgaataattttggggttggtattttacctaagttgaaaatccattctgttctttaaaaagcttatatttagggatccctgggtggtgtagcggtttagcgcctgcctttggcccagggcgtgatcctggagacccaggatcgaatctcatgtcgggctcccagtgcatggagcctgcttctccctctgcctgtgcctctgcctctctctctctctctctctctgtgtgtgtgtgtgtgactatcataaattaaaaaaaaaaaaaaaaaaaaagcttatatttaaataaagagttttattttcttcttaatttggaaaaaacaatatatgtttttcaataaaaggaactgaaaagataatctgaatagtacaaaagtatatcaagtgtagtcttccaaactccttctacttatacaaatgtgtgcttatacccgggagccaagagctctctccattctctttttcctcccttcctttaatttccatttctttccttaaaccaaaaaagaacattcctgttctgtgtgatagtcttcatcttttaaaaaagaaaaaaaatgaacagtgtatcatggacatctttccacatcagtacacctggtttcatatttacagagattgcataatagtccataagaacataaattcaatatacttaaccatcctctactaatatagacatctaagtttttaaaaaatctctcttgtacgaaggactatacagaatcttgcattcatcataatacacttgtgtgtttctgaaggatgagttaatggaagtgaaataactctccacgaggacctctcccttcatctggtttttgcatttgctggaacctagcctaggctaagtgtcaccagcatgataactggaacaagggtcatctttacctcagtgatgagagtatagtgcccagagcttgagaacctgcagaagccaggagtagaatgatctagaagacaggaatgcatttcttactttctatcatatatggaattttgagcatagtttattttttctagatggatagttattgctacccctttggtatcaggtgctctcccgtgtgttttggtgatcagtacagctggactcctaaagcagatgaataatgagctgatttccttgggggtttgtgctttcagtgctccattagtggtgaataatggtgaataatttgtgcttcagtaaggcatcttttcagtgagtgttgtcaatcagatttactatgtagcagatagttttgaaataatgacccagcaagttcattactatagattttttaatttgtagaatttcaagtggataaatctgaagataattttaagtttttagaaatccaaaaatggtagagcttacatttcatgaagattactatttttcttggctaaaaagtagttaaaaggaggtaagaaaacctaagttgttaaccataaaatggtgttagagtagtgcctcaggaaggtccttgccacaagaggataatgaaaacaagctgcagcctagttccagagtatggaaatcctcacaacacagttggagacctgccagtaaatatttattaagaactttccatttgccaacactgtgtctgatctgggactataacaatgaataagaaaaacaatatccctgccttagagtagagagagagatgttttgataagaacatttcataaacactcttaattactgtgtatggataaagtacagtagatgcacagagtagaaactcctaacctgggcttggaggagtcaggaagttttcccaaaggaagaactctctaagctaaggcctgatggatgagggaatagcagatggagtgaagagaagagaggaaacagaaagcacagactacttttcagttttgaaagaatgtcaggggggatggtataaagttgtagaggagattgttatagtgtagcaagaattatggttagaataagaagaatccagctcctagaagtccttgtaaggcttgttaaagatttggacttctctttaaaaaattggaaattaattgtaagcaggagagacgtgattggatttgtgtcttagaacagttgctctgagtgtcatgttgaggcaagagtaaatgcatgtgttctgcttaagaaagggagagaacttctctctttctctgtgctgggtttggtggctacttcagaaatgctgcttatgtctcccagtttcctagagtccattattaaaagcagttccttagatatttgcctacattccaggagtccctgggaactggcagggtgaaggcagcaggaaaagcagaaaaataagaaagtataccttcctgtactcacttgtattgacaaagatatattcccagaaacaattatatgtcacaatttattctcaaatacattcccacatttgcttctcctcatttccactcctctctgttttcttctctcttcccctcctgcactgtctgggttttatttgtttgtttttttcctcctttacatacacaaacagtctggtacgtagagttttagaagtcagtagcaggaagaaagactgcaaatgagtagatgaatgaaaagagaaattagagcttcatgaaactcagcagattattaggtgagtgagaaagggttagttagcaaagagaatcaagtgatgataaatgctgattcttgtgaacatgttttgtatttttctgtgctctattttctgagattctctgagaaatgtaaaaaacgcttgctttggcaaataccagccacataaatgttaaattactctactatcttgtatgtccaaagacaaacggaaaagccacaaagctaaaaaaacaaggaatttggcttttatgtcctctagaaagtgatacataaaccaaaaggtgaagaaaacaagagccacttttaaaaattacctgaatgttctcaaaaagctagaaagcatgaattgccaagataccattttcttgatgagacattatttttaaataactttttatcctgttaaatgcatgtttattgtaaagagtttggtaagttgtggctattgaaatcaacctaacgaatcatgagagcaaagacatataatagaaagtcctggtgtacattatagtcggggtactgttaaatgaatataggtatgttctcggtcataatataacgtgtatttcttactattgttcatgatttaaactgtctgggaaacagaaatgaattgagaatatacacaacaaagacaaaggacaaaatagagtcacctgtgccctaaccactctgaaatagccactgttaacattttggtatttttcttatgttttctgcttattcaaaacaaatactctaaaaataatttctggcttcagtagttaccaggatggagcctccctaacatgtttttggtttttttttttccccagtgtaacccctgagtagatcatttgccttctgacctagggttctttcatggtgccttttaagtttcttgtagagcaatgttcacaaatttagatgtttggatcctctaacaaactgataatttgtaaaaggtgttcatcttgattttcctgtcccttatcctagttaatttctgttttaaagctctttggtgattatctcattattaccaaatggtaactttaagctacagttccttactttatcaactgctgttaccatttaccatttggtaacttctgttaccaaaggaaattagctcatttgtagctttacaatctttcttcttcctccttcccctccttcaggtgatttttgggtttttttttttgttgttgttgttgttgttggtttgttttttgggcaggttcctgtattggatacctttgtaactttaaataatataccattgtttcttattctgtcaacaccagtaagcatttcttaactcctttccttgtgcagttgttaaagtgtgcccttaccttccgttcactcctactccctccttctgtttatcatctctttaactttcatggagtcaagcttgaaaatagtttctagttttttattaacttcccatcttttttgcttctttttaaatcgtatattaggtgggaggtaaaaacatatagtctattgtcttgaaccccctggttttcatattagtatcttagtgtgtaaaacggttcttgtgatatgaacataattaaagatttgttattttaaccaggagagtaaataagttgatttctaaaaagattcttacaagtttttgaaattctctatgatgttttctactttgcaggtacggaaaacttttttcaccttagcattttgtgacttttgtcgaaagctgcttttccagggttttcgctgtcaaacatgtggttataaatttcaccagcgttgtagtacagaggttccactgatgtgtgttaattatgaccaacttgagtaagtaatcaaaaaaaaaatctcttttctgtctacattttacttttcttgatgtaaaactaacttatcttagggtgtgggaattttctgtgcagtagaattacagagaatttagaaaggatggctagagaatactgtgtagtatagccagtactactctcccaaactgtaattctgattcagctatacatttcatttatgtcttatatcttcttttaattaataaagatcccttgcccatagacatcatttttagatggttttgggaggagaatgggtgtgattttaattaagttaaccgttatatttttggatcacagatcagggattctggttctgtcactattttataaattatatatattctctctctctttctctctccttcaaccttccctgcccagtttatttaaaagtaagatataaaatctttgtgcccaatgtatctctttctaaaatttctatttgctttatgccagagtttctctaagagttaagactgagtctgttgtaatcttttgttgttgctgctgctgctgctgctattaaggtcttaaaacttttcttaatcacttgagcaaagagaatttaactctatgaagtatattataatgacatcatctaactgagatggtagataaattttcttctaattccgtaaccataaagcatgatttaccccctctaatatgtagaaatttgctgtatagagaattagttttagattatgagcttgcctctaaaagtacaatgtaatttttaaaaaataagcctttaggcaaaaatacaactaattgaatttaacaatcatttctgaaagtggaatttgatctcaaatatttcttttaactctatatgtattctggtgtatgcagcttctgggttttgcacaagttaggtttgttttgttttgtttttgtttttgcctcacagtttgctgtttgtctccaagttctttgaacaccacccaataccacaggaggaggcctccatagcagagactgcccttacgtctggatcatccccttctgctcccccctccgattctcctgggtatgatttgactcctgctgttcaatgacaatgctatttgagctggtttcttttggatttgttggttaattataaacctttccaacacttggattattgaattaaggatttttctttgggtaacttatcccataggcaaatatgtacatccatttggtaagatacctaagattaaaactaacatagactgcttctggaccagtcttaaaaaaatcaagtaatatcatttagagtccctggcttgtgctcatcactgtattaggtaatatcaaatataaaagtttatgattcctatttagaaaagtagcattgacccatgagacaatggcaaacattttaaaaatagtgtataattagtttgcaaattctgacatggtattttaaacaatgtgaggacataaatagtatcatgtatactatagacggggaatagtgagagtagttaataacatttaatgaagatttgaactggaccttcaagaattcagaattttaatgagcccagggaataaaaagcatggtgatgaatgatgggatggtaggggggaaatagcataaacaaagggaatggaatggtagagttgagaggtggtgaacagaggctaatcttaagttgagacgtagcatgtatgagaccagattatgaaaagtgttgggtttcaagttggaatttagacttaatagaggtggggggagtaaaagtttttgagcagaggactaatctggtgaaaatggttttgaagaaagaagcactaccggtgatatacagatagattaatggacaaatatctagagtgaagaaggctaattcagatgctgttcacagtgaagaccatgagaggcaataaagacttgaactgggatggtagcagtgtggagaagggatgggtatatataaagtgtaaagaaaatatttatagtgtttattagttgtggaggataaaggagaaaactgactttaaagatgatccagggatccgtgggtggtgcagcggtttagcgcctgcctttggcccagggcgcgatcctggagacccgggatcgaatcccatgtcgggctcccggtgcatggagcctgcttctccctctgcctgtgtctctgcctctctctctctctccgtgtgactatcataaataaatttaaaaaattaaaaataaaaattaaaaaaaaaaaaaagatgatccaaaggcatcatatattcaagcttcatagtagggaaaaaaagatgatgaatttaacagatattagtgttgtggagaatttcttacataggtctacatactctttcccttttggatttgaggcagaataaattaagctgtatttggaagcatgtggtttaaaaaaagtcctcacatggcattctgttctataatattccagagataattagttaagatcagaaatatttattgtcagtttgaactctacttccttcctctcttcacaatgtaatgttatcttttccacctagagacaaaaaaaaaaatggttaattccaagacgtcaaattttacaaaaccaacctttctagaggtctcataggctacaaaatctagaaaagatttgtattaaggactagatgtttctatctactttggaacactagaaaatggaaccattaaatttggggtgggggctgcagtggtcaggttgtaatagttatctgctctggtggaatccttttattcctgctgtctttattgttagttttatgcacctaggtcgttcttaggcaggcgctcaaagtcttgtaattgtttgttttgatttttaattaatcttttggctatggcatttactcctgacaggttctgtaagaccatgatgcctttctaatggcaggtcccaattctgtgagcagagttacagaacaccacaatgaaattaacttggtctctctgaaggtttataatggtaatgatccaggacagttaccttccactgaagggatgctgtcttaaaattacaaaaccatttgcctatgccatgagtattttatttataactaaactgaagtgtacattctggtcttgagcaaatgttaagaaagatgttcaaacttttgttttccttatgcttttatgtcatgtgtctatatgtgcctatgaaaagatagaagccagtcttattctatactgctgtttctttgcattttgttaatagtccttatgacaattacagtggtcataaaatgcatataataatgaatgtaaccaagacatattggggaagtgatacattgactaattttcaaacaaaagttagaggtacctccttttctgctttttaaaaatgctgtggaatgtatgaatgtctgaaaagtcaacattttaaaatttccatactgctgaacagaagtagtcactttgcttcaaactcaatattctttaaaaaaaacttttaatattctagaggcacctggatgtctcaatgattgagcatgtgcctttggcccagggcgtgatcccagggtcctgggatcaagtcccacatcaggctccctgcagagagcctatttctttctctgcctatgtctctgcctctctctatgtgtctctcatgaataaattagaaaaaaaaaaaaccttttagtattctagttaaaaattagatctaaactaactagtaatttgccaaagggttttagaggtaaaattctttttatttttaatctaatactgttttaaagaggtttgcagcaagataagtaaaggtatcatcaggagaaacccagtttgctggctaccaacatacttcacagggtataaactgccttggtccaggtaaatacatcccagtatggaaactgaattttatcaatccctagaaatgatgagttattagaaatacactgatttacagtgaccagaattagaaaagactgaatcagaactccaggtaattaatttccaagagaaatagagctgttattgtagaccaaagttcttatgttggatgggccagcactcagtctacttgtagaagatatttatgagaaaccaaattataatctaaagattgcttttgttaagcatgggattaggacattactcagatgttttatgacaccaggatataattacttaaagttttctccaaagttacatacttgctgtcaagtctaggtaaattgactttatgggccaacagatgccttctactgggtaatggtagataccaactgccaaatatagtctttctgattttgtgtaacctcacttcttaaatatccattcatttctttaggtaataaaatttatcatatcaaatcccagtttttgtttttcctccttctagaccaatgctctttgaatatacgttctgctttgttataaactacattttgcgatcatagtccaattcagtaactttcaaaaatgacttgctctctcttatgcctgtactagaccgattatgtttattagactgccctatacaactgtagttaattcaagctttttccacttctaagtcctaactcaaatcacgctattgacttgagactttaaatcagcctatagttttttaaaagtagatgtattttccaaaactttttttaaaaaccaattttgtcaggcagcccaggtggctcagtggtttacaacctgtcttcagcccagggcgtgattctggagacccgggatcgagtcccacatcgggcttcctgcatggagcctgcttttccctctttctgtgtctctgcccccccttctctgtttctctcatgaataaataaaatctttaaaacaattttttttggtaattttttcattcttttacctgttcccaaaatcccttagcaaaaggcatatgttggctgcctaaagcaaaaataaagatagctctttcacaaactaaaattttttcaataattaatagaataaaaagaacaagtggagtgtggtaatttttctaaaggccaggtccatcagttttagcctgtaattctcattgtatgatactaagtatttaaagtacatagtattatttttatttcagttctaagtggactccattaatatgaacaaatgagctcaaaatttctttccctaccgtttagagcatgcttcaaagagtaaaactataaaccggtctggttcagattttgttaacatgctttatgcagaacctttaaaaaaaaaaaaaaaaaaaaaagccttttgtcactatttggcttctttggctcttcaaacctccttctattcctttttgtactatatttacttcccattcctttctaaactctgtcccctcccaaaatatctttttactccctccacaattgtctttctgggtgtgtcttaatagccactaaataatacagattattacagagtttgcataaaatggaatagaatgataacctatctctttagagaggctgttagcactgtgtcattcattggtcttgatggagctcaccaagacaaccatacttcctctattacgtgatactcccttcccacttcccttggatatcctttctgagggcattagactcctcacagaggtttacaatcttttcacagaactttattgtattctgtgagttctcggtttatttcagtgcagcgagacatcatagaatttgttatttacctacagtaccaaaatagatcattctgtcacctattttgatgatcttatcaaaaagcaatatatatttttttaatttttatttatttatttattcatgagagacacacagagaggcagagacataggcagagacataggcagagggagaagcaggctccctgcagggtgcctgatgcaggacttgatcccaggactctgggatcatgacctaagccgaaggcagacactcaactgctgagctacccaggcatccctcaaaaagcaatcttttaaaagatttactttaatgcttaatatatgattgtcttgtgttgcttagttattggacatggtaaacatttttgaatatgagagattgacactataaagattttttggaacgcttaaaaaactaagtatttgttaagcgcagttactcctgggtttttaatgttttataaggaaatctttatatcatcctttttaaaatgttattatctatataaagttgccctgaatcttatctttcaatgacttgatttttccaggcccccaattctgaccagtccgtctccttcaaaatccattccaattccacagcctttccgaccagcagatgaagatcatcgaaatcagtttggacaacgagaccggtcctcatcagctccaaatgtgcatataaacacaatagaacccgtcaacattgatgtaagcatccagcattattagaactaaaaaaaaaaaaattgggtgtatttatttttctgctacatctgtaatacagatgagaattgtgtatcgtagctattattagtgatagacttttcctgaattactgatcagaaaagcctggtcatcttctcttctggtattaaatacttaactggtgccaaattgtgaagttgttctgatatttttccttacgacattttttctgcaaaagtcttatgttcatttatttaacagataatcccttggccattttatagtaaataagaatacagttgcaggttttcaatctttatctttaaagattttaagtctttatctagatttcaaaaagtagaagattttcaatctttatctagatttctaaaagtagaactttgagctaaataactcttaacaaccagccattactttatgtacaagacatgcaaaactaccttttaccttccttactttttcgactataacactgtattccatctttctacatatcgtgaatttttgtatattcttttttgtaaagtttctcattaagttttattcatttaaaaaaatgtatcttatataatataatcatcctgtattgtaatctcatctcttcaattaatttaggcacctcctggctggtcactcttgatgtttttactttttcagtttattttgcacactgtaacttaattatgtaccttagtgttgcttcttcaattagatcttcttggcagcagaaaccattccttttttttttttttttttaagattttatttatttattcatgagagacagagagaggcagagacacaggcagagggagaagcaggctccatgcagggagcctgatgtgggactcaatcccgggactctaagatcacggccttggccaaaggcaggcactaaactgaaccacccagggatccccagcagaaaccattcctaacattagttcacttgatacctatcttagaatgaaatacatagttggtcatcaaaaaatatttgtttcagtaaacaggttagaagaagaaacaggaaatttcccatattatagaccaaagatcacaaacttgaagggccagataggatattttagacttatgggtcatatgacctctaatgacagctattgaattttgtcattgtagtgtgaaggcagccagagagaacacagtataaacaaatggatgtggctttatttccgtaaaacttttttttttttttcccgtaaaactttatttacaaaataatctggtaattgaactacagaagagaataatgtgctctgctacctgagctgtggtgggctaggaatccctatctagaattaggtgtcctactcccagaatttgtgctttcaaataaagagtataggttatctttaatctctgtaataagcaacaaaacaggaaactcaccaagcttgtttgccttggaaagtaattatattaagaaggagttaaaatcaatcaattaattaattaaaataaagtaaaaataaaatggaagttcgaactgtttccccaaataagcagggtactagaattagtgaggtggttttttgtgtgttagaagataggaattcttctcattatatgagcattccttatgtgagtcctcatagttataaggttgagttagagaagcaaaaattgtactaaaaggaaaatgttcacttattaacagactaatgttagtccccatacaaacattcttccccatctaatgccagtttgcattccaatctccatacatcattatccaacatttcaatgaaaacaatgcagattgattgcaagtctggcaaaaagaagaaagaaagaagagattttaagggttttatgaagacgaaaagggactttggagaactactaaaattgtaaaaagccattgaaaaaaaggaatacagaattaaattagtctaattaacaatagaaaagatccacagagatgacaatataataggctctccaaatgaaaataacttgagtattaaaggattgatagagtaccttcagggaactgatgaagccctctaaaatttttataaagatttatttatttatttatttatttatttatttatttatttatttatttattttagagaaagggggagaggatgggcagggaactgggcagagacagagggaaactctgctgaggggcttgaaactagtttttcctctagctaacagatctgaatacaaaggaagaaaaaagtagaagagggaaatatgggaataatgagattgtgaaataagaaatcaaatcttacttctgagttagatctattctcattatgttcaaaattatagctgtgttactttcaatattaacatcctgttagactatttccctctagacttttcctctcagtgttaccaatgttctcttgttttatgaggcttgaatatgtcggtgtcatttttatttatttatttttttaattatttatttatttatgatagtcacagagagagagaggcagagacacaggcagagggagaagcaggcttcatgcaccgggagcccgatgtgggattcgatcccgggtctccaggatcgcgccctgggccaaagacaggcgccaaaccgctgcgccacccagggatcccggtgtcatttttatattttgcctttttgtcctcgttttttaaccagatagtcaaagggtatcagttcttccttttgataactcatctccaatttgtccttattatttctaccctctgggcccatatttcttcccttgaattacttattacattaatttcctaaatagatttcctgccattggtttctctatccatttctatccattctttttttttttaagattttatttattcatagagatgcaggggagtgggggagaggcagagacacaggcagagggagaagcaggctccatgcagggagcctgacgtgggactcgatcccgggactccaggatcacgccctgggctgcaggcggcgctaaaccgctgcgccaccggggctgccctccatttccattctttatggtactgtcctctgtcatcatctgttccttgttccaaaaatttcatggacttcccactgattataagataatatataaattcttcagcatattcattcaatttcagtcatttcattcagctccctccaataattacttatcatttattattttagtaaacaagatttttttttttactttttttttttttgacatgacaatattgacactggaataggcattatgacataattggaaattaagggtaaattagtatatgatctttggcctaaaagaggtcagagtttaatggatgaatcagacatataaataaatagacacaaaaatatatagtggtaatttataaaagagggtaaaaaacttttaccttgttctatataggaatacttaatagagaaaggacaatatttaagtcatattgtaaaagaatgtgtaggagttgattgagcacacttggtgagggttttaggaagaaaagacatttgttaaggaaatagtatatgcagaagtatggaagtacctttacattggatgtaattaatagttgatattgtaggagtttagggctcaggagaagggtaggaaatggtagaagacaaaactagatgggtagagtccatactctgcccgcatacaccctacctgtatctgctttaaggtctctgtaggacctggtagtcgggagtttgaaaatttttaagtggtagataaagtggtcagctctatgttctggaagaatcttttgaattaatgagatatttgtaggcaagactgttaggaatgataccgaaaatggctactgtttatgaatgcttactctatgagaagtactgtactaagagctttacataaattttcttactctgtccccataaacagtcatgggttagtactatttttattcttattttactattgaaaaaattagattttgaggaggttaagaaactttccgagatcacactactgataaatgattgaaacggggtgtaaaatctgccctttcttgcttatttcttctatgttctgtagagtcatagatgaaagaggtgaaagagatgaggggtagaatataatgcccagatttctctggcttggatgtatagatagtatccattccagtcgttaggataggaagtaccgatggaagaacagtacttataaaaatgaaactggtaactattattttgacgatactgacattaagatatctgagggactttactggtttagaggtccagtagcagttgaatatcttatataaccttgaatagacatacagcttttggagtaattatcatatttgtcagttaaaatatgaatgtaaatgatactactcaaagagtttgtgtaaattgctgagagggtagaatatgcagccccattgaatattattaatattgagacagagactgttgttctgtctcttttgtggtctcctcttcctaagtctaatttcagctaagtaagagaaagtagtgttgcagaagccaacaggagaaaagggacttcaggaatgctcaagtagttgatgtcagttgccagtgagattccaatgataaggactgaaaatgactattggactatgtcattttgtcattgtaacttcattaagacctgttttagagggctggtggaggagagcaaggcaggtgaagatgtgggttaagtgtcagttttgagaagcttggctgtaaaggaataatgaaggtgctgtagattaaagcagagttgagggacagtttttaatgtgagagtcttcagcatatttgtaacttgagggaaaaaaacaagttgaagagagaggttgaaaataggcaagataggcaataagtgatatagtaaagttcccttatcgtggaaacaatgttcctcagaggctatattaaaagtggacatagctagagctctgcagctctttacaggctctataatctaggacacataacttaaccttgaagctagtagtgtctacctcttacagtggattaagaatgattattatgccttgcacatagtaagaatttagaatttatagttaacatgactgtgcaaatacaggtataaggacagaacagaatttttttgatggcttctgttttctctttcatgctaaatggaagagatacaagtaagaaaggaactacaggagatgaggaagagtttgaagtagctagtattagaataatagaagatcttctttaacctagaataaataagaagaatgtcaaagagctttaagaatggattgagggacgcctgggtgactcagtggttgagcatctgccttcggctcagggcgtgatccttgagttctgggatcgagtctcacatcaggcttcctgcatggatcctgcttcttcctctgcctgtgtctctgcctctctctctatgtctctcatgaataaataaaaaatcttaaaaaaaaaaaaaaaaaagaacagattcaaaactggaagtatgtggtgcagtaagagtggagaaagccaaataagagtggagaaagccaaaattggctgatccctggttatggggagtcacaagtttggtgcagcaaaaaggaaagggatgtggcctttctgcctttgtcttgttctttctgttcaaaaggtcctttgcttccctcttccccctgcattcctatctatcccttaagccccaactcatagctcatctcttgaaatgctttacctatgatcaggtcttaaagttatttatcctttgaacttgtgtagcactaagaatcttttcacacatactatttatttggcagttaactaagcatctttggtacctcttttattgttttcttaagctatttttcttgtatcatttatttccgtatgtgcatctcttccacttagattaaaagcttttgggcagccccggtggctcagtggtttggcactgccttctgcccagggcgtggttctagagacccgggatttagttctgcgtcaggctccctgcatggagcctgcttctccctctgtctgtgtttctgcctctctctctgtgtgtgtttctcatgaataaataaataaaatcttaaaaaaaaaaaaaaaaaaaagctctcaagaatacagaaagttagtgataccaccaggattttatatttattggtagtcacacaacccctaatacctagcacgttgtttgctagatattaagagtatttattggttattctgttgaaggctttatataatacattaaacatgatagaaatcatctgtaaaaaatttactctgggtcttagcctataaaagaaaagttacatatttagaaagagcctttgagtctgtttagagacatttgtggccattcagtcccttatatttaagttaaactggataaaaacctaagttctacctattaaggtccaaattgccttattgccagtagacttttccctccccactccccaatattatctattgtaaaaagaatcataaaggtagattttaacaaaactcttgtttgagccattttgattttgcttctagtgattccttatacatctgtatttttataattgcaatcaatgtacatttaattttattttttatcatactgtaaatatgtttcccaatacaagcttcctagctatttcagtagctgaacaatacttattatattgatgcaccataatttacttaacccaaatgtcaataggtctataatagggaaataattgttagtatgatggataatgctgcactggacatctttgtgcatatcattttttcttctgaattatttccttaggaataattcccagaagtggaattataggatcgaagggtatgaacattttatggctcttaatatgtgccagaattttttttttaagaattggtgcagccattggttttgaatgggctgataggaatttcaatatttgatcaggactaataatctcttcaatataggattaggttggcaagtgcttcaaaatgtttattttatcatgtttaccagctaacttcacattctttctgttttgtgaaatcatcattagacaggtattttgttttttgttttaagattttttttatttattcatgagagacagagagagaggcagagacacaggcagagggagaagcaggctccatgcagggagcccagtgtgggactagatcccgggtctccagaatcaggccctgggctgaaggcagcactaaaccgctgaaccaccctggctgccctagataggtattttgaattaacctttctgttaatgtctagtgaaaagttgagtgttgttattactttcttaccatatgctttgaataaacatccattcctgtttgtttgtaggacttgattagagaccaagggtttcgtagtgatggaggtaagtagtgatttcaattttaaaaaaacctcagaaaaatgcatttgctttgctgcttgtctcctttatatttgcttctttcatgaaacacagacacagagaaaaatgcgtagagaaaagccccaaatatttttttgttttcttattatttatcatttatgtctaatataattttaactgatttataagatgaatagagaaaaaggatataaaagggagtcttacctgtactatataattttaaaatgctgttatcatcaaagagagagcctgaattattttacatgctttatggattcctgtcttcataactcaagacgctaaatccaggagtcttcacagtaccagtaatatttaacgattgtgcttagaatcagttaaatcctgattttcctaaatcattcttagggagaatcccttagattgcctgtcacaaattttcagtttgttagaattggctttctagaaggatttggaatggaccctaatagaaagcttttatgtcctcttaatagtccagtaaattaaataagtaaatgaaaaattgggttgaaatttcttacagaatttttatcagatattcactctagcactctgtgaaaattggggacataaagcattgcttaagaaagtacttttaaagtaagattacccctatataatgttttcccagaaatgatatttgaatctatcttgaatattccgtccagatggtcttccatgcttactttttaagctgtcttctaataaggaagacctgcaaaacccatttatcttattgagagttagtctcttcactgtgacctgctttatcttcaaaatatctaagcccagacttaaagatattttatattgaaaaacattgtaaaaactgtcttttgttatgtttctgtatacttgtgaacctgccagaatgataaccctaccttatatattggatttagtattcacagtagcatgtagtttaaaaagttgtgggttttttccatttgttattccataaacctgccaattaatgttgctgccagtttgactttcatatgtcttaataactgtggcttttgatgattttgcccagtacatccagcatttaaatgttgccgtcatgttagcatcacagagttaacttagtcataaaaactgcctgcttagtaccaaaaatcaaatggcatttcttgtcctttttatgagtcactttataaaactcatttggattttatgaaaacaagcagaattttttagacatccagaatgattttgtgaagcaggcttcagctcatttactcccatgatttctttcagttcttctgtgtgtctgtcttcctgtgtttgcctgcccctctctttctcttctaacagcccctttgaaccacctgatgcgctgtcttcggaaatgccaatcccggactcccagtcccctcctacattctgtccccagtgaaatagtgtttgattttgagcctggcccagtgttcagaggtagttgggctcttctttcttgtttttacccaaagcaaactaaatataaactatagatactgtttgtgcctcaccctcacagtgtgtgtttgtaagtgtgagagttttcagtactaaatttctgcttggcctggctggaatgctctgaatgtgcttctcacacatactaccacaagctttctgtatgctatcctgttgtaaatttttaaaagcaagaaaattccaagctaagatgtcccatcttgttttttgttatgtgacttactttttggtcttgataacttcttaaactcagtgggtggcaataaattttatatatatatatggagggagttgtccttcaatctgcattacttttattttaatccatttaggtggtagccttcttgactattaatatttattaccaatttgtgtggttttaaaaaactaaaaatggaaatcacaattatgagattttgctagacttttggctttatttttctgtaatagcagtggtgtttttttaaatgtaggacatgtacattttatattatatcaaaattagggagccttaaaaaaggtttgtaatatatttggggtccttcagcacactttagaaaatttgagggttgacactctcaattatgccctaatacaagtggaaagctaacagtataatttcaccctcctgttttatcttgccttactttaattagtagttaaactgatttttaaagatttaaagaattggatttactgggttttgctagtgactgtagtgaatgtagtggtggtgtttcagatttttgctctcagccaggctgtttctcataaaatccatagaatgggctagaagtcctatggtgctgagtagtataactgaagtaagtagtacagtacagtcctgaggattatgaagcctgagcaagcagtttaggctttagaatgttgaaagttatcatcagaaatggattatgctctagttacaaacattccagataccattttcttttcgaaaattgcatgttggatcatggaaattctacatgatgacacatgtaatatactaatgataaaattctatacagtttttttttttttttttaatttatgatagtcacagagagagagagagaggcagagacacaggcagagggagaagcaggctccatgcaccgggagcccgatgtgggattcgatcccgggtctccaggatcgcaccctgggccaaaggcaggggccaaactgctgcgccacccagggatcccaattctatacagttttaacattgaaatgtagacagttggatccttattctttgaaagattagtgatagctacccccaaatagtaatcttctttccaaaaaacaaagattctctggaggtctttttgttgtatatgcagatattactggatatctcagttctacttagctatctatactcaggccatactaggagtccttagcttgaacccctctaaatcaggtaaatgaatttaggatttctgcttagtcagcatttggaaaagtatgcagcccggccagctgagtctactttcagtgtatcctatgggctcacttggctgataggatatttccatttgtagtaacttgacaggaggccataacacgcacacgcactttgctcttcctttatatgagtagcagtttttcagaaagtcttgagaaacctgcttggctttgtttactttaactttgttatacaagtaataaataatttttgtttagtgggataggaaaccaggaaccagtgaactgagcaatatttctaactatttgttaaatagtatatagttaaatagtaataggatttttaaagttttagtggctaaaatgatagagccagatatttttaacattaaggccatttttattatatgtttacttttgggagtttccatctgtttttatgtcgattggctgtttaggtgacacaaaatagttatacgctttttttaaatacatataagcttttaaattgatttcactttttttaatcaaagaaacaaaaacttgggagtattgtttcagtattcctctcttagtttagaagcattatttttattctggactctgtactaatcagtaaagtttgtatctgtaacttgatattttaaaagtcattaagttgaccagaaaactaaaagaaattatagtgcaaatcaccaaatgaggccgatttcggcctttcatttctaaacgctgtatatttaaacatacactgcatttttaaagaagtcttaaatttctccccaccctctgccccaataaattaatatacttgatcctcctttaaaagatatatttttcatttcactaatttaggatcaaccacaggtttgtctgccaccccccctgcctcattgcctggctcactcactaatgtaaaagcattacagaaatctccaggacctcagcgggaaagaaaatcatcttcatcctcagaagataggaatcgaatggtaagggtatataatatcttttttcttcttgagttcttttgttttacatatcatagctatatgtaatgtcaccttttagattgtatgatatatataatttgtcagtatgtgacacagtattcacttaatgaaataaatataagagctgatagttttttgcttaaaacattcccacatactgatttttttttcctctcttctttttttgctttggcttgacttttacacaattattctcaagaaaacacttggtagacgggattcaagtgatgattgggagatacctgatgggcagatcacagtgggacagagaattggatccgggtcatttgggacagtctacaagggaaagtggcatggtatgtatgtaatacagtgatacacaggatcccctatgtagcggatccctgggtggctcagcggtttggcgcctgccttcggcccagggcgtgatcctggagtcccaggatcgagtcccacattgggctccctgcatggagcctgcttctccctctgcctgtgtctctgcctctctctctctcatgaataaatgaataaaatcttaaaaaaaaaaaaaaaccagggcatgtttaacaacttctattaaaaaaaaaatatcaaagggaatattccctatttgcatcattaagctattttgatcagattcaagagtcttccaaagggtttctaacaaaaatcatgggaaataaaaactggattcacagcaacgaggactcttatcattagtgtaataatttttaaagatgacaattttctaagcgagctatactccaagatttttttccctaagaagtctttttgtgattttatttgtattataaaccacaagtaaacattaaaaatgtacttaccaaaggaggaaatagccaggaaacttacaatgtctagtctcattagatatcaaagaattacaaaataaaaaactagaaatcatttgtctctcaagtggacagattttttttttttaattgatggtgtttatagctgtgtttgtaatactgctggtaggaaaatagttacaaattttctggagggcaatttatcaatatgtatgttaagccaacaagtcgatttgtagaactaccaactaatgggacagaagcaaacagacgtgtatgcaagtacattcattagagacttaatgaaaaatgttcattaacagaaggcaggataaattgataacctccatatatgattatatagacatgaaatagtgtagatctatacttattgatatagaaaggtgtccgtggtttttaaattttacaaagcagcatgtgtaatatttcatattatggtacctaaaaataaatatataacataaaatttaaaatgactgtcagtattttagtatgtatcttttaggttatctttcatatgtatttgtaagtattcacattgtatacatacaaaagcctaataggatgtgtatcagtatgttaactgatcatttctgagtgacatgattataagagattttaatttttttctttatacttttaaaatgtttttcactaggagctctgtatttattactttgataatcaggaaaaatctgtatttatatcagagtataacatacctgcaaaaaagtgcgagattcttctgtattattgtgtgtggttgtaggtcattctctatgtaatattcattgtgtgattataatatgatttagttttcgatctctactgtatgatgagcatttgaatagtttccactttggagctattatgaatactgctgctatgaatattcttagtacatgttgtttggtgaacatatgtttcatttttgctgggtgtgtgtcagggaaagaattgtgggaccactggatttacatatgttcagttttagtaattgctgccaagtgtttcaactggttgtaccattttacacatctactagctatgtagaagagttctgcctgtttcacatgaaaattgtgggggttttaaattataatttaagtaattttccaggggatccctgagtggctcagcagtttagtgcctgccttcagcccagcgcatgatcctggagtccagggatcgagtcccacatcaggctccctgcatggagcctgcttctccctctgcctgtgtctctgcctctctctctctccctgtgtctctcatgaataaataaataaaatctttaaaattaataataattttcctaagcaaaagcaggtgatccatggatttgaaaaatatgttaagtactcacttaacatattttttctttctctgatgaagaaattttgcagttttctctttcagccaagtttcactgggtctgtcattggtaaatttaacattcagttagtaaaatcatctagtttaatattgttaaataaccaagaaaataccatgggaatgtaattggttaaactaaatttccacaggaaaaacaatgctgggtatttttttgaattggtgttttgaaatactagtataggagtatcattatttagtttttatccttagaaaattaatgcagtttaggaagttaagatatcatttatttgtactttaaagtttaaaatattgaaatgacatttggaatgacaattttttttaggtgacgtggcagtgaaaatgttgaatgtgacagcacccacacctcagcagttacaggccttcaaaaatgaagtaggagtactcaggtgagcctgtgtgaattactgtttttcagagaaattatcttctttttcccaatgatagctgctggtggctttacagatttactaaaaatggataaaacaaagcttgtatgagtatattttgtctttaaccatattctgtaagtggtatgcaatactgtagtgtgtgtgtgcatatagtgtgtgtgtatatatatagtgtatatacacatatatattggtataactgatggcaaaattaatcctagaatgaattaatagaaatatagtattctgatcatgggaaatcataccaccacttagtaaaataatactctcaaaattctaaccttaccagaacacatttaaagactgcatcacaccctgaaagttgaggaatattaacaaactagagtatatccaggcttggacaacttagatgaggagaattctggaactataacttaggaggataaaagtcaacagaaccgaggatatttgggcccattgaaggccattatgttcaagagggtatacaaactttgttttattccagaaagcagagcaggaaagccagtggtggaagtggaggggaaatgatttttttctcaaccccaaagcactttctaagactagaactacttaagagacaaaaaaaaagagtgtggggggtggaggtggagtgagggaaataagctacatctcagtgatgagcttcgcgtaattgaaggtatttaagcaacttccagttgcctatctgtgaggtgtattaaaatgtaagacctttctgaaaggggtaggatgttagaccatacaattcctaaaatcatttctaaatttatctttccattgtctttttttttttttatgatttacttatttatttattttagagggagagggagtacacaaacatgcagacatgagtgggaggaaaggcagagggtgagaatctcaaacagactccctgctgggcatggagcccactgagcacagagtgggagacaaggctccatctcaacaccttgaggtcatgtgagctaaaaccaagagtaggtcgcttaaccaactgagccacccacgcgcccctccattgtcttttttaaaatacttttttctaacaatgtcttactgataattagttagctttggcccactttttaaagatgtgctgaagtttattgacctttactatttaccgatattcttctttttacaggaaaactcgacatgtgaatatcctactctttatgggctattcaacaaagccccaactggctattgttacccagtggtgtgagggctccagcttatatcaccatctccacatcattgagaccaaatttgagatgataaagcttatagatattgcacggcagactgcacagggcatggagtaagttccattctgttaaatctcttataaattatttttgaagaccactgatggtattttaaaggttttagctaatattcttttgggttgctttttaaaattacttgtctagtaacataaggacacattttaactaatggctagcagtatgatattaggaaataatatctctctagtatagtcttcaaaaacatatatcaagtacttaattgcaaatgcgtgactacaaatttttttagcatgcagtgatttaacagtgaaggttcagaaaacaaaacaggcaaaaaaaaaaaacctattgaaaagtcattaacaaaaaaaaaaaaagaaaagtcattaacagttactgaagtgtacaaaatgattgttaagttatcctttcagattattttctccatgtacccatattttttattacaaaatggtcatctgaatgaaataaagttaaatggaaaatctgcattctgatctttttggtgggtttcaagctgagtttttcatagccattgaattttgctatataattaaaaagctcaaagattacttgagtagaatttattgtcagtcaaaatagggaactaattgggaaggataaattttgcatacaactcttaagtacaaatgcagaaatgaaccaagaagtaaaacttaataaattttgaaagcacttagatgaaaaattttaagtgagtgaaatacaagtttgaaagctctatgggtagtttaaaaatatgaatgtatattacatttacattttatagtatacacgttgcttacatgcataaataattatgtcatttataaagtacatatttttatttgcttttatagataaggaaacagaattttaaatgactaagattgcacatttaataagcacagcaaaagtatcaaactcagaatgtgagtccaaggcattattctttccccatattcactatgctactcttacattttaaaattagaattaaaaaataaaaagaataaaagggtaaaagtatacctatgtatttgacacctagagctctgacttaagatcagttaaagcaagtaagcatagaaccaggactcaaaataaaatctggctcttgtatcttcccactaaactatcaccctctctgtctccaaacaggacagtatatgtgtaatttagattgtttacaagcctgtgttcagccaaaaatgcttattgcagcaaatgatacctattcagtaacatttccatttatccactagacttaaaacaatctcagcctttaatataagttagaatctgaatctgttcaaatctgaaaggtatagtaaactcttatagaaaataaactatggcaaaaatagtaaacattaatagggaaaaagcttattctgctcccatcccaccagtttctttgctgtttcttttttttattttttaaatttttatttatttatgataggcacacagagagagagagagaggcagagacacaggcagagggagaagcaggctccatgcaccgggagcctgacgtgggattcgatcccggatctccaggatcgcgccccgggccaaaggcaggcgccaaaccgctgcgccacccagggatccctctttgctgtttcttcaccaagtcaagcctgctccctcctcatagcctttgtactgttctttttttctttgagatacttagttcagaactatccatagagcatcttccttatgtctctgttcaaatggcacattttaaaacaagccttctcaaggcatctgggtggctcagttggttaagtgtctggcttttgatctcagctcaggtcttaacctcagagtcatgaactcaagccccacggtggggcttgaaaaaataaaagaagccttctgtcatcacttatataactaatgttcctaccctacctatgtcattttccacatccctctcctttttttttttttttttttttttttttaagacccagctctttgtgatatagttgtttattgtctgtcacccccactggcatacagtttatatgagatgaggggctttgtttgttcattgttttatccctaatggttagaacagggcctggcctataataggtactcaatgcatatctattaaatgcaaaaaatgaaagaatgattacacatagtaatatatattggtaggtttacattctggaagagagagaagccaaatctttctcatcaaaactgaaatgatgggtagcctgggtggctcagcggttcagcgccacctttggctcagggcctgatcctggagacctgggatcaagtcccatgtcaggctccctgcatggagcctgcttctccctctgcctgtgtctctgcttctctctctctccctctctctctctctctctccctctctctctctatctctgtgtctcatgaataaataaataaaatctttaaaaaaaacaaaaactgaaatgttgaggcacctaggtggctcagttggttaagcgtctgcttttgtctcaggtcatgatctcagggtcctgggatcaagtcccatgttgggctccttgctcagtgggaagtctttttctccctttgctccttcccccacttaatgctctatatctctctgtgtgtcagatatcaaataattaagtaaaatctttaaaatcaaaaacaaacagtaaggaaaggaaaaatagtcaaagaataataaaagaataacgaaaggggaaaagctttggctataaatagaggcaagaacaaaagacagtattagaaattgtttttagggggatccctggatggctcagctgtttagagcctgcctttggcccaggacgcaatcctggagtcccaggatcaagtcccgcgttgggctcccggcatggagcctgcttctccctctgcctgtgtctctgcctctctctctctctctctctctctctctctctctctaagtctatcatgaataaataaataaaatctttaaaaaaaaaaactgtttttagaaacagtgatggagggggaagcacctgggtggctcagtggttgagggtctgcctttggcttaggtcatgatcctggggtcctgggatgcagtgccacatcatgcttcccacagggagcctgcttctccctctgcttgtgtctctgcctctctctgtgtgtctctcatgaataaataaataaaatcttaaaaaaaaaaaaaacaccagaaacagtgatggaggggtgactgggtggcttagttggttaagcatctgccttccactcaggtcatgatcccaaggttctgggatttagccggcctccctgctcagtggggagcctgctttccctctcctgttcccctgcttgtgtttctctctccgtgccccccaccccgtgaaataaataaataaaatcttaaaaaaagaaacagtgatgaggcagtgaagtgccagtattgataaagacaagaagtgaccatagagagaacattatcaaattggagtagttctatagtctggggaatagtcagttaagaggcagtatgttcctattgctttacagtgttaattctgtaagcatgaccttgatgtctatctattccaaaatacttggctcactttttttttttaacttggctcacatttttcggtaactgactatataatgagtataccttgttatgaatgcagatcatcctatcagcttatcctagattttaatagctaaaaaaagtattaattgtaaattttaagccttctagcaagagttcttatattaccagtcttaaccaaagaagatacagttcctacttttcctttctgttcataataagacaataacatacaatgcagtggttaaataaactaacacatttttagtttccaaagggctttggcagatgtgggttttcagttaaggtgacagatttttgctatctttaaaattccactaaaactataatcaaggaatttttttaagcatgaacatataaggacagagacaacagagaggaaggagacacaaacaacattttgaaagccagaaagcagatggagaagtggtaatgaacttaatatacctaaaaaagctaaattcttagttagcaatggagaaatgaaaatcaacctgatttataccaaaaattcctcaaaagtctcagaaattggcagtgccaggtatcttcagaagagggaaggtacataagtgagaaagcatatctgaaaatctctgaatcatgtctgctgctctctttgactggatgactgcttcatcccttccctggcagtagactgatactcttttagagagaggaaaaccagttatgtccaaatgggggacacagacatggatattttaccagaaacaggattgagtgactccttcctaaaggatgcatggagagttactcaagcactctcttttcaataattcccagaatgctggcagtcagaccttccctttcaggcaggagattgggaaagccttctctgaggaaatttaccagccgaggggaaagacccagaggtagcaacatcagggattccacctagatcactgtacagggaagttgaaaaacaagaagcccatctatacactcaaacctttcaggcactttgttgaaccttcacttagtctgagtaagacaaattgtctctggatcatcaaaccttagatgtaaaatctcaaatgccagtgacagagaccaaaacaaatgggagttaaaaaggtaactagggggatccctgggtggcgcagcggtttggcgcctgcctttggcccagggcgcgatcctggagacccgggatcgaatcccacgtcgggctcccggtgcatggagcctgcttctccctctgcctgtgtctctgcctgccccccctctgtgtgactatcataaataaataaaaattaaaaaagaaaaggtaactagcaataaacaaacagtgcaaggagaggggatgccctctcccccaaaaacctgtccgtttgctcagagataagagtttttattcatgaagcaagaacaaatggtttataaggaacatttggataataacaacatcaacagtgttaaaatgttgaaattgtaatccagtgcaaattatgtgctttgcagctatttaattctaaatgtctgatccctccttccttgtcctccaccatcatttcacagaagacagagcaagtgagaggtacctaaccactaagtccttagactatctaaatagcttcctaataatgtacttttttatgaagccctcattcaaaatagcctgccccttgcatgttttccagatgttcttgatcttttctgactcctttctaaaatatattttgaaaaaagaaaaaaataaactatattttgaagtcagaataagagcatataactcattgtataaggtgctggggttacagtcattcataggtgtaataccttcagtaattaggaaagttgaatcattaggacttgaaatgtgtcttttatattggattacagaatatagggagatgggtgacatcttttcgttgtggttttgttgtctttactcaatgaaggatcatttctgattcctcggtttccctgtagagagcagaactattctcatggtagataatgagagccccactctttaggaatcagggtcagtatggtatgtttatcatcttcttacctgacagtcattgattcagcctaagggccaaggtattctaagaatagaataaagaaatactaaagaaatttcaaaaaaacatatttcagtaatgatcaactaaagaaatttcaaaaaaacatatttcagtaatgatcaacttgtcatgccattttttttgcagaacttttaatttcagtataatattgtataatttttaatttagctttgtcagaaaatatactaaatctcaaatcaagacaaagttgtgatctgttacagacaggggttataatctgaagaaagagaataaaaaatggagataagtcctcatgatttacttcttttaaataatgaaagagtatgccttccttaattaaaaataaacctaattgaaagaaaggatagattaaagctgaaaagatggtgaacatgtactaatgattgaaaacaatttcaattgaccaaagaataattatagtgaacattattttccagtggataagaacagtaacagaaaagaggggatactgataatcagaaaaatacaaactaaaagaagtaccacttaaatgggcaaaatacctaaattctgacaaaatcatgtgttggtaaagattggggaattgtttactggtacatacttgatgcagacaaccatgctgatggcatttcagaagtatctgttaaggggcagcctggatggcttagtggttcagtgccatcttcagcccagggcgtgatcctggagacccaggattgagtcccacgtcgggctccctgctaaataaataaaatctttaaaaaaaaaaacaaaaaagggcagccccgggggtggctcagcggtttagcgccaacttcagcccagggtgtgatcctggagaccgaggatcgagtcccacgtcaggctccctgcatggaacctgcttttctctctctctgtgtgtgtctctcttaaataaataaaatatttttttaaaaaagtatctgttaaaatggaaaatacctgacttacccccaagaaacaccagcttttgttctaaaatgatgtgtataggaagagatgtatcacaggaaaatatacagtagtgaaaaattggaaacctaaatgtccatcagaggacttgaaagaaaatatatcaaaaataattacatatgtatcaatatcagctttaaaatatggtatcaagtgagaaaagcaagctacaacatgagaccacttaaatttttaaaaacaaatttgtatatttggttgcatttttttttaggagtgatatacatatgtatgcaaaggtatcaaaataagggttagcagacacacaccaaattcagtagtggttgtctgccagaatggcagaaggggtcaggatggagtccgctatcaaggagagaaggcctttagttttacctataacattctgtttgtttccatcttttaataaacaagacttcacagctgtattttactttcaaaactttttcataagtataaattttgaaaaaagacaggccaatgcgtaaaaattttaacaggattgattatggtgagaaatatcagagatggctacagtgattgtctttgatacagtttttcttaatttcaaaataaaagttgttatagaaaaatatatggtcatcatagaacactcaaaatgtaaacataaataagtaccagtaatctgcagagagaatcaccattaatattctgatacatagtgtgtatcctttcagaattctgtattttaatacattcacagtgggttattactgctttaagtatggaattcattggttttacctttttcactcttatttaaatatctttattggtcaataaatatattgtgatcccgcacctctgctacatacatactagtaacacactcttcctctgttttttataccaccagaagacactggtgtttttaatataacgcagtaatgaatgtagcaggtacttactatttgccaggcattgtcctttatgtatcctaccttatgtaatcctcagcagccctatgaggtacatcccattattattcccatttacttaagaagaaactagggtgtttttttgtttgtcttttgtttgttttttgttgttttgttgttgttgttttagaaactagggtttagatcacataacttgcccctgacactttggctccaggatatataaataaatgataaatataaatgaatatataataaatataaataaataaataagataaataagatcttaaaaaaaaacttttcagaatagttgttgatattcactctaatttctcttctcttctctccccttttacaagcctattccagtcaggcttttgccccgatactccaccgaaacctcttagtagatccagtggttggattcacagtcctccatctgtttgtatggcagttgatcacatccttctccagtgaaacccatccttaatcctttgctactcagaatctggtatagcatctgtaccacctgggatacttttagaaatgctgagttttaggtaccaccccagatctactgaattggaaactatattttaacaaaatccctaaatggtttgtatgcatattaaaagttgagaagcactgctttagtatgtattctcctgattttccttctactcactgctctttctcggctaattcttcctcagcttttaacctctctaaatgttggaaacatccggaatctccattgccttggtgatctcatccagtttcatggcttgaaataccattcaattgccatttatatgccaatctccagccacatcatttcctttgacctccaagctgtcatatccaactgtctcctccagattacttgattgtctaatggatatctcaattttagtatgtctcctactgaggacctgaccttttcctcaagctagatcttcgtatattccttttatctcagtcgctagcaactctgttctgtcagctgcttatatctacattttgtagttttctttgggtcctctctttcttataccatacaaccaatctaatatcaactcctatccactccatcttttaaagtatatcagaatcttacaagttttcatcacctattgtgctattcaccctgatggaagccatcattatttcttgcctggattagtgccctggttccatccttacatagcacccagaatgtttggggacttacattatgttactcctctccccttccagtgtttcacatttcactcagaaagaaagagtccttacaatagcacttcctcaaagtaaccaccaccacgaccctccctcctttcttattacctctcccaattcatttccttctcttttccctcttgcttcctctcttccaaaccagtttggcctcttaccattattagaatatgggccctgccttagggccttgccattgctacttcctttgcctgctatggtcttatcccatataggcatgactcccctctctcctgtctttaatcaaaagatcctttgaaaagaggccctccctatcctaaaattatagccccctgcaactcagtatttcttgtcccctacttacagaatactatatattctactgatacatctttttcttttctctcttctatcctgaaatgtaaggacctcgaggtagggattttctgctttatctccaaaacttagaacagttgttgcttagtacacagtggactttcactaaacatgtgttaaaagaaagtatcccttcaagatcattattacctccattttttaggttaggaatctaaagctaagaaggattatgtaatattgtacccaagatcatatggctagtaagtgataacagttggaattcgaatcccagtctcctgtgctcaaaacccatgccctttccactacgcctctttgttatgtcacttaagtcttctgggctctggttttcccatcagcataagtagcattattacctttttgcttgcctcacaaagaaacatgaaagttaagaaacttactaatgcaaaaatatcctaaaaactttaaaatgctgtatgtgtacatgagggtggtggaaatttttttcttttagaagtttcataatttttgcttttagtttaaggtcttaaatccacttcagattggtttttgtgtgtggtgtgaggtaagggctgaagattttttccccatgtgaatatcccaatgatattcattgaaatgactattctttcctccatagagctatcttgctcttataaataaataaatattatgcttctggttttggacttgtgaatgtttgagatgaatttttaaaaattaattacatggtttttgatatttcatgtttaaaatacattttcttttatttttttaggtcactattttactttagaaggtctaattttccaaagagggattataccctctataatgcaggtcccagctaggaaacaaaaaaatacatagaaatatcatatttgttaattagaagattcaatattgttaggatgtcatccttcccaaattgatctatagacttattgttgagtccagtcaaaattttagcaggcttttttgtagaaattgacaagttgatcataaaatgtatatggaaatgcaatggctagagtagtcaagacaattttttaaaaattgaaggacttctagtcttagaattcaaaactttgtatgaagctactgtaatcaagaccatgtgatattggcataagaataggcatataaatcaaaaaacagaatagaaagcctagaaatatatccattagatctacggtcagttggtttttcttttttttttttttttttacaagataccaaggcacaattcacacagaaaaggtagtcctttcgactcatattactacaacaactggacatacatatagaataaataagcaaacaatttgcccttagccaacacaagacattagtattaccttgagatgggttgaagattaaaagctgaaaatctaaaacttctagaagaaacatatgagaaaatctttgtaccctaaagaaaggcaaagatttcttaggacacaaaaggtaaggtaagttggacttcatcaaaatttagttgctttttgaaagacactgttaaaaaaaaaaataaaaaaaagaataaaggacaaaccacagatttgaagaaaatgtttgcaagatacattatctgtttaaaagacttatatctgaactatataagaacttgtacaactcagtaataaaaactacccaacaagaaaatggacagaagattggagtactttattgaaaaggcataagaatggcaaatgagcacatgcaaagatgttcaccatcattagacattagggaaaggcaaattaaaactacaagacaccacaacacacccactagaatgcttttaatcaaaaccaagtataccaagtattggcaaggatgcagggcgacaagaactcttacacattactagtaggatcttaaatgagacaaacactttggcagtttcttttaaagttaaaattatcacgtgagcaattctaatcttatttatttacccaaaagaaatgaaaacatacatcaacaaaagagacttatacttgaatgtttgtggtatcttactataattgccagaagcaaggaacaacccaaatgtccaacaggtcagtggttaaattgtggtatatccataccctgtaataccacatgcagccagtgtgtgaatcttagaatcattaggcctggggatccgtgggtggctcagtggtttcgcgcctgcctttgaactccaagtcctggagtccctggatcgagtcccgcatcaggctcccgacatgtgggctgcctctccctctgcctgtgtctctgcctctctgtctctctctctatatgtctatcatgaattaaaaaaaaaaaaaaagaatcatcaggctgagcaaaagaagccatggatttttgtttatatgaaattctagaaaagacaaaactaatgtatagtaacagaactttattatccatggttgcctgaaccaaggagggtgttagtaattgcagaaatacgtgaagaaaattttgtgggtgatagatattcttcttgttggttgtggctgctcattactgtgtatgtttgtcaaaactcaaaactatacataaaattgggtgcattttatgttatatacatgttatacctcaataaattagtttgttttttaaagtaaaagaaactgaaaactttaagtaaaaatagaatttgggccactttggaacacaactggaaaatgtgtgtacttcatgtcttagggattttaaccattttaaattatattgacagcagaggcatagcagaatatcattcccttcaatcttcctcactttctacattgcgattatgaggggagtggagggcatcaaagtagaagggagactgtaattgaaactgtttggatcaaatatcatcttgtcactaccatcatgcatatcagtccagttggctgttatattctctgtagtgtctgaggagagattgaactttctgtgtgaagtctgataaactttttgaaattgtgtagtattagtgaaagaggactgaaatactagtgtctatgtgaaccagttctctgcttttaaaaatagttgaaaatacaagaaaataacagtaaacttaagtgcgagacacttggaactgttgtaaatagacaagactgataaacatttgactatggcaaatgtagccataatatttgagttatagcaaggcatttgatacttctcatgatattggtgaggataagatcaagagatacaacatgcatggagaattcaactggatttataactgtatattgatcatacctgaagttgttaagctaggagatttttgtttcattcttctcttgagtaagactagcatatttacaaatagataagggcttcattagtacactggtcttttaaaacattttttagtgagaactaaacttttaataagctggagctataggccatgtctacaaaataggacatccaagtccaaaaaagagagaaatacaaatagaggatggggaaactgttttgtaagagtaatgcaagaacaactcagtagggttgtaactgaatgtaattttagtaggagttagtagagtgactgtggctgccatgataatcacaataatgggcgataaaaatttttttaacccaccatgaggaagatgaattttatttttgtcttgtttttggactccacatgtaagtgtgatcatatggtatttgcatttctctggcttatttcattatgtataatgctctcagggtccatccgtgttgttgcaagtagcaaaatatcctattttatggctgcctgatatttgtgtgtgtgagtgtgtgaatgacattttctttattcattcatgggcactaggttgttgccatgtctttgctattgtgaataatgctgcaataaatgtgaagcgcagatgtctctttagatcctgatttaaattcttttggatatatacccagaatgggattgctggatcatattgtaatcctgttttttaagtttcttgaggaattattttccataatggttgtactaatttatgttcccttcaacagtgcacggagttcccttttctctacattcttgccaccatttgctatttcttgtctttttgataagagccatgctaacaggtgttaagcagtatctcattgtggttttgatttgcatttctgtgataggtagtgagtgatgctggacaccttttggccatctgtatgcgttccttgggaaaatgtctgttttttaatcagattgtttgggggttttgatagtgaattggacaagttttttatattttttggatatcctcttattagatatatgatgtgcaaatattttctcccatttgataggttgccttttcattttgttggtggtttgtgtcctttgctgtgcagaagctttttagtttgatgtaatcctacttgtttatttttcttttgttgcctttgcttttggtatcaaaaaaaaaaaaaatcattaccaagaccagtgtcaaagagcttgctacctggacacctggggtgttcagtgcctttggcttcagggtcttggggtcttggtcttgagtcccgcatcaggcttcccacagggagcctgctttttccccctgcctatgtcactgcctctctctgtctctctctctctctgtctctctctctctctctctctctctgtctgtctctcatgaataaataaaatcttaaaaaaaaaaaaaagatcttgctacccctgtttttttttttttctaggagttttatggtttcaggtgttcaaggagaaaattaagatgagagttactctagtttatgttgatcagatcagatctagagtattatcttcactcttgggacatcatattaagctcttaaaagaatgttaaaaagctgaatcttgggggcatctagttggttcagaggattaagcggttctgctttccactcaggtcatgatcccagggtcctgggatcaacctccatgtgaggcaccctctgcctgccactccatttgcttgtgcgcacaccatctcccttgtcaagtgaataaataaaatattaaaaaaaaagaaagctaaatattggataataccttgaaagaagcagagagaacagattttactagatgattgcccagctttcagattaaatgattctctaggtggatacttttttttttttgtttttcaaggaaagacactcctaagataaacctatcgagaggatatattcatttggtttatagcatgttttctctctctcttttttttttttagattttatttatttattcaggaaagacagagacataggcagagggagaagcaggctccatgcagagagcccgacgtgtgacttgatcccgggactctggggtcatgccttgggaccaaggcaggtgccaaaccgctgagccacccaggcatccctatagcatgttttttcttactgctacttttataaatctataaggttctctttaacatcttagaaaagttaagaagggggtcctatttaatttccttaaataatgggtatacttctgtatcatatgagttaatttcttattgttcccttccttccataccctcagttatgtgtcttttggcatctttgattataactcattgttgcttagaaattatgttttagtgtcagcctatgtagaattatatagagtagtaattactctggaacattatatcatgtagagatatggggtataatactcattccatttttccatctggggaatttgagtaccttttttttttttttcaaaccatccattatatagacctggatttctgccttttcactggttttgtggcagagatgttacagaaagagaattattacatgagatagaagttaagaaagcagcttgtaaacaataaaagataagattacacaaggataaggtggtgctatttcaagttgcagctgtatacaacttcagcctataataaatttatgctatctaccttccagtagggttctatcactagctcagtaattgtactcatatcttgctaattatctagtactttcctggtgagaaattttttctcctcaccctctagttgatttctgtctaccttattcttgatatatttctttgtatcagatttgttgaaaagattgataccatcaagaacacctttatcttcccttataacttcatcatgcctccacatattcatatctaccacctatttgttgtttttaatttcaaaggagacagaatcttttgtcaaagactaacacctattatgtgcactaagtaccaggtctcagaccaaagcaccaagctccaccactgaacagcatcaccatttgttacccttcctctctagtggagacaaggtctccctatcttgaaaaagcttgtggttgaggctcctgttctccatctgtatcattctgtgtcattgcagaaaaatttttaagcaaatgacacctataacagacatcacattttcttccaatgtaagagagaccatgtgatgtagtgggacaagagtgtggtaaatactatggaggaaataaacatagaagtaagagtaattggaggagaaggggttcctttgtataaatcatttgaaatgaaaattttatctgagaagaaagcttcaaaatcttgatctctgggagaatgaggaagtataaatgaagaagagaaagcataaataaagtttaattgttatgaatcttcaatttttaacagtgtttattacataattttgattatatactatttggactttgaaagtgtcactttaaagcaaaatttacttcctaattaatggtcattgtcaaattttagaagtctcatatatctattctttccagttttaaatgaagaaaaatagctttgactaggatattatatgcatttcgttgtagttgattcaacagacatgttgaacacttaccacatgccaggctctgtgctaggcactggagattgaaacagaaataaaggaggcacagcccttaccctcagtgaacccaactaaaccagtttttagggctcacataggtaatttaaacttaccattgactataagatagatgcactgtggatattatgtagcaggtttgctcctcactatttttgtttagaaattttattttattctgcattattttctcttatatgctattttaaactaatcaacaatggagttattcactgtggccattattaactaattttaagactaaatgattaacatgtgtgataaagctccgtatttttaagaagttattttaagaatgtataatactacctttaaaattaacaatcaggggcagcccggatggctcagcggtttagcgccgccttcaacccggggcctgatcctgggggcctgggatcgagtcccatgtcgggctccctgcatggagcctgcttctccctcggcttgtgactctgcctctctctctctgtctctcatgaataaataaataaaaatctttaaaaaaatttttaaaattatcaatcagattttatattctagtggagtttacattctgaaaagggaaccataggcacaggaaaaagaatacatgaatcaagctattacagtaattacgatataactcaacagtgaaagattacttttggttgagttagtcttagcaaagctttattcagaagatagaatgtatattcaatcttaaaggataggtatgaataggtggagaatagaatgaaagttaatttggggatgggtttggaagggaagtattgatcacaggtgggattaaatatcagggtgtatttaaaggacatggataaataggtttgactggaatgaaaggtttgtattggtaaatagtaaagtataaagcaaagtcttttaatagaatacaagatttgggctctaccataatagtaatatgtttctataatcctaaattcatttacagtttaagatttcttacataaagaggttatcacaatgtacagtttgcaattgtgtcatgattttgaataagtctttatttacatccctaaatatgttctatagattttgaggccagagtctttacccctactcaggttgaaatgatgttttgtttttcagttacttacacgccaagtcaatcatccacagagacctcaagagtaatagtatccttcctgaaatttgtctgcgaagtttgaaaacatcctgactttttcttctgcattttgtcttcacattatgtaaaaacagttttcatgctaagttcaaaaaatgtaaagagaattaataaatgggtttgcatgagagcatagtataaagcagggtgctgcaacatgcttttggtttctgaacatttgagtgttataaaaaaaaaatccaccaagtctttataatagtaggactcttttatgattgtgtgcttacattctgtcaacacccataaaacaggtagctatctacccatttctttcagtgctctgatgctactctgcagtagttgaaatgtaactgaaggttctaacttgcataacaaggaggtgagaaaaaacaaaatttttcttcttccataaacccagatcaagatcctagccgtcaaccaacttcattccaatgaagaacctttgtagctttcctaaatgctaaagcatatctgggtccatgttactaaaatctaacgggaacatttcagggtttcccataaacataggttggaaagcatctcacctcatcccatcatatttcaagcccccaaaatcttagaagcaggtcacatatgccaaatagaaccatatttttaaaaacatattcattaactttaagaggaaatatgaagtactatactttttaaagaaatatcaattttaccaaaaaaatcatattatagaattacagaaattagatctcttacctaacatttttgtaatgcttgctttgctaggaaaatgagatctattgttttcctttacttactacacctcagatatttttcttcatgaagacctcacagtaaaaataggtgattttggtctagccacagtgaaatctcgatggagtgggtcccatcagtttgaacagttgtctggatccattttgtggatggtaagaattgaggccatttctccattaattaaatttttggaccctgaggtgctactgagtgactagaaaatctttgaaggtttcgactagtattttcataattcccagtattcacaaaaatcaatgttgatcttattttttatgtaaataaaatttaacttttttctttatccttaaaaagagtattatgaaatcagtttcagtatacttcaaacaaaaaaatagtattttataaacaactgttgaaattttatccagaattgtctctttaaaatattttgtgttcaaaatgttctgtgtgcccggtttcaaaaagaaaaaaacaagtgtgcagtttcagcaggtacgatctgcagtatttcttaaggttttactaaaagaactactccttttaagagaggctgtttcatgatgttatttaagcctaaattggaaagttacttcctttagactagaaagaacccataattatggggcagctgggaaagaaaagacataaaaaaaaaaaaaagaattttaacatagccatgtgctgccctgcatagctctttgttgctttttctttattcctatttctgctttgtgtaaaagaagggaatgaaaacttctgtgccattactttcagcaggctgtgaaaagggcaggtataggaatcatcctctaagctagcagttctcaaaaggggccctaatacccttttggaagtccccaaggtcatccctgtatgagaccagatgtttctttatagtcttgaaccagaacaacacatcacaacagatcgaatgccagaaccaaaatatgtgactatagctggctgctcttaaaccagtcaaaaagaattgcagtactcttctcactgaacttttttttggatttgaaaatacacttttcattaaaaatgggtaatctttgttaacatctaatggctattacttatgaaattactaaatattttaacagtttcccagttatgtttaaatatgagaaatacccatagatataatccacataaaagttccttggggtcttcagtaattttcaagactaaaagagttttgagatcaaaaagtttgagaaccgctgctctcagctgaatgccggatgtccctaaccagtagtgcaatttgaagctttaaataattgaatctgcactaaggctttgggacactctgctggaaacagagaagctgcaggggctcttgaagccatgtttgcataaattccctgaactatctgtaagagcctccaagaggggaattccaggatagatgtgaattagtcaaataaacttagggttaaattcagctatgctttcttataaaaataactggagtgaagacaagtggtaaggtggaaggggtacaatggacctaggagatctgtactctatagtcctctgtttaactctcaacctgcacatttcctaacctccatgtctctccatctttaatgttggtcggactagtggatttctaacaccttttttgttcctaagattcagtaattagagctaactaggtttgtttctttaatatattttaattgttttaataaatcattttataaataggaggcttattagtcattgtaattgggatctcatgcacctttaaggcacacaccttgctataagatagcagtggattctaggatgcagcaggctagcacacactaagtgtgagactgtaaaacctcaaattaaaaagaaacaatacatgttttaaaggtgtatctattttaactagtgtgggggagatgctcagttaaactccaggtttgtcctttttctaaaacctagattattatacccaaagttttatattctaatttattttttgttgatacataggtagattggttcatcctttattctatcaagctgagctttcatataagagattagctagtagtacacttacgtgttgaaaaaatccttttgggttgataattgccttcagaaacaaatgcggagcatctgtgtactagtagagaagagtttggtcagaactaagattatgtaattctctgaattcttctcaaccttgttctatttatcttagaggggaaagaaacctttgtgaaaatttttacagattaaatcactaaattagttgaggatcaatcatgcatttcttgtccaaataaatttcatcaagccatctccaacaacagtagaggtaaaactaagttttaaaaagatggttagttcctcaactgatttattattctgtccctcgcaaaattgtattctggcttttaaaattatgcctatatcataaccagcctctcagtattctagcttgttcatcacatcacagttgtatgtggtttattttgtactgtattactaaaagcagctagcaataaaatgtttgacaaaaaaagtaagcagaccccatcttctccttcacctcccatccttcatcaggatctcccatacgtcttctgtcagtggttctcaaccttaattggacattaaaatcatctggggagctgtaaaaaccagcaatgtttggatcccatccccagattctgttgttatattattggcctggtatgtagtctggacattgagaggttaaaaagtctacacagttgattgtgatttgcaagtaaggcttagtgccattgcctgctttttgagatagaccagtgaccaggaatcctgtcccaggtcctccagccatttatgcagttagttcaggttttggtatcaaaataatgctttcatggaacttttatttacactctgcagtgtctttagaaacaattcttccaaatgaaacagtacagaagatgcaagggaaagaactcttttaagcgtatatttaaccaaccatttattgtatttcatgaacacagccaattaagaaattaagtggtagagtacaatgagtaaaatgtgtaaaaaatgtagcaaatatgatttggtctgtatctttgcccaggaagcaaacagccagcattctttatgccatcttctaaattgacctcctatctcttggttctgttaataaagcacccatggcatattgataatgcttttggaggcaggaaaggggtttgagtttgtcagtgtcgatgttttaccagtattgtctaaggtgccctgaggcgagaatcattgtactaatgggaaaacttaaactctaaggcaattattagcgtagtggttatgctacttccaaacattttaggactgcaggtcttttatttctttactgaatcaggaatggaaattggaattgattcttgatagagctaatcctgggaaggattcaaatagcaatggcagtattttgtacagaaagaggatggctgataaaattataaaatcttcataatacatgtggaatctcaggtaatactgcctctttcagggcttactgagcatttttttttcttattaatcagttttatttctttgccccttaataagagaattgattgcatattaattagatggattatttaatgacgaaagcattgttctgggaattgtaatcatttgcttttcagtctttttagttgaagaataaacaagaatatttttctttttgtgttctaataggcaccagaagtgatccgaatgcaagacaaaaacccatatagcttccagtcagatgtatacgcatttgggattgttctatatgaattgatgacagggcagttaccttattcaaacatcaacaacagggaccaggtaaatatttaccatctcttggggtttattttactgtttgtataaagactccagtttgttgtagaaaagtattgagtccagagtaggcataaaggatggatatcttaatctttgttaccagttttggaaacctttggtggactgttggcagtgatagcagcatgtaaggcctttccttgaaatgtcagtgttgtcacggaatgcagttgctgatggtctgactagaaaccagagctgccacagtggagagcgcccatgtttcctgaccacacacaaccagaaggagctctgctgccatggcatctgagcttttgtgccttttattctgtcataggttgtgctgagcaggcactcagctatgtgcttctttatatccatttctttggaaagtattagtaagctttctccatttttaaaactaggtaaatggaaagaccattgtatcatgctttcacttttctatactaaggctttcaataggagggaattttttttacttccttctttctaaattcctgttctctgtccttccccaaagttcctgacagagcctcatgcagcattttgccttttctatcccatccacgttgagttcaccaggaccttaacactgactaagaaaaaggaattgtctttactgacattatattgtatacctcaagattttaaaagtgtagggtgggttggggggtgggggttatagagacattgcagcagttccattcttcatatattagaaaacaatgatcaaccatataaactgtggttaaataatatttatattttatactgtggcttgcatcagttttacctggggaatttgcttaaaactggagattttgtctaaaactaaaagagaggtgcctaggtggctcagtctgttaagcatctgactcttgatttcagctcagatcatgatcacggtgtcatgagatcaagccccacatcaggctccatgctgagagtggaatctgcttaggatctctctccctctctcataaataaataaataaataaataaataaataaataaataaataaataaataaataaataaataagtgtgtgtgtatatacatatatatatatgtgtgtgtgtctgtgtgtgtgtatatatatatatatatatatatatatatatatatatatatatatatcctagagtagattttgattccatggagtgagatccaagcatttacatttttaataagtttcccaggtgattttggtatataccaaagttaaaaatcaccaaggaattaaaaaaaactataaatggggcacctgggtggctcattggttgagtgtctgccttgggctcaggtggtggtcctgggttcctgggatcaagtctcaaatgtcaggctccctgcagggagtctgcttctccctctgcctgtgtctctgcctctctgtgtgtgtctctcatgaattaataaataaaatcttaaaaaaaaaaaaaatacagtagggatccctgggtggctcgtcagtttagtgcctgcctttggcccggggcctgatcctggaatcccggcccgcatcaggctccctacgtggagcctgcttctctctctgcctgtgtctctgcttctctctctgtctctgtgtctctcatgaggtaaataaataaaaatctttaaaaaaactgtaaatgttttgtgttttatcataaactaatgaaattatagtttttaatgtatgaatatgttacttcatttaagtagtgtgtgtgttttggtcatcagctcaaatctcagtctttttggaaataagctctaagtatctttcttactaagaacatcagctatcatatggtttcagagaaataatctcagttgtcatgtacatcatatatacttactgaatcaccatatggtcccttacccagagtggtcctaattttgtagttaaagaatttttcctaaatgactgtgtttcggggcacctgagtggctcaggggttgagcgtctacctttggctcaggtcctgctcctggggtcctggaatcaagtctcgcatcaagctccccacagagcctgcttcttcctctgcccgtgtctctgcctctctctctctctctctctctctcattaataaataaaatcttaaaaaaaaaaaaagactgcttctatccaagtcagtacagagctaattggttggtttctgtttggtcttcctaatcactacacagctttagaatgttgaatcctacttttgagtgttaaaaaaaaaaatctgtgacctggttcctaattacctgatattttaagcctgcatgaaatttagattcaggatagaccaagctactactaaggaacatagtagaaacattgcaattaactggacttgtgacagatcctgggtaactgagatgatagatttgatgatggcagtctgaagagcaggattgtttctaatttgcaagcttcctgtactctgccttttatagtctcattaactagactatatgacataatactctgtgccatgttcacttttcctttgcatattacttttttttttaagatttttaaatttatttttagagggaatgagagcatgaatgggggtaaggggagggagagggagcgtctcaagcatactctcgctcagcacggagcccaacacttggttcggagcccaacacttggttcgaccctacgaccctgagatcatgacctgagccagaatcagggcagacacttaaccaactgagctgcttaggcaccctgcatatttcttttctataggaattacaagagaattttagtttagttttagttgatttatcagagacaccataaatatgaatcaaagatctggatgaaatccagtgaatatctttgttttaataaaataataaaattctgttatggaaatgggtattattatctccaaaatacagataaaaacatcccagagttctatactcatccacgtcttagcattttaggtgctttatcttccatgcgaatataataaataacgtggcaagggctttctctccataagagaaataagtgagtgaccaacagaagggtaagggctttactagttaattatttctggtagtatagtgagagccttctggaatttgccaatctgaattgtgtttgatgaagaaaaagaaggtagaagaatgatacactcttcctatcacccctagtctaattcattacttcttagtctgctttcctcaaatggcactttccacggaactccctcttgggaatcagtaactctagttatgttcaatgaagtaatcccaggctttgctccatctacccccttgatttgatttttcttttcctcttttactttgatctccatggctctcagattggacttctgtttgaaaatttaggagtactcttattcatgggagataggactacttttttactctgcactataggaaccactcctgtgcgctctcacccccatattctgaaatggtagcttttgcctataaacttaaagcaggactctggttaagcagctcggtataaaggccaaccttggacttgtgacctgcatgacctggtcactttacttcctcaagtgtctgtattttcttccctttcatttataaaaataagaaattatactaaagagggacaccagggtggttcagtcagttaagcatctgccttctgctcaggtcatgatcctagcctcctgggatccagccccacatcccgctccaggctcagcaaggagtctgcttccccagcttgtgcgtgtgtgcgcgctctctctttctcaaataaataaaatcttaaaaaaaaacaaatcagactaaagaattccttgtgtttacaaagatcatattctttactaaagcacatctcttagctcttcaggggatggacatccttgttactttgcagaacagtatgccttttgtttaccttacccgatgctgaggtctaagaaattggtgtcattttttccccctctttatccttccatttcgctctctaatcaccctgttctgggcagcccaggtggctcagcgatttagcgctgccttcagcccagggcatgatcctggagaccagggatcgagtcccacattgggctccctgcatggagcctgcttctccctctgcctgtgtctctgcctctctgtgtctctcgtgaataaataaataaaatttaaaaaaataataataataatcaccctgttcttccctcttccagtgcagttttgctaagttgggattaataatgcattcatatgaactaggtatattgacactctatcatcatgattttctttccttagagcagaagtgctatgtcatcttgaagaaaataacagaaacttctgtctcattttaagtatatttcagttagtattgttgtataataaaccaccctaaacatagtggcttaagcagattatgaatctgcagttcaagctgctgtcagtaaggaaggtttattttctgtgtcatatggtattgcctaagatacattaattgggtgttaaaggctccacttccaaaatggctcatccacacattctacaaggtgtggtgctagctgtccagggccaagggcagggggcttcagttctccacatgggcctttctgtggagattttgttcaaagaaagcgtggacttggttacaggctggtggctgggttctttttcttcccctggtggctggttctaaaacataagatctcaagagataagcgataggaaatgaaagtaccagtgttttatcaacacgcaaattattttaataaaaaatttttaaagcactatgtgatatcgagggtgacaaagacgaataaaaacacaggccgtgctgtcactggaattcaccagtctactgaggcagtatagggggcgatattgaggatgagcagacactgtgtcctggtaagacaggagtgaatgaaggtgcagagatgccaggagggctaaatgtgggttgagcatgaaattagccagtcagtcccagaatttcaaattgagctgctgattttatcatgtccatgctacttaagtctagtatacatgaattatcagggtaagtcttccctttaagtaattgtaaagattgtgatttcctttatattttattaacatttcaaaataaagctgttaaagcttcttttaaatgtatccaatgaaatctaaaagccatcctgacttgatacctaccatctgtgtaaaaaaacagattcgataacttttggaagtttttctctttgaagaaacatttttatgctttcaaggcttttattgatcactcaagttatttatatctctgcaacaaaaataaggatttaaatttagaattaaaaaatatatatatttcctgggactcctaggtggctcagtggttgagcatctgcctttgtctcagggcatgatcgcgaagtcccaggatcgagtcctgcatcgggctccccaacagggagcttgcttctccctctgcctgtgtctctgtctccctctctctgtctctcatgaataaataaataaaatctttaaaaaaaaatagttacatttacaaattttttaaaaaagaaaatatttcttaatgaacaacatgggtttttctcatcagttcagttatatatttgtgggtaaatagggttttgctaaagtgaggtaggacttagcatcagttcctttccttgttttatttttaaggaagtaagcactgaaaactcctgttcaattagatacaaagatgagaaattcaggcgtttgttgtacccacatgcacagttcagtggtccccaagaccatactcagattcagccattcagtaagtctcagaactccctccaagttgttagtacttaaagttactgtttattataacagtcaagggaagaggcacataggacagaattgaagaacattccatgtgtgtaggtcctagtgttcttctctcaacgtaattgtggacagcatctgaattcttctggcagcattgtgtgacaacgtgtagagtattgccaaacacagaaacttgaaagtccagagttatcaccagggctcggggtcaggtaaatatggttgaccacctctgtggctgaccttagtttttagcccctctggaggtcaagctcaatgtcccttctataaatcacatttatggcatagaccatccagcatagccaaaggcctccaggtaaacaaaaggactcttatcaggcagggcattccagaagtatagggattacttcccaggagccaaaggcaaaagccagccctccctctgggcaagattaattctttactataaagtatatcactcagttatttgaactcacttctacaatgtacccaaaattacataataatttgtcagtaaattttataattagctgacaattcaattaatcttgctgcaagcaaagaattggaaaccggctgtcttgcaaagggatttgttacccattcattagagcctagtaaaataccagtatccgttaagtgtccgcttggtacccagcagttttacctcttggaacaatgtgtactatagttaaattttatgttgggtaaaaaaggtgtgccttttataaagtaggaaaagggcagttatagaggggagatgggaaaggaggatatatacctccactttagtggctcttgggcaaatcagcttatagttaaaagtttgcaccaaacaggaaataatataaagttagggtatcagacttgactgtttctaaagtttataaactctgccctgtatgtaacaggatttcttttgtgtgtgtgtgtgtgtgtgtgtgtgtgtaacaggatttcttacaggctctctccacctaccctctagtctgtgcctgtatgacctgttccctctaagatcaaaatcccctctcttcacagagtcccaagacgcctgatgtctgtttctgtccgttctcaaaacaggcaaaaatgagaaaattaaatcagaaacagtgttcatctctgcacctagggaagacaagactttttactttattaaagagaaaacttaattcccaaggtggtttatcttctttttcttcggaggaggctctgattctcagacctacattactacatgccccatttctaacatttcctcaacaaacgaatttgttgccaaaacaactgctaaaaagcttagcttttttggttcttgctacatatatatagagaggacttgtgatgcccaacaagtactcaataaatactcaataaattactttaagtagaatagaggcacagtccaatctgattaccctggcagtgggaaatggataaggacagactggaagcctggagagcaataagaactttacagtagtctagaggtcctgaagatttcagcttacagcccacaatatgatttaggggatgcattgtgtttctttttattgaagtacagtgtacatgcagaagagtacacaaaaatgcgctgcttgttgaattttctatgtttatgtatttttcagttcatcaacaaggttgtatgatttattgttttttttttcccttgcccagataatttttatggtgggacgaggatatctttctccagatctcagtaaggtacggagtaactgtccaaaagccatgaagagattgatggcagagtgcctaaaaaagaaaagagatgagaggccactctttccccaagtaagaagctttatgttacctaagaggataggctaatactcagatgtagatttttaaatagtttctagagcatacagtttgtattttgtgaatttggattccatgtgattatgtgcaggcagcagataagtaatcacataaagatgattttatgaatttgaatggtggtacgtctagaaatttggaatctcagtacaaaatattaagaaagactttgattttgtctagcagttctcagacttttttggtctcacaatccatttacacacttaaaaattattgaaaacccccaaagaacttttgtttatataagtaataattgccattttagaaattgaaactaagttttaaaaatatttataagtttaaataaacataataaaaccattacatgttaaataacacttttgtaaaacaactgtattttcccagacaacagaattaatgaaaagaatggcatcgttttaccttttttgcaaatctctgcaaaaaaaagacatctggattctctgcatttgtgatgttgcaatattttgcatgatgtagcctctgaaaactccacttacacttgcaaaataatgacagtaaaaaggcaataataccttgctgttattatgaacatagtttttctacatattacactttgagaactgctgcttcagttcctcctggtggtaaaacattcccagcttccctttaatactgagtctgaaaaaaaaaaaaaaaaaaaaaaacactgagtctgttggaggagcattttcaaatagggcaaaaagtgccacctattggcactgtctgcctttctctgattcttggaaaattaccatcgctttttatcttttcatggcataaggctaaagcccttacgttaacaatctttaataagcctcacaaactgcaactaagtacatgaagaagctgagaaagtttcgatatggaagatattgtaatataaattcagttttttgtgatgctcaggaagttctgatagcgctgtataaacaagtcttgataaatcagtacaattttcagaggggtaggactgactctgtaattactagctaccataattactgagttggtctttcttgaaccttattatgcttttaaatacccactcctagctgaatctcctaagacaaaagtcactgttacaggaatacatgaatctgttattcccatctaacacattaaagattgaatttgtgttttaacaagtctctaaggggctatctcagagtgggtagatacatgttttacatattagaattctaagttactatttcttcgaggaagagttgtttttttgtttttttttttcaacccagaaaactgctggttgtttaaaactatcaaagtaaaaggaccagtgttgttggccaatatcagcaggactctggtatcacattctgttttcatagtccaggtactagagtttcctgtttaacatgcttgatgtatattaataatgtaaattatatccacaggcccacttttttattttaagattttatttttttaagtaatctctacacccagatttgggctcaaacttacaaccccaagataagagttgcacaccctatcaactgagccagccaggcagcccttatagtccttctttttaagtggtattaaaggggacaaaatgtttatacttctaaatataaattctttgtgtggtatctgaaaatgaaatggatatctgaatttttcagattgctgaggatgctcgttttaagtattatttttaacttttcattacaagatctcttttcacactacacagaaacacaaatttcagatggctaaagatttatatatgtgaaaaacataactataagaagtcatttttattgaccttcaggtaggaaaggctttcctaataataataagcaagaagcgaaaatccagaaatgataaaggaaagatcagcaagtctgaccaccttaaaagtttttaaaattctgtgtaataaaaatactattgaaaagtaaacacaaaaaataattatagcatgtataacaaagcataaatactggtattatgtaaaggcctcctacaaatcaataagtaaaaaagataacagaaaatgagcaaagcatatgaactggtgattcacacagaaatacaaatggtgaatgagcttaggatgttaaaagttctgagacagtcaataaaatgcagagtaacagtgaaacacgttttgattttctctctcagattggcagtatccagcattgggaaccgtgttgggaagtaagaagggccgggccctccttataaattttttaacagtattgttgggattgtaaattggtttggtctcttcaaaggacttggtacaattcataaactccaaatatatatacattttgctctggtaattccactcttaaaaacgtatactacagaacctgcttccacaagtgctcagatacatacgagaatgcttaccataataggaaaaattaaagacatagagatcaaggtagctctctagatggcatggaagttagcgaggggaaagaaaagcaacacagaaaaatatgtgtagtatgataccacttggatggtggtgagggtggggatgggctctgttttcatatgtgcatgtttatgcataggaaaggtccagaagaaagtatagactctttaacagtggttacccctggaaggtaggacttatactttttctgtattatttgaatttttatttgtgtgttatgcttgtaattgaagatgttttgtaacattgcttctttgccttatactaatcatagagattacagtttaactttttaaaaagttttggtagctcagaaacataataatagtaatggctgctaaagttttattgagttcttactatatgccaggcattgtgccaagcctacacgtatgtctcatgattatgaacatccattttactgtctgccttcaagagtcataacccgtttctctgtaccttaaactatgtggagtttctacgttttgtgtgttggtgtctatttgtcgtttgcctgtcttttccactcttggattgtcacctgtcacttctcactcctatgccactagcagtgtgggtcatcaggtcctacttttaattatgttgtggaagacattcccccattactaagtttcttaaagaatgagttgctctgatgattgtgtttgtgaagtcacccactgctttactgattgatgcttcatcttcttcccaaaagggactgcggtctgggagaaataggtatataatcggtttaaaactcagggcagcccccgtggcgcagcggtttagcgccgcctgcagcccagggcgtgatcctggagacccaggatcgagtcccacgtcgggctccctgcatggtgcctgctgctccctctgcctgtgtctctgcctctctctctagctgtgtctctatgaataaataaataaaaaatattaaaaaaaaaaaaacttattattatttactgttccaaggctggcaaatgatctaaaatgtaaataaaagctaactataggttattttccccattcattttacaagattaaaattataggagtttggtcgttctcttttctttgtaaggaaaaataccagtttcttaagtattttttcaggcatttaggataaaccaaaaacaatgtaagatttcaggtgctttcttgtaaagtatgatggggattttaaagattaataacacctagattttcattctaccttttttctttttttccttttccttttttagattctcgcctctattgagctgctggcccgctcattgccaaaaattcaccgcagtgcatcagaaccctccttgaatcgggctggcttccaaacagaggattttagtctctatgcttgcgcttctccaaaaacacccatccaggcagggggatacggtgcgtttcctgtccactgaaacaaattagagtgtgcgagtatagggcatagggaccatggaataaaaatatgtttgtttatatatttgctaaattgaataagatcttttttaaaaacgatgagccaaagaatgaataggtttttaaagactaggtatcattatttccaaatctaaaatgtaaatttagcattggattttcagcatccaagcattttataaaatgcacaagacatggctgaacatttgtagtacctctttcagaggctttacttcctgttccacatctgtttatgggcttattctaattattaaacttcatttaaacttttctcatgcaccttttgttgttggctatcacatgtccaagggagtcccaagaacagcaccactcctgtgcatggatttgcaggtggacggtgacagtagattagcctgtgttaggcaaggcaatccaaacagatctaattaaaaacttatgagagttgaataagttatttttattcttaatattattttctataactttttattataatttggaaaatatggatgtcctttattcctcaaagcagtagactgaatttctttttacaaattacaagtgcaggtaaccaaagatattgcttaggaatgacatatttgacatgagtaatatggttgaactttggatttttagtgacgaatgttaaaaattggagttctttttaaataatattttcccatacattaaggatgtcttatacacatagaagttgaatttctttacagatgcattgtttcctcctcccccagaaaagcttcatagaattctgtgagaagtttggggtttgtttgtttagatacctttagttaccaaacaaccaagtagtggacttcttaaaaattattattactttagattgtacatacctctcatgatacctgttttaacaatcaccttatccatgtgtcataaaattcttgtgctccttatattctttgcccagcagggttcttatgaatgaatgctattcttttttttttttttttctgtagcctatgtagcctactgtgtgaagtatttattagtgctaatatttaccttttcttgtgtagaaaatgactgtactgtggcagtcagtatactcttactatgagccagaatcttactgggaatcaaaagtgaaaggagcagtatcactgtcatcttaccaaagaaattatcatgtctacttcagagtcagcaccaaaagccagccacttaaatacattttcatttatttaatttagaactgtggtgatttttaaatataactggtggttcggggctatacaggcacatagacaacatctgtgtttaatatcatcctttgaagccaataagaaaattgggaagactttagtgataatttagtttttaaatatcacaaataagtttttaaaatactttattaatttgaatggatgagatttcatacaggaaaactagtcagtgagaagaagcatggcataccttttgcctctcatttgggtagtttgaaataagtaatgccattcatgttgctcaaaattgattcatgcctttcaattaggcaagagtagtggaaaagaacctctctcgtgttttcaaaaataagaaagagaaaggcatgaaagaatggaacctgttggaaaaaagattctaaattagtgcaggatttctaaatcctctagtgatattaaagatatgatagtaattactaaatagagttttgtccattacgaattgaatagcaagcatgtagctaacaaagtttttacattggtcactggaatgcacaaaggtttggacattctaaggcacaagagtcaaaggatctttttactgatcgaactctttctatagttcctcagtatacttcactcgtattgtcctttctttgctcactccttttttctgcccctacaattccccacacggaccactgccaaccccccactctctgcatttgactcttttcagtctcctttgcagaaggcattgctgggctcttagaatcttagaacagctcagaaattcctttatgccacctagcagttttctttccaaactgagctcactaggagtgttggtggaaggacatgagcagtctggtagggcaagatgttcattaagtaacatggatgcagagaagtagggagcatattctagacatacggtagtctccgtttctgttgtttgttttatactgcattttagatatattctccaaataaaaatatcattttcctaaccatatgtagagttaacctttctgactacgtaattgaaacagaaagacgttcattctcttatgactgattgcttttattcctaaaacctcagcccacagattttttttattaaagttgatttcctttttcagcaggcacccttttttgtaaaatttgttttatagtttagaagttttactggtcagtgacgggaactaagatttttcttcttgattttaagtagtttagggatcacagcacataaatgctttaggttattttcaggtattaaatgttgtattagccatttacattctaagcattagttaaaatcattctctttaaatagtgactaacacaccagcttaaatggcctctctcaagtgagtccctcaatttaaggaaatgagctgcttttttttttttaatattatgaaatgggtacttcagactaaaacactgtcagttacagtcctattagattgagaattattaagcatttctagagtactttttattttactccatatttcatcgctcctgtggtaatttctcttaaaatttaagaagtccagtgtgtctgagccagaatgtttgtcatatttttgaaacttcgtaaaaggtcaccacaaagaaagaatgagaattactctggtttcaaatgaatgcccccctacatctaaaacagtgatcgatttctgtgcaaattgatttttcacagcatattttatcttgaagaagtatatagcaaattacatgtataatagccactgaaatacattcaattcaggaataaaaggtaattatgtaatatcttaaaactagttagtaaaatccttagatttcaaataaacgaatcttaatatttgagttattctaaatcacagggccttcccaaatgttctctaccttaatcttaaataaagtcaaatggaaaacagaacttaggttattctcaaccagaaaggttaaaaaaaattttttttttcattttggcctctgatttttcaggcaataattaacataccaaaattttcaaaaagtcatgtagtatcctggatatttcacttcaagtaattcttacctcaaataaaataggataggaagtggaatgatagagatttctggatatgcaagaaggagtatgcctttttccccaaaagcagatccattgcctgttatgcccatctttccttaaatttctccgtgatttcaggacatatatagaaagagttttattttattgatccataaactcttgagttctttttgtctttttttattcctggatctaaagtctgcttatgaaataataactgcattccgttagaagcagtagacaatggtaagatggtcttcagtgtttgctttattagtatctgatttggagtttgttggtacgagtttgtactttgggagactgttttgtgattttttttttttaaaggctaaccttttaagtttgtagagtagaacactgttcaaagtgggatgtctaattatcataattggaagcacatgatgattttttaaagttattttttaatgtggtatataagatctgacatagaattttatcacttttgatattctcacttaatagattaaagtctagccatattttaaaaactggaagattttttttaaaaaataaagatttgcaagtaaatcctattttcacattgacttcctttaatgagattcagattctgcaggatacagagactactaattagaatgttgaaaattaccttaaagaaatatatgaaaatactagaaaaaatagcaaccttcaagccacagtgtacttaaaccataagcaaaggtagtgtgaatgtttatagatttctagtctctgttcacaaaatagtcttctgtggttatattcttcatacttggctctctgtggtcttagtgacttggctttttttttttttttttttttttttaatcaatgccatactccctccctagtcttaactttaaatatgaggtaatgattcttttatccttccttgatcttttgactcacaatactgtggttacttgcttggtgaaaggaatccttaaatagaaaagagatagtatcaccatctgcaatccaattagtattaatactcagagtgggaaaataaattattattgaaaattatttcagggtatattcattgttaagaccacccccacccccagacttgcatcgtaacagtgccattttctagctgtgagatatcaggcaaattactccaccattctaaacccacgcttgctcctttgtaagtttagagaagtaatagcacttgccttatagtgattatgaggcttagatgcaaatatatataaagcattcagtgaggcacctggcgtataacaggtactcagatggttgctaatattatcagcctgtgatctgggggttcagtcagccagtcattagcaatctgttgttgttgggggaggacataccagtgtcctgtagtagccagttggcagtcagagagtggacaggtcactcctagatgaaatcctgtgttggtgtcagtgactgcttggacttgcatcattataataatggaaatgagttgataattgggggaaagtcttcaacaaatcaaccttactattttacaatccatatctgagaaattttgttcattggaatggtttttcagcttgattagaaccatagcatgtcaaatccaaatgggaaattacctgtagactcaatatttaattggcaaaactgaagcatagtctatatactataaattatcttcttttatggatttcggccatttttgcttttaaatcagtgatgatgttgctagctttgtaatagttagcgagtcccctttcagtgaggtttcagtgaggcttttctagatctcttggttatttgagataacttttttaaaattagctcttgtcctccctctacaggagaatttgcagccttcaagtagccacaccatcatggcaacaactactcttatttcttaagtcttgtgttcgtacaatttgttaacatcaaaacacagttctgttcctcaaatctttttttaaagatacagaattttcaatgcataagctggtgtggaacagaatggaatttcccatccaacaaaagagggaagaatgttttaggaaccagaattctctgctgccagtgtttcttcttcaacacaaataccacgtgcatacaagtctgcccactcccaggaaggaagaggagagcctgagttctgaccttttgatggtcaggcatgatggaaagaaactgctgctacagcttgggagattggctgtggagagcctgcccgtcagctctgcccttctaaccgccagatgagtgtgtggctggtcacctgacagggcagctgcaatcgccaagcatcgttctctttcctgtcctgggattttgtcgtggagctctttccccctagtcaccaccggttcatttctgagggatggaacaaaaatgcagcatggcctttctgtgtggtgcatgtccggtctttgacaaatttttatcaagtgaagctcttgtatttaaatggagaatgagaggcgagggggggggatcacgttttggtgtaggggcaaagggaatgctgcatctttttcctgacccactgggtttctggcctttgtttccttgctcactgagggtgtctgcctataaccacgcaggctggaaagtgctggcacacattgccttctcttctcactgggtccagcaatgaagacaagtgttggggatttttttttttgccctccacaatgtagcaagttctcaggaaaatacagttaatatcttcctcctaagctcttccagtcatcaagtacttatgtggctactttgtccagggcacaaaatgccatggcggtatccaattaaaagcctacaaaactgcttgataacagttttgaatgtgtgagacatttatgtaatttaaatgtaaggtacaagttttaatttctgagtttctctattatatttttattaaaaagaaaataattttcagatttaattgaattggaataaaataatacttcccaccagaattatatatcctggaaaattgtatttttgttatataaacaacttttaaagaaagatcattatccttttctctacctaaatatggggagtcttagcataatgacagatatttataatttttaaattaatggtacttgctggatccacactaacatctttgctaatatctcatgttttcctccaacttactcctacactacatcctccatcctctttccagtcttttatctagaatatgcaacctaaaataaaaatggtggtgtctccattca

SEQ ID NO: 8 - DNA, 77288 bp, Canis lupus
  source 1..77288: mol_type = genomic DNA; sub_species = familiaris
ttttgtttaaacactttagctttgctacttgattgtcatatctttgagtgcaggttccatgccttatttattggacctcacttgtattttttatatgttacataacaaacccatgactaaagtatactactactcaagtaaagagtataattatttatttctcataaatcttaagcacccaattgttgcccagttaagagctaattttaaaaattgagtgtaattattcagtaaatattggctaaggaaacagactaaatcagaaagagagccatagcagatactaataataataactaataaccctgtagtaataattgttgcagctttaaagatagaacataggagcacttgtgtgggtcactcagttaagcatctgcctttggctcaggtcatgatcctggggtcctgggatcaagccccacattgggctccctgctcagtggggagtctgctcctccctctgcccacccctctgcctacttgtgctttctctctttctatcaaatagataaataaaatattttttaaaaaataaagatagaacatagaaaataatgcagtgaatgaaagattagggagtaaggagagaaggagagaaagaaagataaatcagcaaatttatgcaagtaacctgatgatacagactgaatgttttgtccctccagaattcgtatattgaaagccaatccccactgtgatgatatctggaggtgaggcctttgggaggtgattctgttatgagagtggagcatccatgaatgggattagtgcccttatagaggagacccggagagcccgcttgcccctttcaacattgaagatgtagcaaaaagatggtcatcggcgaacacagaagcagactgtgatcagacaccaaatctgcctgcatcttaaccttggacttgtcggcctgcagaactgtggaaaataaatgtttgtcatctacaagtcacccaggctatggcattttgttatcacagcccaaatggactaagacacataacaacaattcaatctttaccaaactaaatcaattctctatcctttgctgttttattggccctttttccagatcacttttctctagaaaatcagaagtctttttggcaaacaattccaaaatggtactgagaatatttttaatcaattttcaactgggtatacaagagatagatacatttctggttgctctttaatcacttccctgtcagagaaaccaaaacaacaattgtacccaggtcaatttgagaatagctagtgctattcagattgctcccttagtgaatcaaaaagtccaataggctgctcatcatgaagaggacagtggaggtgaagacaaacttagcaagtattcatgagctgaagatgcttactctgtggctgcagaataaaggtagctggataaatagaaaaagaggagatacttaccttaatgctggaaagaaggagtgaaggtgggggagaagggatggaaggaaggagagagaacaggaggtagaaaggaagaagaatgagagaaatcttagcaatgaaggaaatatctaataattcaaaatgaagtacaggtcagttagactagtggcatggggatggcctggtaatgagtacaaagagttttcccaaagatcagttaagagcaggctctagcagtagagcaagaccagtgcctgatagtagtaaaaagcaggagttatcaccacaaagttagatggcaatctttcaacctgtttttttttttttttaaactagcatctgaattcagttttattttgaaggaaaatcccaaagagctctccattttaagagaaatgtggaaaagtgggaaaaacatagtatccatcaaattgaggtacatgtattagtagtcaaatttagaaagaaattagtagtcatatttagaaagaaaaacaacatattagagaaaagctaagtattccatatatctaaaacaagggcatttaaatatggaactttcctaatagttggaggcaaattagtttgtgccattatgtggtcagcctaagtgttccatttcaagtatacattgaagaaatctttccctccaagtaacctcaagtcagtcttctttccataagttgattatgcaactggaaatgatttagcatgtgatctcaccatagctttaacctattccaatatatgatgtgcctgaacatttagaagattgaaatattcattaaaatgaggaatattaattccttaatatttatttaaataggaaaaaagaaaccttggtggcacaaatgtagattaattgaaataggaacttatatctaaaaatgcgactgacctcctttccgtaaatatcagacaaggaattcaaactaaaacacaacttattttgtcagaaattacaaaatgtgcatttttttagcagatactaattgtaccgatgagatttatctaaaccactctcatctgggatatggttaattcttatacatcagcacatctattctgtctctgaggcttaggcagaaaagtacatgctacttccacccctgcacgaagtgaagggaatacatgggactgttagttctgtggttacctgtcaaggacagactacgttacattgcacttttctaggcaatataggcagttgcctgtggttgagaaaataattcaaacactcatgaaacattttaagggtaagacaacagatgatgactagtacggaaagtacaattagagaagaagaaagcgcccaacaagttactcactgtagtaaatagaaattattccaaatgaactagataatggggaggcaaaggaacagaaggtttactgttcgaggtttaatgaccattgctaagaaaacccaaggatacatcctgcccaccaacttctgtaggtttctctgggcccatgtagccctccacagtagtaatgacaccaatagatttacgctacgtgttacacctaccgaagaactttcatatgaccgaaaagtcagggcgtgcactatcatcactactgcccagaggcaggaacgaaactcagtaacatcatgtgactttccgaacatcacccaaaagcaaaatgtaaggcctccaagtttctgcccgtggccatgacactactaaatcaagatgtgtggcctaatgaagacggcatgttggcaaatgacaaaggaggaaggcaatagaaatgattttacagcatccaagtgtcagatggactcctagggtaacgcttaaaaaatcactttgaagaaattatccatcacttgttcctctgagttacaggaaagggaggaggggaaaagaccacgaggtctttattatgagggattttcctttctggctatgtgacagtgacccggattaaagggcatgctttttcctatactaggtatccttctctgtgtggagtgggcacagagtcccccagctctgggcataaaatgaaaggaaaagctgtaaacaaaaatgctcaattgaggtggtagcaacttgcttgtatttatagagaaaatatgaaaagccagttacacttagggaccagtccaacagtacagagcaaagtgtattgtggatatacttcacagaataggcacatgcaagtggattgttccctctagaccggacttttgtcctaagtaaaactgtaaaataatttgcatttatcctctttgtcagaagaagccagccaaactcataggggaaaaaagttcctttgcaaatgaataaaatcccctttccaagggagacatgcatttttaaaatttagtcaatgtggtggacctttgggtatctcctctgccctaaattgtcttatttatcctttcttctgcctttcatatgctacccaacagggtgattccacaaagagtgcagatctatggggaagaacaccctccaattctccctctctctttctctctctcctagatttatgggatctgcagaccctaatgggagacaaaatgcacttttccgtcggtgaaagtcatcaaaaattaccaaaaggtacacggggaacaaacatccatgataagaagataacatattcatacggtcactttcttatcacaacgaagcttgtgaggagaaggaaaagcctggaaaactcaaggatacgaggaaaggttacagaatccactcattagaatacacagcatgtcatcttggcgctgcctgctgcactccacagtggtaatgcttttccttccaccgaacagatccatctccagagccctcttggaactgtcaatatagtcattatcaattatgtaatgcaggggggagcaaagattaataattcagagctaaggcatcaccatgatttataatcttgcatgtaactgtctacagctagcattgggaacctgtcattaaaagacattaacttgagaaaattagagttctgtattgatcaccaaacatgtatagtttatcgtggagtgatgctttaatttatattccaagcaaggctctaggtgaatatcatttgcgtagggagggctgcatccctaagccatctggttccacaacatgacagtagctgcaggtaagcatcctctagacaagcacagtccagtcgcgacaggcagacgttgtacccaaacaccgcctgtgcggttcgggcacaattattaacgaaaagaaaggttttatcctgaacgtgcagaagtagggccactcggtgacttgatggatgatctcaaggggatattacctttccacattcctatccttaggtaggtgaaatgcctctctgctcttctatttcctgtacctcactagcaatttgccttccttaataaaattagaaggaagttattaaatgacttggcccaatccctgagtttctgcaagtgaatactacaggattgtgaccgcatgatgtgatctctgggaggactggctgtgagggtgcattaactcaggtaatcagactgtcatcaccaacctttagcaaaagtgtctcagaggcccgagctgaactttggaggaagctgacaggcttttacaaacaccaccttgatgacgagccaggcagaaatgcacagtaacagtgggttaatgatggttttcaaaccttggctgcatattataatcatctggggagattttatagaaaatactgatgtctgagcctcatgcagagattctactttaattggtctgggatggggaaccaggcagtgcaaaatttgttaagttccctaagtgattccacagtgtgccaattttaagaagcactggttggggtgtgttcaagtgtatgtgagtaaatttaagatagaaaggatggaacacattgaaacggctaggatctgggtataaactttaaaatggtgaatatgaaaaacaactgaacagtaattagcaaattagcatgtaacacaaaaatacaggtgcaaagttttggtttggttttggtaccagtcttggctgcttggcaggtaccttggagggaacacaatggtgtgatggagcacagggtgcccctctccttgtaccagacttggatgccagccgctgaggggtgtggtggatatcagctgagtccggtggaaattagatacgcctttcctggttatgaaacatagggtggggtgtgggatcattgtggacactgaaaatcagattttgccactgtgaggttagaaatgtaagatcattcctctaaaactgtctttgagaagctgatggatgtccagtgcatgtttcaccggggcttctggggaaatgttcactgggtcacatccacctttcatctgtgcactcagatctggctgcaaacaaaaaggagaggttgatagtaggagagcctgtagccagatacagtgcaatgtgataaagggagattaagggtgtttttaaaggctcagaacctcagagataatcatctgacaaagagcattcaaggcaaagaacaatagattagagaggtagctttgataagttttcatgctgaggaaacaaaaagaataaagaaataaaccatctcgaaggagcccaaagatctctctgtgcttatgcattattaataactaaagaggaataaaaaataactaatcttgacaaatagaatgatgacagctttgatgtttctgtgctagtcaaggatgaatgaagaatagataacttctatatacaaagaaatgcataaattgagataattacattatgcaaggtgttagcactctctctagatctaaacggagtctggtttacatgttagtaaacaattctgggcgtattacagggggtctgagcactcttttcggcttagtggttagtaccatatagatttgttcctaaataattcaagacccttgagatgtccagtctcccagaatctaccagattctgaatcccagtggaatgtttctcctctaaccttctgttcagccaacagagaaactgttagtacttgctgagccatggaaagcaaacaggtagcgaacttttcttcacctcctcctgttgtgatagcagtaatatgttttttgtttcaatccagagcctgcagagccttattatctggggggaaatgcaaaggaagaagagaaaaaacatgcctgaagtggccccattgaggaagctatgggccatcatttcttgtgattagatcaaagaaatctcagaacacctcaaagcaatttacagaaaaacacattcctatgtacctggggaacagtgactcaggtcacaactcccaaattctagtcttattgccttaaatttgattaatggggcatattgcttcacattaagctgcgtcaagccaggatgtaatgagatacgatattgactattgacggcgactttgctacttcagctgaccagttcagaggagagcgcttgtcttaaaggaaatacactggtttgaattcctttctagtttctttccttagaaccaaatgtttatatccttaagatctacttccatgttgtgtgtacccttctttgtctgcactgtcaaacaacattctaagggggtaccaatccatcagttcaagaaagaggaagatctcagtttggattctataatgatttttgaagagctgcctctaaacatcttttttttttctttttagaacaacaatccctaagctttagccctcccacatccaacaaaataatagctaatatttaattgacacatcatgcttctgcctaggctcacacagctactgaataatagagcgccatttagttttttcatcaacacttatttgctcaagatcttttatgtgtcaagaagcactataataggtatatggaatatagcagtgagaaagatatagaggtaccctcatagagattttgtaatgaggggagaaggaccaatgcaaaaaagagacaaataaaatattgacaaattgtgatgtcatgagataaaaaataaaaggaagtccttctttagagaacatggtcaggaaaggcttttctagtcctggaggacagctctatcaccatagggtgagttggggaagggtggaggtagaactggaactcagagaagatgtgccttgagcccatattcctgatggccatactacagcctctctttgacttcctgcagaacaatttaaggtattcatttgtctcaactcattctagccaatcccctagtccacactccacatttagggcttactatttctacccacgtgtctatctctcccgtcaaaggggaagaggtgtcacttgtcacttcccattttgtcaccctagaagatccacttggaatcacacccctgtgactcaccagaagctttacccctttgtctgctttttatcttctgtatctctcttctctgccctcttgattccttccttacagtgcacaaacatgcatgagtttctcatattaagatagctctcaaacaaacatctcccttctagctactatccctgaaaataatgctctataatcttttccccctttttcccagttctcactcattctatagaccctatgaactggctcggcctcctttactccaatgaacagaaaacatagtagaatgaagagaacagactttggattcaaatagaaatgaattacagacccatgtttcaatctttagctgtgtgcccttgggaaagatatttaacttctctaaacttgagcttatatatctataaagtaggcacagttataataatacctgcttcacaggttataagaagtattaaaagaaacaatagataccaagcacagcccaaacattgtataagatcagagtacaagtgctaagtcattatttaaatcatcccctaaactgtgctagcaaagacactagtgaccaactgagcgccaaattcattgggatattttcagatctagtctttgttataactttcttgtgatgtttgacattattgaactgtcctttcatcttaaaatcctctactgtcttgacctccaataacaataccttattctggttctcattctctttctattactgtccttctcttcccatgaccattccttcttggcatgctttgctgattcttcttcctcctctacttcctcttgttcccttcttttgtcactcacttgtcttatcattctgggcccccttctggtcatttacatgaccttggttgccatctctgttaattactctcaaaactaattttctattgatagttgcttcataaactttagatgggtggatggattcccctgctcacaggacttctccacctccacatcctgtagaaacttcattgtcaacacagaaaatagtttctcttccctcggttttctaacatcttctgttaccaagatccacctgatttcccgatggaagaaacatctgtaatatcttcagctgcttgcctggggatccaggacaaagacccagatctcttgcctccaaactcagaactttttgactccgagatctgtgattagcagtttacagcatgctctttatccaagttcataatagcttgatattattaccttgtgatagttaattttatgtgtcaacttggctagattcatgatacctaatttttggtcaaataccagtctagtgtgaaggtattttttagatggtgattagcatttaaatcagtcattttgagtaaaacaaattaccctccatagtgtcagttggagaccatccaaatcattgaaagcctcaagagaaacacagaggttccttagggaaggaaggattttgcctccagactgactgcctctggattcaagcttcaacacgtgtctttgacatgctggcctgccctgaggatttccaacttgccagcacttacactcatgtgagccagttccttaaaatcaatcactctctctctctctcttctatttattatgtttctctggggacccctgactactgcacaccctatatcccttcctctatcattagaaacatatcattagcattttgccagcttaacttctcttttgcatattggtgttatttattttagcataaatagatgtcgtgtatgtgatcagtaacctactctagatacatggagtctgggaatacattcagtcatgtgtcgagtaggttctttacccttgagaagacaaaacatctttcctcttttctctctccccacttctcttcatcaaagccccactgaacttgtcctgccaccctccaaaccagaccataaattaatgtcacccatctcactcaccctagtgtttggcaagggtttgtgatccaagtatgttgggcttctttgataggaaaacaaacttgattgctatctcaagatccaggtggtagaacaaacttcaaacccaaatatcacaatttgtgtcttttttttccttctcagagtcctacttgatttttcctaacttgattaatttgctatccataatggctctcaagttataaacagaggattgtaagagctttgcacgtcaccagcaattcagcttctcagaaacaagggacaaaaacctgatttggagtgaaatctctaagggattaattaactcagtaaagacatttatagcactggctaacagttcaatgatggactgaaggttgagttcattccttatgtgccactggctaaaaaacgcaattaatctagtatattaaatgaatatttaggtcatcaaaagaagacaaaagtgttttgtttgttcatttggattttgtaaccaaaattacagtccctcttccttcttccattgaaaggaggttaatatctaaaaaatgcttacaaggaataggctaagctattcatgttaagcttggtgtcttaaaatgatattcagtgattctagtcacatttgctaaactgccaaaacttttaataaacacatatgctttggacttctagtttctggtttctggggattacaatattttcctaagttagccagccttctatcttggacaaggctgtatgactatttactcaagtaacttatctcaggagctttaaatgtttccccatatttcatgccctgttcctctgaatgtctggattttggaaattaacattcagaggagagtagaagaattttttcatataaaaaaggtaaataatatggtatcacccatatgtctagggagaggaatgtaaatttggtgtctacagaggctctctcttcgttcctatttctctatttatgtgttatttttttaagtttcttttattactcatcatttgataaatggtaaatattttactcccctacctatatcctccttcaataatatttttattttgacaaactcaaggatatccaaatataagaataaaataaatccttcacgtttctgaaaagtccagtaatttagtggcatgtccatttctgcttgtgcacacattagcatggagacttgttttagacaaaggttcatttatttttactgaaaaggtatcactctcacttaagaaaaaaaaaaacctcatatgcattctctctttgcagttccataatatctatgcaaataatacacattgtgaaaggaaacataaaacaaatagatgtgggtgggggtactttaaaaatctacttaggatgatttagatgcggtttattctaaagccagagcaatggatgaaataaccttttaaactacccttcttgttctagaattgtaagacaaaagaaaatacattctgaccacagaaatggcattttttggagtaggatatctgcccaagagaagaaagacaagttctgatgttgccgtaaaatttacatgttttgctccaaccttaaaatcctaataggtatttcattaaaattatgacgtaattattcattaggtataatacacttaacatttcaataatgaactgctatagcaataggagagaaaatctgatttgtttgtttccactgttttaaatagctaactattcttgccatcatctagagccactccaaaatgtcccaggtactggcaaacaacaccaattatttaagagctgagtctcaagataaagattttcccttccttcctttcaaatgcccattgaaggccagcaagaattgtatttctagtcattctctgtgagtctgtctgtctgtctctctatctctctctcctttccaccttcccaccatgtttccctctccatccctccatctctctttcccctacagaatatgcagggctttagaagattcaagacagagtatgataatacaaaacactagtaataaaatgtgtatgcttttctgatagttttgagatacataaaaacttggaaaattcatacacacagacacacacacaatttatgtcatcaagtaatgctacctcaacaactagaaaaagttttctgagccaatgaaaaggtattttttaataatatgagaaatctcctgtgtatttctgtagctcaccagcctccaagtagagctctccacagtaaatatgattcaaaagctcaatcattaattctatttaggcttaatgtgtcataaagctaaggaatgcagggccattttcctctctgctggggcacaaaaagtatattaactattccagccagtactgtgaatgatttattaattcaattttggaaagtgagactgtcagatcatataagcacatattataaacagatgatctctctgaagattcttattccttctcttagctaaatgactgaaaatgcacttcaccactgagatctctctgtctctgtctctctctgtgtctctgtccctagctcctagatatacacacatactattcaaataggagactatccactcatccttgagacattagaactaactcaacataaataaaaagaagcacctggactgacatgaaggaattttcccaaaaacagacctaaaaattactgagtcaactgtgatgcagtgatgtactgaaaagaacatcagtgcacagaactccaatgacctggattctggaccacgatcttccaggaagtacacatgaccttgggcaggtcacttcacctgtgtccttagccaggaagaggaggacctgtgatctctacaatttctaactttctatgaacctgtgttgtcaacatgaatgaaacctgatggcataatgatggtctacgttccactcagcagcaagattctttaactgtccataccttagagcctttcagtgttcagcctgttccttctaaataacacatcatggggcaaatttacatttcctaaccttagaatcccaatgttgggacaacatcagaatgagtgtgtgggtgtgtgtgggtgtgtgtgttttcttggattaaattcctagcatttgccaaccacccagaaatgatttttaaccaagcctcttttagaatattgaatattgctagtgttttagggttcttaaaacatttttatttttattttttttaaagaatttatttatttattcacgagagacacacagagagaaagacagagagacacaggcagagggagaagcaggctccatgcagggagcccaacgtgggactcaatcctgggcctccaggatcacaccccaggctgaaggcggcactaaactgctgagccactcgggctgccccttaaaacatttttatttcctctttctgtaaaacttgtaaaccccttctaccattcctttttacatacagaggaattgggttttattcccataggaactctgagaattaaaatatagagattattttaatcatgtgatggtgattcatttcctaagatattcaatgtgtgataaatatgggtgaaatcaagggataattttcaagatcagagagctgtttgtaattttgcaacaggttgaaagtacactcacctgtttcctacccaaattatgtcaatgggaaatgtttatttcttgttcttgacaaaaaccttttatagggaatctgcccatttgtggctcagacaacacctcaggtatgaagctgttaaagaccagatcaagaaaaagtggtgcaaagggaaggtgcagaatcaaacttgacttcttaaaagtttttaaaaaaaaaaaaaaaaaaaaaaaaaaaaaaaaaaaaaaccacacacacacacacacaaatggtttaatataaaaattcatcatttattcttagcaaaagatgtatcatgatgctttctgtgcaaggatttgtttcacttgcaaaattatgttccatactatctgtaaaacttttcgcctggggcactcttttataggtatcaccctcaaaactagttacatattatagtccaaacattgcagataaaatgggcaattgcaatacgaccatggccttttacaattataacacagaagccacattagaatttagtttatttaaatttaaatacttttttttacttagttttgttcattggctttttcattaaataggctgtatttaaatcatttattatattcatgcctgtcatgcattaagcagtatcccccaaataattcatacatgagtacctgtgaatatggccttatttggaaatagggactttgctgataataaagttaaaataaagtcatcaggctgggtcctaatctgccaccatgtccttatggaaaaggaaatttggacatagaaacagacatggggggatccctgggtggctcagcagtttggcgcctgcctttggcccagggcgcgatcctggagtccctggatcgagtcccgcatctggctcccggcatggagcctgtttctcctcctcctgtgtctctgcctctttctctctctctctctctctctctctctctctctgtgtgtgtctatcataaataaaaataaataaataaatctttaaaaatatgtagttaattattttattaaaaaaaaaaaagaagaagaaacagacatgggagggagaatgtccagtgaagataaagacagagatgggatgatgtatctacaagttggcaatctttggtaccagaagctaggggagaggcctgggatggatcctctcccaccttgatctctgacttgcagccttcagaactgtaagacaatcctttttggtgcttaagcctctcagtccgtagccctctgcttaatgcagccctagaaagctaatacaatgcatctatatattactcgcaagggtgtttgcttttattcaaacatgagagagagctgcaaatgctcaaaatgaagaacatagcttgaaaagaccaacttcatgaattgggtgaacttcaggtgaaagggagggatgtttcctcagcagtgtacctattttgctgttgtcctagctgctttaggttcagcatgttacactgtggtgagagtattgtgtccattgatagtgagtgttgtacccatggacttgaacccaaaaaatttgaattaactgccaattagaaaatgaatagatccttcccaacctaccttcctttccctctttatcactcctcccccactgtctttctcttttgcagtttacccttcaaatttcttcctgcatcaacaagatgctggccatttccatgaggagaaattagccccagtgtctctgaggcagctgttcagttcctcagacttgctgccactctcttccaggcctcgtgtgtggaatacaaaacacggtgaacagagggctccatgaggctaggcatcggattctctttcagaatatggaggacgggcctcctgcatcccttgcattctcctccagacctcttcctgtttcatagagtataaaataacaagcccccaggtcactcgcatgccgttctgtcatttcctcagagaccggatgggtaatcctgtaataatactatttcagacacttctctattctccagatagaaaatagataattaaatgcatgtttctaccattattctctatttatccggggttgtgaatagtgaattaatatctccaaagatcccactcataataaagaaaaactggataagaattgatttgtatctactgctattgtgcttaaatcgctatgtttttctatttgcttttcagtctcagacaggagtagtccaggaagaacgaataaaaagttttaaatgtgagcagattttcccccaatatctttgcttccaggacactgatactccattatgaatagaatttgatgggttttcagagtctacttcataatccatccactgccccgccaggagaaaaacttcacaaaccaataatatattttgatcctcatctaaacctactgggtcagcaaaaccaaactgtaaatttctctataactgcttctcaggaaatttccagcatttctgaatgggccagaaatcagtggaaatggcctctgtcttgccatattttggcatttggactacaagaaagaaaaaacggcatatagatttatatggagctttaccaccattttccatcaagccacaataggccctagcacctgattattacatgagacttctgaggcttggcttagtgctagtgaagcatcagtgagctacagaagctgggccttacctcccagcattttcataacaatggtggacttggaagatgttgcggttaactcatttcccatttggctcctctggaaataactgtaaacttgacttcctaaggagaagacatgaacaagagggttattgtgccttttctaagattttcaacacttaggaaactggctggaatcacttttctttaaaaaataagtggtaaggataaagctgctcactcatatttactctcaaagtaacagatcacagaaatggaataggagaaaggatattttttataacacatctttggggcaaagtcccttctaaggaatacggacaaatattttttgtggggcctccgtctatataaacattttgagtcaccccaaattgctaagtcagcacctttgtgatggtccaatctaatacgctctgctaagaaaggaattaggctattgcaatgagattgatgggctctctaggctaccctccagaaagtgatgtggttggggttgcatgagtcccagggctctaccaggtatctggtacaccccataggtaggtagggttgactgaagaacaacccacccaggagtttgggctcttggcaccctgcttgagaggtacagctgccctgataattggaagctcatcattggtggcacttagaaaatttcagttagtgctccaaagaggagggagtgaggtcagggaggccctttgtgcaaactggcatgaggccccctgcccacacccaacagcagcacgcctttggactaacttattaaaatgatgtaattcaaatgtatgacacacaaaagagaattttacaaatattgatatttctcatacaatagtgagagaggaaaaatcacttttatttgatttaaaagacattctctgagtgttgttcaaatatttaaacagctgtgcttttgtacacacctatatactgttctatttaagaaaataatgacttacataaatgactgtgtatgatagactatttgcaatatgtgtttcacaaagtagaaatgaaatggctaaaccatatcgaatattggaacatacgttgatagtaaagtgttgttggaaaatgggctatttttatagaagttcaaaaatgttgcagtgattattggtgttgctacagtttcaaatttgttttataacactacatggtaagtgttgatcttcatataaacttctctgtttgtccatttttaaggaaagagaacaagtgtaaataactcccatttttagcactaggccaactagtaaaaagcagagtaaaaggccataaaatatgtgataacatctttttatatacacatttgataaataaggacataaaggaaataaaagatatctcagaagccaaatagataatatactgaaattcaggcccttaacagagcctgataattctgctgggggaaggggggaatagtaggttcaaaaacaagaggtcgtgggttttagtggccaacagtgtcaagggaaaacttcttgaatttgggcaaggcaaagtgggaatgaaactctcccccttttaaagccaggattgaaaagttatattctcaatgaaaatgttcactatataaacacacacacacacacacacacacacacacacacacacacatgctcaccagcacaggaaattataaataagatttcctatcttggtccagtcactggttttaaaaaagaaaacagaataaaagctctctccagtatctaaaaccagaagtctgcccttatatgagtgtagagcttaaatgatcctccttacttcctctataccattgtctatccctcctcctctaaagcctcttaccattgaatcccacgtcattaggaacccatgcactcattccttcttcaagtcacacacacacccctggatttttcccataattccttaagatttttagctactggctcacattcactctcttcaactttcaacacttctctagtcacaattcttgtaactacaaatagaatacagattatttttccaatacaatgcatcaatacaattgatgtccttgtaagagacctcagaaatattttacccccttccatcaagactggtgtctgtgaaccaggaaacggcttctcaccagatactgaacatattggatacctgatcttgggcttcccagccttcagaagcatgaaaaaatttttccattgtttataaatctatagtaattttgttacagcagcccaaatggactaagacagaagttggcaccaggagagaggtactactataacaaatacttaaaaatgtggaagctgtgatcacgagtaacagatgaaagagttttgagattcttgctaaaaaaaagtcttcattgcctgacagcctgttaagagtgattctggtgagagctcagaaagaaaaaagagcttttggaggaagtacctaagtaatgttgaacagaaatggggatggccaagaccattctgatgaagtttcagatggaaatgaggcacatgttattggacaatggaggaaaggctgtctttattataatatagtaaagaacatggctggattgtgttcatgttctagtgttttgtgagaagtagaagttgtaagtaatgaaatgatatatttagctgaagctatttctaagcaaagtgttgaaggtatatatggcttggcttctcctgactgcttacagtaaaatgtgagaagagagaaatgatttaaaaacagaattgttaatgaaaagggaagcagaatatcaaagatttagaagattctcagcctatctacctggtaagaatactaagggtggggccaagggaccaaatgataagaacattagtatggatgagccacctcaacagaaagcaagagctattctccaaaacaatggaagaatgactctgaaggcaattcagagatgatagcactgacacccatcacagttccagagtgcaaggcctgggggcagagtagtttcaaaggatgggaaactcctgcctgtaggaccctggagcccaccatccagcaccatctcactacaaggctgtgactaccacattacactctgttgccttgtttcttggctgccccagctatagatctggcaggcctcaatgaagcacaggccatggtggcacccttctggaggacatgggcagcaaattttggaggcttccatgcagtgaaagaacacagggtgcccaggttcttagggcacagctgcctctacctagattttaaagaacaggaccaacagccattggggtgggtcccggcaaaaagcactcacaaggtctggctattcaaagattgtggggtcacaaagacccgaagtaggaaaagagggctgcctaaagccctcaggaacctaaccactgcctagcaaagctgaagggactggacagctgctcccatgggtccaggaggtgtgaccattgccccagcatgtctggaaagcaagacaagcctaagatatctgaaggcagccccttccctagttgtcctggaggacagacagagcattaagccaaagacgattactaagaaggtctagggtttagtggtgtttgccatgctgggttttggacttgcttaaggaccttttttctttcctatttctcctttttggaatggaaatacatatgctccaccattgtattttggaagcacataccttgtttgatttcacagatatatagttagagagcaatttgcctcaggatgaatagactcttgagtcttgctcatgtctgatttagatgacattaagacaatgcagagttagcatagtgatataaaaatagactaattgagaggaatagagaactcaaatccagatccatacaaataaggaaatatataagaaagaggcaacaatatggatcagtgaggaagggaaagactattcaacaaaacgtactgagatacttggctatttacattaaaaaatatactatcataccgtaaacatataatatgatgccttcattatgaaatacggaatttttagaagaaggcattagaatgttttatatgctcaggctaaggaaggaaactatcccatctccctttcctctaaaattctccatcttttagaaggcagcaaatgggtcatcttctacctccactgtttgggttttttaggtctccttcaccttctcccattattcctatcatacaaagaacagtttattttcaatacttttccacattctatcttgtacccacaacatactattctattttccatccatacacctgttaaaaaaaactagatgacaaagaactctcattttgcttgctatctaagcagcataaacctataaatagaagtccccctctgaaacttgcacacacacacaaaataaaattgtgaccaggaatcaaaaagcagaaattaaactgcttttagaaaacaaactcatagtaataagacacaaactactatataaaacttttgtactctagttgataaacttgtttcccatgggaacataggttaagaattctagaatcactatatgcatatactgtaattaatcaatttaagcaatggatggaggatggagtatccaggtttttcaatgttgaagttaagctttcagataagcaaggacatgaagcttgaaccctttatatgataatgaataattagataattgataactagagttggagacatcagcatgaactcacatataacttaatatacatacagatggttacacataggaatatttatagatgtggatatatatttatatgcattagcacgcacacacatatatttccttgctctgtcaaccagagggattagaaacaatgacactccagtagcaatgagtacacctagcacccagatcttagtttctaatactatctctaataagagattaagtattagtttctaatactatctctaataagagaatcaggtcttctaaaagaaatggctaattttaagactatagcagaaagtttacaagctgagtgtggaacaccttagagtgccagaaagtaaagaaattctcggaagaaaagaaaaaacccacaatgatggtaatatgtcaaagggaactcaaagagccaactgtaacatctcccactggccaaagctgaaaaaatttaagtaacaaaataaataaagtaataatgtattataattgaaatatcaactaagtattcttgacttcatgcttatataaatgattaagtaaaataaataaagagaacaatctcctgtgcatatttccaaataaattatgtacatactccaccctcaagttggcaaagcctacctccctactcttgaactgtggactgtcttccaaataacagtgcaagaaaagagggcagggaaaagagcaattttatcatggggaaacctgatggataccaattcagtcaggtgatcaaggctacatcaacaatgataactccgtggcttgtgtgttgttaataagatctaatgagaattacactttacttctgtggtctcctcccaaaaacaagtccagtttattcgttataaaaatcatcaggcaaatactgatttgggggccattctcaaaatacatgatcgatactctacaatattcttaagatcaccaaaaacaaggaaatcttagaaattgtcgtagcctcatagccccaaaggagccatggcatcaaaacgtaatttggtatcttggatgggatcttggaacaaaagaaagaagattaggttaaaactaagaaatatagtatggatttagttaatgtgtcaatattcattcattaattgtgacaaataccccatattaacataagatacaaacaattatggaagattgttgtgaagcatgtgggaactctctactattttcatgtttttttctgcaaacttgaaactattctaaaatacaaagtttattaaaaaaataaacgaagttctccaaaatcagtgttgaatatcttgggccttttgacaaaggatcaaatgggggtgatggcaggtagggggaggggatcctctggggtcaagtacagcccaaatgtcatctcagaatttccctaatgatttgatctaacataatttccatgttactcgttgccacagttctttattttattctcttctcagaacttagcacaacctaagtagtcttgtaaacttatctcatgtaccttctgtctcctgaagtaaaatgtaagttccatgagaccaggcaccaagtctgtcttatttaatcctttgctttgaacagtatctggcattaggggggtactcatcagatgttcgctggataaatgatctttaaaattactcagaatcaggaaattatatgcccacacaagatgaaaccaaaacacttaccatgtcacctcaggagtatgatactctgtcaagcttcttaccccacccggaaatattgaccagacacgcatgtcattttataaatgaggtgtataaataagaaatcacccattttaatgtctttgaccttaggtaaagacatattattgtattaataataagagttatggaattcaaagggcactcctaaccatgaattcaaggggaaagattaaataattatttgctatttatatctcagtgtctgtggcagtactcagtaccatataaatatggaactaggagagacaagttttatgggatgaaaccacaccaactttctaagctcagttatgagtttctttccctatgtttcctgagagcaagaagtgagatttataaataaaataatttacccactgaaatctgtaaactgtttttgttattggatattgtgtttcttcatttaaagtatgagatatgtttttatgagaattaaatattctcacttatactgccaaataaattctctagccttgtattagtgtaccttcccctctttatattatgcagattcattagctagtcaaaacaaacaagcaggcacatacaccagccagtggctggatctggttttctccttgcagaattgcaaactataaccagttcaaaactaggcacaatataaagccttgtttttttcaccaagggagaaaaaataatatcttcaacatccactgaaggcttactatgagtcaaacactgtcctaagcattttattactatttttaaatttaatcttcacagcagcccagtggaacaggttcagtaattattccaattaagtaaattaagaaacagaaagtataagcagcttgttccaagtcacacctctagtaagtgataagattcagaaccagacattctgattctattattctacagaataaatgcttaaccagtatataaataatttctttgggcctattccaactgcaatgatgagaaagccacagtgacaaaaatagagtcttattgaagtttaagttcatgatctttctcactttgccttctttaagtaaacatatgtctccaatcagtggaaatctgaatgtataccaatttgacttttaaaaaagataatgagtattataaatgtggaatgaaacaagtaatttgttattaacaaatttctgatgatcagtcaagatgtataaaaatagaaatcttcttcattcccatgaggtattgctagattgattctcttatggcacaaattaattatatcagtactgtattcaaatgctacagtgtctttaacttattgaaggactactgaacagattgtaggatccttaggattcagggtccccatatattgacccaaatttacctctccattaattacttacttataaatcacaccacgggcaatccaaacatgctttccagtctcctatttctaccccattgcttaagacagatcttcttcccaaaataatctccttccaatgtccatttggagaaatacccccagctcttcaacagccaaatttcatgagcctgattaggtgtctgaaatcagaaaagtcaccttcctcctaactctcacagacctgtgttcacacttactttatgctatttgcatatcacacaaagtacgatgacatgtacatatcacccacctaaagataaaatcttcagaagcataaattgtgactttttataacctgtcacccgagttcactatcttatatttgctctatgaagatctgtgaagaaattaatgaaatatgtatacttggatgcactatcattgtcatctccatttatctgatgaaaaatactgtattagaaactataaaaagtgaagaatatagaataaagaaatttcttaccatcttcatgttatttatatatatcagtgatcatctctcccttgacaaaattattaatttaattttttattaaagattttatttatttattcatgagagacacagagagagagaggcagagacataggcagagagagaagcagggagcctactgcaggactcgaacccaggactccaggatcacgccctgagctgaaggcagacgctcaaccactgagccacccgggcatcccaaaattattaattttaaattaactattaatattactcatgactgtccattaattcccaatgtggaactaagaaaacacatggatacaaactaaagacaagtaagtcttcacgtaaataaaaagtaaaatgcaattcaaggctaacatattaatcaggggaaaaggctataatctaaagtttagtggggtcttaccttaggcagtcttcatgccatgtatatattaatatgagacatgctcagtcaatatacaataagcaccctagttttatttagttacaatatttgatatctattactattgtcattatggtttaaaatttggataaagtgttttgagtggtaaatcccttagcatatacttttttaaatcttttttaaaatttttatttttttaattccagtatagctaatatgcagtgttttattagtttcaggtgtacaatagagtgattccacaattctatacattactcagtgctcatcgtgataagtgtacatttaatacccttcacctatttcacccatcctccatcttcccctcctctcacctctggtagccatcagtgtgttatagttcagagtctgtttcttggtttgtctctctcactctttcttttccttttgctcttttgttttgtttcttaaatttcacatgtgcgtaaagtcctatggcatttgtcattttctgacttatttcacttagtgctatactctctagcttcatccatgctattgcaaatggcaaaatttcattattttaaggctgggtagtattccattcatcttacaagtaatccacatttatagtggaaaaaattagaaaatgtgaacaaacaaaatgttcaagaaaagaaaggcattattacacctgttgcacaaagagaattgctgggcacatcctattatacattcttacattatacatttattatgtatatcaaaaactaagatcacactacaaatacagttttgtaaactgtttttctcccataacacatgaatattttctcatactgatgaatatcgattgccataattttaatggctgaatattcatgaattctttttgtatgcctcaaaggctgtttttaactatataataaacaacactaatatgaacatcctttacatgcatctttggttatttgtccagatatctcctcaagaaaaataatttgatgtgaattattgcttgaatatatataaatattgtaataatatatgaaacattgctaaagctctctgaaagactgcactggtttaattttataagaggtataaaactgcccatttgctccttgctaacactagatgtcaactctaaatttaacatgtgcttactaaaaggagaaaatattctctcatagctgttttaattcaggcacagttgtttagccaaattaataccataagtgcctctaattatgagggataagacctaaatcagacccttcagctcccaaacagcaggaatgatttccaagtggcattttcatacttcaacccagactttaattaactcttctaagaaagacccatttttttttaatcaaataacaacaaaaaaaaaactaagccatgaattggagtcttgcctcacagccaacctcattgcaatgagaccagcatggattctttcggtctttctcgtgttcttatacctttgggcttctgagacttcttgttttacatgagtctcatccttggaacttgctcagttggaaggagagttatgattaaaagcaaactcggcccctccatcttgttggctgtgtacctttcctggtgccgtctctccgtctactttaatgatcttctcctagcaggtgggcaagggccaggataagccatgccactgtagaattcaaggaacaggatctgtcaatataattggcaccctatctgtcagaggatctcttagaaactgacaagtaagagacacataattacagggcacaatcttctccttgaaagtaaagaacttaataaacataatgtccatagggtgcacagacaaaagcaactctcctggttcccctctgagtatttcactgccacttcctacccccatgcatccggtaataaagtgaccctcaaacaaggatgaataatttattatatttttcaagttgtcataaggtagagttgccagttgttacctaatgctgactggcaaagtgaagtctaactttggagaaactaataatgtttcttttccttgtgcttcaggctttcttcagactaaccccacccctaaacttcctaaaactaaacaggaaaaacaacagtggtaatgctctgtacaggatagaactggtcaccagcacaccttagtattgcctgttatgaaaaaaaaaaagacagacaaacaaatagaaacctttactgaaatgagcaaaggctcacaaattgaaaggcaaacaggacgaaattaataacagataaagccagatctccaggtttcacttcttagctgtgtgacgttggacaagttaattatctttacaatgttctttcctacaaaatgaaatgataacagataagagaaaaaactagatggagggaaaatgagtagtttatagtcttctatattcactcatggtgagctatctttggcttccgatggttgattccgtttttctctttaatattatttttccataggtcatacattcacatggcataaaatatgaaagttgcaaaaggaagagcccacaggaaaaactatctctccggtcctaaactccagacacccagttttccttctctaggtagaaaaaatttttatcatttcccttatgttgtgccaaaggtatcatatatatatatatatatatatatatatatatatatatatatatatgatatacatgcaaatatatttaatctttttaccctatattatatatatattaggataccaatgcacctgtgtgcatcttgattttttcatagttaatgtggaaatctttccatatcaggatttaaaaactcttcttattcctttatatagttccatagtattccctttgggggatactacataatttctccccattgatggatattaaggttgttttctaatacttagctatttcaaaaaaattactaccccgcataaactcagatatatcatttcccatacacaaagtacatctttagatataaattcttagaggtagaattgcattttgttattttgcatttgtcattttgatagctatttcctattttcccctagagattataacaatttgcattcctatccaaaacatctcagagtgtctggtcccccaatccctcatcaacattgttatatatgccaaactggtatgtcaaattggtatctcggtatagttttgatttgtagttttcatatcttgaatgaggctgggcattttttcacatatttgagccatttatttttctctgaactgtctttggtcattttctctattgggttattggagagtttttcattagaaaatcaatttagaaaaattcatcctttcaaatatgtttggtttgttgtcttttgacctttgttcatagatggttgtgatgatgatggtgataatgataatgatgatctagttgagttgatcaatattattcacatataaattctgaatatgtaataattatatttaattctatgatatcttctaaaaattagattttagtttttatattaaatctatgatacatgtgtaacttttttggtgttaagttgtgaagttggaatccaacttcagcttatttttccaaatggccactcaattgacttagtatcattcactaaattctaagggttatatctcattcggtactaacacacatagtttgaatctggaagtgtatatgagataaatgaaaaatacaggatggtggagaaagcaattggctgagagtcctgagtcccagtagctaagcttagattcaactctaaattatttgattttttttaagctttatttatttattttggagaaagagtgagtgtgagtgtggggaggagcagaaacagatataaagggaaagagaacctccagcagactcctctctaaacaaaaagtgtgacacagggcttgatcccaggaccctgagatcattacctgagccaaaaccaggagtcagaggctcaactaattgagccacccaggcacccctctgacttcattgattaatcaggttgttttatttctgctttcagtatcaccctcttcatatatttatacatacatgctacaagactgatttttctaaaagacaaatacaataatacaattattttcctgcttaaacactgccaatagttccacaacacattatagaataaagtctcaatttttaatataatattcttcttgaacaaatgttctggctacttgttagattacttctctgatattgcattttcagtttaaattttagcaaatgtaattatataccatatctctccaagaagaatacatgctctgtgaggggaagggccaaaaatctcatctaccacatttgaatatatgataggtattagaaaatagatttttgaactaaagttagttctcttatctattcatctaatgcctttctatgcaactcttttagcgaaattcttaagcatcaggtcctatacttgcagaaaaggattgggtaattgtatctgcctcctacccatcttataaggaagaatactggctaaaatatttatttatagccttgcaatatgaaggcaactgtaagaaattccataataatacatagaggttaaaaagaaagcctggtcctttagatgcagacttttaatatctcacacatatagaaggagtagtaatatttctcagtctcctagtgactaaaagaatcacatttctgataagatgaatatgcaaatatcctcaaatagacgttatcagtagcctaatgggttacagaataaccgtggaaatgtagcaactacaaatcagatgtccagagcttgaaatctgcattaattttctagactgctgcttccaggcaatctcagaaggatggtactttctattctgtcctcaggtcacccagatctcattctttctgtagatgaagagctgggacatactgcaccgtgtcatccttttgcctgatctgtgagcagttttaacggaatgacaggacaccaccgaaacccgatgctctaggacagatggccacatggtccctgccagcacggctgagaacacatgttactgccaaggaatagaggctgcaggggaagctcatgagctgttattccagggactcttgctcgaagggctgtcacctttcagtctatgcctgggtttccattctagtcacagctgccagtaagtgaataactctacaactcatcaaattcacttgatatctaccactcccagtcagaaaaggctgcaatgatcgaacttacacactgtatttatttcaatcgccacctggcatcagactcaactgacatatgcaaggaaaccacccatccagaggggttaacacaggcttgattcttgccataacagcagcagaagccaaatcacgcaatttctggagggaaaaagggaagtgtgagaaaaggcacaccaatttttgggccaccttcttaattactgctggtcctttaaaaacaacaatacatgcaaagtttaaatcttcatttggtagactctaactttttatttttatcagattatatgactcctctcgttaaaactcaacgatcgcaattagaataaaatgcaaaatcctcatcatccatatagaacaaatggtctgcatcatcttgaactcttcccttattttccatactctggcacactggttttctttcttttgcttaaatatttgaagagcacttactggccccaagcacttggtgcttggacttcccataactttctctcacaattcaggactcaactcaaatttgatttgagctttcttttattaccctgtccaaaacagccctcttcttcaggtctccaatctctatttcaatttccatgtgtcatgtccctaatagcacttgatgtgcccgggcacacaagtgcacacatgcacacatacacacacagaccccattatcaggtgcacggtgggtactcattaatatgaaatcaatgcatcagtaacaaatagttttaatattttgtttagacaagcagatatttccaagatatgaaatgggtaggcacagggcagtaatgtctttgaaaaggagggggaaggctcctttaaatagcacagagccttgtgcttagagctgaagtggccactgcatcactttgccaagcggctcacttcttttgtcactttcttcagctcttccttttggacagctcataaccaaaagaagttagtgagaaacaggattcagtaattagattataaaatgtgtttttaaaatagtcaagcacttcaggtcattgctccaaaaaagtatatatacaaacaacgcatactttcaaaaacccaaaaatagttctcttctaataataattctacacaaataatgtgttctttttgtaaagcatttcaaatcatagccactatttttcattccaagggtggaaataagagttctattgtttctaacaatgctttttggtcttcacaatttcaaaagtagaaccattgttgttgttttttcaaattaaatgtagcaggactgtgatcctatcatattgtcctttatatgccagaaaggaaatagaaaatgacaggttattaatggctgaaaattagttcacttggtctatatggggcaactctcctcctgcttaaacacttaaacccatattcatgtcaggataataaatcacagactaatcactgaaaacatgaagaaacaacatatcaagaaaaaattcattgaggaagaaggatccatgttatcttcagtgggtctgggttgacctctctggaatgctgatggttagctttatatagcacaacattctgtcttccttcctctcagatgaatgtgtaagcgaggatgatgacctcttggtatacacaggagcaagaactttaaaagagatgtcagagtaaatcaccaaaaggctgctaaatatgaaatcaggagataaagggtcaaatcctggctctacttgctatagatgagtggcctggggcaagttatttaacttctctaaaaactggtctgtctacagatacatatcaagaaggatttcaaagggaatgtaataagatgattaaaaaaattccaacatacagtctagtatatagattatcaataaaaggtttccttcctctttccatcccttcctcaaaacagattctgtagaggtacctggggggctccatcagtaaagcagctgccttcagctcaggtcatcacccaaggtcccaggatggagcctgcatggggctccctgcaaggcgggagtctgcttctccttctgtcttatgccccgcccctcacgtctctctctctcaaataaataaataaaatcttaaaaaaaaaaaaaaacccacaggacagactctgtagagttaatataaaatcggtgattttaaaacaataagtagattttaaaacaataagtataaaatcaatagaatttcatagatttgtgttgaatatttggggctgtacctcctataattgaaaataatctgcttgtctaagtagaaaaccattataccttaaaattataactatttcccttatttttagccatacttgttgttttatagagatttccagtgtgtggaaatgtagatttttctaaaactatgataatatttatgtttaagtccaatattagttttaccatcacatcatttcagcaaatatttaaagaacatgactatgtagcccatgttgtatcacaggcaagaaacgataagtccttactctcaaggagatctggtctagtggtaaaaaggatacagaaattagcgattgcatagcaatgaggtaaatattttaaataagggcatggaaaaagcgatgctgaagcagaatgggaacacataactttggctgaataagtgcagagaggaaggatttaggaaagttcccttagatagaatgacctttgagccaaagttggcagatgggagcgtggatctgaaaatcagaagaaacacaaaaggttgaaatagacatttagtgtccttaatgttttagtggtaagagtacggataatattgttcaaggtcagtgtgtaaaatgagaagaaaaacaccctaagattaaattattggtttaccagtcagcttttcttattacaacaaataaccctaatatcagtaacttacataaaaaggcacttatttcttgctcattcaccttataattagggcttggttgacctcagctctgctggtcattactgagctaggctgggtttgactgggccccatcaatcttttcaccccaggactcagaatgaaggagaacctataacccgaaaaggcttttctcttggtagagagtaccagtaaaagagacatagtaaactcacacaaatacaattaaatcttcagctcagatgtgacaaatgtcacatcccatttgccaaaatgtgtcaaatgatcatgctcaaagccactaaggcagaggcagatgaacacaagtaaacaggcaagttaaatgtaattgggaagatacataatcattggaagagaagaatcattggaaacaataatgcttttttaaaatttttatttaaactcaatttagttaacacagagtatattattagtttttgtggtagaatttagtgattcatcagttgcatataacatcaagtgccttccttaatgcccatcacccagtttccccacccacctcccctcaaataatgcttaaaaaaaaatcataatctaccttttcttatgttccttttcatttctgaaacttgtatttgtacctttttattcttccttattaacagttttgataaactctgccagcgaacatactacttcaaatataatttttagggcctcattcctaaatactaatagacaatcaagggtcatcagatactaattctaacacatgaaaaaattaacacaaattttaaaaacttgaggaaaatagaatgccaatattagaaaaaaaattttaagactttggctcaggttgtgatcccaggatcccaggatcgagtcctacgttgggctcctggtatggagcctgcttctccctcctcctgtgtctctgcctctctgtgtgtgtgtgtgtgtgtgtgtgtgtgtgtgtgtgtgtgtgtatgtgtgtctatcataaataaaaatataaataaataaataaataaataaataaataaatctttaaaaagaaagactgtgttgtatatttgcaagtttctaagaaagtagatcttaaaaaaaagcacacgaaaacagttattaatcttctcaaaaatcaatacttcatttacaaagcagagggtattaaaaaaaggaacacttcaaaggaaaaaaaagacagctcttaacacaaatgtcctttaaatagttcctgaaataacatgatggtgaaagtatatattgtagtccaaaatagcgtttgagagaatctctttctgtagtagctttctactggttgctctaggtgtcacgttatatacgcatcacctaatacagctcactagtgccatcattttaccattttgaatgaaatgcagagagcttattctaattatggcccttaattctccccatttataataatacttgtcttaaatatttcctccacatacgttttcaacccatatcagggggcattcacttcaactaccaatcataattgagaaaatcatagaggacaagggaaaggtatgtatatttattcctcccattgttctttctgtgtcctgaccttcctaaatacttcattttattactttgttttcctgaagtttgtctatcctgtgtcttgctggggatttctttagagaagaaattaggcaatgcctttttcaagatttgcccagttttttcaatctgcaggtttatgcattttgccaaatttgcatctttaggcattatttctctgagcacttttttcaactccaacattctgatgaaataaatgttatatctcttgttatactctcctaactatctgaagctggtttatttttttcagtctgttttctgtttgttgtttagatgagatgattttgatttttgcattttatagttccttggttttttacactgtcacctgcagtctgctgttgagcacatccgttgaaggttttgctttttgttttttttcaattattatatttttccattctaaatattccatgtgttgtgtgtgtgtacgtatgtatgtatgcatgtgttctatttctttgctgatactatttttcacttatttcaaatatatccatccataactgaccactgatgcattttttatgctagtgaactttaaaagttttgtcagagaatttgaacattatcttggcatcatttggttgtgatgatagtggtggtgatgttttcatgtaacttgaaatttttctagttcttgccatgacaagtttggttttttttttaattgaaacctggaaacttagagtattgttataacactctggatctcatttaaaccatcgattttatctggcttcctttgacataattcaaacaaggaaagtggcaaactacttgtagaagtcttggttccccactcagttctcaatgatgtctgagctggggactcattactgtttgataagagttctggatctccagatggcttccatctgtgaggaggagaaaggtggccctgcttccactagatagtgataagaggcttgattctccactaggtatcctgtgacagactactagctgggatggggagggacatctcattattgctggaagtagggcaaatccagattcctcttatggtctctcctgatatcatagggaaatggaggactcattatcacccagtgaggatgaaagtctcaactcaatcttctctgacaccaccctgatgaatgattagggttcctggtggtggtttgcaggtgggtgggggtgtgtggtgtttgactgaggtagagttttactgtctaaaagttttctatcttgtagccggcccttttcttggtcctttgactacagacaggaggcttttgttgaaactttttttgtctacagtcattgacatttcaaggtttctaattccttcagccccaagtctaagatacagaaggcaaaaagtgaggagactctaggaaattcatcaacatgtcatttcttgggcccaaggattctaacccatctgcctactttccatctttctgagttaccttaatatatttttatggcatatattataatgagatataatacagtattatattttatatgacatgtatttattttcattctatggttcgtagctatattgagaagaatagggaaagtatatctactccatgttcccagaagtggaagcctcccttgagatttcaatttcggttaccattttcacttttaacatcttactgactttccttcattgttaatgaccatacttgaatttttatctattttaactttttgattacttttacatttttaccttttcagattattttcctcactcaagtgcttagaataccaatcctgcaattcattgtatctacaaattcccttcattctggttcatttcctcctgtggtctgtaagttttgaatgagtgtttatcttcagcctgactattttctttcctgagtgaatccctttcctcttatattatagaatccctagagagcaattttgtggggtacatgggtggctcagtcggttaagcagctgccttcagcttgggtcatgatcccagagtcctgggattaagccccacatcaggctccctgctttgtgagaagtctgctccctctccctctgtgccttccctgcttgtgctctttctctgtgtcaaatgaataaataaaatatttaaaaaaaaaacaaaaaaaaagcaatgttgtttcactgctattgggcacctctgagaattcacttgtccaggatgagtatttacatttttaatatgtcagtttaaaattcatttctatacatggagaatgaattagtattccatacttaggcatagtgtggtatctgaatatcagttcttaatttaggcattttttcccctactccaaacatagaggagataggcttctatgtcatttcctgggacttatgatgttttctcacttatctgacaaactgggcagctcgaaagggttctcagctttgtgttggtgtctgctctcacctcacatgggcctttggtcacatctcttatcctgtatggacattaaaaccctagcacctagccactatgtcttctatctgtttgtatatctttggttcagtttgacatctcttatttgtctagctataaattatttctcacaccctgggattgtgttaatgtgtgctgatgctagctcctgcaggctggcaagggttaatggttacattttcagcaatttgtaaactgatcattataaacatgctggttaaaatacgcatttctaaatatttatgttagatgttttctgttaattcagaaagccatgatgccaagatcagaattttgtcattgattaaagcacctgggtggttcagtcagttaagcttttgccttcagctcaggtcatgatctcagtgtcctgggacagagccccatgtagggctccctgctcaggtgaaagtctgcttctctgtctctcccctcattcgtgtgctctttctctaataaataataaaatctttaaaaagaaaaaaggattttgtcattgatttaacgaagtcagaaactacttcgaaaattattttacttttcatccagggcaatatccttccaccctctccctctcccttacaaagatgagaaaaagaaggctcagagatcaaatggcaagagtctgtacaagggatttccctttactctctatttctccactgctctttctaatatttcatcctgtggtcctgcctgtacttgtttatgtaaaagattatttctccacttttcttctaaaacaatagaaggatgaagatgctatgcatcccaataaaaggatgaagatgctaagtctctttcttccaagtggcagggtaatggaagcatcattaagagctgtctaggccctggtagctcccttgagatacctcggttggagcaatcagagaagccacttctgtaaattatttttttaagatttatttgttttagagagagagagaggaaaagagtgtgaaagtaaggggaggggcagtgggagagaatccttagaccccccccactgagcgcagaacccaacacagtgctcattcccaggaccctgacatcatgacctgagccaaaatcaaaagccagctgcctaaccaactgagccacccaggcacccccgagaaatcgcttctaaaagacgaatatctttctgcatttccccagaggacactaacacttgtctaacagcccccgtgccaatgaaagtttctagaagtcagcttcaccaagcttttatatagccagacagatgtaattttgaaccatttctattttttctcattttctaccttaccctttcctctttctcttcttttcctttgcaccttcttcatttcacagattccaccaccaccaccaccaccaccaccaccatcatcattatcatcatcatcatccttattattttactaaagcaatcatcgaaatgcctatctaaatattcattaacaagccctatgaatgcattgtttcccaaacactgcattttgtaagaaaatggtaaatttctagttagtagcaggacatgtcttaccaaggtactaaaaaaggcagtggctgaagacatatgtcaagaacatacaaaaagtcaaaacctagaaatgccatcaattagtaggacatttgtaatcctcactgtagataactcttaaatagatccagatcaacatgcaactatgatgctaatgggacttatgttcaggattgttgaggatccagggattattcagttcaactccttttcctgaaaaacgaggaaggaaaactaagaaagcaagactcaactctattttcaaactcctagattataccactcttctatgttaccctcttcaagcatgtaataaaatctccttgtcactttaatgatacaagctgaattcacttgaatgtcatttctgtgacaaaaatcatttaaaaggtcatgctctgtggtccagacagccagtattagccaaaaagtaatacgttgataagtatttgatgcttctgttcctatatgaaaatatgccctccatgattagctgtcacaatgtcatccccataaaaagaaatgttcttaacagaatagtgtaagagttacattaatacctctggaaccagactgcccgggtgtgaatgttattttaatcatatcctagctgtgtgactttgggcacgtcactgatgttctctgttttacttttctcatttacaaaatatggatgatgatagtaactacatttgggttgtcttgaatatcgaatgaaccatacgtctaaaatacgtagaacactttctgatgcaaagtaaatgccacttgaagagatcagtgattataacagattatttttttaaatgcctgaaatgatctctcttcctatcccgatgctccttctagagtgacagtacaaaaacttccatgaatggatggactctatttccccatccttggaatctgacccggccttaccacttgctttggctagtagaagtgataatgtaccaattttgcacatcactcaagagtctgtgcttccataacctcagccctctgttccactgaagagacaggctgagggtagcctgtggaacactgtgatccacagataccagagccaggtctgcccaaactgaggtcctggagacaccaaagtcagcacagttgcccacagatgcatgtggaagcacagcaaagaattttataattcacaccctccaagtattggcttatatttccaaagaaacatccctaaagagttctctcaggatacaaggatctatggagtagaaaaggaaacaaaatgtcatgcagaaacacactgatgttttttaaacttcacccaattgaaggaagttttgagactttatatttttagattgctttaccatttcctttttttttttttttttttaattttttttttctttttttttttttttttttttttttaccatttcctattggtgagtaaatgtcagagcttgaggacaaagaacagaggtcattcaatattctcactgctgccttcttttatggtgctaaattcccaaatccttcactagggaacaaatttctttttttttttttaatttttatttatttatgatagtcacacagagagagagagagagagacagagacagagacagagacataggcagagggagaagcaggctccatgcaccgggagcccgacgtgggattcgatcctgggtctccaggatcgcgccctgggccaaaggcaggcactaaaccactgcgccacccagggatccccgggaacaaatttctgaaagcacttcaccagaatgacagatacatattaaacacccttcaaggcaggctatgtttttgtgcattttcccactaatggatgtaactttagaagaggatgtgtcatctagatttgcacctagtagtttgaagaagacggtacagagcacctgttctgagatcccacagcactgagttctaccactggctctaacgttcacagactataagattttggacaaattgtctaattattgatgccccatttcctcatttataaaatatgagcaataatacttatcttgagggacccagagaagatggagtatgtaaaagcatctagtatcacctcacatagaacaactgttcacttctcattggagcttacatttaaaaccaaccaagtttcacatcaatctagctacctggataaattatccaatgccctagtgtatccaataacaaaacaagattttaaaagccattatataattcctgtaaagttatgtgaaagctttttatttttttaatgactgccctatgctttatttacagtaagctttgtgcttgaatgtgccaccactggagtccagaaacactgttttttttgtttgtttgtttgttttgttttggggcttttttgtatatattttattgcagttcaatttgccaacatatagcataacacccagtgctcatcccatcaagtaccaccctcagtgcccgtcacccagtcaccccgtccccctgcctaactccccttccactaccccttgttcgtttcccagagttaggagtctctcatgttctctcactctctctgatatttcccactcattttctcccctctttaatccctgtgaaagcttttttaaaaaaatataaggtacactacaaatggaagagaggataatggtgattatgaacatggttttgaaaattgatgacaataatgatataatttgggaattattggtgaatcttctgtaggaatctcagcaagctccctaaatgtcttttggaaaaatatatgtagatgttattgttactacataaaaatatcaaagccaggacccactctgaacaggaataaaggggtaaaatttgtcattataaacaccacctcaacacacatgcacttgcaggttcccaggtttaacaaacacacttcctgttctgctctatttccctttttcaccttccccccccccccattctatacccatataagctccaatatacccaggtttttcatcatgttctgaggcttttccatctgggatcacacacttgagtggaagttaatttgtgaatttctaggtgtgatgtaacttgagatattgtggtgtttctggcagagtgtggaatttgtaagctgaagatcccaaaacacaccgcattggctgcagaatgaactggaaggaaggtaggagaaaatagagacggcattttaagaggtatagatgaggagaaaggagagtttaggaagtaactaaagggattcttattcctgagtaatgcttaacagttaacaaactaaaccaaatgttcaccctgctttagtgttctgtgacccgtttggcagaacctcgaaccctctgcaagatattgcaccttgtgagcctccacttcctcctctgtaaaatcggaataatatatatagtggtgtgctcctaaatgtctaacaatcaactctcggggggaggcgggggaagccctgatttgtaacatttggccatttttgtggtaaatagaccacaaaaagaccaatttcaagctaccaacagaactctattaaacacaaagttgggaagagatgcaccatagaacaacagtttataatatttctaccatacataatagatttaaatagcctgaaaagcatagataatagtaaaatatagtaaaataattaggaagtaatgagcttgagttttcattaccactgtttccatggaacccattttgagttaactttctatagtaatttaatttttaacaatgtctacattttgcaacctgttctcaaaactcctggaaatgtaacaaccagctcttcctagcttatatggaccaactctagcaaatcactggttgcatctgctaaaagagaagactcttttgagaattttcatgctacatttttatttaaatcttaaggaaaccatatttaaagctattatttctatctttccaatttagcagtcccaaataaccttatcagtaactctggatgttgcagaaactgatattatttgggtgttaattgcattgtccctgtggctcagtcttgatgcctgccatcttgtcagaggtaatggcaggaagtctaagagctccagacatgactagagagtgtgatagtgatggcattaattacaacaaaagagtctaggggggagaatcaatcatggtggcccacattcctttcactgatgtattccatgcctttggactccccatgaggagggaaatgagctggtcatctggctggtcaaagagtgtcctaaaatccatgtgactgatgtgtgtcagttatgtctagaatggtttcaggagttccctctgtcaggcaggtagattgaagacactggttagcagattaaaaggaggaagacaatagatcagagaaacaattggtgatgagaaagaatacctaagaagtggagaaaggaggaaaaaagattggagacagaaagagagactgagagagagagattcatctagttttgttcctgagactaaaatctgatcaagtttattaaatggaaggaatctatttttggaatcaggtttccaggaaaattcttcagagtaaaattgtatcttaaccaagcaacacctttctgtctggaggaaataagcattaacaaagatccaaagacgggatatagatcttcagtcaacttaagagattcctttcattcttttactcttaataccaacagagaaagtctttccttgggcaggcaaagataacattttattttattttttttaagtaatagaatatgcatttaagacaaaagacaaaagctatgacaaatgttgtaagcaagtgtttgacaaagagtatgtgttgagtgttaatgtcaatttacttatttttcttaccttccactttctcaagtcttgaaaaaatgtcacccattagttgatggggacagaaaaatctcaaagtaaattaacctggcagggttgctcatcaaggcagctggaaggaagatggagaagattctaaagctcagtcagagcactcccttttattcttagcagaaaatgtgtagagtcaagtttgaaggtctagacacatctgaaacataggtaagaacaggaaagagcaagatatgggaagcaaattgtgttctagaactaggtcaagatgactgctcataatgtagtgtgtgtacatgccaagttaggagtaggagacattcccctttcccccactcctcggtgaagggttcttgtagctgatttaggaatatcactggcatgagtcaggttaacagcagaaaaaaaaaaaaaaaaaaacccgaagttttattttatgcacacagaggtccaatattaaaatggataaccagagaaacgacttggtgggcagttttttatacattttagacaaagagacaataaatttgtgaggaattgacaggagaaagaatacacttgtttgggaactttaattaggaaagaattataaatagaatttaggctgaaatagactcacctgggtggctcagggtgagcatctgcctttggctcaggtcatgatcccagggtcctgggatcgagttctgcaccaggctccccgcagagagcttgcttctccctctgcctatgtctctgcctctctccttgtgtctctcatgaataagtaaataaaatctttaaagaaaaaaaaaaagaatttaggctgaggtagatgagaaaaaaaataaggtttgtttcgacagctttcttggctttaaagtccaagtgataagaatatctttttactttttgctttttacttcccagtgcagaggcagatatttttcataagggggatttgttttctgctcccagggggacacaggagggtctgagtgtccttgccccagctgttttccaagtagtttcaattcaaaataatcgctctaaaataataataataataatcgctctgtcattgcggcatattctgggtgacctgtcctgggaccctacacccacatgcacacctggacacagacactcaaaatgtaattcaagtcagcagctgagagtggacaattcatatatttttttaatattttacttatttattcatgagagacacagagagagagaggcagagacacaggcagaggtagaagtagactccatgcagggagccccacgtgggactcgatcccgagtctccaggatcaggccctgggctgaaggtggcgctaaaccgctgagccacccgggctgccctcatattcttttcttgtggataatagaaagagaagactctccatttccttaaatgatgaatagagaaaacatcccaagttgttcctgtgttgttaggatttaagtagaacttagcacaactactaaatatgggggaaaggtagcagaagcacagtcgtctattattattaattattctgaaatgtagatctaagaatgtccctgtgccttttcaaataggctttttcttttgaaacgtataaaaaaataattcatcaataactaaccccatatacatgctgagaatgatggcaaaatccataactacagaaacggactatgttacgagggagaaatgcaaaagataataacaaaaaaatgaagaaattatcaagtatgttagaggtacagtgtctccacctactgtagagcagaaggattcaaaccctccatgcctcgttgtttgcgtttttttgcgttttttttttctctgatcttaattttcctgctctccaagctcatgcttcagaggaaaatatgcttgtttaaggtgccaggaagccacaggcttttctttcatttgtggacagacatcatcaatgaagccaggatgagattagtttccctgcccccccccccccccccacgcaccaccaccagcctgttcatttctaggcactctgtttatttcaatggtcttctctttgaattctgattttacttttcagaaactcataatttgatcccagttctacgactctggcaaatatcagaatctggtgaggacgaagaatccttgatctggtgcctgatcctgggtttagcatataatttcctcaaaggggagataataaaagatgtgttccctttagatcacaagaaaaccaagcctggcaaataaggtcttggagaaagtatatttggtcaaccatgagctggactcactaactactgtagtgtagtcccaggatgacccaacttctcaaggcatatagtcagacaaatgctgcagggcaagtcacatctcccaaaatatgattgctaatttatacactaaccagagtccctaagactgtttcttttagctctgtcttttgctgagtacatacaatgcatctgctgctgtccaggcaggaaagtagtatgattccagctttgtgggaatttataagggtcccaagggtcagcctcaccacacttggaagttgaatacagaaaagtacctccaggggcagattcttgtcagactcattctccggagtctgacatgccatcttccctgcagaaatactactctccaggactgcgagaaaaaagaaatctatgcaaatatttccaatatgctcaagaaagctgaaggctcccaggagcaaacttcatttgatgcaaggtattctttcaccattcccgtctcaatggaccacaatttagagaaggagtcatgtctaatagctttctggattgagctggggcattgtgggataagcgaactcagaatatatcagagtgattttaaagaagccagagtttttacaagaaccagaaatgtgaagttggctgacctcagtttgggtctcgattctactaccacttggatgttaactgtatgaccttgggcaaataacttatcctttctgaacatcagctttcttgtccatggaatagaaatgacatttccaacctcacagagttatttcaaagactgagatgaacttgggaaataattcataatttgcaaagtgctttctactcctttattgttcttaacaacatttcagaaaatctactatatagtccctgccaaagatatttcttgatttagagcattgaggaaagtcaacaaactcccaaatgacaccataacctgctttaaaaaaaaaaaagaaagaaagaaaagaaaagaaaaatcaactaaacagaatggctattttcataccagtggaaagtataattatttgccttccttcctctttgcctgatacctgaagtttcactattcctattagcaaaggtcagagctttaagctaatgctgttgctcaggggtgcttacagattattaatcatttatcctttcacttgagagggtttcagatactgtaccagaaggaagggctactaacacaagtgctattgggtattaaggaccagagccccacctaaggtgtcaatgagaaggaagtttctatcagacaccatgaaaggacttcatcagaccccaaaactcctgtcccagaggattatctgaataggaaagacccacagtacacagaggatcttattcaaggcctctcttgaagcctgatggagaaggtggcattgccacccaacacagctcctcaaatgatgaccttacacctggtagaatcagaaccttgtggatcggccagtccttttacatccagcaaggaaccacgcacaggtgtgagttagagtagggaatgctcaattaattaagagattgcgtcaacaacaggctttgctagtgcaatcgatcttttggggcataagctcttgcctccagccttgacccagctgctactttattaagtcctctccttccttagtgaggaagagacactgggggagaagggactctgtcctcagaactgttgctgtcaaggtcattgctgagctgtgcaacagtgaggctggaggcaaaagaaaaaacagtaacacggagcctgtcgttattgaagattttgataattggttcactgtggatttcttttttgcattaactttgttttttaaaaccattgcattaaaacggtatttgtctcagtgactggatttttttggcagcccctatgaatgcctcacttgccccactccatcacagtccggacttaggatccccatcctcagaggagaaaacgggtaaccaaagacaggcacatgtacctgcctgcccaggcatcacagtcactgtgatggttaatttcgtgtgtcaacttgacagcactacagggttcccaggttaaacactatttcagggtttgcctgtgagggtgtttctgggtgagattagtatttgaataggcatactcagtgaagcaggctgtcctcctggggaaggttgagggtactgggtggaggagggttgagggggatgggcatcacccagttgagggcctgaatagaacaaaaatcttcacctgcctgctggagctgggacatcaggtctctcctgcccttacactgggatttacagcattggattccctggttctcaggctttaggacatggactggattggatttacaccatccgttctcctgggtatcctgcttgtagacagcagaccgtgggacttctcagcttccgtaattgtgtgagcccattcctcataataaatcaccatatatatgtgtgtgtaatttatattatcttatatatgattttatatataaattatatgtgtatttattcaaatatacagttatttattcatttatttacttatttatttatgtccaattgcttccgtttctctgcagaatcctgattaatataatcaccttccagactcaagggtaacagggacatccctccaagttggaatgggaggtcttcctttgagagcaatgggtctctctcttggggaccacgggggtttaaacccagaccacgtgcagccaaacacagggctccctcctgctcgttccttgtcccagcctcagctaagggtggtggtccaattctctgtccactttctgtgacagaaattaacgtgccccagtgctgcccttctcttctacccacacagagacagcaaattgagaactgaaagtatcacttgaaagcacaccttatttcaaaacctgcacatgttaacaaactcaacaacccctcctgcccctaaccccctgctaagagaaggggatctgataatacatgcacagtcacatctctaattgtgttaattccctcatttcagaagggaggagggaggctgtaaaaattcaggtctcctctgaaggagactccaagtccagttcagcagttgatacaatattgttcatatatccgaactatacaggaagtcaaggttggggcaggggatggaagctcagattcgatctggtgccttgtcccagaccaccccttgacttttctcctcttctatttttaacttctccctgcccccccggcccccttattcctctgcaactctctgagagtctaatttacaaagaggccaaaagaaggtgttgcaccagtgtcaggcacttgagctgccatttctgatttccctggtagatatggtagattgaagaaagaacaactgtagctgaagattagatttcattttctccatgtgttccaacccttgacacagtctgcagcaggtcacagctaacctcctgccttcagcagaacgggggaattgcattggctcccagactggggaggtcaatttctcagtcacgtcagaccctctctgcgagcttgtcagctctttccatatcgttcgcaataagtcctgtatgtcctcgaagggaacaaacacaaggaggaaggcagtgaggtatctttgggaggcagaatgtgctagaccgggcacattaggacttcaactgaatgttacattggttttctctgactcctgattccaccatcatagattccactggcttaagggttttgcaagtaaatatacttactccataggagcatcccaaaagtccctccacactctccattcctattaccttattctgcaagaataattatattttggtagtccttaacaccttagtatgagatgccataaaaatctttttggtattgaaaggaaaacagaatgtggaatggaggggacactttgtaccaaggaaagtggagaggaagagagaggaccagttttgtggccattcaccttgtgtcaggctctgccgtggggaaggggtggggaagtcggtcacatataatatttttaagacatttatttattttggggaagagaggcagaggagaaagattcttaagcagatatttcactgtgtacagagcttgacacggggctcgatctcaggacgttgagatcactacctgagcccaaaccaagagttggacacttagccaactgtgccccccaggtgccccctatgaaataaattttaaataaagaaaataagacatctccttaagaaaagattgaatggcaaacagaattagaaagaaagaaagaaagaaagaaagaaagaaagaaagaaagaaagaaagaaagaaagaaagaaagaagaaagaaagaaagaaagaaagaaagaaagaaagaaagaaagaaagaaagaaagaaagaaagaaagaaagaaagattaaagagaatgaaacaaacaaaaaagccaaaccaatgtagataaaataaagctgcaatggtttctgataaatgatctagtataggatccccttttaattgcttctgctccttgaagttttcttgctaaaaatcatgctgtattttggcttatgctcaaatcttaaaaccactttgcatttgagaaaacctgagtgggacccaggcacccatatatatctgatataccgaaggcaggctgagggccacttagaaaagtgcactgtccagagcctctaatagcaaacggcacagcctgcaggtgggagagattatatcggttttagtttcttgttttgtaagatgaacacagtcatcatgcaacagattcttctgaaacctaattcgagatgcccggatttccatccagtttccatctttaagcaacaagtacccattagagagttctaccattgtccccaagagactgggatggaaatgaataaatgaacttttaaagataattggcttccaaaatgccctcctgttcctccttctctatattttgacaacccctgtgggaattgcatggtgaaacaacttgggattctttgggtggatgatgtgtggcagagcgccagtccatggaaacgaatctcttaggaattgaggtgtcccgctactgggctgcttcagggtggtaggaatttaactagctctgctcgaaagcccgcttctgaccttcttctgctggattcccctcatccatgttctcctgtactccattctagctgcttattctagcacagttcaaaatatgcattttgaatcctcaaatcaaaaggttttcctaaagtttatttctccgaggctgcatttttcccacatgttcccatttaaagtgatggagtgaactgtttgcaatctgtcttcaggaaaccctcaggatacttaactctttcacttaaaaaatatttgcagatcactaataattgaggcattagtccagttgttccttgaaaaaaaagcaagtaatacccttgctagtgaagagcttgtgttctagcagagggacttagaaaatactcaaggacaggagtaataatttcaaattattcgaagtgccggaactgaagtaagcagggtcctatgctagaaagcaactgacaagaagtatttgctttaagagagaataacagagtgcttccattttagatctggcagtaaaagaaggcacctcttggaaaggtaactttttaactaagatatagaaaatataactaataagccatgcaaaggtccggggaagaagactttaggtaaatggtacaaatgcaaaggacaaatggactgaactaaaaagacttagggtgtccagcaggaaggattctctcacgaggacaacatgacaatggagagggaggcaggagcttgtttgccattctaaggagtttgaatgatattctaaaaataatgagaaaacattttagcaggtacataatcaccagcacacacccaccattagttgcacactaaatgcaacctgggatcctggaacagaaaaaaaaaaaagatatgtgtgtgcacacactcagatacgtgtgtgcatagccaggctcaggggacccccatcatatagttctgacttcttctctgccacagaactcctgctgaaatcaacaagaggcttaaaaaagggggaggtggtgagaggtatgaatgaacatctctttctgcatcattttccaattaccctattcaatcggctcatccaacatggccaaaggttcctaaaacaacatcattttccagtgttgagtatcatagagcattcccctaacctagaccaatttggatggctttggggtatgttattggataattttgtcaatgataatttttaaaaagttgctgtttaattagcagggaagaagccaacatttgcagggagataataaaggaatccaataaaagtccttgatgaccgtaacaggagatggctgtccccaagtgcttccaattctgagtattgctgtcggcaggctgccatggaactgctcagcaagaagaaacacacctccgctggacacctagagaaaggatcagctataaagggataggagtaaactcgtaataagaatactttgaacttttattataattcctgccagtagatgtttcctagatgatgcaggtgatatatagaatctgagtagctcaatagttaagagtttgggctttggagcccaaactatagaaatagtttacaccctccctgccctacacttgctagcattatgaccttctggactggccttcttcctcatctcagcaaacggcaccttattcttccctccttgactcctctctttacatcccatgatctattatatcttattgaatttgacctacaaaatatacccggaacccaacccaactatctcccaccacctccacagctgttaccctaggccaagccaccagcatctcttgctagatttgtgcagtggtttccaaattgatctctctcccagagagacctagtaaaccattaaaatcaagattggggagctgtaaattcattcacccccttctgagtgtgagctggcctgaatgactcacttccaaagattaaggaagagaaagggacaaaccatcacttcaaagtggagaaaccaggtagatgccaccttaaccaagtatccatgctcccatcatcatgataagtctggtggatgtcacccattctctgatatactgtgatgagaaagagactttatctctgtggcattcttcatcaaaaccataagcctagtcaaactgagaaaacattggacaaccccaagtagagcgacattctacaaaatacatgcccaggactcttcaaagtctcaaagtcaggaggtacaagacaagactaaggaattgtcagagttggggaaagactgaaacaggatgactaaatgcaacctgggatcctggaacagaaaaaaagaacattagtgggcaaactggtgaaatctggctaaagcctctaattcagggagcagtgatgtaccaatgttaatgtattaattaagacacatgtaccatcattagctgagatgttactattagtggaaatgaggtaaatgatacatggcaacactgtgcactatctttgccatctttctctaagtttaaaataattccaaaattttaaaaatatattaaccagcaaaaaaaaaaaaaagagagagagaagtcagtcagatcatttcattcctctactcaaaactcctgaaaggcttcttaccttcctgactgcatgacctggcccatgatcacctctccgacctcaggagttcattctcatcacactctcccctaattattcctccaacatgacatgttcctcatctagggtctgtgcatttattttttccacttcctgaggtgccctttctccagatgtgtggttggttcccaccatttctttaggtcccttttccaatatcattttgttagggcttcccctgaccatctccctccaaaatttatatctccctccgacacttcctttttttttataaagattttatttatttattcatgaaagacagagagagagagagagagagagagggccaaagacacaggcagagggagaagcaggctccatgcagggagcctgatgtgggactcaatcccaggtctccaggatcaggccctgggctgaaggcagcgctaaactgctgagccaccaaggctgccccctccgacacttcttaatgcttgtctctgcatcatttctctcctcaacacttattacctctatcaggctatgcattttatttattttgttaatctgtagcctctcctactggaacagaagctctaagaaacagaaatttctgactactatgttcactgcgatattcccaattcccagggcagtacctagtactccacgagtattcttctgagaattaaaatgaccttgagtatttacttaatctctgaattttgttttttacatacgtaaaatgagaataataatatgaaaattgtttacataataagttccttataaagcctaagtatgataaagaatgagtgttttgtatagtatttagcatgtcaaatccttaagaaattatatattatcactagttaggtttcccttactagtattactactgtcactaattactacttctgttattgattatttacaagtggctataggtttgatatcaaattagaactaacctgaaagctctgtgatctctgattaacccctaacacatgcaggtccctacctatgggaaaatccatagctccttcctcccattagaataaatgattttctggttttttgttttttgttttgtttttaattgtgtgtgtgtgtgtgtgtgtgtgtgtgtgtgtgtgtgtgtgtgttgaatgagaggactaagaataaagattttaaatggaatcctaaggttttatttctgagtctaaaaacaatttgagtcatttttcagagagttgcctcccctaggaaatctctgaactccttttcaaaatagcaacaagaacactcactctgatttctgaaaatgagctcaacctagcttgtgcctttcccatatctacttcatatataaccatacattaggcactctctgggctggagtggcttataatctagtttaggggcatatgtgggtgaaaaggagaataaatttcatgttatcatacaaacaagataggatacacactgtatgcctttgtcctgagaaaagacaaaggaaaaatatcacatagagtcagaaggatttcatcatgtctgtccattgcttttaggtccttggaaatgggtaatgatgcattacatctcttttcttggctcttgatattgatgataatgataaaaatatttattattaatattcttattatgaaaggcccaggggtagagatagaatgtattaccgtattatatttacttgtagtcataaaattattatagttttatcattaaaatctaaagaattaaaatctaaaaaatttcaaaaactcacaaaaataaaactgtacccaactcagtgaatgcctcgcttttataatagtcacaacaaggttacaagacaggtactatttttgtccttatttttaagatagagaaattgaagttttgcaaaggggaggtaatgggcccacagggacctgccaggagatgggagaaaatctgatctactcaaacccagatctactaggctccacagcccgcaccccatccctctcctgtatttggagggtctgaagtagagaccgagtacttggaacaagactatttatacatgctaattgggattaggtttacatttattctgctgaggtcacaaatcttattgtccaggcacacacgtcaagtctgatcaggaacaactagagcttggaattgaatttcctacgctctttgtcctcaacagcttgcagttcgagctgaacgtatgtgttatccatccctgcagctgtgctccaggaacattcatagtccagaaagttaaatagcaccggagacattcttgtccaaactggactcttctgtctcctttactcagacatctgatgacatccaaggaaaccatgtgcaaagcaaggtgaagatatctttccagggtgttctctggggaggaagaaagaagaaagggatgcctagatagaagcatgatcttacaactttcattgtctccttctctttctgtaagaagaaactgagaagcaaggtaatacttatcagttgggagacatcagacacaactagatctacgctgctgcataaacaaatatgggtttggagttcacagattgcccctgcagaataagaatgttggcccacagctcaaaacgctaggtgactgacaggatgaccttgggcaaggtcaggagggagccaagccagtagagttctgtgcgtgtgctcagctctgacagtggtcagaagcctcagacagctccaccctgtagtcgcccccacatgtagtgcattctgatttcacactagttggctttgtttttacaggtctaaagagggaacaagaatgaagaagaaacttgggctcctgggtgactcggtcggtagtttaagcatccatctcttagtttagactcaagtcaatctccccacctctggctctgtgctcagcagggagtctgttcccccctccctctctctctctctctacctaccaccaatcccctgctcatgtactccctctctctcaaataaataaacaaatctttaaaaaaaaaaaaaatgaaaaagaaaccagagtaacaagagcaacagaaaccataattgccagggattcaacacactattgcatgtcaacagagaaaagacagaactactcttaattggtttctaccatttctatcaaggttaactgtttaaaaactagagagggtgtggaaggaaaacaaaactgagaaggaattataccccaagctagaatgatgcctcctaacatgatgtaagttggtatgtccaggggaagctcccaggatttagatggggtcacagaaatgctgtgggttgactctgtcgatgatgaattttgtatagtcccatgttgcatgattactcccgtaagttttgccccagttctcagcaggaaaaattaagaacctagtgatgttatttcaccaacccaattccagaaatactatggtttgtattaatgagcaagtgaagtatgagaacagagggagctttgcaattttccaggaattattacctgtttcgtaaacccaaccatccacgtctggccttcccttttgcatggtggtactggagctcttggagagtagtggtaaagatgctgcacacgggcacaaacaccatgctgaggttaagaggacagagcctctcacagtgacttcatggagaagagagaaaggcatctctgaatgatgaatcactaagttaaaattattagcagacaaatgtcagcccatatggcctgcttctgacctttgtcttattgggttcaatgttttttgtttcatgctattttttttaaaaaatcaatcactttgaaaatggtttctttcgtctgatttgcagatgagacaaggttgaatatcaaactaataaaaaatatggaaaaactagatttgaaaatgtctgccctgtttgtaaagtaaaatcatgaattaaaaaatcattattaagaaaaatatttatgagaaataccacaggacactagaggatattcctaatctgtaaaggctactcacaaaaaaaaaaaaaaaacccaaaaatcaaaaaccaaaaaccctacaactccaatagatgtaaacatgcaatccacaaaagaaaacagggttgtggacccgggtggctcagtcattgagtatctgcctttggctcaggtcgtggtcctggggtcctgagatcatgcccagcatgatccagctcaccaggaagtctacttctccttccctctctgcccctccccctgctcatactctctctctcaaataaataaaattatttaaaatgagagggcccctgggtggctcagcagttgaggaactgcctttggctcaggtcgtgatcctgtggacctgggatcgagtcccaatcaatctcctcgcaggaagcctgcttctccctctgcctgtgcctctgcctctttctctctgtctcttatgaataagtttttttttaatctttaaaaaaaaaaaaaaagggaaagaaaacaaggttgctaaacttcttaaagaaatatgttcaaccacaccaacaaaatctgaaaaccataacatactatatccatcaattaaaataacaaatttcaagggaattttggtgtctaactttagggataaacttaacataaagaaatttagctatctcctccattgccttgaggcactctactcttatcctttgacacattatccacttctagaaatctttcctaaagatatcaacattcctaaggataatataaagggatgataattcctggaaaagagcatgggcgccctctgctttcatgctttacttgttcattaatacacatcatagtatttctgaaaagggggactgagataacatgtttaggttctttatctttcataatggctaagatttgcatgctaaggtgttcatcctagtattaaatgtgtaagtcaagagaaggaggaggaggaggagacagaaggggtcagtgtggaaggggaggggagctgggactaaagacagtgaagaaaggaagagcatggtcagggagagagatgggagagagaatgatgtgaaagaagaaaacaaacattcaaatttaggaaaatggttaattaattattagacattcgtttagtggaactttatgaccacaaacccagtgcttcgaaatatgttaaatgacatgagaaaatgctgtgataaaatgttatttaaatcctaggccaaatttcacattaatctctaccaaacatcaactgtaccaataacattgagaaaaattgtgattcgacaaagatttataagtaacacataaataattgactccctctactacaccatgagcaaccttccaagcgagggagacagctttcagcacattatctggaattcatggaaagcattagtaaatgtttgtagaataaatgattacagaatcagcatgtggcttagttcaagttttctagaagacagagctacagacatggtatatgtgctgttcctttcacagcaatgctaggccagcatgaagggagaaagcaaatgaaagcttatgcatatcatgatgtccataacttcacaaggaaacttagctggttttttattcaccccaaatgtcactatgcatttttgactgattatacctaagaacgatccattaggagaaagggaaaaaaaatctactgactcccttctatctcccatttatcattgatcaaactttgtctaggggggcatgaataccttcatttatctgtctgtccctcctggccctctgagcagccactggagaagccagatctcttgcccatggcacttcatctgagtcctgaagaggtgagttgaactgcatccaagtctctgttgctccagatgggttgagtctggtagtactgggctgagacttctgccataatgggaacaacagaggcaatgctggacaccagcctacactggggaagcagagatagctagaaatgtcatgagaggagtcagttacattaggactcaagagagcaagtggcagagggaaccaaggagcaagtgggccaagtgaaattggaaagatgtgtaacttgggtatgatgccacttgtactgctatcttgatgtcacaaaaattttttttttttaaagattttatttatttattcatgagagacacggcggggggcggtggtggcagagggagaagcaggctccatgatgggagcccgatgtaggactcgatcccgggactccaggatcacgccctgggctgaaggtggcgctaaacctctgagccacccaggaatcccctgatgtcacaaatctaatatatttatccataccagtagaaattcttgtcctggacaaattccatgatcatccttctgtgactcattaaagtcaaaccatagatgaagcactgtattcagttcttattgtcctaaatataaagggaacaagtaacatcagacaagaatataaaaagataaagataaacataatgaggaagaatctgggaaacttgtgattatgtgccaagaaggaggaaagggagggggaattagtgatccctaaattctctattaactgttatattgcagggtcattttgaaatccaactgagaagtctggaaatatgaatttgaagactggctaattaagtacaaataggatcaaaattgaaactttgtatcctttgaccagcatctctccatttcccaactttcatcccctggcagccaccaaccattctactccctgcttttatgagttccactttgaggtgatggttgaatatggccatcatatatatatgagatatatatatatacacatacacacatatgtacacacacgtatatatctatcaaaacatcacattgtgtaccttaaatttacacaatttttatttgccaattattcctcaatagagctggaagaagtacaaatactatcaacaaagtgtgtttctcattcagagatgctaataaaatttttactattgctttctatcagttacctctgacaacatatattgatatttccagaaagaagataaaaattgtaaagtaataaaaagaaaaacacgaataataagaatagcagcaacaataattaaataatatgattgttttgcacacagattgtgtatgagaggtacaaagaatatttatatcctgtatatatatttttgttttgttttgtttttacaacactcctagtttgagcagaagagctgaaacttggatatcaaaggagaaaggattggtctggaatatgggaacagcaaaaaaaccatcacctcaagaataaaaaaacagaaattactggcttcacgctgagttcacaggaagtgacatctgtgttcttgggttcagtgtgagaacctcaggtcatctacttcctggactgtcaactgcaatgcccatgctgggctccaaatcatcattttcttaggtttctaagtaacatcagtcatttctgatgggaccatttctacaaacctcctttagtatttgcatgaaatttgagtactgggcatcccttcatcccagccatttttcttgcccccagtcaggacttggattgctcctccttccttgactgatctccagaacttagcatcttatctacttcttgttaatgctttcaaaaaatttctccaaacctgctttacccaagatctatgaggtatgttgttagtcagcctggcttcaaacttaaattattcaaattgaatcaattacctttgtcttttttttctttttttaagttctgtgtggttaaagtgaccggtgatcttttaacctcccatgattaaaattgaacaagacatttcatctccaagaaactatgccctcctaataaactgggaattaactgtgtttaaatactctattctcataccactactcagataaaacctcttataggatcccatttcctgcagaacaagatgtcaagtcctgagcctggcaaaccaggccctctagaatttgatctcaggcttcctcactcctgctcctttacctgtacccttcatttgcccagactgaatttgtcttacatcattatatttcccacttctgcaccttttatttgtaccttccctctttcccatgatgttatcttctgcccatgactttctaaattctaccaatcctgcaaaccctccaggcagccctccttgattcctttacttcacaatgatttctccctcatcagagatacaacatttatttgaggggccattcatataatactattataattatcaagttctgttttttttgcaaagtaacttttcatatgtatatgattcatttttgtctactagatgaactctacatttccttataatctttagaagtcttagcacagagacccctgggtggctcagcggttgggcgcctgccttcagctcgggcatgatcccaggatccaggatcaagtcctgcatcaggctccctgcgaggagcttgcttctccttctgcctatgtctctgcctctgtctctcagtctgtgtctctcatgaataaataaataaatctttaaaaacaaacaaaaagaagtcctagcacatatttgtgcataaaactcaatttagaattattcaggtgtttgcttatatcactaattatgccaaggtgtttatatatataaaatcatctatattttgcatataaaattatatataaaaatcattaaaccttagcataagtattaaaatatatatgaatatatatatatatatatatatatatatatatatatttatgaattacatttagaagtggggtataatggtctttgggatgcctggaagaaaaaattatttaccaagtgacccaggtcttaattatttcatccatatcatcttatcaaatttaacccccacctactgaatttaacccccaccttaaatcctaaagtgtaagtattaacctccctcagtctcagctttctcatttgtaaaataaagataagctatttgtctattgttatacaaccagtatataatggggcaaaggtttgaaccaaacttctatttgaaccaaaatccgctgccctttccactatgttatgctgcctccaaagggaatgagggaatataaattctaggcatgcctgggtggctcagttggttaagcatcagactcatggtttcagctcaggccatgatcttcattgggattctctctctccctctccctctgttgtacccaagatcgcgaatccgagaaaccaccgaggagccaacaccgatgcaaacacaggagggtttatttacaagctccagcttgggtccaagtatacccgatacagcggagcagggacttggaccccgaggtgggttttagcttagttttaaggtctggtctaggggaactccagaaggggtggagcaattcctcaagttctgtttacatttggatatggggccttcaaggtcattgagctctgttcttattctaatatgaggcctcctgcccctggcttgggctctgttctcattctaataggagcttcctgctcttggcttgggctctgtttttattctaatatggggctttctaggacattaagctgtaaactattttttctgtaactgaagtaatgtaaatttcaggctgggatggctgtacttgtgctaacgctgaccttaaagtggaatggccttcattttctcggcctccacacctctgctcctctccctctttctctctctctctctcaaataaataaataaataaataaataaaatcttaaaagaaagaaagaaagaaagaaagaaagaaagaaagaaagaaagaaagaaagaaagaaagaaagaaagaaacccaaattttaaacctatctttggccaccaaccttctggatgacctgggtcaagtcacttcaggttcctgagagctcagttgcctgggttctaaactaagggaggctaaactacaccaatggctttaattttttttaaaatgaaccatagcaagaaataatacattaaatggcaacactctatatttctatacatatagatgcatatgcacagatacacatttatttatacagaataaaaacatttctattaacatttactatatgaattcaatctattttctattttattcttttattatcttttatatcactttttagaattttaacctacaatccactaaattgatttcacaaccagatcagaacctagccctagagcagaagacctctgaaatccctttttgctatacaggccaaggctctgactctcagctatgtgagaactgcagcgttgaattcaagcccaaggactcaatctgctttcacccaccaatataaatacaggtcagcttcctgacagagccccaggccacaagcttttatccaagttcaaccattgacatatcctgtgaactaggccataccacctgcatctaaacctttatctataagttaagaagaatgaaaacccttcactgacctcacagaattgccacaggggaaactcagaatgtctgcaaagccctttggaaatgccaaatgacttctttggaactacctctttttatgcagcaagaagtgttctatagcagataccatcatgaaagtgatatagaaagtacactgtctgaagctcatcaatttctgacctctgggtctttgacaaaaactgagtagatgttttcatagggtacctatcctcaaaaatacatgattcctttatactaaatggaagggaaaggtggagagatgaggaaagggggtcctatttcacaagccacacagaatgtcctaaaaaaaaaattacattgttaaatctattagggattttgaagatcttcccaatatttcacagagaaacagagctccaaacaggtaaatttttttcagtttattttttgttaagattttatttatttatgtaagagagatagaaaacacacacaagagagagagcatgagcaggggagggggcggagggagagggagaagaagactccctgttgagcagggcgcaagatgcagggctccatcccaggaccctgggatcagggcccaaaccgaaggcagatgcttaaccaactgaaccacccaaaactgccctgagttttcatgcagtttaattcagtttactgacttcagaagaagagagctacaattgaaggtagagtttcctatcacagctggtgtttaagtcaagggtgagtaaccatttggtgaagacatagtaaagagcactgaaatgcacagtggggacaattaggtgtcctttaaggtcaccaattcatctggcaaaaatgtattcagaagcaacatctattttatgctaattacaaagatagtctcaactatcccccttgtcttgccttttgccctacaacttcacatctattcccatcaaaaagtggtcttcccccttcccacctccttgaatctggactgattttgtgacttactttggccaataggatatggttgatgtaacctgtgtcagttctgagcctattcttcaagaagcctgttctcttaccaccctcaccagctatcatgagtaaaaacttggatcatcctggtggatgatgagtgataatcatcccccatcataccagctcattgtcatccaaccttaagacttgccagtgaggtcatcctcagccagccagctgaacacagggtataaaaagactcactgaaatcagcagagctctacctcgatcagcagaactaatggacccatagattcacatacaatagcaaacatttactgttttaagtcactgagtattgggggcgtttgatacaccaccataactacccaaccagaactgggctagacagcattgcacaagatgaacaacactagaaatccttttctaccctaagagaaaatgatttggaggcacccaggtggcacagttaagcctctgactctcggttttggctatgttcatgatctcagggtcgtgagactgagtccagcgtcaggctccacactcagcatggagtctgcttgagaattttctcaacctctcccactgctcttccctcttgtgctctctctctaaaaaaaagtaaataaatctttaaaagaaagagagagggcagcccgggtggctcagtagtttagcgctgccttcagcccagggtgtgatcctggagacccggaatcgagtcccacgttgggctccctgcatggagcctgcttctccctctgcctgtgtctctgcctctcttgttctctctctccccctctctcttatgaataaataaagaaaatctttaaaaaagagagagagagagagagaccgagagagagagagaaaattatttgtataaaatcccaggacatactaccttttttgagtatataccaccaatatgtatatattttcatatttatatattttaatcgaattaaaaattaactataaggagaatattcatatattcatgtatgcaggccgatgagcaggagcaactgctgcagagactactgctctagtctgcttcagaccttttgtgtttccacaggtggcactgtgctgccagcacctgggagaggcatgcagccatccctctcactcctgccaggtgggctggaaaccaccagctctacagagcagctttgccattaccagatgtcctcactcaccctgcggtgctgctgaactttgcctccacccaaaaagcattgatgcatgttcattctgccctggagaatggaaatccaggtctgctatggacagactagtgcctttgcatctcccttgagcccaggataatgtacatgtgcctttgaagggtataattaagagggtggcttaggatgctataccttctaaagcaaactagaaattctctcactctctctctctctcttcctctctctttcttttctttcatctctggattatcctatcttcttcctagccaggaacattagagtattctaaaaaatgtgtgttgtgctatgacaacagagacagagcagtgactgagagacaactgaggtccccttggcctctcccacttcaggcatagtaagccctggggctgttgaggcgtctcttcatgtttcactttgttcttggcttcctactatactcttagcatcccaatgctctccggtattgacttttcatgaaatgatgaagaattagattaattctcactcagatatagaaaaagtatccatcagtgagttgctctgctctacctaaatatgaagaaatacttaatattttcaggcatggataaagaaggaagaaatggacaggatgcacaaataaactttgtggtttaagtgaattcaaatccacttgacatttctcctatgctctccaaaggacagagcttgtataataaacttgttaccagccaccttcctgccactgcctttcaggagtttggaaattaatctccgatggtattctcaatgttgaaaaaacagtttcatgtaatattttaaaagctaaatagttctaagccctgaataaagggagaggaagtcaaaaaactatagaatacttactcattcattcaagaaacaattttaggcatacatcacatacaagtaccctataggggctagtcaccacagaaaacaaagctctaggagatagaaaaatggacccacccattctctactccctaccctaggaagaaggctctaggtcaaggaatatttatgatacagctcaggaaaggagcaggaaactttggcgtaatagaaccagagtcatagtttacaagtaaaaatattactaatagccactgtaaatatctatattgtgtcatataatagcactgctctaagatgctgtattatttttattaattagatatcagattttgtcctaaatgcttttgtcagattggtcacatgatcatcacaatgagcatacaaggtaggttttgatttcatacatatacattctatatagatgtagaaaggttactaaatatttcatgctcaggttcttggtgaagtcacaagtggagcacaggccactcgactccagagcttgtgctctacattctaatggctacaacacactgccctcactctgggtctatttgacccacacagagtcaaagatatggtactaggagaaatatcacttttcaatagatcataaagagagggtttcaggaggaaaccacttctcctaatcctcctagtcgagagatatgatagattttaaagaatcaattgaaatactagcatcagctcagagaaacagacacttttgggtggccacttactaccatattcactaaactggagctaatgcctcctacctcactgtccccctggcacactcagggaaatagctgacaggaagaatgaataaaacagtcaccatgagaaagagaaaaatcactaatatttataatgggtatactattttggaaaaaaaaatgatttctttttctgtccaagatctgattagtatgtggcagagctaggaattcttagcagttcaaaacactgtaatattttgcctctctaaagtgaaaataaaaataaacaccctttgtagcccaagagttgaggaagagcaagacacaagcagataaaagttgagagaaaggtgccccgattttcctgctgtgatctagaataggaggaaacactaagagaagattgtatagcagagcagaggaagggtctttccctactgccatttcagtttcttgtcaaccattacctcaaagcctccacacacatctatccaggagtgtcttcattgggtaaaagttactgtaaaatagagctgggaaaagagaaaggatgtgaatctaaatccccagattctctgttcacccacatatggtgttagtagaattcagaaatccctgtgtggcctctataaggatttctaaggtaatgagaaaagaccagcgctggtttccagaactgatcagatattcacacccagacctcaatgttattacaagctgacttgtaataactgactgatgctcacaggccagttggttctacttttggatacaaatgacctcccagaatactactccaagggagagagctctgcgatgatgataaaacaagatgaaggtgactgtgcaacacaaagcacctaacatgcccctacgctaaatgaacaactgctacttctctagaattatagctttattctcactgtagtctcttccctacagataagatttcttgaaataccccatcatgaaattacctttgctttttgacaatatccaatctaaaaggagcctctgcttccttgaactctttaccaaattaaccaatggaataagttctataacaagttatttcaagtactctttaaatgagacactctatagtttcctcaccatgagaaagttcatctacaagtgtgttcctcctggcatctagccaatagacattaatagtaggtaaagagactgggaagcttccctgagagagttgccatattgggggtgaaatggtttatcagttagcttttgctgcataacaaaaatagcagcaacaaaaaacctcaataacacaaggcaataagtacttattaacttatggatacagtgctccggtcgctgatttgggctgcacttggtaagatttctggggtaagatgacttatggctgatctaagctaggctgggctgattagttcccttggctctgctccatgtgtctctcatcttccagcaggtcagccctggcatattatcatagtagtggcagagacccaacagagcaagaggaaacacacccagcctcttgagggcctaggctcagaactgacacaacatcatcctgactcattttgaaatgaagaaaagaacctgtatttaaaacaacgattcaacacaagtcataaaattagatcagattgggagcagaggtaaggaggcatggggaatagagtccaccccttttgtgtcagcatgtacaaagccaatggcaaagggcctagataatgggaagggtgaggaatagaagctatcattacaatctactccagatgctttttagcctgcttgggctggagctgaatgacaattataaccatatttcaagggacttgtaatgccagtgaaagccaagttgaggcctgagcatccagaagttcccttctccaagccaagtgtaaccaaccaaactgcagttcacaccagtgaacatgaattccagcagagcaaacaacaccaaatagaaaagatcatgaatccgtgtttcactaaagcctaaaggcatgtctctaaggatccaagtctctattctaccccaagtctctgtaagccacatttctcccccatatccttagatacattctcagggtaagggaactaagggagaaaatatttactttctggagagaattgacaatactcaaagggtagatttaaatcattaatatggaccaatattacataggattgaatcaaattaatttttgtcctcattaaccaggagtgtggtatcaggataagacctgaacaggcaaaataaagaaggtatgtacactatgtaaaaatttgtaactctcgatatatttcacactagaaattgtgacgttttcaagaaatctaaaatgttcaaagactttatccacaaagctatcatattttcccccatctctggcctatgtgagacagcactgaagaaccaagaacttttcttttaactttagggcttgccttaatttgtggttgttatctttgttttatttctcaaatgaaactactttataaatacttcacaagctattgtgaaaatcaaattagacagcaaatatgaaagtttttcaaaaagctcagaaatgtcaaagaaatgtaaagtgatttgatgcgctgttacaataacaatcaaagtgtgctcatgggcactgctttctcattatccaggcagactggtatggtagaaataacaccaggctaagccaagattctcctttctggattcctgctgtgccaccacaagctgatatgaccctacaacaagacccttgcccactccagaccttaatatttttgttaaaaaaataaggaggtggggctagaggatttctaaggtctttctgagttctaaataatttcttgttgatacttcatccaacacaatttttcaattatttgaggaagaaaaaagattgaccttatatatgaggaagcccctttagaagcctgcttttaaaataacaaaataatccttatcctagagttaacttcatttctgccactttgaatttatttgcattcatgctgatatatttctttgggcctgtctgactgtggcaactaatcctcacctcctccatgaagccctccctaatctcttctgtgctgctttatcataagacctactactatcatcattgttgccacttgcaggatgacaatcttcacttgatctatccactctatagtaggcagcatgttataaaagctaatgcaacatctctgatgctcccaagaactctgcacagtgccttaaattctcctggttttacagatcaagaatttgagcccagagaagtgaactatgttgatcgaatttatacaactcagaaagaggcaaaaccagatatcacactcatatctgcctcgaggcaaagtcctggtgtctcttgaattctcctatatcagaagtctttgtactgtgaatatttacttatttgtctctcttctagattatcagcaacttggtggttgaggatgtttgttcttttggagttctaatacctaacacaatacttgtaacataaaacaataaatgtttgttaagtaacatataccagacagatgaccattttctcctgagtattagattcaatatgcaatattatgattcctcataatcacacacaccatttactactgatactttaactcaagttcttagacctataaatcaaatgggatcaaatttcttgaagactgtagatattctaaaagtattctcaagttccaaaggaaatcttttaaattctcaaaatcaccataaatcaatgtcttcaaataattgatgtcttataaatcaatgtcttcaaataattatatacttcactatatcatatgtgcataaaaaataactataatacaaagaattatatatgtcgtgggtcttctagtccccatcttcagattgtttaattgccaatctaatctctttcttcaactccaatcatatttttccaagagtcttctggacatttttattcaatcttccaagaatgatttctaaatttacatgttaaagtaaatcttaacatcttcttcctactagagtagttttggagagatgtcttttattcttcctactgcttgatattttaggagtttaatagatatctgatttaactaaacaacaccaaataatagcactgattctgccactgatttttcactttatttatatcacttgtaaatgaagaataatatgatttatatttattgtctacgctcaggactcagctaagtaaaacttgagattgctacaccaatatagacctttctactcagttacatcctatatataaacctgaatatctactaagtgcatattatgcactcagcattgtcaaatactatagcaaatataaaaagcagaagacatgttctctggtcctgagtctacagagacaagagtcatacagaaaaaaaaaattagtagagaacaatgcaaggcagaaaataagcaagcactaatgtgtttgaaaccaataattatattgtagcacttccaaagaggaagaaattatgttttcactattggagcccatgaacacatagaaaagcaatggagtcattcttaggctaaagcctcctccttgaaagtaaactattctgaatcccaaagcacagcatctgttgttattattgtttcttatttgttttcatagatattgtttcttcttatgtctccaaaatgaaggcattctcaagagttacgttccagaactgtattcatttttatcccctctctctggtagggaactattttgatcccaggtccatggaatctgtgccagatcattgcctacacattgccaatctaacctttttctcaaacacaaatcacattctcccaaatgtctttggacattttcatccagccttccaagagctatttctaaattactggtgtaaaatcaaccttaacatctcagtctctacaagagtttccctcaccttggtatttctatcaggtatttctatatcttaacctctagatttctcacatatttcctctctcccatattgttctggctacagtcctcattctctctgaaatacactttaactggcctccctcctttctcccagacttcctttttctcccaccagagcctttctctgactgtctgactttcaccagaaactcatcacctgtgatatattcagaggaagacagtgagctagaacagaaaaagcacctgccttagcctcaaatcatgaagaactgagttctaatctcagccctacatcattatatgtcctttaaaatctgtgagattttttccattctcatctctaaactgggaataatactaccaatcgagaaatagttaggatttaaatgagataatatgtgaaaccacagcacaaaataagagctttataactagcaactcttataaagattattcttctttttataagattgagtagaatattcaaaatctctcgagagataacttcaatctatagtttttggatgactttaatacataaactaagcatgtctaccttattccttcttcttatactatttaccataaatgagatctcttctcctcatagttcttcccaccagaacaaatgctatttattcttcaagacacttttcttgtcactaacatcctcaattaggtctccagccgcttcctccccagttcagaatgagccctcctaacaaattactgaatcattactgaatatttgttttctatactagcctttgaatttaaacacataatggcttgttatatatcttttttaaaaaaattcacattaaatgaatgtcgttgtttttggctcccaagtagaatacaaagaacatgagtgtagggaatatcatttgtgcctataaacccagtactgtgctcagataggctacttgataaatatttgttgaattgataaataaataaaggacttcattaataaaacagagtggtaggagttggcacaaaaactaatgaaagttgagtcatatttggtgggagctgaatgctaagaaaagccagtggcatgattaaagtggaaagtagggaagtctgatatatcaattctgcctacaaatggattatggctgaggacttgagaagcacagagattaaccaggagtctttgttaagaattagaggaaaggaaatgattttgaaacaatgagaatgcaccaatgggagaattcagtgtgtatttcaagacaagaaaaacaagacaatgtgaatgaataaatagactttgaagtataagcaaaaaataagacaggtgacgtttcaagactggagaaagcagactgtatgaaatgaatgtggacactctgaagacactctgtagagaatgagaattcagttttgactatgctattattgaacatgcaaatatggatgtccaatggacaagctttgcagtcaaagtaagaaaaattcttataaactgccagtgactgagaaaatcccccaaagatagaatactacctatctttctgatacttctttagaaaattgtgtgatctttgtatttgttcagctctgagataagaaaaagtcccataatcttactatgaagctaccttcttctattcatgtaggtagcctagaaaaccctagaggacagagaatgtgaataagatagtatcattcataaaagtaattctctcatttttaggctttctttttcttttaagctttatcttattttacttttttttaaagattttatttatttattcatgagagacacagagagagagaggcagagacgcagatagagaagcatgctccatgcaggaagcccaaaaacaagaaacatgcacaaaactgcaaaagtatatcacaaattgcttaaagccaataagaaagaagaaaattcaacaaaatgtcaacaagatttttatagatatagacaagttattctaaagtttatatggaaaggcaaaggaactagaatagttataattattttgtgaaagaatcattctacccaatgctaagacttattatatagctatgtaatcaaaaccagcacaaacatgcccacctgatttttgacaaacacctcaaaagtaattcaatgtgggaaagacagtcttttcaacaaatgctgctgcaacacttgacattcatagacaaaaagatgaatcttaatctagacatcacaccgtgtacaacagataatttaaaatggatcatggatttaaatgtaaagtgtgaaactattaaactttaataagaaagcatagggcaaaacctttggaacccagaattgggcaaagcatttttagacatgacaccaaaaacatgatccataaaagaaaaataaatgatgaattggactttattgaaataaaaaaaaaaaactttaattccatgaaagatcttgttaagaagatgaaaagcaaactatatatttggaaaaatattcacaaactatgtatctgtcaaggatcttatatctagaacatacagttaaaaaaaatccaattagaaaataggtgttaagacttatacataagtcttcatagcatcccatttataatattctcaaactggaaacaagccaaataaccaccaaaaggtgaactgataaacacactgttatttttatgcattaggaaattactcagcaataaaaaggaatgaactcatgctgcatggggatgacatgggtgcatcctaaaataattgtgctgagttacagaagccacacagaaaaagaacatattgcacaatcccatttatgtacccattatatatccataatgtcagtggttactgatctgggagtaggaatggggcagatgagagaaatataaaagggaatgaaatgtttgcgggtgatggatacatatgtccattctgttgatggtgacaatgatttcatgagggtatatacatatttagaaatgtattaagttgtacatttaagtatgtgtggcttattgtatgtgaattgtgcctcagaattgttaataaaaaatgttaccttgtctcaaagtaatatcattatgatgataacttgcaaatttctaactatgattgatacttgttgcttaaatggcagggtcacttgcctatctccttactcaacatctgcatgtggatgtctaatggatgccttaatttaaactgttcaaatcgaaatactgatgtcctttctcaaatatgttccactcacagttatcccattttaatgtctgggaattctaattgttggtcaaattattttctatcttatcttttccttatagaaaatccataccaatccatctgcaaatgtttactctgtcttcaaaagatgcctcagtctgtccctgtatcactgtacccactgatatcaacctggtcaaagccctcgtcattattgccatcaccacctaactggtttcttttttcttaccttgccctcctatacttcatccttatcacagaactttctagaagatgtcagatgtttcctttccacacctccaatggacttcattttgccagggttggaaaatcaaagtccttaaaatcactgccctataagaccctacataatctgccccatctccattcctctctggattctctttttacttttccccaactcatttcactccatcctttcttgccattcatcaaataaaccaggcacacttcctcctcaagggttttgcactagcaatttcctctgcctagaatgctttttccttaaatatccacatggtgccaagtggtcaagcaatccctaagcagcagctgaaatgctgggctgactcccataaatttggtcagtaaaaaaacaagcccaaatgggtttagacagttcattactcatagcacatagcagacagtatacatgtgatgctggcattagttcccttgctcatccaagtcccacgagggccataagaacagcctcgggctggtgcaatgcacacctggctgtgtcacagtcgaggcatactccatttaggacccctggtcttatcaggggattgctataaaaatcactcaccctcccctccagaaagagatattaccatattattaccctgggatgtaagcaaatcttctctggagagaggaagaagactttagtattctagaaattccccagggaaggaagatattgtccatctttatcatgaggatagagaaatggatccatccaactcagtgggacatgctatttctagcttccaaggctttctttactcttgtccttgaaaagatagtccaagatgtttggaaatatgtagaattttctcctaagacatggctcctttttccttttcctttgaatctctatccaaatatcaacatctcagagagacctacctcaaacattctatttattattgtaatctaccccattgacaccccttaccaacctgggttatttcatgccaggatagtcacaaaccacttcaaaatgacatccaaatttgtggcctgctttctcccacgtggagaacatgtattgcatgagggcaggcattttatgctttatcgctgctttacccagtatctacaactgtcggctcctgataagcctcttatgaattatttttaaatttgagagaatgaagcataaaagatgttaactcttccttgcaatgcttcacaaagaattctagcctttgaaggactagatgcccaaataaaggttacaacaagtttaaggggaaaagaatcattaggttaccaaagactgaaggcccaaccttccttctgaagaacctacagcagggaacatagatggttgacactggttagacaccacatctttgaatcccactgcagtgtctacaccaggccatgcttcccctgggttgctccttaccagcccctgagcatcatgatggatttgaagtggagaatcccttcccaatagaaaatccttctgctggcaactttagctcaggactccctacggatgtgggccaagattttcacaggactgcactgcgtctgaggctttgttcccacctcttcttcccacctacccctgtcactgtttctgtaggtgtcacacctgttttgtagtctgacggctctctgtgattccttctgttccctcaaccttaatccttcacagagatttcccagaagaaagctcttcttgtacttctaatccagtctttgcatctactgcttgaaagacttgaactaacttatggagg

SEQ ID NO: 9 - protein, 5 aa, Canis lupus
  source 1..5: note = sub_species familiaris
NVTAP

SEQ ID NO: 10 - protein, 4 aa, Canis lupus
  source 1..4: note = sub_species familiaris
LNVT

SEQ ID NO: 11 - protein, 6 aa, Canis lupus
  source 1..6: note = sub_species familiaris
LNVTAP

SEQ ID NO: 12 - protein, 6 aa, Canis lupus
  source 1..6: note = sub_species familiaris
NVTAPT

SEQ ID NO: 13 - protein, 4 aa, Canis lupus
  source 1..4: note = sub_species familiaris
TAPT

SEQ ID NO: 14 - protein, 6 aa, Canis lupus
  source 1..6: note = sub_species familiaris
FLTQKQ

SEQ ID NO: 15 - DNA, 34 bp, synthetic construct
  source 1..34: mol_type = other DNA
aaattaatgcagtttaggaagttaagatatcatt

SEQ ID NO: 16 - DNA, 21 bp, synthetic construct
  source 1..21: mol_type = other DNA
taaagccaccagcagctatca

SEQ ID NO: 17 - DNA, 17 bp, synthetic construct
  source 1..17: mol_type = other DNA
tgggacaggaccaacct

SEQ ID NO: 18 - DNA, 19 bp, synthetic construct
  source 1..19: mol_type = other DNA
gagtggcctcacctttctg

SEQ ID NO: 19 - DNA, 20 bp, synthetic construct
  source 1..20: mol_type = other DNA
tctcttcctcccaccttctt

SEQ ID NO: 20 - DNA, 19 bp, synthetic construct
  source 1..19: mol_type = other DNA
gctgacaggttgccacata

SEQ ID NO: 21 - DNA, 44 bp, synthetic construct
  source 1..44: mol_type = other DNA
caggaaacagctatgacaataggtgattttggtctagccacagt

SEQ ID NO: 22 - DNA, 22 bp, synthetic construct
  source 1..22: mol_type = other DNA
gtcactcagtagcacctcaggg

SEQ ID NO: 23 - DNA, 27 bp, synthetic construct
  source 1..27: mol_type = other DNA
aataggtgattttggtctagccagaga

SEQ ID NO: 24 - DNA, 22 bp, synthetic construct
  source 1..22: mol_type = other DNA
gtcactcagtagcacctcaggg

SEQ ID NO: 25 - DNA, 20 bp, synthetic construct
  source 1..20: mol_type = other DNA
ggaggagctggagcttgatg

SEQ ID NO: 26 - DNA, 24 bp, synthetic construct
  source 1..24: mol_type = other DNA
cactgatcttctcgaagtcgtcat

SEQ ID NO: 27 - DNA, 17 bp, synthetic construct
  source 1..17: mol_type = other DNA
cctttctcacccagaag

SEQ ID NO: 28 - DNA, 16 bp, synthetic construct
  source 1..16: mol_type = other DNA
tttctcaccccgaagc

SEQ ID NO: 29 - DNA, 17 bp, synthetic construct
  source 1..17: mol_type = other DNA
cctttctcacccaggag

SEQ ID NO: 30 - DNA, 39 bp, Canis lupus; identical to SEQ ID NO: 1
  source 1..39: mol_type = other DNA; sub_species = familiaris

SEQ ID NO: 31 - protein, 13 aa, Homo sapiens
KMLNVTAPTPQQL

SEQ ID NO: 32 - DNA, 39 bp, Homo sapiens; identical to SEQ ID NO: 2
  source 1..39: mol_type = other DNA

SEQ ID NO: 33 - DNA, 39 bp, Canis lupus
  source 1..39: mol_type = other DNA; sub_species = familiaris
gcctttctcacccagaagcagaaggtcggggaactgaag

SEQ ID NO: 34 - protein, 13 aa, Homo sapiens
AFLTQKQKVGELK

SEQ ID NO: 35 - DNA, 39 bp, Homo sapiens
  source 1..39: mol_type = other DNA
gcctttcttacccagaagcagaaggtgggagaactgaag

SEQ ID NO: 36 - DNA, 39 bp, synthetic construct
  source 1..39: mol_type = other DNA
aaaatgtttaatgccacacctcccccacctcaggactta

SEQ ID NO: 37 - DNA, 39 bp, synthetic construct; identical to SEQ ID NO: 36
  source 1..39: mol_type = other DNA

SEQ ID NO: 38 - DNA, 39 bp, synthetic construct
  source 1..39: mol_type = other DNA
aaaacgttgaatgtgacagcacccacacctcagcagtta

SEQ ID NO: 39 - DNA, 39 bp, synthetic construct; identical to SEQ ID NO: 36
  source 1..39: mol_type = other DNA

SEQ ID NO: 40 - DNA, 39 bp, synthetic construct
  source 1..39: mol_type = other DNA
aaaacattgaaagtgtttgcacccacacctcagcagtta

SEQ ID NO: 41 - DNA, 39 bp, synthetic construct
  source 1..39: mol_type = other DNA
aaaatgttgaatgtgacagcacccacacctcaccagtta

SEQ ID NO: 42 - DNA, 39 bp, synthetic construct; identical to SEQ ID NO: 1
  source 1..39: mol_type = other DNA

SEQ ID NO: 43 - DNA, 39 bp, synthetic construct
  source 1..39: mol_type = other DNA
aaaatgttgactgctccaccacccccggccttccaaaat

SEQ ID NO: 44 - DNA, 39 bp, synthetic construct
  source 1..39: mol_type = other DNA
ggagaggtggcagtgacaatgttgacacctcagcagtta

SEQ ID NO: 45 - DNA, 39 bp, synthetic construct
  source 1..39: mol_type = other DNA
aaaatgttgaatgctccaccacccccgcccctccaaaaa

SEQ ID NO: 46 - DNA, 39 bp, synthetic construct
  source 1..39: mol_type = other DNA
aaaaagttgaaagtgaaaatgttgacacctcagcagtta

SEQ ID NO: 47 - DNA, 39 bp, synthetic construct
  source 1..39: mol_type = other DNA
aaaatgttgaatgctccaccacccccacccctccaaata

SEQ ID NO: 48 - DNA, 38 bp, synthetic construct
  source 1..38: mol_type = other DNA
aaaatgttgaaaggacaatgttgacacctcagcagtta

SEQ ID NO: 49 - DNA, 39 bp, synthetic construct; identical to SEQ ID NO: 41
  source 1..39: mol_type = other DNA

SEQ ID NO: 50 - DNA, 39 bp, synthetic construct; identical to SEQ ID NO: 1
  source 1..39: mol_type = other DNA

SEQ ID NO: 51 - protein, 412 aa, Canis lupus
  source 1..412: note = sub_species familiaris
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDVAETPPRPRTPGRPLSSYGMDSRPPMAIFELLDYIVNEPPPKLPSGVFSLEFQDFVNKCLIKNPAERADLKQLMPPPRGPEGRAATRRSQWKSVHAFIKRSDGEEVDFAGWLCSTIGLNQPSTPTHAAGV